(19) United States
(12) Patent Application Publication
(10) Pub. No.: US 2005/0125852 A1
(43) Pub. Date: Jun. 9, 2005

(54) Novel kinases

(75) Inventors: Sean Caenepeel, Woodland Hills, CA (US); Gerard Manning, La Jolla, CA (US); Glen Charydczak, Princeton Jct, NJ (US); Igor Grigoriev, Walnut Creek, CA (US)

Correspondence Address: FOLEY AND LARDNER;SUITE 500, 3000 K STREET NW, WASHINGTON, DC, 20007, US

(73) Assignee: Sugen, Inc.

(21) Appl. No.: 10/840,512

(22) Filed: May 7, 2004

Related U.S. Application Data
(60) Provisional application No. 60/469,014, filed on May 9, 2003.

(51) Int. Cl. C12Q 1/68; A01K 67/027; C07H 21/04; C12N 9/12

(52) U.S. Cl. 800/18; 435/6; 435/69.1; 435/194; 435/320.1; 435/325; 536/23.2

(57) ABSTRACT

The present invention relates to kinase polypeptides, nucleotide sequences encoding the kinase polypeptides, as well as various products and methods useful for the diagnosis and treatment of various kinase-related diseases and conditions. Through the use of a bioinformatics strategy, mammalian members of protein and lipid kinase families have been identified and their protein structure predicted.

### FIELD OF THE INVENTION

This regular U.S. application claims priority to U.S. provisional application Ser. No. 60/469,014, filed May 9, 2003, which is incorporated herein by reference. The present invention relates to kinase polypeptides, nucleotide sequences encoding the kinase polypeptides, as well as various products and methods useful for the diagnosis and treatment of various kinase-related diseases and conditions.

### BACKGROUND OF THE INVENTION

The following description of the background of the invention is provided to aid in understanding the invention, but is not admitted to be or to describe prior art to the invention.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function.

Protein phosphorylation plays a pivotal role in cellular signal transduction. Among the biological functions controlled by this type of postranslational modification are: cell division, differentiation and death (apoptosis); cell motility and cytoskeletal structure; control of DNA replication, transcription, splicing and translation; protein translocation events from the endoplasmic reticulum and Golgi apparatus to the membrane and extracellular space; protein nuclear import and export; regulation of metabolic reactions, etc. Abnormal protein phosphorylation is widely recognized to be causally linked to the etiology of many diseases including cancer as well as immunologic, neuronal and metabolic disorders.

The following abbreviations are used for kinases throught this application:
ASK Apoptosis signal-regulating kinase
CaMK Ca²⁺/calmodulin-dependent protein kinase
CCRK Cell cycle-related kinase
CDK Cyclin-dependent kinase
CK Casein kinase
DAGK Di-acyl glycerol kinase
DAPK Death-associated protein kinase
DM myotonic dystrophy kinase
Dyrk dual-specificity-tyrosine phosphorylating-regulated kinase
GAK Cyclin G-associated kinase
GRK G-protein coupled receptor
GuC Guanylate cyclase
HIPK Homeodomain-interacting protein kinase
IRAK Interleukin-1 receptor-associated kinase
MAPK Mitogen activated protein kinase
MAST Microtubule-associated STK
MLCK Myosin-light chain kinase
MLK Mixed lineage kinase
NEK NimA-related protein kinase (═NEK)
PKA cAMP-dependent protein kinase
RSK Ribosomal protein S6 kinase
RTK Receptor tyrosine kinase
SGK Serum and glucocorticoid-regulated kinase
STK serine threonine kinase
ULK UNC-51-like kinase

Protein kinases in eukaryotes phosphorylate proteins on the hydroxyl substituent of serine, threonine and tyrosine residues, which are the most common phospho-acceptor amino acid residues. However, phosphorylation on histidine has also been observed in bacteria.

The presence of a phosphate moiety modulates protein function in multiple ways. A common mechanism includes changes in the catalytic properties (Vmax and Km) of an enzyme, leading to its activation or inactivation.

A second widely recognized mechanism involves promoting protein-protein interactions. An example of this is the tyrosine autophosphorylation of the ligand-activated EGF receptor tyrosine kinase. This event triggers the high-affinity binding to the phosphotyrosine residue on the receptor's C-terminal intracellular domain of the SH2 motif of the adaptor molecule Grb2. Grb2, in turn, binds through its SH3 motif to a second adaptor molecule, such as SHC. The formation of this ternary complex activates the signaling events that are responsible for the biological effects of EGF. Serine and threonine phosphorylation events also have been recently recognized to exert their biological function through protein-protein interaction events that are mediated by the high-affinity binding of phosphoserine and phosphothreonine to WW motifs present in a large variety of proteins (Lu, P. J. et al (1999) Science 283:1325-1328).

A third important outcome of protein phosphorylation is changes in the subcellular localization of the substrate. As an example, nuclear import and export events in a large diversity of proteins are regulated by protein phosphorylation (Drier E. A. et al (1999) Genes Dev 13: 556-568).

Protein kinases are one of the largest families of eukaryotic proteins with several hundred known members. These proteins share a 250-300 amino acid domain that can be subdivided into 12 distinct subdomains that comprise the common catalytic core structure. These conserved protein motifs have recently been exploited using PCR-based and bioinformatic strategies leading to a significant expansion of the known kinases.

Kinases largely fall into two groups: those specific for phosphorylating serines and threonines, and those specific for phosphorylating tyrosines. Some kinases, referred to as “dual specificity” kinases, are able to phosphorylate tyrosine as well as serine/threonine residues.

Protein kinases can also be characterized by their location within the cell. Some kinases are transmembrane receptor-type proteins capable of directly altering their catalytic activity in response to the external environment such as the binding of a ligand. Others are non-receptor-type proteins lacking any transmembrane domain. They can be found in a variety of cellular compartments from the inner surface of the cell membrane to the nucleus.

Many kinases are involved in regulatory cascades wherein their substrates may include other kinases whose activities are regulated by their phosphorylation state. Ultimately the activity of some downstream effector is modulated by phosphorylation resulting from activation of such a pathway. The conserved protein motifs of these kinases have recently been exploited using PCR-based cloning strategies leading to a significant expansion of the known kinases.

Multiple alignment of the sequences in the catalytic domain of protein kinases and subsequent parsimony analysis permits the segregation of related kinases into distinct branches of subfamilies including: tyrosine kinases (PTKs), dual-specificity kinases, and serine/threonine kinases (STKs). The latter subfamily includes cyclic-nucleotide-dependent kinases, calcium/calmodulin kinases, cyclin-dependent kinases (CDKs), MAP-kinases, serine-threonine kinase receptors, and several other less defined subfamilies.

The protein kinases may be classified into several major groups including AGC, CAMK, Casein kinase 1, CMGC, STE, tyrosine kinases, and atypical kinases (Plowman, G D et al., Proceedings of the National Academy of Sciences, USA, Vol. 96, Issue 24, 13603-13610, Nov. 23, 1999; see also www.kinase.com). Within each group are several distinct families of more closely related kinases. In addition, there is a group designated “other” to represent several smaller families. In addition, an “atypical” family represents those protein kinases whose catalytic domain has little or no primary sequence homology to conventional kinases, including the alpha kinases, pyruvate dehydrogenase kinases, A6 kinases and PI3 kinases.

AGC Group

The AGC kinases are basic amino acid-directed enzymes that phosphorylate residues found proximal to Arg and Lys. Examples of this group are the G protein-coupled receptor kinases (GRKs), the cyclic nucleotide-dependent kinases (PKA, PKC, PKG), NDR or DBF2 kinases, ribosomal S6 kinases, AKT kinases, myotonic dystrophy kinases (DMPKs), MAPK interacting kinases (MNKs), MAST kinases, and the YANK family.

GRKs regulate signaling from heterotrimeric guanine protein coupled receptors (GPCRs). Mutations in GPCRs cause a number of human diseases, including retinitis pigmentosa, stationary night blindness, color blindness, hyperfunctioning thyroid adenomas, familial precocious puberty, familial hypocalciuric hypercalcemia and neonatal severe hyperparathroidism (OMIM, www.ncbi.nlm.nih.gov/Omim/). The regulation of GPCRs by GRKs indirectly implicates GRKs in these diseases.

The cAMP-dependent protein kinases (PKA) consist of heterotetramers comprised of 2 catalytic (C) and 2 regulatory (R) subunits, in which the R subunits bind to the second messenger cAMP, leading to dissociation of the active C subunits from the complex. Many of these kinases respond to second messengers such as cAMP resulting in a wide range of cellular responses to hormones and neurotransmitters.

AKT is a mammalian proto-oncoprotein regulated by phosphatidylinositol 3-kinase (PI3-K), which appears to function as a cell survival signal to protect cells from apoptosis. Insulin receptor, RAS, PI3-K, and PDK1 all act as upstream activators of AKT, whereas the lipid phosphatase PTEN functions as a negative regulator of the PI3-K/AKT pathway. Downstream targets for AKT-mediated cell survival include the pro-apoptotic factors BAD and Caspase9, and transcription factors in the forkhead family, such as DAF-16 in the worm. AKT is also an essential mediator in insulin signaling, in part due to its use of GSK-3 as another downstream target.

The S6 kinases (RSK) regulate a wide array of cellular processes involved in mitogenic response including protein synthesis, translation of specific mRNA species, and cell cycle progression from G1 to S phase. One of the RSK genes has been localized to chromosomal region 17q23 and is amplified in breast cancer (Couch, et al., Cancer Res. 1999 Apr. 1;59(7):1408-11).

CAMK Group

The CAMK kinases are also basic amino acid-directed kinases. They include the Ca²⁺/calmodulin-regulated and AMP-dependent protein kinases (AMPK), myosin light chain kinases (MLCK), MAP kinase activating protein kinases (MAPKAPKs), checkpoint 2 kinases (CHK2), death-associated protein kinases (DAPKs), phosphorylase kinase (PHK), Rac and Rho-binding Trio kinases, a “unique” family of CAMKs, and the MARK family of protein kinases.

The MARK family of STKs are involved in the control of cell polarity, microtubule stability and cancer. One member of the MARK family, C-TAK1, has been reported to control entry into mitosis by activating Cdc25C which in turn dephosphorylates Cdc2.

CMGC Group

The CMGC kinases are “proline-directed” enzymes phosphorylating residues that exist in a proline-rich context. They include the cyclin-dependent kinases (CDKs), mitogen-activated protein kinases (MAPKs), GSK3s, RCKs, (dual-specific tyrosine kinases) DYRKs, (SR-protein specific kinase) SRPKs, and CLKs. Most CMGC kinases have larger-than-average kinase domains owing to the presence of insertions within subdomains X and XI.

CDKs play a pivotal role in the regulation of mitosis during cell division. The process of cell division occurs in four stages: S phase, the period during which chromosomes duplicate, G2, mitosis and G1 or interphase. During mitosis the duplicated chromosomes are evenly segregated allowing each daughter cell to receive a complete copy of the genome. A key mitotic regulator in all eukaryotic cells is the STK cdc2, a CDK regulated by cyclin B. However some CDK-like kinases, such as CDK5 are not cyclin associated nor are they cell cycle regulated.

MAPKs play a pivotal role in many cellular signaling pathways, including stress response and mitogenesis (Lewis, T. S., Shapiro, P. S., and Ahn, N. G. (1998) Adv. Cancer Res. 74, 49-139). MAP kinases can be activated by growth factors such as EGF, and cytokines such as TNF-alpha. In response to EGF, Ras becomes activated and recruits Raf1 to the membrane where Raf1 is activated by mechanisms that may involve phosphorylation and conformational changes (Morrison, D. K., and Cutler, R. E. (1997) Curr. Opin. Cell Biol. 9, 174-179). Active Raf1 phosphorylates MEK1 which in turn phosphorylates and activates the ERKs subfamily of MAPKs. DYRKS are dual-specificity tyrosine kinases.

Tyrosine Protein Kinase Group

The tyrosine kinase group encompass both cytoplasmic (e.g. src) as well as transmembrane receptor tyrosine kinases (e.g. EGF receptor). These kinases play a pivotal role in the signal transduction processes that mediate cell proliferation, differentiation and apoptosis.

STE Group

The STE family refers to the 3 classes of protein kinases that lie sequentially upstream of the MAPKs. This group includes STE7 (MEK or MAP2K) kinases, STE11 (MEKK or MAP2K) kinases and STE20 (MEKKK or MAP4K) kinases. In humans, several protein kinase families that bear only distant homology with the STE11 family also operate at the level of MAP3Ks including RAF, MLK, TAK1, and COT. Since crosstalk takes place between protein kinases functioning at different levels of the MAPK cascade, the large number of STE family kinases could translate into an enormous potential for upstream signal specificity. This also includes homologues of the yeast sterile family kinases (STE), which refers to 3 classes of kinases which lie sequentially upstream of the MAPKs.

The prototype STE20 from baker's yeast is regulated by a hormone receptor, signaling to directly affect cell cycle progression through modulation of CDK activity. It also coordinately regulates changes in the cytoskeleton and in transcriptional programs in a bifurcating pathway. In a similar way, the homologous kinases in humans are likely to play a role in extracellular regulation of growth, cell adhesion and migration, and changes in transcriptional programs, all three of which have critical roles in tumorigenesis. Mammalian STE20-related protein kinases have been implicated in response to growth factors or cytokines, oxidative-, UV-, or irradiation-related stress pathways, inflammatory signals (e.g. TNFα), apoptotic stimuli (e.g. Fas), T and B cell costimulation, the control of cytoskeletal architecture, and cellular transformation. Typically the STE20-related kinases serve as upstream regulators of MAPK cascades. Examples include: HPK1, a protein-serine/threonine kinase (STK) that possesses a STE20-like kinase domain that activates a protein kinase pathway leading to the stress-activated protein kinase SAPK/JNK; PAK1, an STK with an upstream GTPase-binding domain that interacts with Rac and plays a role in cellular transformation through the Ras-MAPK pathway; and murine NIK, which interacts with upstream receptor tyrosine kinases and connects with downstream STE11-family kinases.

NEK kinases are related to NIMA, which is required for entry into mitosis in the filamentous fungus A. nidulans. Mutations in the nimA gene cause the nim (never in mitosis) G2 arrest phenotype in this fungus (Fry, A. M. and Nigg, E. A. (1995) Current Biology 5: 1122-1125). Several observations suggest that higher eukaryotes may have a NIMA functional counterpart(s): (1) expression of a dominant-negative form of NIMA in HeLa cells causes a G2 arrest; (2) overexpression of NIMA causes chromatin condensation, not only in A. nidulans, but also in yeast, Xenopus oocytes and HeLa cells (Lu, K. P. and Hunter, T. (1995) Prog. Cell Cycle Res. 1, 187-205); (3) NIMA when expressed in mammalian cells interacts with pin1, a prolyl-prolyl isomerase that functions in cell cycle regulation (Lu, K. P. et al. (1996) Nature 380, 544-547); (4) okadaic acid inhibitor studies suggests the presence of cdc2-independent mechanism to induce mitosis (Ghosh, S. et al.(1998) Exp. Cell Res. 242, 1-9) and (5) a NIMA-like kinase (fin1) exists in another eukaryote besides Aspergillus, Saccharomyces pombe (Krien, M. J. E. et al.(1998) J. Cell Sci. 111, 967-976). Eleven mammalian NIMA-like kinases have been identified —NEK1-11. Despite the similarity of the NIMA-related kinases to NIMA over the catalytic region, the mammalian kinases are structurally different to NIMA over the extracatalytic regions. In addition several of the mammalian kinases are unable to complement the nim phenotype in Aspergillus nimA mutants.

Casein Kinase 1 Group

The CK1 family represents a distant branch of the protein kinase family. The hallmarks of protein kinase subdomains VIII and IX are difficult to identify. One or more forms are ubiquitously distributed in mammalian tissues and cell lines. CK1 kinases are found in cytoplasm, in nuclei, membrane-bound, and associated with the cytoskeleton. Splice variants differ in their subcellular distribution. VRK is in this group.

TKL Group

This group includes integrin receptor kinase (IRAK); endoribonuclease-associated kinases (IRE); Mixed lineage kinase (MLK); LIM-domain containing kinase (LIMK); Receptor interacting protein kinase (RIPK); RAF; Serine-threonine kinase receptors (STKR).

RIPK2 is a serine-threonine kinase associated with the tumor necrosis factor (TNF) receptor complex and is implicated in the activation of NF-kappa B and cell death in mammalian cells. It has recently been demonstrated that RIPK2 activates the MAPK pathway (Navas, et al., J. Biol. Chem. 1999 Nov. 19;274(47):33684-33690). RIPK2 activates AP-1 and serum response element regulated expression by inducing the activation of the Elk1 transcription factor. RIPK2 directly phosphorylates and activates ERK2 in vivo and in vitro. RIPK2 in turn is activated through its interaction with Ras-activated Raf1. These results highlight the integrated nature of kinase signaling pathway.

“Other” Group

Several families cluster within a group of unrelated kinases termed “Other”. Group members that define smaller, yet distinct phylogenetic branches conventional kinases include CHK1; Elongation 2 factor kinases (EIFK); Calcium-calmodulin kinase kinases (CAMKK); IkB kinases (IKK); endoribonuclease-associated kinases (IRE); MOS; PIM; TAK1; Testis specific kinase (TSSK); tousled-related kinase (TLK); UNC51-related kinase (UNC); WEE; mitotic kinases (BUB1, AURORA, PLK, and NIMA/NEK); several families that are close homologues to worm (C26C2.1, YQ09, ZC581.9, YFL033c, C24A1.3); Drosophila (SLOB), or yeast (YDOD_sp, YGR262_sc) kinases; and others that are “unique,” that is, those which do not cluster into any obvious family. Additional families are even less well defined and first were identified in lower eukaryotes such as yeast or worms (YNL020, YPL236, YQ09, YWY3, SCY1, C01H6.9, C26C2.1).

The tousled (TSL) kinase was first identified in the plant Arabidopsis thaliana. TSL encodes a serine/threonine kinase that is essential for proper flower development. Human tousled-like kinases (Tlks) are cell-cycle-regulated enzymes, displaying maximal activities during S phase. This regulated activity suggests that Tlk function is linked to ongoing DNA replication (Sillje, et al., EMBO J. 1999 Oct. 15; 18(20):5691-5702).

BRSK Subfamily

The BRSK subfamily family of kinases includes the mammalian BRSK1 and BRSK2, SAD-1 from C. elegans, CG6114 from Drosophila and the HrPOPK-1 gene from the primitive chordate Halocynthia roretzi. SAD-1 is expressed in neurons and required for presynaptic vesicle function (Crump et al. (2001) Neuron 29:115-29). BRSK1 and BRSK2 are selectively expressed in brain, and HrPOPK-1 is selectively expressed in the nervous system, indicating that all members of this family have a neural function, specifically related to synaptic vesicle function.

The NRBP family includes mammalian kinases NRBP1 and NRBP2, as well as homologs in C. elegans (H37N21.1) and D. melanogaster (LD28657). These kinases are most closely related in sequence to the WNK family of kinases, and may fulfill similar functions, including a role in hypertension.

Additionally, where BRSK2 is classsified as a member of the CAMKL family (p102), it should be further classified—i.e. “into the CAMK group, the CAMKL family and the BRSK sub-family”.

Atypical Protein Kinase Group

There are several proteins with protein kinase activity that appear structurally unrelated to the eukaryotic protein kinases. These include; Dictyostelium myosin heavy chain kinase A (MHCKA), Physarum polycephalum actin-fragmin kinase, the human A6 PTK, human BCR, mitochondrial pyruvate dehydrogenase and branched chain fatty acid dehydrogenase kinase, and the prokaryotic “histidine” protein kinase family. The slime mold, worm, and human eEF-2 kinase homologues have all been demonstrated to have protein kinase activity, yet they bear little resemblance to conventional protein kinases except for the presence of a putative GxGxxG ATP-binding motif.

The so-called histidine kinases are abundant in prokaryotes, with more than 20 representatives in E. coli, and have also been identified in yeast, molds, and plants. In response to external stimuli, these kinases act as part of two-component systems to regulate DNA replication, cell division, and differentiation through phosphorylation of an aspartate in the target protein. To date, no “histidine” kinases have been identified in metazoans, although mitochondrial pyruvate dehydrogenase (PDK) and branched chain alpha-ketoacid dehydrogenase kinase (BCKD kinase), are related in sequence. PDK and BCKD kinase represent a unique family of atypical protein kinases involved in regulation of glycolysis, the citric acid cycle, and protein synthesis during protein malnutrition. Structurally they conserve only the C-terminal portion of “histidine” kinases including the G box regions. BCKD kinase phosphorylates the E1a subunit of the BCKD complex on Ser-293, proving it to be a functional protein kinase. Although no bona fide “histidine” kinase has yet been identified in humans, they do contain PDK.

Several other proteins contain protein kinase-like homology including: receptor guanylyl cyclases, diacylglycerol kinases, choline/ethanolamine kinases, and YLK1-related antibiotic resistance kinases. Each of these families contain short motifs that were recognized by our profile searches with low scoring E-values, but a priori would not be expected to function as protein kinases. Instead, the similarity could simply reflect the modular nature of protein evolution and the primal role of ATP binding in diverse phosphotransfer enzymes. However, two recent papers on a bacterial homologue of the YLK1 family suggests that the aminoglycoside phosphotransferases (APHs) are structurally and functionally related to protein kinases. There are over 40 APHs identified from bacteria that are resistant to aminoglycosides such as kanamycin, gentamycin, or amikacin. The crystal structure of one well characterized APH reveals that it shares greater than 40% structural identity with the 2 lobed structure of the catalytic domain of cAMP-dependent protein kinase (PKA), including an N-terminal lobe composed of a 5-stranded antiparallel beta sheet and the core of the C-terminal lobe including several invariant segments found in all protein kinases. APHs lack the GxGxxG normally present in the loop between beta strands 1 and 2 but contain 7 of the 12 strictly conserved residues present in most protein kinases, including the HGDxxxN signature sequence in kinase subdomain VIB. Furthermore, APH also has been shown to exhibit protein-serine/threonine kinase activity, suggesting that other YLK-related molecules may indeed be functional protein kinases.

The eukaryotic lipid kinases (PI3Ks, PI4Ks, DAGKs and PIPKs) also contain several short motifs similar to protein kinases, but otherwise share minimal primary sequence similarity. However, once again structural analysis of PIPKII-beta defines a conserved ATP-binding core that is strikingly similar to conventional protein kinases. Three residues are conserved among all of these enzymes including (relative to the PKA sequence) Lys-72 which binds the gamma-phosphate of ATP, Asp-166 which is part of the HRDLK motif and Asp-184 from the conserved Mg⁺⁺ or Mn⁺⁺ binding DFG motif. The worm genome contains 12 phosphatidylinositol kinases, including 3 PI13-kinases, 2 PI4-kinases, 3 PIP5-kinases, and 4 PI3-kinase-related kinases. The latter group has 6 mammalian members (DNA-PK, SMG1, TRRAP, FRAP/TOR, ATM, and ATR), which have been shown to participate in the maintenance of genomic integrity in response to DNA damage, and exhibit true protein kinase activity, raising the possibility that other PI-kinases may also act as protein kinases. Regardless of whether they have true protein kinase activity, PI3-kinases are tightly linked to protein kinase signaling, as evidenced by their involvement downstream of many growth factor receptors and as upstream activators of the cell survival response mediated by the AKT protein kinase.

### SUMMARY OF THE INVENTION

The present invention relates, in part, to mammalian protein kinases and protein kinase-like enzymes identified from genomic and cDNA sequencing.

Tyrosine and serine/threonine kinases (PTKs and STKs) have been identified and their protein sequence predicted as part of the instant invention. Mammalian members of these families were identified through the use of a bioinformatics strategy. The partial or complete sequences of these kinases are presented here, together with their classification.

One aspect of the invention features an identified, isolated, enriched, or purified nucleic acid molecule encoding a kinase polypeptide having an amino acid sequence selected from the group consisting of those set forth in SEQ ID NO:115 through SEQ ID NO:228.

The term “identified” in reference to a nucleic acid means that a sequence was selected from a genomic, EST, or cDNA sequence database based on it being predicted to encode a portion of a previously unknown or novel protein kinase.

By “isolated,” in reference to nucleic acid, is meant a polymer of 10, 15, or 18 (preferably 21, more preferably 39, most preferably 75) or more nucleotides conjugated to each other, including DNA and RNA that is isolated from a natural source or that is synthesized as the sense or complementary antisense strand. In certain embodiments of the invention, longer nucleic acids are preferred, for example those of 100, 200, 300, 400, 500, 600, 900, 1200, 1500, or more nucleotides and/or those having at least 50%, 60%, 75%, 80%, 85%, 90%, 91%, 92%, 93%, 94%, 95%, 96%, 97%, 98%, 99% or 100% identity to a sequence selected from the group consisting of those set forth in SEQ ID NO:1 through SEQ ID NO:114 or encoding for amino acid selected from SEQ ID NO:115 through SEQ ID NO:228.

The isolated nucleic acid of the present invention is unique in the sense that it is not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2- to 5-fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC 19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level this level should be at least 2- to 5-fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “kinase polypeptide” is meant 32 (preferably 40, more preferably 45, most preferably 55) or more contiguous amino acids in a polypeptide having an amino acid sequence selected from the group consisting of those set forth in SEQ ID NO:115 through SEQ ID NO:228. In certain aspects, polypeptides of 75, 100, 200, 300, 400, 450, 500, 550, 600, 700, 800, 900 or more amino acids are preferred. The kinase polypeptide can be encoded by a full-length nucleic acid sequence or any portion (e.g., a “fragment” as defined herein) of the full-length nucleic acid sequence, so long as a functional activity of the polypeptide is retained, including, for example, a catalytic domain, as defined herein, or a portion thereof. One of skill in the art would be able to select those catalytic domains, or portions thereof, which exhibit a kinase or kinase-like activity, e.g., catalytic activity, as defined herein. It is well known in the art that due to the degeneracy of the genetic code numerous different nucleic acid sequences can code for the same amino acid sequence. Equally, it is also well known in the art that conservative changes in amino acid can be made to arrive at a protein or polypeptide which retains the functionality of the original. Such substitutions may include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making amino acid exchanges which have only slight, if any, effects on the overall protein can be found in Bowie et al., Science, 1990, 247, 1306-1310, which is incorporated herein by reference in its entirety including any figures, tables, or drawings. In all cases, all permutations are intended to be covered by this disclosure.

The amino acid sequence of a kinase polypeptide of the invention comprises an amino acid sequence substantially similar (preferably at least about 90% identical) to a sequence having an amino acid sequence selected from the group consisting of those set forth in SEQ ID NO:115 through SEQ ID NO:228, or the corresponding full-length amino acid sequence, or fragments thereof, preferably consisting of at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through 228. A fusion polypeptide comprises a kinase polypeptide of the invention and a heterologous polypeptide.

A sequence that is substantially similar to a sequence selected from the group consisting of those set forth in SEQ ID NO:115 through SEQ ID NO:228, will preferably have at least 70%, 80%, 85%, 90%, 91%, 92%, 93%, 94%, 95%, 96%, 97%, 98%, 99% or 100% identity to the sequence.

By “identity” is meant a property of sequences that measures their similarity or relationship. Identity is measured by dividing the number of identical residues by the total number of residues and gaps and multiplying the product by 100. “Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters, for example Gapped BLAST or PSI-BLAST (Altschul, et al. (1997) Nucleic Acids Res. 25:3389-3402), BLAST (Altschul, et al. (1990) J. Mol. Biol. 215:403-410), and Smith-Waterman (Smith, et al. (1981) J. Mol. Biol. 147:195-197). Preferably, the default settings of these programs will be employed, but those skilled in the art recognize whether these settings need to be changed and know how to make the changes.

“Similarity” is measured by dividing the number of identical residues plus the number of conservatively substituted residues (see Bowie, et al. Science, 1999), 247, 1306-1310, which is incorporated herein by reference in its entirety, including any drawings, figures, or tables) by the total number of residues and gaps and multiplying the product by 100.

In preferred embodiments, the invention features isolated, enriched, or purified nucleic acid molecules encoding a kinase polypeptide comprising a nucleotide sequence that:
(a) encodes a polypeptide having an amino acid sequence selected from the group consisting of those set forth in SEQ ID NO:115 through SEQ ID NO:228 or an amino acid sequence having at least about 90% identical to a sequence selected from the group consisting of SEQ ID NO:115 through SEQ ID NO:228; (b) is the complement of the nucleotide sequence of (a); (c) hybridizes under highly stringent conditions to the nucleotide molecule of (a) and encodes a naturally occurring kinase polypeptide; (d) encodes a polypeptide having an amino acid sequence of at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through SEQ ID NO:228; or (e) is the complement of the nucleotide sequence of (d). Additional domains encoded include, for example, CNH, PH, phobol esters/diacylglycerol binding (C1), protein kinase C-terminal, PDZ (also known as DHR or GLGF), kinase associated domain 1, UBA/TS-N, UBA, armadillo/beta-catenin-like repeat, POLO box duplicated region, P21-Rho-binding, immunoglobulin, WIF, leucine rich repeat, SH3, MYND, EF hand, and bromodomain.

The term “domain” refers to a region of a polypeptide whose sequence or structure is conserved between several homologs of the polypoeptide and which serves a particular function. Many domains may be identified by searching the Pfam database of domain models (pfam.wustl.edu) which provides coordinates on the polypeptide delimiting the start and end of the domain, as well as a score giving the likelihood that the domain is present in the polypeptide. Other domains may be identified by specialized programs, such as the COILS program to detect colied-coil regions (www.ch.embnet.org/software/COILS_form.html), the SignalP program to detect signal peptides (www.ebs.dtu.dk/services/TMHMM), by visual inspection of the amino acid sequence (e.g., determination of cysteine-rich or proline-rich domains), or by Smith-Waterman alignment shows a high level of sequence similarity in the region containing the domain, it may be concluded that the domain is present in both proteins within that region. which serves a particular function.

Domains of signal transduction proteins can serve functions including, but not limited to, binding molecules that localize the signal transduction molecule to different regions of the cell, binding other signaling molecules directly responsible for propagating a particular cellular signal or binding molecules that influence the function of the protein. Some domains can be expressed separately from the rest of the protein and function by themselves.

The term “N-terminal region” refers to the extracatalytic region located between the initiator methionine and the catalytic domain of the protein kinase. Depending on its length, the N-terminal region may or may not play a regulatory role in kinase function. An example of a protein kinase whose N-terminal domain has been shown to play a regulatory role is PAK6 or PAK5, which contains a CRIB motif used for Cdc42 and rac binding (Burbelo, P. D. et al. (1995) J. Biol. Chem. 270, 29071-29074). Such an N-terminal region is also termed a N-terminal functional domain or N-terminal domain.

The term “catalytic domain” or protein kinase domain refers to a region of the protein kinase that is typically 25-300 amino acids long and is responsible for carrying out the phosphate transfer reaction from a high-energy phosphate donor molecule such as ATP or GTP to itself (autophosphorylation) or to other proteins (exogenous phosphorylation). The catalytic domain of protein kinases is made up of 12 subdomains that contain highly conserved amino acid residues, and are responsible for proper polypeptide folding and for catalysis. The catalytic dmoain can be defined with reference to the parameters described in a “Pfam” database: pfam.wustl.edu. In particular, it can be defined with reference to a HMMer search of the Pfam database. In the N-terminal extremity of the catalytic domain there is a glycine rich stretch of residues in the vicinity of a lysine residue, which has been shown to be involved in ATP binding. In the central part of the catalytic domain there is a conserved aspartic acid residue which is important for the catalytic activity of the enzyme. See Accession number PF00069 of pfam.wustl.edu.

The term “catalytic activity”, as used herein, defines the rate at which a kinase catalytic domain phosphorylates a substrate. Catalytic activity can be measured, for example, by determining the amount of a substrate converted to a phosphorylated product as a function of time. Catalytic activity can be measured by methods of the invention by determining the concentration of a phosphorylated substrate after a fixed period of time. Phosphorylation of a substrate occurs at the active site of a protein kinase. The active site is normally a cavity in which the substrate binds to the protein kinase and is phosphorylated.

The term “substrate” as used herein refers to a molecule phosphorylated by a kinase of the invention. Kinases phosphorylate substrates on serine/threonine or tyrosine amino acids. The molecule may be another protein or a polypeptide.

The term “C-terminal region” refers to the region located between the catalytic domain or the last (located closest to the C-terminus) functional domain and the carboxy-terminal amino acid residue of the protein kinase. See Accession number PF00433 of pfam.wustl.edu. Depending on its length and amino acid composition, the C-terminal region may or may not play a regulatory role in kinase function. An example of a protein kinase whose C-terminal region may play a regulatory role is PAK3 which contains a heterotrimeric G_{b} subunit-binding site near its C-terminus (Leeuw, T. et al. (1998) Nature, 391, 191-195). Such a C-terminal region is also termed a C-terminal functional domain or C-terminal domain.

By “functional” domain is meant any region of the polypeptide that may play a regulatory or catalytic role as predicted from amino acid sequence homology to other proteins or by the presence of amino acid sequences that may give rise to specific structural conformations.

The “CNH domain” is the citron homology domain, and is often found after cysteine rich and pleckstrin homology (PH) domains at the C-terminal end of the proteins [MEDLINE:99321922]. It acts as a regulatory domain and could be involved in macromolecular interactions [MEDLINE:99321922], [MEDLINE:97280817]. See Accession number PF00780 of pfam.wustl.edu.

The “PH domain” is the ‘pleckstrin homology’ (PH) domain and is a domain of about 100 residues that occurs in a wide range of proteins involved in intracellular signaling or as constituents of the cytoskeleton [MEDLINE:93272305], [MEDLINE:93268380], [MEDLINE:94054654], [MEDLINE:95076505], [MEDLINE:95157628], [MEDLINE:95197706], [MEDLINE:96082954]. See Accession number PF00169 of pfam.wustl.edu.

The “Phorbol esters/diacylglycerol binding domain” is also known as the Protein kinase C conserved region 1 (C1) domain. The N-terminal region of PKC, known as C1, has been shown [MEDLINE:89296905] to bind PE and DAG in a phospholipid and zinc-dependent fashion. The C1 region contains one or two copies (depending on the isozyme of PKC) of a cysteine-rich domain about 50 amino-acid residues long and essential for DAG/PE-binding. The DAG/PE-binding domain binds two zinc ions; the ligands of these metal ions are probably the six cysteines and two histidines that are conserved in this domain. See Accession number PF00130 of pfam.wustl.edu.

The “PDZ domain” is also known as the DHR or GLGF domain. PDZ domains are found in diverse signaling proteins and may function in targeting signalling molecules to sub-membranous sites [MEDLINE:97348826]. See Accession number PF00595 of pfam.wustl.edu.

The “kinase associated domain 1” (KA1) domain is found in the C-terminal extremity of various serine/threonine-protein kinases from fungi, plants and animals. See Accession number PF02149 of pfam.wustl.edu.

The UBA/TS-N domain is composed of three alpha helices. This family includes the previously defined UBA and TS-N domains. The UBA-domain (ubiquitin associated domain) is a sequence motif found in several proteins having connections to ubiquitin and the ubiquitination pathway. The structure of the UBA domain consists of a compact three helix bundle. This domain is found at the N terminus of EF-TS hence the name TS-N. The structure of EF-TS is known and this domain is implicated in its interaction with EF-TU. The domain has been found in non EF-TS proteins such as alpha-NAC P70670 and MJ0280 Q57728 [1]. See Accession number PF00627 of pfam.wustl.edu.

The “UBA domain” The UBA-domain (ubiquitin associated domain) is a novel sequence motif found in several proteins having connections to ubiquitin and the ubiquitination pathway [MEDLINE:97025177]. The UBA domain is probably a non-covalent ubiquitin binding domain consisting of a compact three helix bundle [MEDLINE:99061330]. See Accession number PF00627 of pfam.wustl.edu.

The “armadillo/beta-catenin-like repeat” is an approximately 40 amino acid long tandemly repeated sequence motif first identified in the Drosophila segment polarity gene armadillo. Similar repeats were later found in the mammalian armadillo homolog beta-catenin, the junctional plaque protein plakoglobin, the adenomatous polyposis coli (APC) tumor suppressor protein, and a number of other proteins [MEDLINE:94170379]. The 3 dimensional fold of an armadillo repeat is known from the crystal structure of beta-catenin [MEDLINE:98449700]. There, the 12 repeats form a superhelix of alpha-helices, with three helices per unit. The cylindrical structure features a positively charged grove which presumably interacts with the acidic surfaces of the known interaction partners of beta-catenin. See Accession number PF00514 of pfam.wustl.edu.

The “POLO box duplicated region” (POLO_box) is described as follows. A subgroup of serine/threonine protein kinases (IPR002290) playing multiple roles during cell cycle, especially in M phase progression and cytokinesis, contain a duplicated domain in their C terminal part, the polo box [MEDLINE:99116035]. The domain is named after its founding member encoded by the polo gene of Drosophila [MEDLINE:92084090]. This domain of around 70 amino acids has been found in species ranging from yeast to mammals. Point mutations in the Polo box of the budding yeast Cdc5 protein abolish the ability of overexpressed Cdc5 to interact with the spindle poles and to organize cytokinetic structures [MEDLINE:20063188]. See Accession number PF00659 of pfam.wustl.edu.

The “P21-Rho-binding domain” is one of a group of small domains that bind Cdc42p- and/or Rho-like small GTPases. These are also known as the Cdc42/Rac interactive binding (CRIB). See Accession number PF00786 of pfam.wustl.edu.

The “immunoglobulin domain” is a domain that is under the umbrella of the immunoglobulin superfamily. Examples of the superfamily include antibodies, the giant muscle kinase titin and receptor tyrosine kinases. Immunoglobulin-like domains may be involved in protein-protein and protein-ligand interactions. The Pfam alignments do not include the first and last strand of the immunoglobulin-like domain. See Accession number PF00047 of pfam.wustl.edu.

The “WIF domain” is found in the RYK tyrosine kinase receptors and WIF the Wnt-inhibitory-factor. The domain is extracellular and and contains two conserved cysteines that may form a disulphide bridge. This domain is Wnt binding in WIF, and it has been suggested that RYK may also bind to Wnt [MEDLINE:20105592]. See Accession number PF02019 of pfam.wustl.edu.

The “leucine rich repeat”—Leucine-rich repeats (LRRs) are relatively short motifs (22-28 residues in length) found in a variety of cytoplasmic, membrane and extracellular proteins [MEDLINE:91099665]. Although these proteins are associated with widely different functions, a common property involves protein-protein interaction. Other functions of LRR-containing proteins include, for example, binding to enzymes [MEDLINE:90094386] and vascular repair [MEDLINE:89367331]. See Accession number PF00560 of pfam.wustl.edu.

The “SH3 domain” SH3 (src Homology-3) domains are small protein modules containing approximately 50 amino acid residues [PUB00001025]. They are found in a variety of of proteins with enzymatic activity. The SH3 domain has a characteristic fold which consists of five or six beta-strands arranged as two tightly packed anti-parallel beta sheets. The linker regions may contain short helices [PUB00001083]. See Accession number PF00018 of pfam.wustl.edu.

The “MYND finger” is a domain found in some suppressors of cell cycle entry [MEDLINE:96203118], [MEDLINE:98079069]. The MYND zinc finger (ZnF) domain is one of two domains in AML/ETO fusion protein required for repression of basal transcription from the multidrug resistance 1 (MDR-1) promoter. The other domain is a hydrophobic heptad repeat (HHR) motif [MEDLINE:98252948]. The AML-1/ETO fusion protein is created by the (8;21) translocation, the second most frequent chromosomal abnormality associated with acute myeloid leukemia. In the fusion protein the AML-1 runt homology domain, which is responsible for DNA binding and CBF beta interaction, is linked to ETO, a gene of unknown function [MEDLINE:96068903]. See Accession number PF01753 of pfam.wustl.edu.

The “EF hand” domain is described as follows: many calcium-binding proteins belong to the same evolutionary family and share a type of calcium-binding domain known as the EF-hand. This type of domain consists of a twelve residue loop flanked on both side by a twelve residue alpha-helical domain. In an EF-hand loop the calcium ion is coordinated in a pentagonal bipyramidal configuration. The six residues involved in the binding are in positions 1, 3, 5, 7, 9 and 12; these residues are denoted by X, Y, Z, −Y, −X and −Z. The invariant Glu or Asp at position 12 provides two oxygens for liganding Ca (bidentate ligand). See Accession number PF00036 of pfam.wustl.edu.

A “bromodomain” is a 110 amino acid long domain, found in many chromatin associated proteins. Bromodomains can interact specifically with acetylated lysine. [MEDLINE:97318593] Bromodomains are found in a variety of mammalian, invertebrate and yeast DNA-binding proteins [MEDLINE:92285152]. The bromodomain may occur as a single copy, or in duplicate. The bromodomain may be involved in protein-protein interactions and may play a role in assembly or activity of multi-component complexes involved in transcriptional activation [MEDLINE:96022440]. See Accession number PF00439 of pfam.wustl.edu.

The term “coiled-coil structure region” as used herein, refers to a polypeptide sequence that has a high probability of adopting a coiled-coil structure as predicted by computer algorithms such as COILS (Lupas, A. (1996) Meth. Enzymology 266:513-525). Coiled-coils are formed by two or three amphipathic α-helices in parallel. Coiled-coils can bind to coiled-coil domains of other polypeptides resulting in homo- or heterodimers (Lupas, A. (1991) Science 252:1162-1164). Coiled-coil-dependent oligomerization has been shown to be necessary for protein function including catalytic activity of serine/threonine kinases (Roe, J. et al. (1997) J. Biol. Chem. 272:5838-5845).

The term “proline-rich region” as used herein, refers to a region of a protein kinase whose proline content over a given amino acid length is higher than the average content of this amino acid found in proteins(i.e., >10%). Proline-rich regions are easily discernable by visual inspection of amino acid sequences and quantitated by standard computer sequence analysis programs such as the DNAStar program EditSeq. Proline-rich regions have been demonstrated to participate in regulatory protein-protein interactions. Among these interactions, those that are most relevant to this invention involve the “PxxP” proline rich motif found in certain protein kinases (i.e., human PAK1) and the SH3 domain of the adaptor molecule Nck (Galisteo, M. L. et al. (1996) J. Biol. Chem. 271:20997-21000). Other regulatory interactions involving “PxxP” proline-rich motifs include the WW domain (Sudol, M. (1996) Prog. Biochys. Mol. Bio. 65:113-132).

The term “spacer region” as used herein, refers to a region of the protein kinase located between predicted functional domains. The spacer region has little conservation when compared with any any amino acid sequence in the database, and can be identified by using a Smith-Waterman alignment of the protein sequence against the non-redundant protein of Pfam database to define the C- and N-terminal boundaries of the flanking functional domains. Spacer regions may or may not play a fundamental role in protein kinase function. Precedence for the regulatory role of spacer regions in kinase function is provided by the role of the src kinase spacer in inter-domain interactions (Xu, W. et al. (1997) Nature 385:595-602).

The term “insert” as used herein refers to a portion of a protein kinase that is absent from a close homolog. Inserts may or may not by the product alternative splicing of exons. Inserts can be identified by using a Smith-Waterman sequence alignment of the protein sequence against the non-redundant protein database, or by means of a multiple sequence alignment of homologous sequences using the DNAStar program Megalign. Inserts may play a functional role by presenting a new interface for protein-protein interactions, or by interfering with such interactions.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors.

In other embodiments, the nucleic acid encoding a kinase polypeptide, or fragment thereof comprises a nucleotide sequence which hybridizes under stringent conditions to a nucleotide sequence encoding a kinase polypeptide having an amino acid sequence selected from the group consisting of those set for the in SEQ ID NO:115 through 228; or hybridizes under stringent conditions to a nucleotide sequence selected from the group consisting of those set forth in SEQ ID NO:1 through 114. The nucleic acid may encode a fusion polypeptide comprising at least one domain of SEQ ID NO:115 through 228, and a heterologous polypeptide.

In various embodiments, the nucleic acid encoding a kinase polypeptide, or fragment thereof, further comprises a vector or promoter effective to initiate transcription in a host cell. Optionally, the nucleic acid molecule may be isolated, enriched, or purified from a mammal, such as a mouse. Alternatively, the nucleic acid molecule may be a cDNA molecule or a genomic DNA molecule. Also provided are recombinant cells comprising a nucleic acid encoding a kinase polypeptide, or fragment thereof; and a method for producing a kinase polypeptide comprising culturing such a recombinant cell under conditions that would allow expression of the nucleic acid molecule and isolating the expressed polypeptide.

The invention includes an antibody or antibody fragment having specific binding affinity to a kinase polypeptide or to a domain of said polypeptide, wherein said polypeptide comprises an amino acid sequence selected from those set forth in SEQ ID NO:115 through SEQ ID NO:228, a hybridoma which produces the such an antibody or antibody fragment, a kit comprising such an antibody which binds to a polypeptide of the invention a negative control antibody.

The invention includes a method for identifying a substance that modulates the activity of a kinase polypeptide comprising the steps of:(a)contacting the kinase polypeptide substantially identical to an amino acid sequence selected from the group consisting of those set forth in SEQ ID NO:115 through SEQ ID NO:228 with a test substance; (b)measuring the activity of said polypeptide; and (c)determining whether said substance modulates the activity of said polypeptide. Such a method may further comprise attaching the kinase polypeptide to a solid support, such as plastic (e.g., mictrotiter plate well), glass (e.g., beads), a matrix, an array, and the like.

The invention also includes a method for identifying a substance that modulates the activity of a kinase polypeptide in a cell comprising the steps of: expressing a kinase polypeptide having a sequence that is at least about 90% identical to an amino acid sequence selected from the group consisting of those set forth in SEQ ID NO:115 through SEQ ID NO:228; adding a test substance to said cell; and monitoring kinase activity in the cell, a change in cell phenotype, or the interaction between said polypeptide and a natural binding partner.

The invention includes a method for treating a disease or disorder by administering to a patient in need of such treatment a substance that modulates the activity of a kinase substantially identical (preferably at least about 90% identical) to an amino acid sequence selected from the group consisting of those set forth in SEQ ID NO:115 through SEQ ID NO:228.

The treatment methods of the invention include the disease or disorder is selected from the group consisting of cancers, immune-related diseases and disorders, cardiovascular disease, brain or neuronal-associated diseases, metabolic disorders and inflammatory disorders; and the disease or disorder selected from the group consisting of cancers of tissues; cancers of blood or hematopoietic origin; cancers of the breast, colon, lung, prostate, cervix, brain, ovaries, bladder or kidney. The treatment methods also include the disease or disorder is selected from the group consisting of disorders of the central or peripheral nervous system; migraines; pain; sexual dysfunction; mood disorders; attention disorders; cognition disorders; hypotension; hypertension; psychotic disorders; neurological disorders and dyskinesias. Treatment methods also include disease or disorder selected from the group consisting of inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity and organ transplant rejection.

The methods of the invention contemplate use of a substance that modulates kinase activity in vitro, including kinase inhibitors.

The invention includes a method for detection of a kinase nucleic acid in a sample as a diagnostic tool for a disease or disorder, wherein said method comprises: contacting said sample with a nucleic acid probe which hybridizes under hybridization assay conditions to a nucleic acid target region of a nucleic acid sequence selected from the group consisting of those set forth in SEQ ID NO:1 through SEQ ID NO:114, said probe comprising the nucleic acid sequence, fragments thereof, or the complements of said sequences and fragments; and
detecting the presence or amount of the target region:probe hybrid, as an indication of said disease or disorder.

The invention further includes a method for detection of a kinase nucleic acid in a sample as a diagnostic tool for a disease or disorder, wherein said method comprises: contacting said sample with nucleic acid primers capable of hybridizing to a nucleic acid sequence selected from the group consisting of those set forth in SEQ ID NO:1 through SEQ ID NO:114; selectively amplifying at least a portion of a nucleic acid sequence selected from the group consisting of SEQ ID NO:1 through 114; and detecting the amplified DNA as an indication of said disease or disorder.

Such detection methods include a disease or disorder selected from the group consisting of cancers, immune-related diseases and disorders, cardiovascular disease, brain or neuronal-associated diseases, metabolic disorders and inflammatory disorders; a disease or disorder selected from the group consisting of cancers of tissues; cancers of blood or hematopoietic origin; cancers of the breast, colon, lung, prostate, cervix, brain, ovary, bladder or kidney; a disease or disorder is selected from the group consisting of central or peripheral nervious system disease, migraines, pain; sexual dysfunction; mood disorders; attention disorders; cognition disorders; hypotension; hypertension; psychotic disorders; neurological disorders and dyskinesias; a disease or disorder is selected from the group consisting of inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The invention includes an isolated, enriched or purified nucleic acid molecule that comprises a nucleic molecule encoding a domain of a kinase polypeptide having a sequence of SEQ ID NO:115-228.

The invention includes an isolated, enriched or purified nucleic acid molecule encoding a kinase polypeptide which comprises a nucleotide sequence that encodes a polypeptide having an amino acid sequence that has at least 90% identity to a polypeptide set forth in SEQ ID NO:115-228.

The invention includes an isolated, enriched or purified nucleic acid molecule according wherein the molecule comprises a nucleotide sequence substantially identical to a sequence of SEQ ID NO:1-114.

The invention includes an isolated, enriched or purified nucleic acid molecule consisting essentially of about 10-30 contiguous nucleotide bases of a nucleic acid sequence that encodes a polypeptide selected from the group consisting of SEQ ID NO:115 through SEQ ID NO:228. The invention also includes an isolated, enriched or purified nucleic acid molecule of about 10-30 contiguous nucleotide bases of a nucleic acid sequence that encodes a polypeptide selected from the group consisting of SEQ ID NO:115 through SEQ ID NO:228, consisting essentially of about 10-30 contiguous nucleotide bases of a nucleic acid sequence selected from the group consisting of SEQ ID NO:1 through 114.

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence.

Various low or high stringency hybridization conditions may be used depending upon the specificity and selectivity desired. These conditions are well known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the full-length sequence.

By stringent hybridization assay conditions is meant hybridization assay conditions at least as stringent as the following: hybridization in 50% formamide, 5×SSC, 50 mM NaH2PO4, pH 6.8, 0.5% SDS, 0.1 mg/mL sonicated salmon sperm DNA, and 5× Denhardt's solution at 42° C. overnight; washing with 2×SSC, 0.1% SDS at 45° C.; and washing with 0.2×SSC, 0.1% SDS at 45° C. Under some of the most stringent hybridization assay conditions, the second wash can be done with 0.1×SSC at a temperature up to 70° C. (Berger et al. (1987) Guide to Molecular Cloning Techniques pg 421, hereby incorporated by reference herein in its entirety including any figures, tables, or drawings.). However, other applications may require the use of conditions falling between these sets of conditions. Methods of determining the conditions required to achieve desired hybridizations are well known to those with ordinary skill in the art, and are based on several factors, including but not limited to, the sequences to be hybridized and the samples to be tested. Washing conditions of lower stringency frequently utilize a lower temperature during the washing steps, such as 65° C., 60° C., 55° C., 50° C., or 42° C.

The invention provides a method for identification of a nucleic acid encoding a kinas polypeptide in a sample comprising contacting the sample with a nucleic acid probe consisting essentially of 10-30 contiguous nucleotide bases of a nucleic acid sequence selected from the group consisting of SEQ ID NO:1 through 114, and isolating a nucleic acid that hybridizes to the probe.

In other preferred embodiments, the invention features isolated, enriched, or purified nucleic acid molecules encoding kinase polypeptides, further comprising a vector or promoter effective to initiate transcription in a host cell. The nucleic acid may encode a polypeptide of SEQ ID NO:115-228 and a vector or promoter effective to initiate transcription in a host cell. The invention includes such nucleic acid molecules that are isolated, enriched, or purified from a mammal and in a preferred embodiment, the mammal is a human. The invention also features recombinant nucleic acid, preferably in a cell or an organism. The recombinant nucleic acid may contain a sequence selected from the group consisting of those set forth in SEQ ID NO:1 through SEQ ID NO:114, or a functional derivative thereof and a vector or a promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a kinase can be inserted into a vector by cutting the vector with restriction enzymes and ligating the two pieces together.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence selected from the group consisting of those set forth in SEQ ID NO:1 through SEQ ID NO:114, which encodes an amino acid sequence selected from the group consisting of those set forth in SEQ ID NO:115 through SEQ ID NO:228, a functional derivative thereof, or at least 35, 40, 45, 50, 60, 75, 100, 200, or 300 contiguous amino acids selected from the group consisting of those set forth in SEQ ID NO:115 through SEQ ID NO:228, the catalytic region of SEQ ID NO:115-228 or catalytic domains, functional domains, or spacer regions of SEQ ID NO:115 through SEQ ID NO:228. The nucleic acid may be isolated from a natural source by cDNA cloning or by subtractive hybridization. The natural source may be mammalian, preferably human, preferably blood, semen or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a kinase polypeptide, to which a particular nucleic acid sequence can hybridize under lower stringency conditions. Examples of lower stringency conditions suitable for screening for nucleic acid encoding kinase polypeptides are provided in Wahl et al. Meth. Enzym. 152:399-407 (1987) and in Wahl et al. Meth. Enzym. 152:415-423 (1987), which are hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a kinase polypeptide that is not present in a sequence coding for any other naturally occurring polypeptide. Such regions preferably encode 32 (preferably 40, more preferably 45, most preferably 55) or more contiguous amino acids, for example, an amino acid sequence selected from the group consisting of those set forth in SEQ ID NO:115 through SEQ ID NO:228. In particular, a unique nucleic acid region is preferably of mammalian origin.

Another aspect of the invention features a nucleic acid probe for the detection of nucleic acid encoding a kinase polypeptide having an amino acid sequence selected from the group consisting of those set forth in SEQ ID NO:115 through SEQ ID NO:228, catalytic domains, functional domains, or spacer regions of SEQ ID NO:115 through SEQ ID NO:228, in a sample. The nucleic acid probe contains a nucleotide base sequence that will hybridize to the sequence selected from the group consisting of those set forth in SEQ ID NO:1 through SEQ ID NO:114, a sequence encoding catalytic domains, functional domains, or spacer regions of SEQ ID NO:115 through SEQ ID NO:228, or a functional derivative thereof.

In preferred embodiments, the nucleic acid probe hybridizes to nucleic acid encoding at least 12, 32, 75, 90, 105, 120, 150, 200, 250, 300 or 350 contiguous amino acids, wherein the nucleic acid sequence is selected from the group consisting of SEQ ID NO:1 through SEQ ID NO:114, or a functional derivative thereof.

Methods for using the probes include detecting the presence or amount of kinase RNA in a sample by contacting the sample with a nucleic acid probe under conditions such that hybridization occurs and detecting the presence or amount of the probe bound to kinase RNA. The nucleic acid duplex formed between the probe and a nucleic acid sequence coding for a kinase polypeptide may be used in the identification of the sequence of the nucleic acid detected (Nelson et al., in Nonisotopic DNA Probe Techniques, Academic Press, San Diego, Kricka, ed., p. 275, 1992, hereby incorporated by reference herein in its entirety, including any drawings, figures, or tables). Kits for performing such methods may be constructed to include a container means having disposed therein a nucleic acid probe.

Methods for using the probes also include using these probes to find, for example, the full-length clone of each of the predicted kinases by techniques known to one skilled in the art. These clones will be useful for screening for small molecule compounds that inhibit the catalytic activity of the encoded kinase with potential utility in treating cancers, immune-related diseases and disorders, cardiovascular disease, brain or neuronal-associated diseases, and metabolic disorders. More specifically disorders including cancers of tissues or blood, or hematopoietic origin, particularly those involving breast, colon, lung, prostate, cervix, skin, brain, ovary, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis, and amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, hypertension, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, bone disorder, psoriasis, atherosclerosis, rhinitis, autoimmunity, and organ transplant rejection.

In another aspect, the invention describes a recombinant cell or tissue comprising a nucleic acid molecule encoding a kinase polypeptide having an amino acid sequence selected from the group consisting of those set forth in SEQ ID NO:115 through SEQ ID NO:228. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the kinase polypeptides.

The polypeptide is preferably a fragment of the protein encoded by an amino acid sequence selected from the group consisting of those set forth in SEQ ID NO:115 through SEQ ID NO:228. By “fragment,” is meant an amino acid sequence present in a kinase polypeptide. Preferably, such a sequence comprises at least 32, 45, 50, 60, 100, 200, or 300 contiguous amino acids of a sequence selected from the group consisting of those set forth in SEQ ID NO:115 through SEQ ID NO:228.

In another aspect, the invention features an isolated, enriched, or purified kinase polypeptide having the amino acid sequence selected from the group consisting of those set forth in SEQ ID NO:115 through SEQ ID NO:228.

By “isolated” in reference to a polypeptide is meant a polymer of 6 (preferably 12, more preferably 18, or 21, most preferably 25, 32, 40, or 50) or more amino acids conjugated to each other, including polypeptides that are isolated from a natural source or that are synthesized. In certain aspects longer polypeptides are preferred, such as those comprising 100, 200, 300, 400, 450, 500, 550, 600, 700, 800, 900 or more contiguous amino acids, including an amino acid sequence selected from the group consisting of those set forth in SEQ ID NO:115 through SEQ ID NO:228; other longer polypeptides also preferred are those having sequence that is substantially similar to a sequence selected from the group consisting of those set forth in SEQ ID NO:115 through SEQ ID NO:228(which preferably has at least 70%, 80%, 85%, 90%, 91%, 92%, 93%, 94%, 95%, 96%, 97%, 98%, 99% or 100% identity to the sequence).

The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term “significantly” here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the kinase polypeptide is a fragment of the protein encoded by an amino acid sequence selected from the group consisting of those set forth in SEQ ID NO:115 through SEQ ID NO:228. Preferably, the kinase polypeptide contains at least 32, 45, 50, 60, 100, 200, or 300 contiguous amino acids of a sequence selected from the group consisting of those set forth in SEQ ID NO:3 and 4, or a functional derivative thereof.

In preferred embodiments, the kinase polypeptide comprises an amino acid sequence having (a) an amino acid sequence selected from the group consisting of those set forth in SEQ ID NO:115 through SEQ ID NO:228; and (b) an amino acid sequence selected from the group consisting of those set forth in SEQ ID NO:115 through SEQ ID NO:228, except that it lacks one or more of the domains selected from the group consisting of the catalytic domain, the C-terminal region, the N-terminal region, and the spacer region.

The polypeptide can be isolated from a natural source by methods well-known in the art. The natural source may be mammalian, preferably human, preferably blood, semen or tissue, and the polypeptide may be synthesized using an automated polypeptide synthesizer.

In some embodiments the invention includes a recombinant kinase polypeptide having (a) an amino acid sequence selected from the group consisting of those set forth in SEQ ID NO:115 through SEQ ID NO:228. By “recombinant kinase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

The polypeptides to be expressed in host cells may also be fusion proteins which include regions from heterologous proteins. Such regions may be included to allow, e.g., secretion, improved stability, or facilitated purification of the polypeptide. For example, a sequence encoding an appropriate signal peptide can be incorporated into expression vectors. A DNA sequence for a signal peptide (secretory leader) may be fused in-frame to the polynucleotide sequence so that the polypeptide is translated as a fusion protein comprising the signal peptide. A signal peptide that is functional in the intended host cell promotes extracellular secretion of the polypeptide. Preferably, the signal sequence will be cleaved from the polypeptide upon secretion of the polypeptide from the cell. Thus, preferred fusion proteins can be produced in which the N-terminus of a kinase polypeptide is fused to a carrier peptide.

In one embodiment, the polypeptide comprises a fusion protein which includes a heterologous region used to facilitate purification of the polypeptide. Many of the available peptides used for such a function allow selective binding of the fusion protein to a binding partner. A preferred binding partner includes one or more of the IgG binding domains of protein A are easily purified to homogeneity by affinity chromatography on, for example, IgG-coupled Sepharose. Alternatively, many vectors have the advantage of carrying a stretch of histidine residues that can be expressed at the N-terminal or C-terminal end of the target protein, and thus the protein of interest can be recovered by metal chelation chromatography. A nucleotide sequence encoding a recognition site for a proteolytic enzyme such as enterokinase, factor X procollagenase or thrombine may immediately precede the sequence for a kinase polypeptide to permit cleavage of the fusion protein to obtain the mature kinase polypeptide. Additional examples of fusion-protein binding partners include, but are not limited to, the yeast I-factor, the honeybee melatin leader in sf9 insect cells, 6-His tag, thioredoxin tag, hemaglutinin tag, GST tag, and OmpA signal sequence tag. As will be understood by one of skill in the art, the binding partner which recognizes and binds to the peptide may be any ion, molecule or compound including metal ions (e.g., metal affinity columns), antibodies, or fragments thereof, and any protein or peptide which binds the peptide, such as the FLAG tag.

In another aspect, the invention features an antibody (e.g., a monoclonal or polyclonal antibody) having specific binding affinity to a kinase polypeptide or a kinase polypeptide domain or fragment where the polypeptide is selected from the group having a sequence at least about 90%, 91%, 92%, 93%, 94%, 95%, 96%, 97%, 98%, 99% or 100% identical to an amino acid sequence set forth in SEQ ID NO:115 through SEQ ID NO:228. By “specific binding affinity” is meant that the antibody binds to the target kinase polypeptide with greater affinity than it binds to other polypeptides under specified conditions. Antibodies or antibody fragments are polypeptides that contain regions that can bind other polypeptides. Antibodies can be used to identify an endogenous source of kinase polypeptides, to monitor cell cycle regulation, and for immuno-localization of kinase polypeptides within the cell.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature 256:495-497, 1975, and U.S. Pat. No. 4,376,110, both of which are hereby incorporated by reference herein in their entirety including any figures, tables, or drawings).

An antibody of the present invention includes “humanized” monoclonal and polyclonal antibodies. Humanized antibodies are recombinant proteins in which non-human (typically murine) complementarity determining regions of an antibody have been transferred from heavy and light variable chains of the non-human (e.g. murine) immunoglobulin into a human variable domain, followed by the replacement of some human residues in the framework regions of their murine counterparts. Humanized antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

An antibody fragment of the present invention includes a “single-chain antibody,” a phrase used in this description to denote a linear polypeptide that binds antigen with specificity and that comprises variable or hypervariable regions from the heavy and light chains of an antibody. Such single chain antibodies can be produced by conventional methodology. The Vh and Vl regions of the Fv fragment can be covalently joined and stabilized by the insertion of a disulfide bond. See Glockshuber, et al., Biochemistry 1362 (1990). Alternatively, the Vh and Vl regions can be joined by the insertion of a peptide linker. A gene encoding the Vh, Vl and peptide linker sequences can be constructed and expressed using a recombinant expression vector. See Colcher, et al., J. Nat'l Cancer Inst. 82: 1191 (1990). Amino acid sequences comprising hypervariable regions from the Vh and Vl antibody chains can also be constructed using disulfide bonds or peptide linkers.

Antibodies or antibody fragments having specific binding affinity to a polypeptide of the invention may be used in methods for detecting the presence and/or amount of kinase polypeptide in a sample by probing the sample with the antibody under conditions suitable for kinase antibody immunocomplex formation and detecting the presence and/or amount of the antibody conjugated to the kinase polypeptide. Diagnostic kits for performing such methods may be constructed to include antibodies or antibody fragments specific for the kinase as well as a conjugate of a binding partner of the antibodies or the antibodies themselves.

An antibody or antibody fragment with specific binding affinity to a kinase polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above. The antibody may be directly labelled with a fluorescent or radioactive label.

Antibodies having specific binding affinity to a kinase polypeptide of the invention may be used in methods for detecting the presence and/or amount of kinase polypeptide in a sample by contacting the sample with the antibody under conditions such that an immunocomplex forms and detecting the presence and/or amount of the antibody conjugated to the kinase polypeptide. Diagnostic kits for performing such methods may be constructed to include a first container containing the antibody and a second container having a conjugate of a binding partner of the antibody and a label, such as, for example, a radioisotope or fluorescent label. The diagnostic kit may also include notification of an FDA approved use and instructions therefor. Antibodies may identify phosphorylated regions of a kinase polypeptide when a protein is phosphorylated.

In another aspect, the invention features a hybridoma which produces an antibody having specific binding affinity to a kinase polypeptide or a kinase polypeptide domain, where the polypeptide is selected from the group having an amino acid sequence set forth in SEQ ID NO:115 through SEQ ID NO:228. By hybridoma is meant an immortalized cell line that is capable of secreting an antibody, for example an antibody to a kinase of the invention. In preferred embodiments, the antibody to the kinase comprises a sequence of amino acids that is able to specifically bind a kinase polypeptide of the invention.

In another aspect, the present invention is also directed to kits comprising antibodies that bind to a polypeptide encoded by any of the nucleic acid molecules described above, and a negative control antibody.

The term “negative control antibody” refers to an antibody derived from similar source as the antibody having specific binding affinity, but where it displays no binding affinity to a polypeptide of the invention.

In another aspect, the invention features a kinase polypeptide binding agent able to bind to a kinase polypeptide selected from the group having (a) an amino acid sequence selected from the group consisting of those set forth in SEQ ID NO:115 through SEQ ID NO:228. The binding agent is preferably a purified antibody that recognizes an epitope present on a kinase polypeptide of the invention. Other binding agents include molecules that bind to kinase polypeptides and analogous molecules that bind to a kinase polypeptide. Such binding agents may be identified by using assays that measure kinase binding partner activity, such as those that measure PDGFR activity.

The invention also features a method for screening for human cells containing a kinase polypeptide of the invention or an equivalent sequence. The method involves identifying the novel polypeptide in human cells using techniques that are routine and standard in the art, such as those described herein for identifying the kinases of the invention (e.g., cloning, Southern or Northern blot analysis, in situ hybridization, PCR amplification, etc.).

In another aspect, the invention features methods for identifying a substance that modulates kinase activity comprising the steps of: (a) contacting a kinase polypeptide selected from the group having an amino acid sequence selected from the group consisting of those set forth in SEQ ID NO:115 through SEQ ID NO:228 with a test substance; (b) measuring the activity of said polypeptide; and (c) determining whether said substance modulates the activity of said polypeptide. The skilled artisan will appreciate that the kinase polypeptides of the invention, including, for example, a portion of a full-length sequence such as a catalytic domain or a portion thereof, are useful for the identification of a substance which modulates kinase activity. Those kinase polypeptides having a functional activity (e.g., catalytic activity as defined herein) are useful for identifying a substance that modulates kinase activity.

The term “modulates” refers to the ability of a compound to alter the function of a kinase of the invention. A modulator preferably activates or inhibits the activity of a kinase of the invention depending on the concentration of the compound (modulator) exposed to the kinase.

The term “modulates” also refers to altering the function of kinases of the invention by increasing or decreasing the probability that a complex forms between the kinase and a natural binding partner. A modulator preferably increases the probability that such a complex forms between the kinase and the natural binding partner, more preferably increases or decreases the probability that a complex forms between the kinase and the natural binding partner depending on the concentration of the compound (modulator) exposed to the kinase, and most preferably decreases the probability that a complex forms between the kinase and the natural binding partner.

The term “activates” refers to increasing the cellular activity of the kinase. The term inhibit refers to decreasing the cellular activity of the kinase. Kinase activity is the phosphorylation of a substrate or the binding with a natural binding partner.

The term “complex” refers to an assembly of at least two molecules bound to one another. Signal transduction complexes often contain at least two protein molecules bound to one another. For instance, a tyrosine receptor protein kinase, GRB2, SOS, RAF, and RAS assemble to form a signal transduction complex in response to a mitogenic ligand.

The term “natural binding partner” refers to polypeptides, lipids, small molecules, or nucleic acids that bind to kinases in cells. A change in the interaction between a kinase and a natural binding partner can manifest itself as an increased or decreased probability that the interaction forms, or an increased or decreased concentration of kinase/natural binding partner complex.

The term “contacting” as used herein refers to mixing a solution comprising the test compound with a liquid medium bathing the cells of the methods. The solution comprising the compound may also comprise another component, such as dimethyl sulfoxide (DMSO), which facilitates the uptake of the test compound or compounds into the cells of the methods. The solution comprising the test compound may be added to the medium bathing the cells by utilizing a delivery apparatus, such as a pipette-based device or syringe-based device.

In another aspect, the invention features methods for identifying a substance that modulates kinase activity in a cell comprising the steps of: (a) expressing a kinase polypeptide in a cell, wherein said polypeptide is selected from the group having an amino acid sequence selected from the group consisting of those set forth in SEQ ID NO:115 through SEQ ID NO:228; (b) adding a test substance to said cell; and (c) monitoring a change in kinase activity or a change in cell phenotype or the interaction between said polypeptide and a natural binding partner. The skilled artisan will appreciate that the kinase polypeptides of the invention, including, for example, a portion of a full-length sequence such as a catalytic domain or a portion thereof, and are useful for the identification of a substance which modulates kinase activity. Those kinase polypeptides having a functional activity (e.g., catalytic activity as defined herein) are useful for identifying a substance that modulates kinase activity.

The term “expressing” as used herein refers to the production of kinases of the invention from a nucleic acid vector containing kinase genes within a cell. The nucleic acid vector is transfected into cells using well known techniques in the art as described herein.

Another aspect of the instant invention is directed to methods of identifying compounds that bind to kinase polypeptides of the present invention, comprising contacting the kinase polypeptides with a compound, and determining whether the compound binds the kinase polypeptides. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include, but are not limited to, compounds of extracellular, intracellular, biological or chemical origin.

The methods of the invention also embrace compounds that are attached to a label, such as a radiolabel (e.g., ¹²⁵I, ³⁵S, ³²P, ³³P, ³H), a fluorescence label, a chemiluminescent label, an enzymic label and an immunogenic label. The kinase polypeptides employed in such a test may either be free in solution, attached to a solid support, borne on a cell surface, located intracellularly or associated with a portion of a cell. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

Other assays can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

Another aspect of the present invention is directed to methods of identifying compounds which modulate (i.e., increase or decrease) activity of a kinase polypeptide comprising contacting the kinase polypeptide with a compound, and determining whether the compound modifies activity of the kinase polypeptide. As described herein, the kinase polypeptides of the invention include a portion of a full-length sequence, such as a catalytic domain, as defined herein. In some instances, the kinase polypeptides of the invention comprise less than the entire catalytic domain, yet exhibit kinase or kinase-like activity. These compounds are also referred to as “modulators of protein kinases.” The activity in the presence of the test compound is compared to the activity in the absence of the test compound. Where the activity of a sample containing the test compound is higher than the activity in a sample lacking the test compound, the compound will have increased the activity. Similarly, where the activity of a sample containing the test compound is lower than the activity in the sample lacking the test compound, the compound will have inhibited the activity.

The present invention is particularly useful for screening compounds by using a kinase polypeptide in any of a variety of drug screening techniques. The compounds to be screened include, but are not limited to, extracellular, intracellular, biological or chemical origin. The kinase polypeptide employed in such a test may be in any form, preferably, free in solution, attached to a solid support, borne on a cell surface or located intracellularly. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

The activity of kinase polypeptides of the invention can be determined by, for example, examining the ability to bind or be activated by chemically synthesised peptide ligands. Alternatively, the activity of the kinase polypeptides can be assayed by examining their ability to bind metal ions such as calcium, hormones, chemokines, neuropeptides, neurotransmitters, nucleotides, lipids, and odorants. Thus, modulators of the kinase polypeptide's activity may alter a kinase function, such as a binding property of a kinase or an activity such as signal transduction or membrane localization.

In various embodiments of the method, the assay may take the form of a yeast growth assay, an Aequorin assay, a Luciferase assay, a mitogenesis assay, a MAP Kinase activity assay, as well as other binding or function-based assays of kinase activity that are generally known in the art. In several of these embodiments, the invention includes any of the receptor and non-receptor protein tyrosine kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors. Biological activities of kinases according to the invention include, but are not limited to, the binding of a natural or a synthetic ligand, as well as any one of the functional activities of kinases known in the art. Non-limiting examples of kinase activities include transmembrane signaling of various forms, which may involve kinase binding interactions and/or the exertion of an influence over signal transduction.

The modulators of the invention exhibit a variety of chemical structures, which can be generally grouped into mimetics of natural kinase ligands, and peptide and non-peptide allosteric effectors of kinases. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries.

The use of cDNAs encoding kinases in drug discovery programs is well-known; assays capable of testing thousands of unknown compounds per day in high-throughput screens (HTSs) are thoroughly documented. The literature is replete with examples of the use of radiolabelled ligands in HTS binding assays for drug discovery (see Williams, Medicinal Research Reviews, 1991, 11, 147-184.; Sweetnam, et al., J. Natural Products, 1993, 56, 441-455 for review). Recombinant proteins are preferred for binding assay HTS because they allow for better specificity (higher relative purity), provide the ability to generate large amounts of material, and can be used in a broad variety of formats (see Hodgson, Bio/Technology, 1992, 10, 973-980; each of which is incorporated herein by reference in its entirety).

A variety of heterologous systems is available for functional expression of recombinant proteins that are well known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

An expressed kinase can be used for HTS binding assays in conjunction with its defined ligand, in this case the corresponding peptide that activates it. The identified peptide is labeled with a suitable radioisotope, including, but not limited to, 125I, ³H, ³⁵S or ³²P, by methods that are well known to those skilled in the art. Alternatively, the peptides may be labeled by well-known methods with a suitable fluorescent derivative (Baindur, et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184.; Sweetnam, et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé, et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

The kinases and natural binding partners required for functional expression of heterologous kinase polypeptides can be native constituents of the host cell or can be introduced through well-known recombinant technology. The kinase polypeptides can be intact or chimeric. The kinase activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes (Murphy, et al., Cur. Opinion Drug Disc. Dev., 1998, 1, 192-199), cell cycle, apoptosis, and growth. Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80).

The invention contemplates a multitude of assays to screen and identify inhibitors of ligand binding to kinase polypeptides. In one example, the kinase polypeptide is immobilized and interaction with a binding partner is assessed in the presence and absence of a candidate modulator such as an inhibitor compound. In another example, interaction between the kinase polypeptide and its binding partner is assessed in a solution assay, both in the presence and absence of a candidate inhibitor compound. In either assay, an inhibitor is identified as a compound that decreases binding between the kinase polypeptide and its natural binding partner. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995 and is included by reference herein including any figures, tables, or drawings.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses both natural binding partners as described above as well as chimeric polypeptides, peptide modulators other than natural ligands, antibodies, antibody fragments, and modified compounds comprising antibody domains that are immunospecific for the expression product of the identified kinase gene.

Other assays may be used to identify specific peptide ligands of a kinase polypeptide, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a kinase gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the kinase polypeptide gene product is unknown and no ligands are known to bind the gene product, the yeast two-hybrid assay can also be used to identify proteins that bind to the gene product. In an assay to identify proteins that bind to a kinase polypeptide, or fragment thereof, a fusion polynucleotide encoding both a kinase polypeptide (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

In preferred embodiments of the invention, methods of screening for compounds which modulate kinase activity comprise contacting test compounds with kinase polypeptides and assaying for the presence of a complex between the compound and the kinase polypeptide. In such assays, the ligand is typically labelled. After suitable incubation, free ligand is separated from that present in bound form, and the amount of free or uncomplexed label is a measure of the ability of the particular compound to bind to the kinase polypeptide.

In another embodiment of the invention, high throughput screening for compounds having suitable binding affinity to kinase polypeptides is employed. Briefly, large numbers of different small peptide test compounds are synthesised on a solid substrate. The peptide test compounds are contacted with the kinase polypeptide and washed. Bound kinase polypeptide is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with a kinase polypeptide. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

In another aspect, the invention provides methods for treating a disease by administering to a patient in need of such treatment a substance that modulates the activity of a kinase polypeptide selected from the group consisting of those set forth in SEQ ID NO:115 through SEQ ID NO:228, as well as the full-length polypeptide thereof, or a portion of any of these sequences that retains functional activity, as described herein. Preferably the disease is selected from the group consisting of cancers, immune-elated diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, skin or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, hypertension, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, bone disorders, psoriasis, atherosclerosis, rhinitis, autoimmunity, and organ transplant rejection.

In preferred embodiments, the invention provides methods for treating or preventing a disease or disorder by administering to a patient in need of such treatment a substance that modulates the activity of a kinase polypeptide having an amino acid sequence selected from the group consisting of those set forth in SEQ ID NO:115 through SEQ ID NO:228, as well as the full-length polypeptide thereof, or a portion of any of these sequences that retains functional activity, as described herein. Preferably, the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

Substances useful for treatment of kinase-related disorders or diseases preferably show positive results in one or more in vitro assays for an activity corresponding to treatment of the disease or disorder in question (Examples of such assays are provided in the references in section VI, below; and in Example 7, herein). Examples of substances that can be screened for favorable activity are provided and referenced in section VI, below. The substances that modulate the activity of the kinases preferably include, but are not limited to, antisense oligonucleotides and inhibitors of protein kinases, as determined by methods and screens referenced in section VI and Example 7, below.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an decrease in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, or cell survival.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism is preferably a mammal. The organism also is preferably a mouse, rat, rabbit, guinea pig, dog, cat, horse, pig, sheep, or goat, more preferably a monkey or ape, and most preferably a human.

In another aspect, the invention features methods for detection of a kinase polypeptide in a sample as a diagnostic tool for diseases or disorders, wherein the method comprises the steps of: (a) contacting the sample with a nucleic acid probe which hybridizes under hybridization assay conditions to a nucleic acid target region of of a nucleic acid sequence selected from the group consisting of SEQ ID NO:1 through 114, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of Preferably the disease is selected from the group consisting of cancers, immune-elated diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, skin or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, hypertension, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, bone disorders, psoriasis, atherosclerosis, rhinitis, autoimmunity, and organ transplant rejection.

The kinase “target region” is the nucleotide base sequence selected from the group consisting of those set forth in SEQ ID NO:1 through SEQ ID NO:114, or the corresponding full-length sequences, a functional derivative thereof, or a fragment thereof, to which the nucleic acid probe will specifically hybridize. Specific hybridization indicates that in the presence of other nucleic acids the probe only hybridizes detectably with the kinase of the invention's target region. Putative target regions can be identified by methods well known in the art consisting of alignment and comparison of the most closely related sequences in the database.

In preferred embodiments the nucleic acid probe hybridizes to a kinase target region encoding at least 6, 12, 75, 90, 105, 120, 150, 200, 250, 300 or 350 contiguous amino acids of a sequence selected from the group consisting of those set forth in SEQ ID NO:115 through SEQ ID NO:228, or the corresponding full-length amino acid sequence, a portion of any of these sequences that retains functional activity, as described herein, or a functional derivative thereof. Hybridization conditions should be such that hybridization occurs only with the kinase genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined supra.

The diseases for which detection of kinase genes in a sample could be diagnostic include diseases in which kinase nucleic acid (DNA and/or RNA) is amplified in comparison to normal cells. By “amplification” is meant increased numbers of kinase DNA or RNA in a cell compared with normal cells. In normal cells, kinases are typically found as single copy genes. In selected diseases, the chromosomal location of the kinase genes may be amplified, resulting in multiple copies of the gene, or amplification. Gene amplification can lead to amplification of kinase RNA, or kinase RNA can be amplified in the absence of kinase DNA amplification.

“Amplification” as it refers to RNA can be the detectable presence of kinase RNA in cells, since in some normal cells there is no basal expression of kinase RNA. In other normal cells, a basal level of expression of kinase exists, therefore in these cases amplification is the detection of at least 1-2-fold, and preferably more, kinase RNA, compared to the basal level.

The diseases that could be diagnosed by detection of kinase nucleic acid in a sample preferably include cancers or other diseases described herein. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

The invention also features a method for detection of a nucleic acid encoding a kinase polypeptide in a sample as a diagnostic tool for a disease or disorder, wherein the method comprises: (a) comparing a nucleic acid target region encoding the kinase polypeptide in a sample, where the kinase polypeptide has an amino acid sequence selected from the group consisting those set forth in SEQ ID NO:115 through SEQ ID NO:228, or one or more fragments thereof, with a control nucleic acid target region encoding the kinase polypeptide, or one or more fragments thereof; and (b) detecting differences in sequence or amount between the target region and the control target region, as an indication of the disease or disorder. Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

The invention further provides methods of using probes and primers derived from the sequences presented herein. In one embodiment, the invention provides a method for identification of a nucleic acid encoding a kinase polypeptide in a sample, wherein said method comprises: (a) contacting said sample with a probe as described herein; and (b) isolating a nucleic acid that hybridizes to the probe, thereby identifying said nucleic acid encoding a kinase polypeptide. In an alternative embodiment, the invention provides a method for identification of a human orthologue of a murine kinase polypeptide, wherein said method comprises: (a) contacting a human sample with a probe as described herein; and (b) isolating a nucleic acid that hybridizes to the probe, thereby identifying a nucleic acid encoding a human orthologue of a murine kinase polypeptide.

Most of these murine genes identified herein are uniquely related in sequence and function to single human kinase genes. Where such an orthologous relationship is known to exist, it is defined by listing the name of the orthologous human gene in Table 1. Where a human ortholog exists for a mouse gene, information on function, expression, catalytic activity, disease association and other biological attributes of the mouse ortholog can be strongly imputed for the human ortholog.

The invention also provides a transgenic mouse comprising a nucleic acid sequence that encodes a polypeptide substantially identical to an amino acid sequence selected from the group consisting of those set forth in SEQ ID NO:115 through 228; wherein said mouse exhibits a phenotype, relative to a wild-type phenotype, comprising modulation of kinase activity of said polypeptide. In addition, a cell or cell line may be obtained from such a transgenic mouse.

The invention also provides a knock-out mouse whose genome is disrupted by recombination at a nucleic acid sequence that encodes a polypeptide substantially identical to an amino acid sequence selected from the group consisting of those set forth in SEQ ID NO:115 through 228; so as to produce a phenotype, relative to a wild-type phenotype, comprising absence of kinase activity of said polypeptide in said transgenic mouse. In addition, a cell or cell line may be obtained from such a knock-out mouse.

Invention transgenic mice and knock-out mice are useful in a method for identifying a substance that modulates the activity of a kinase polypeptide, wherein said method comprises: (a) determining in a sample obtained from such a mouse the presence and/or quantity of kinase activity attributable to the polypeptide encoded by the nucleic acid used to create said mouse; (b) administering a test substance to said mouse; and (c) determining whether said test substance modulates the kinase activity as determined in step (a). Cells or cell lines are also useful in a method for identifying a substance that modulates the activity of a kinase polypeptide, wherein said method comprises: (a) determining in a cell line obtained from the transgenic or knock-out mouse the presence and/or quantity of kinase activity attributable to the polypeptide encoded by the nucleic acid used to create said mouse; (b) contacting said cell line with a test substance; and (c) determining whether said test substance modulates the kinase activity as determined in step (a). Substances found to modulate the activity of a kinase identified using a transgenic or knock-out mouse, or cells or a cell line obtained from such a mouse, can also be used in a method for treating a disease or disorder by their administration to a patient in need of such treatment.

The summary of the invention described above is not limiting and other features and advantages of the invention will be apparent from the following detailed description of the invention, and from the claims.

### BRIEF DESCRIPTION OF THE FIGURES

FIG. 1 shows the nucleotide sequences for mouse protein kinases oriented in a 5′ to 3′ direction (SEQ ID NO:1-114). N's within the sequence indicate nucleotides which are predicted by homology to be present within the nucleic acid but whose exact sequence could not be predicted.

FIG. 2 shows the amino acid sequences for the mouse protein kinases encoded by SEQ ID No. 1-114 in the direction of translation (SEQ ID NO:115 through SEQ ID NO:228). If a predicted stop codon is within the coding region, it is indicated by an ‘*.’. X's indicate amino acids which are predicted by homology to be present within the polypeptide but whose exact sequence could not be predicted.

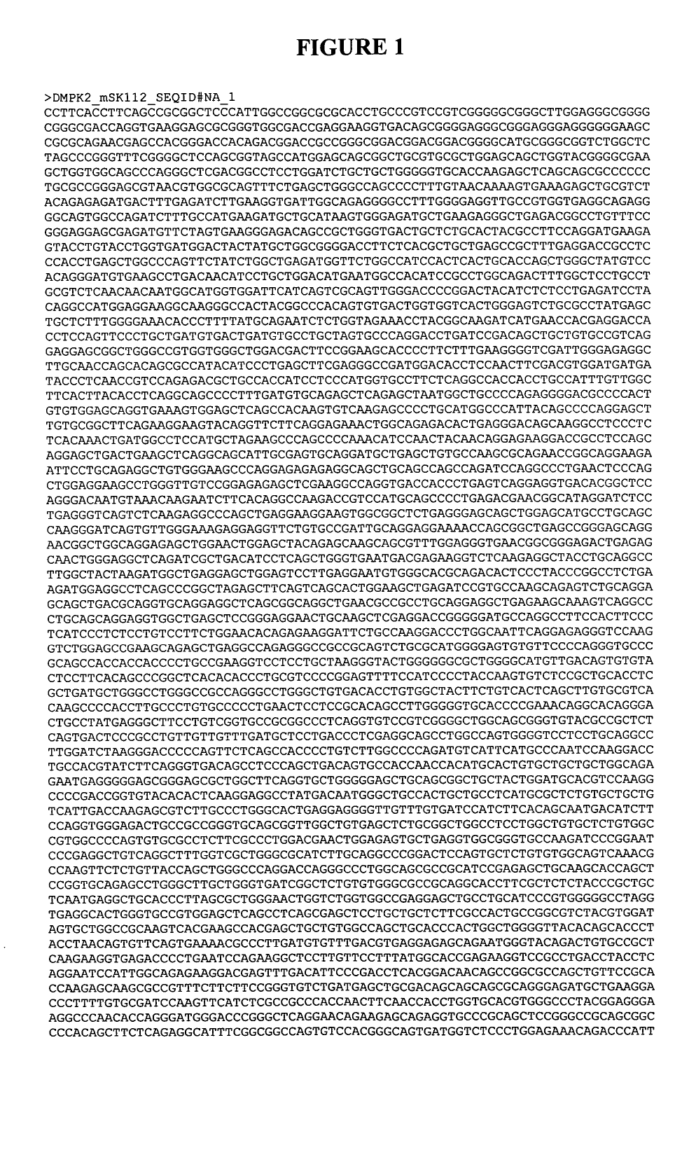

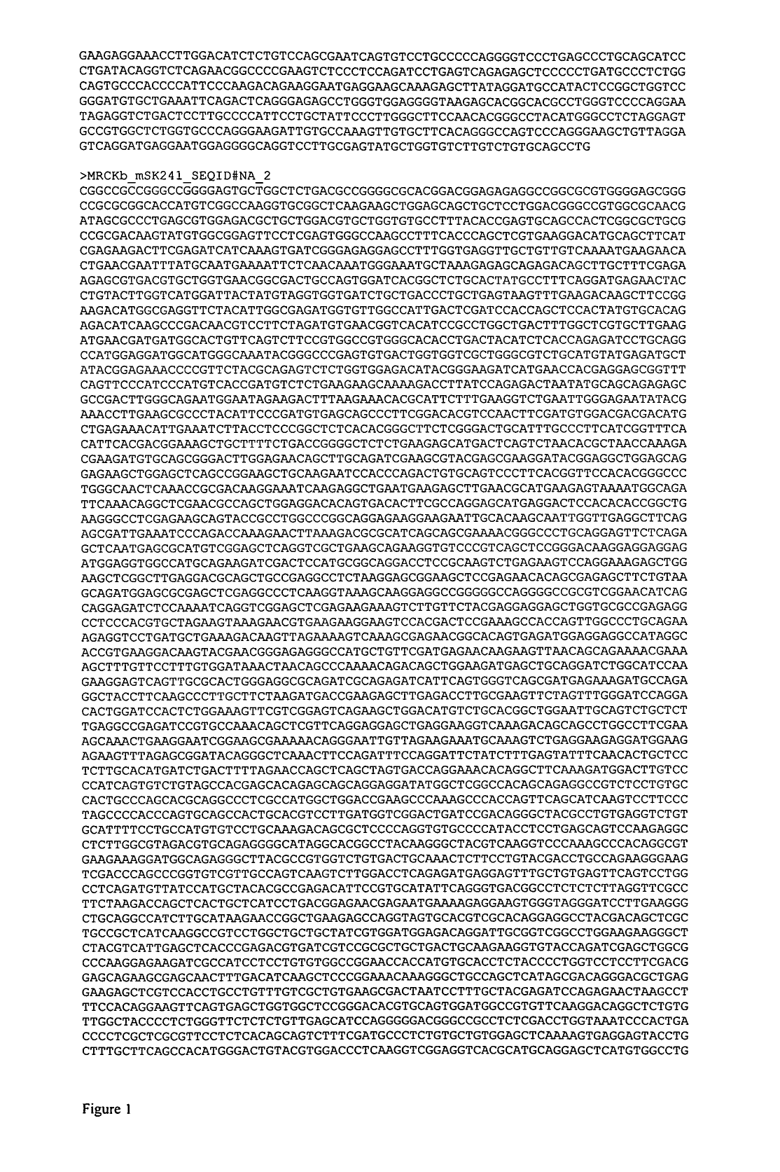

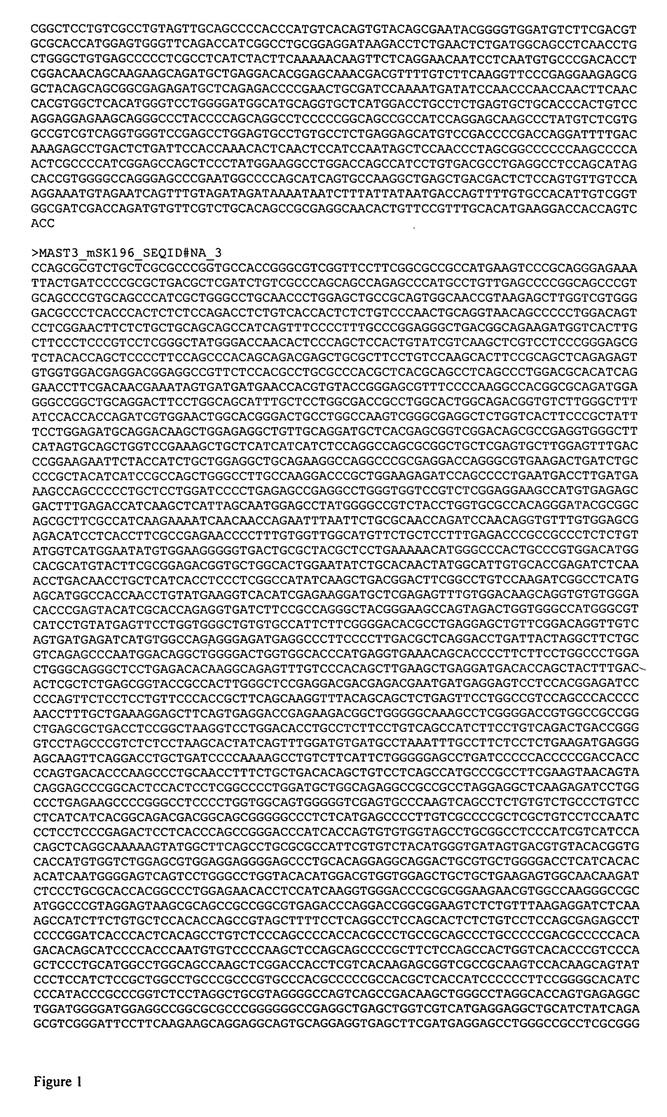

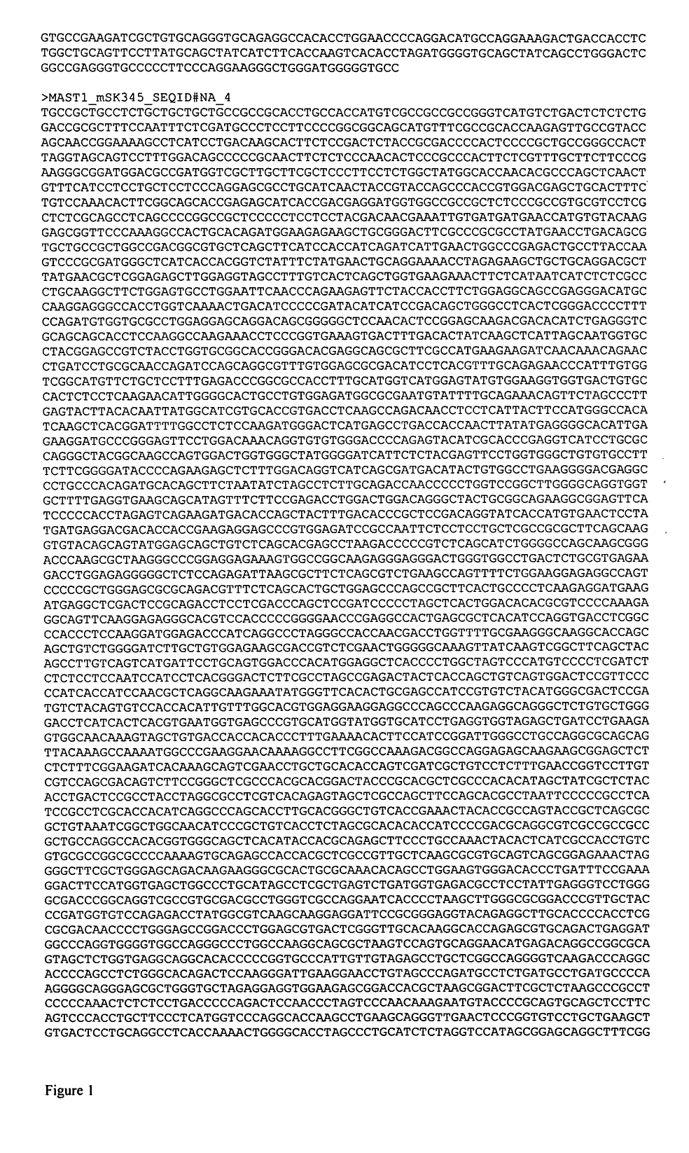

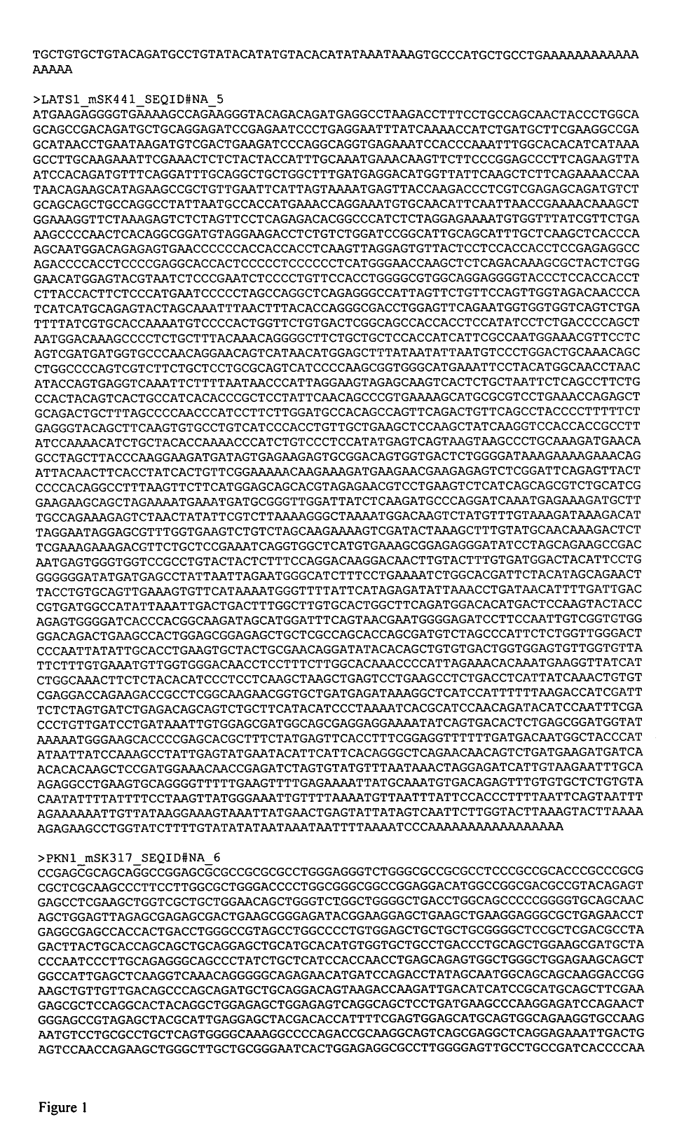

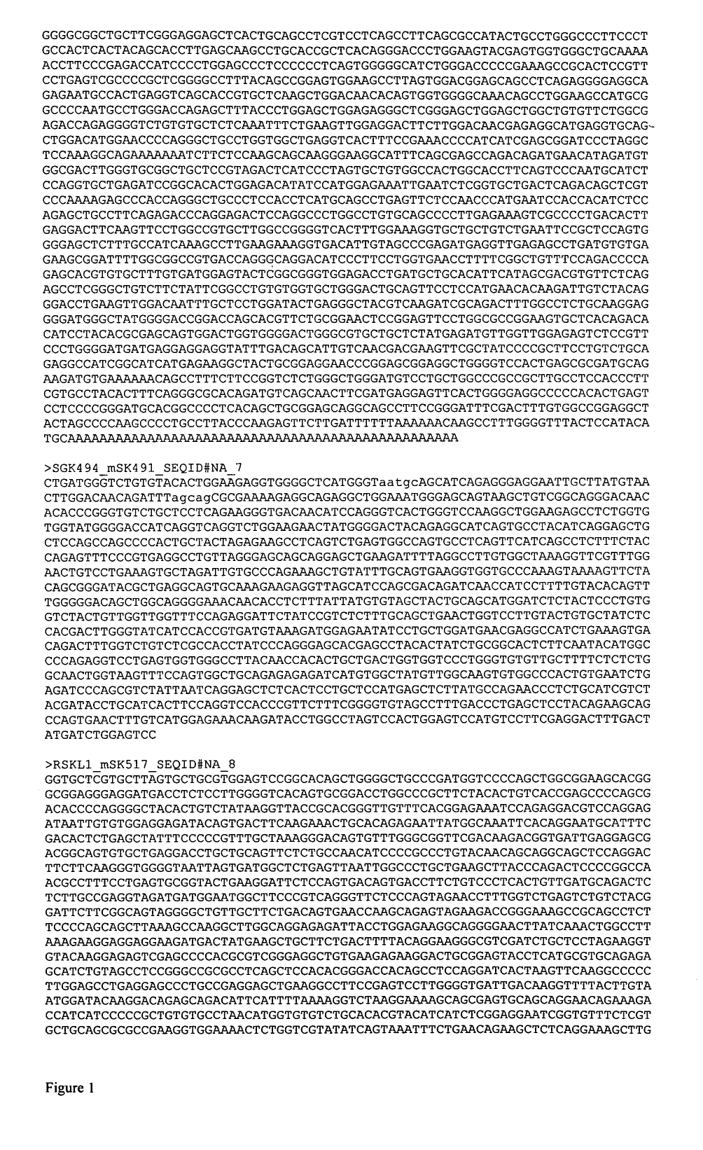

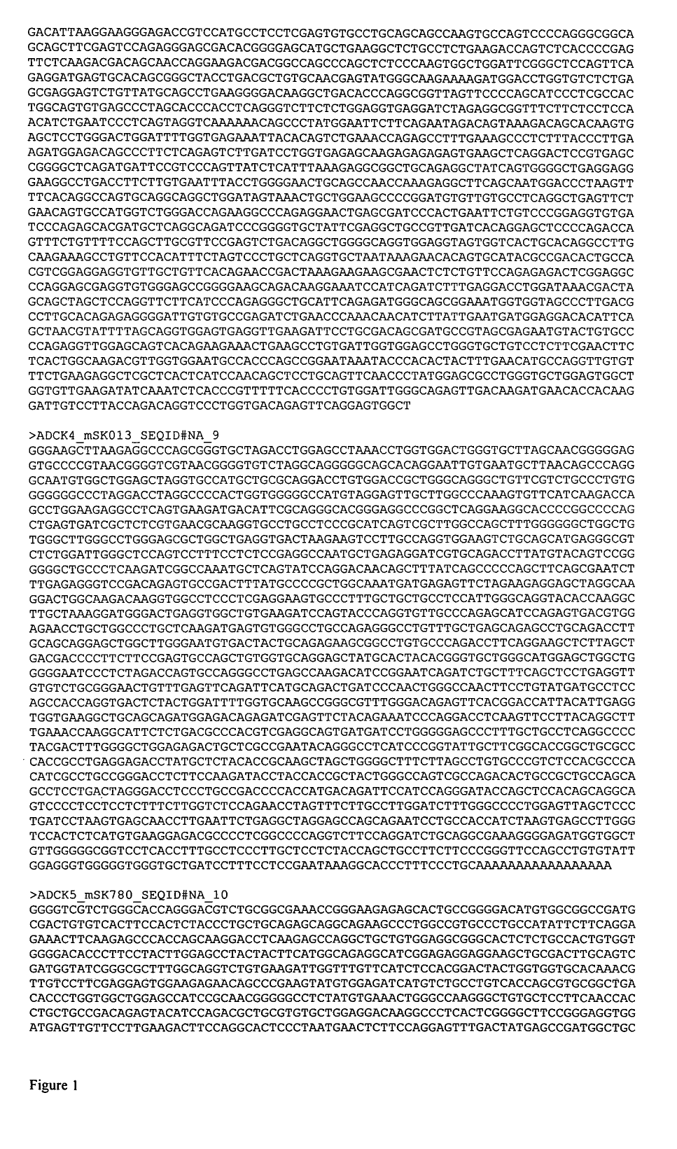

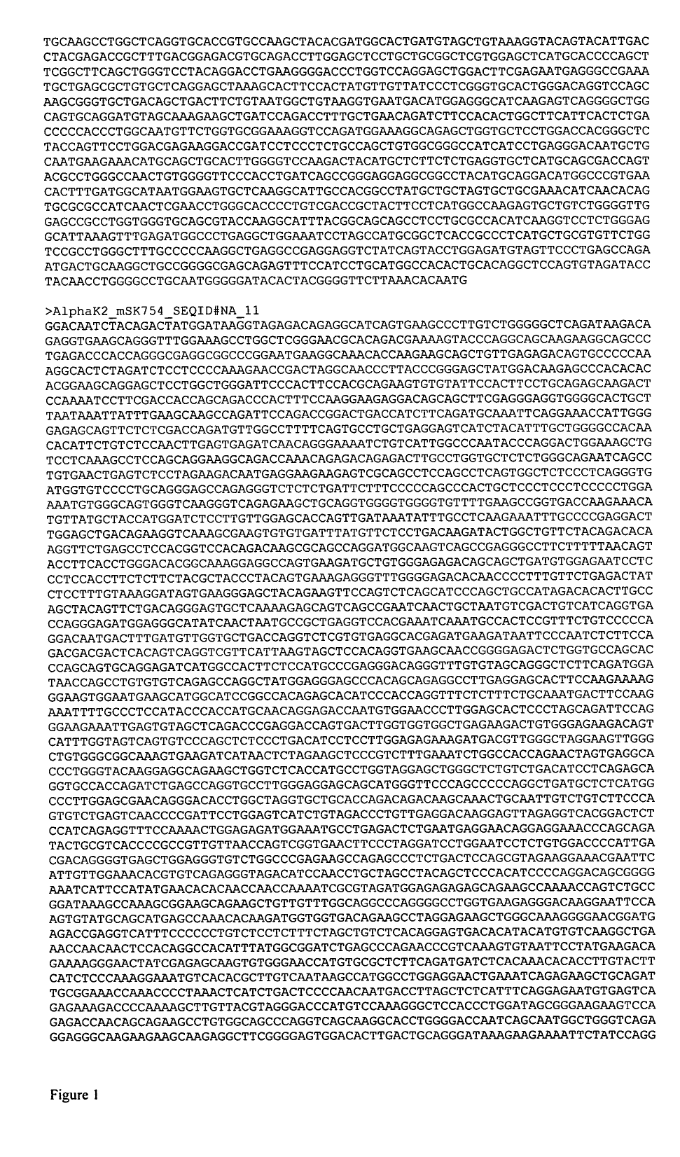

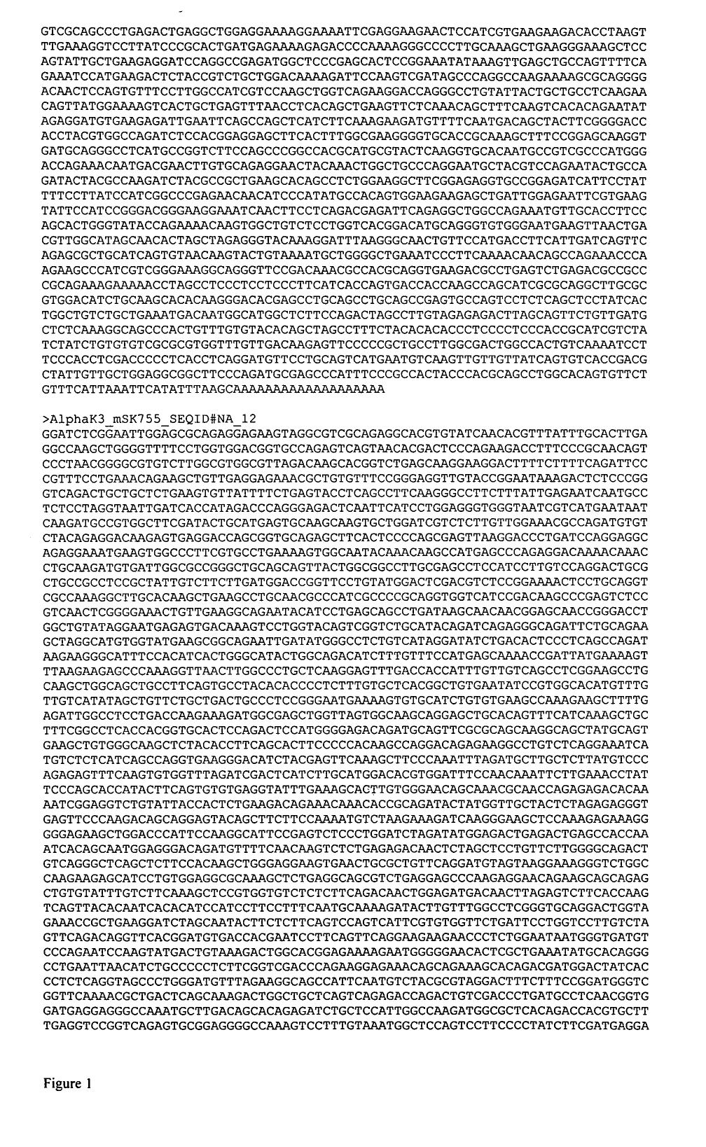

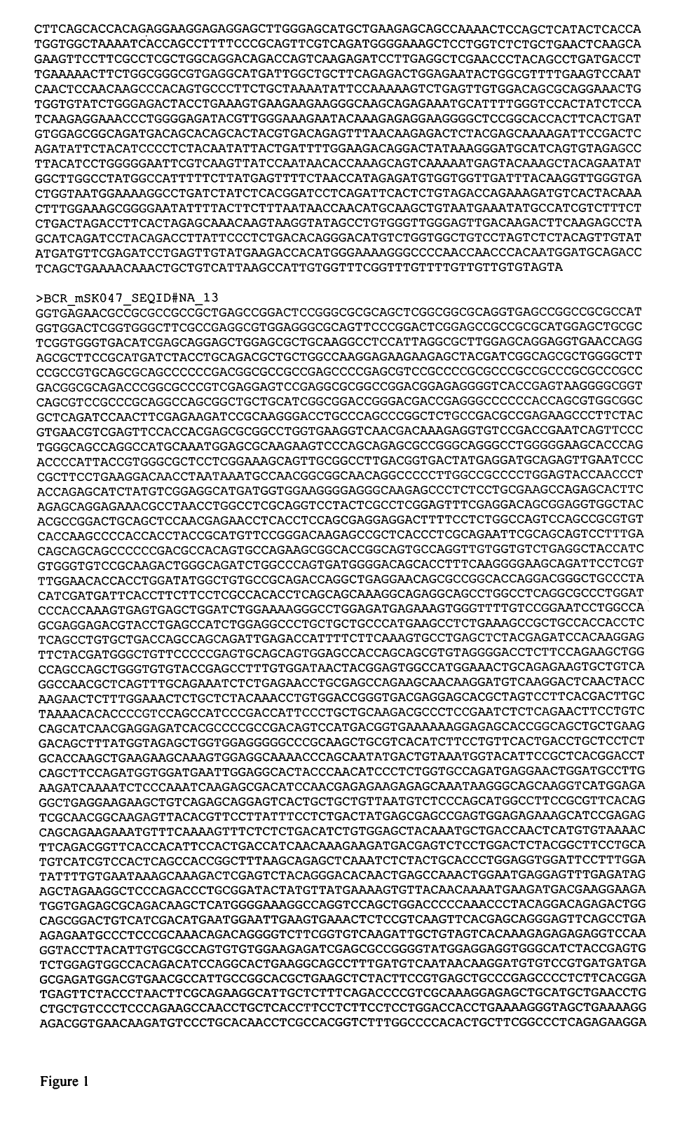

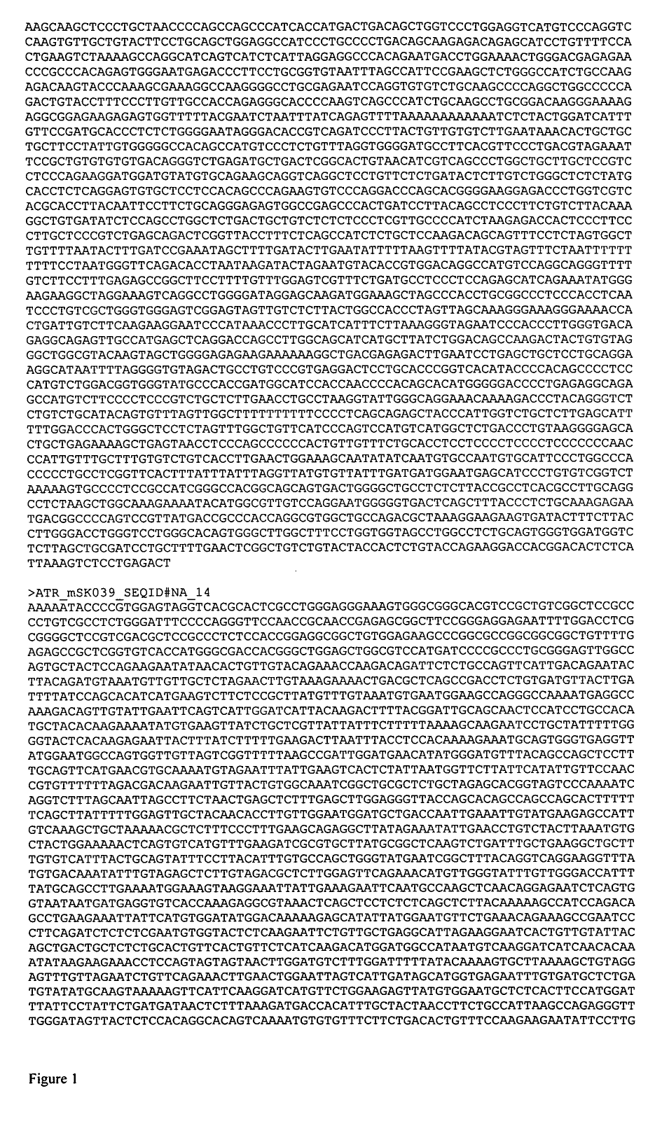

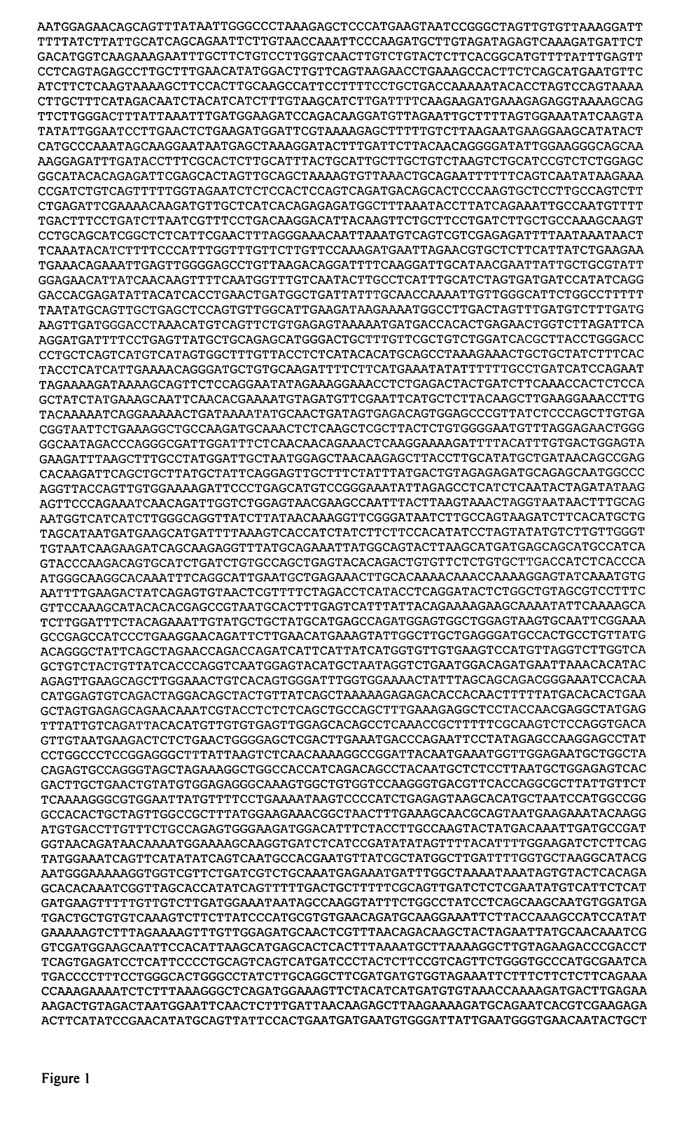

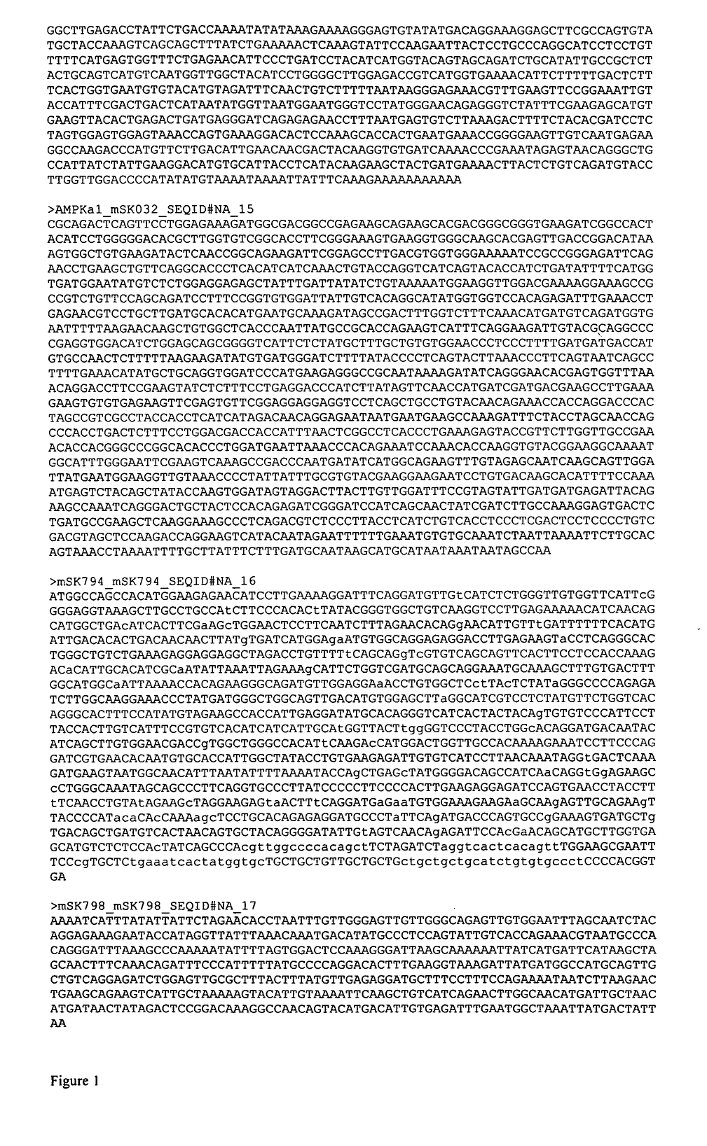

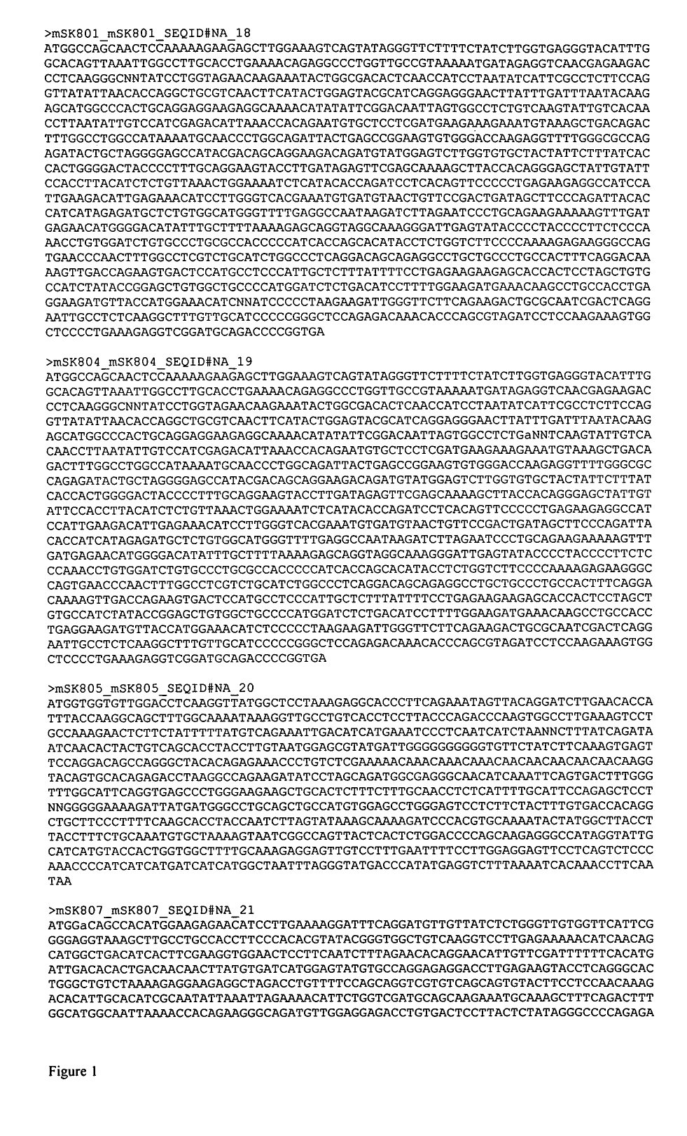

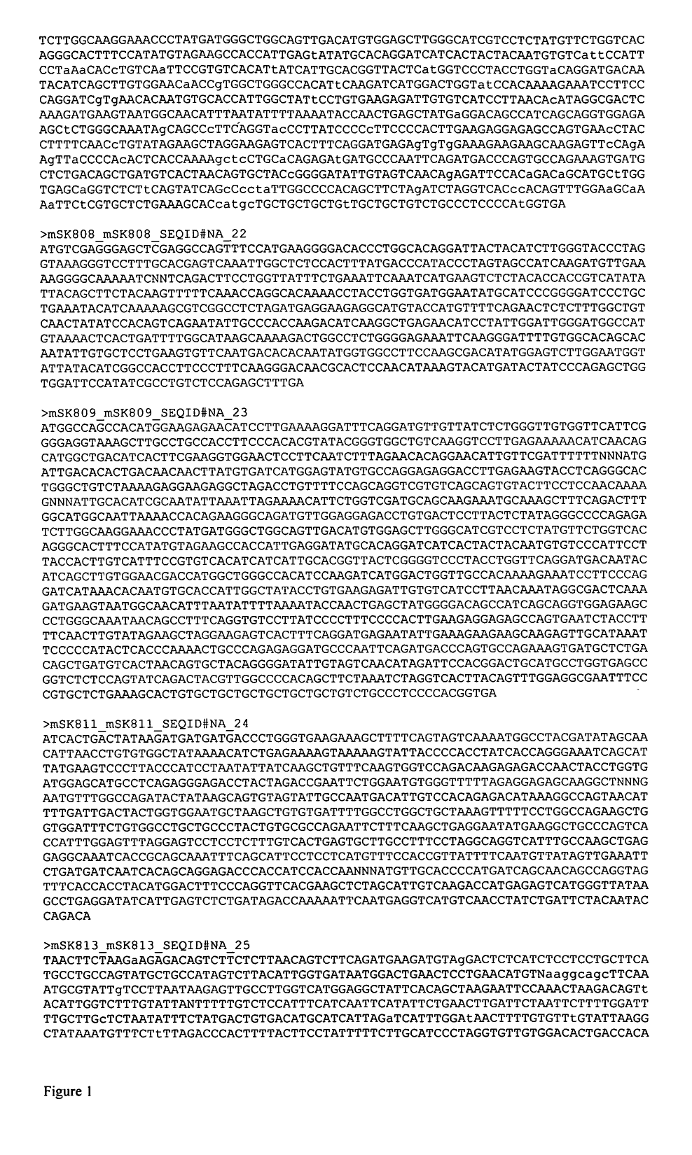

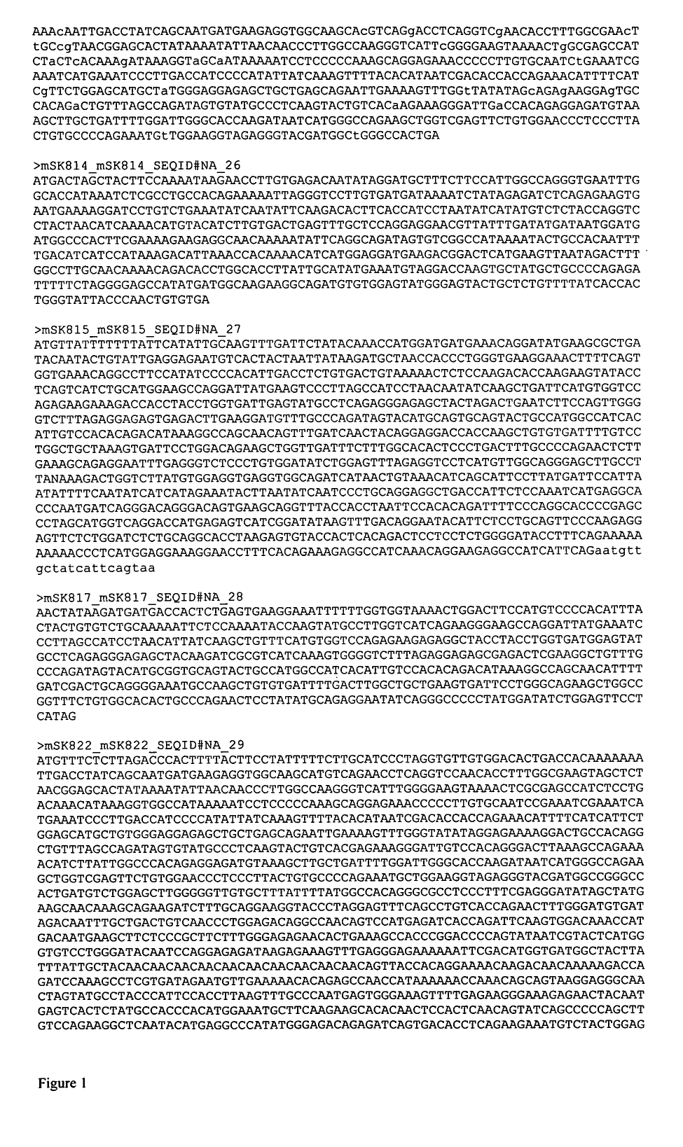

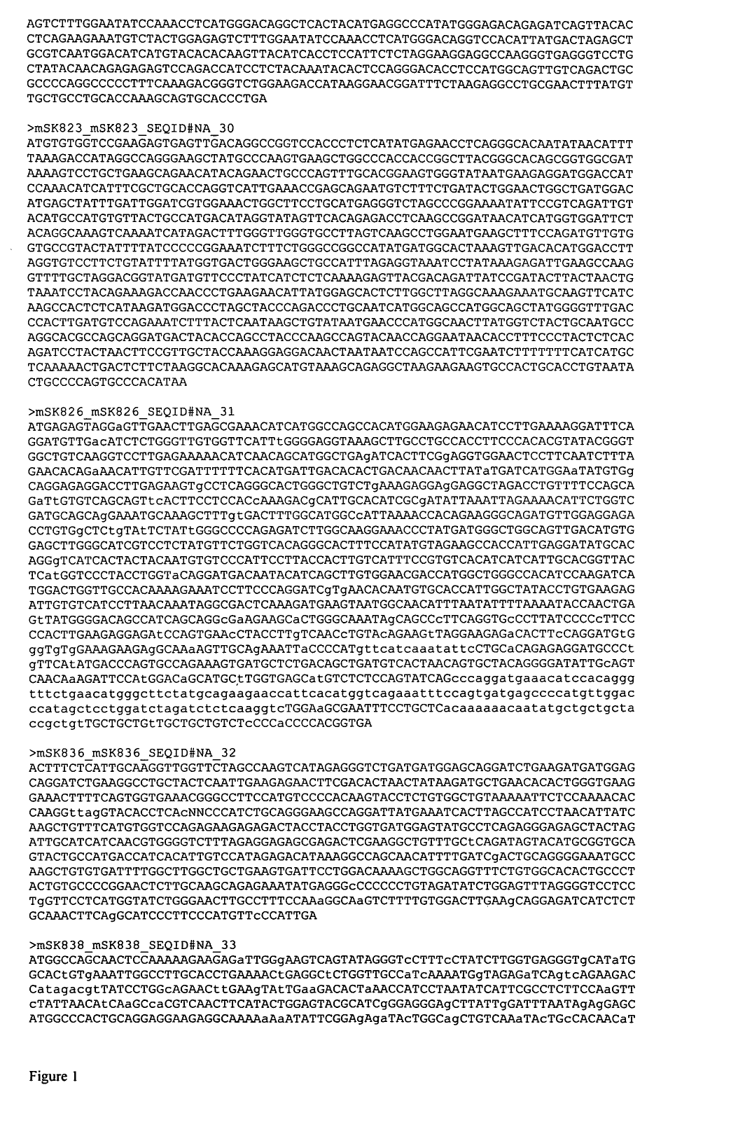

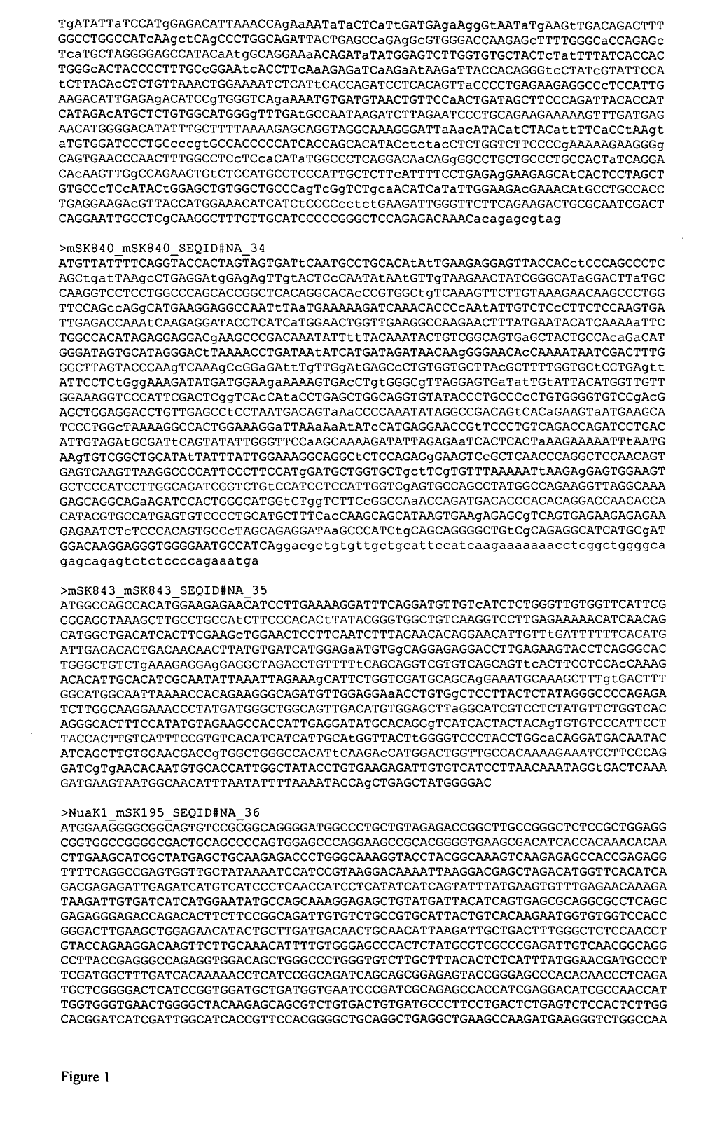

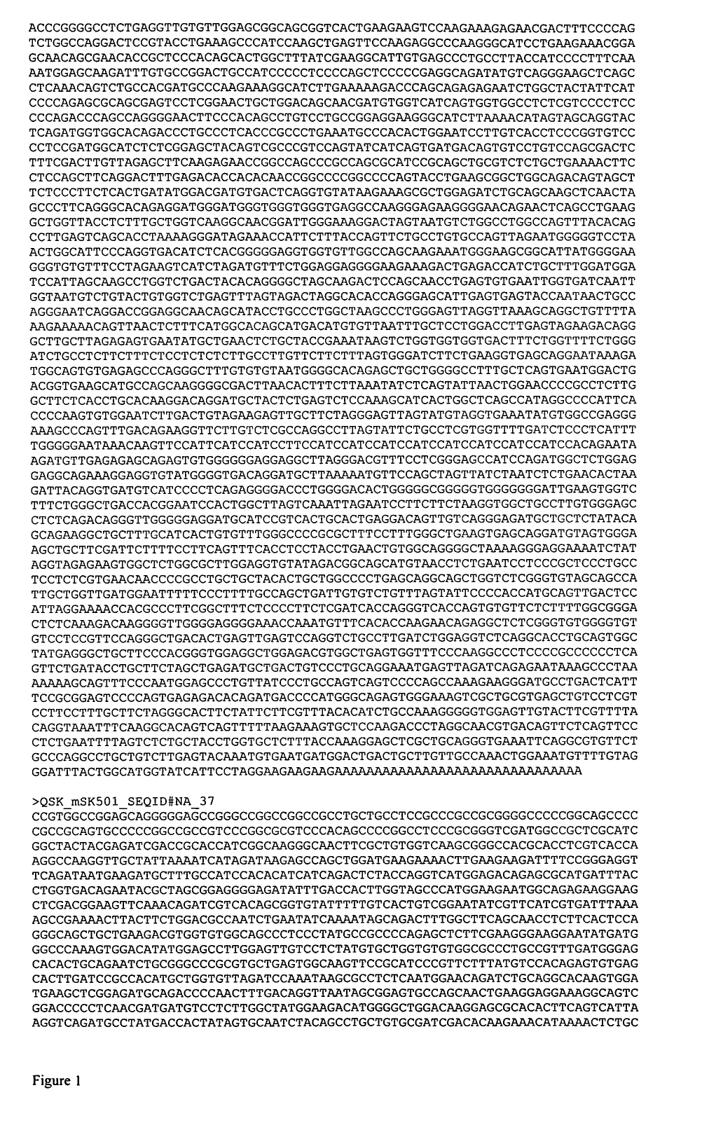

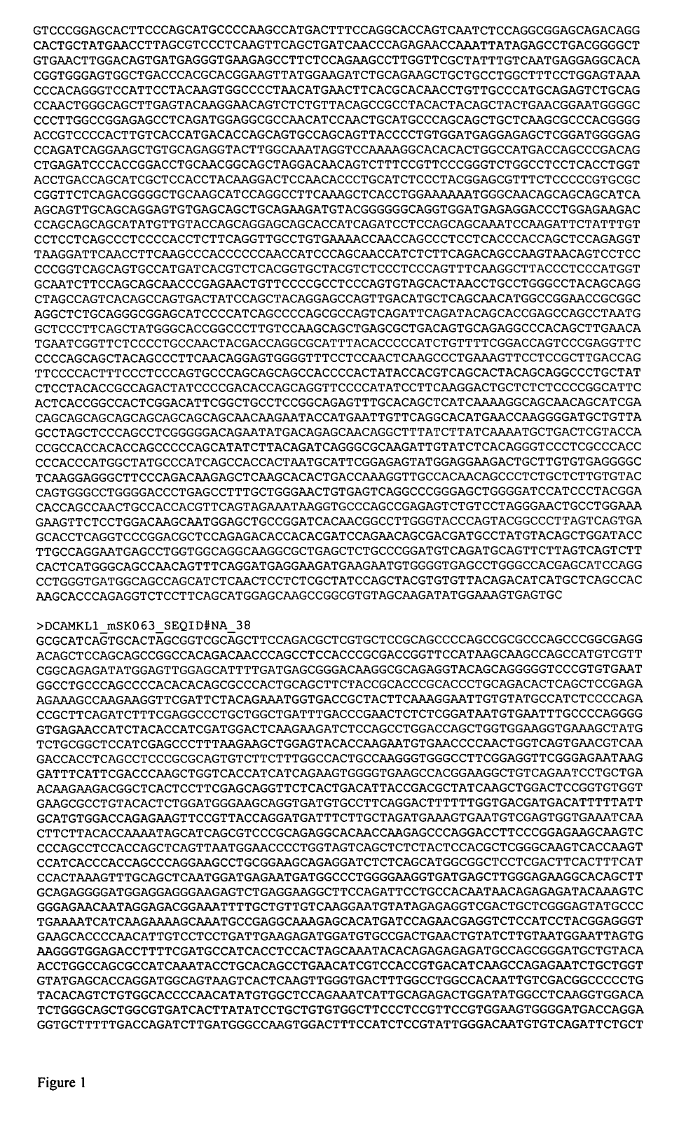

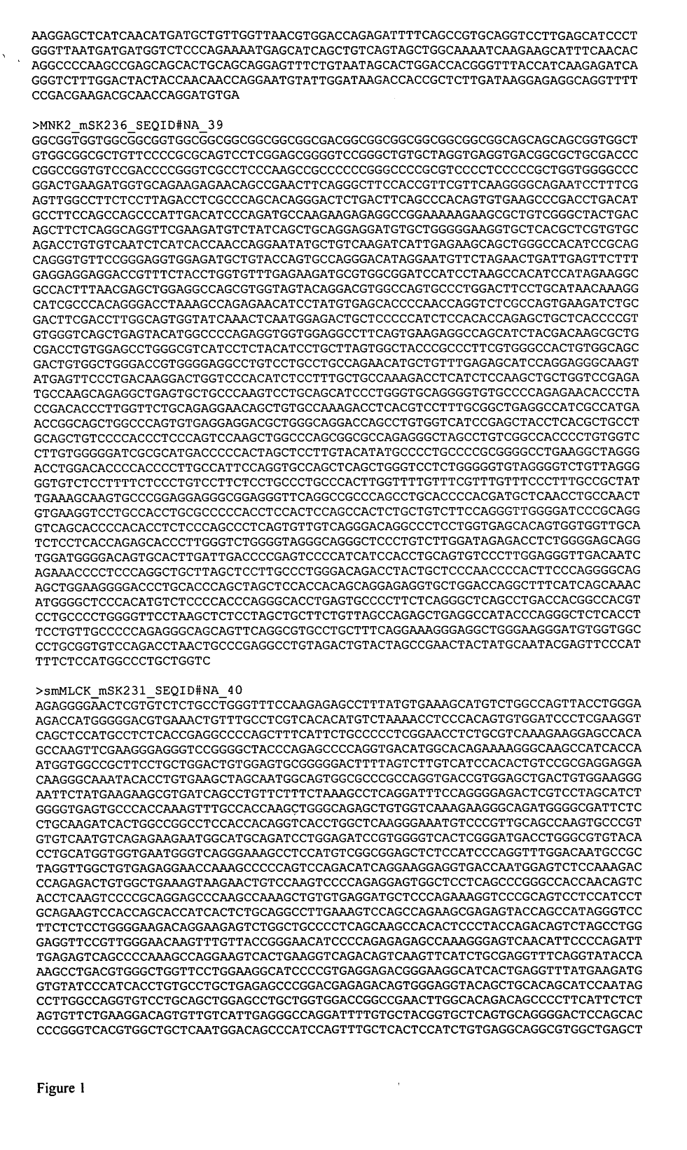

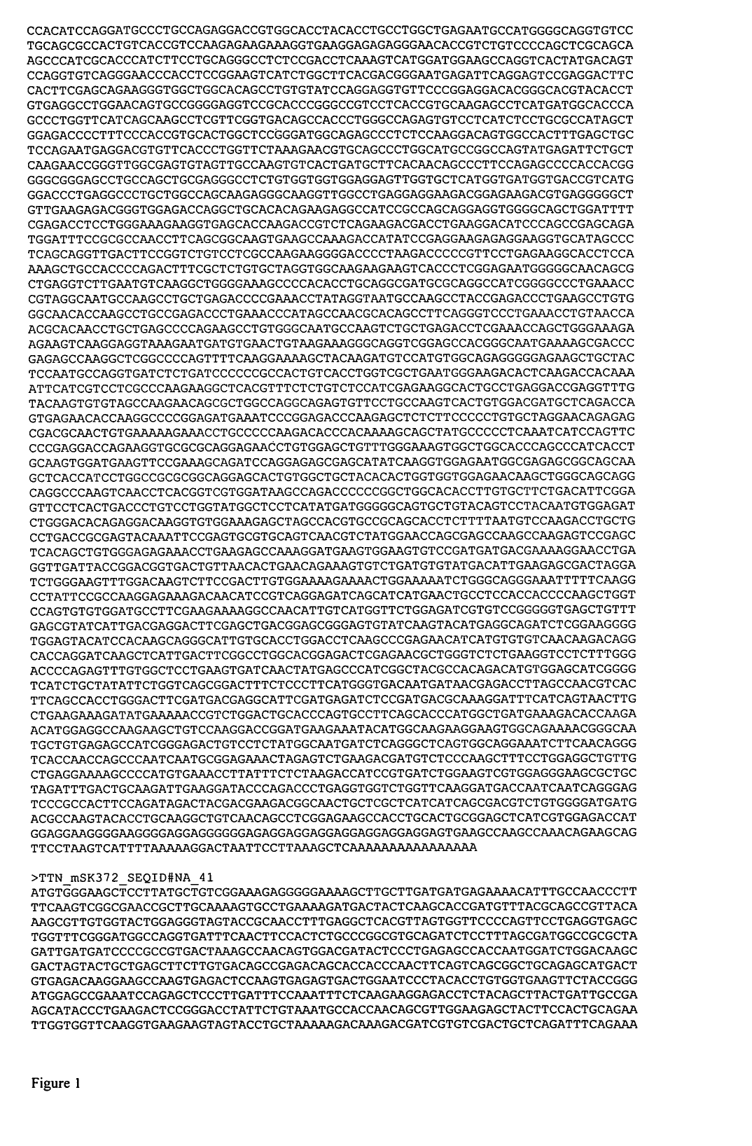

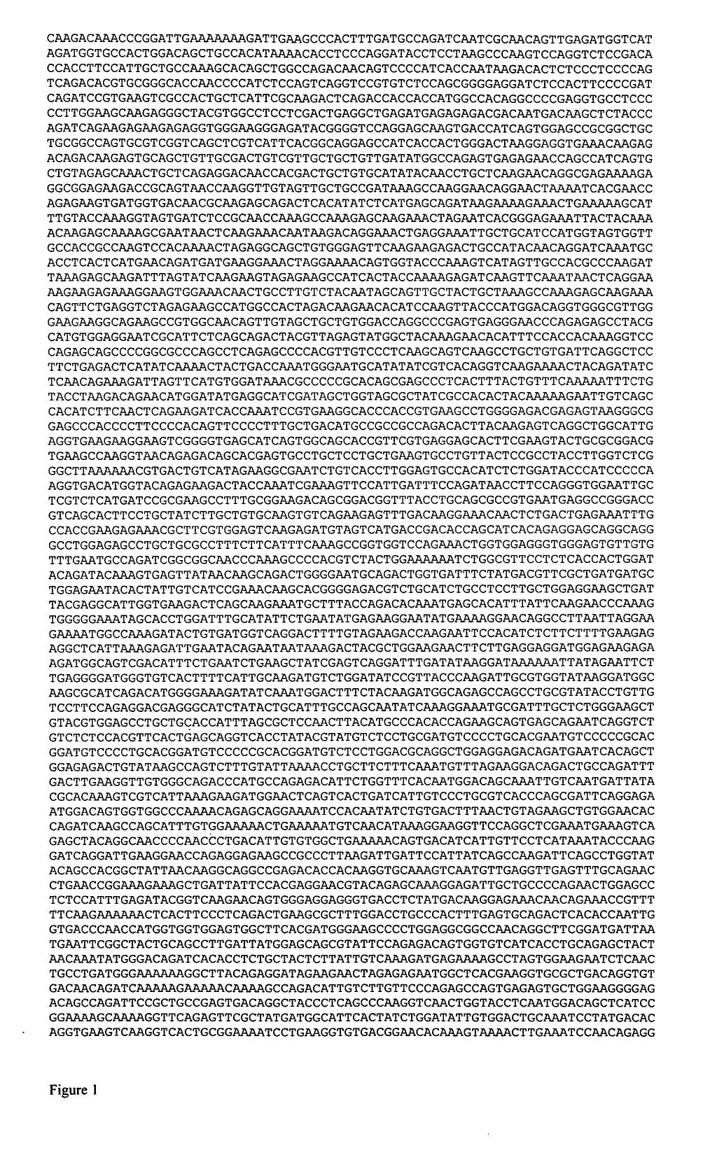

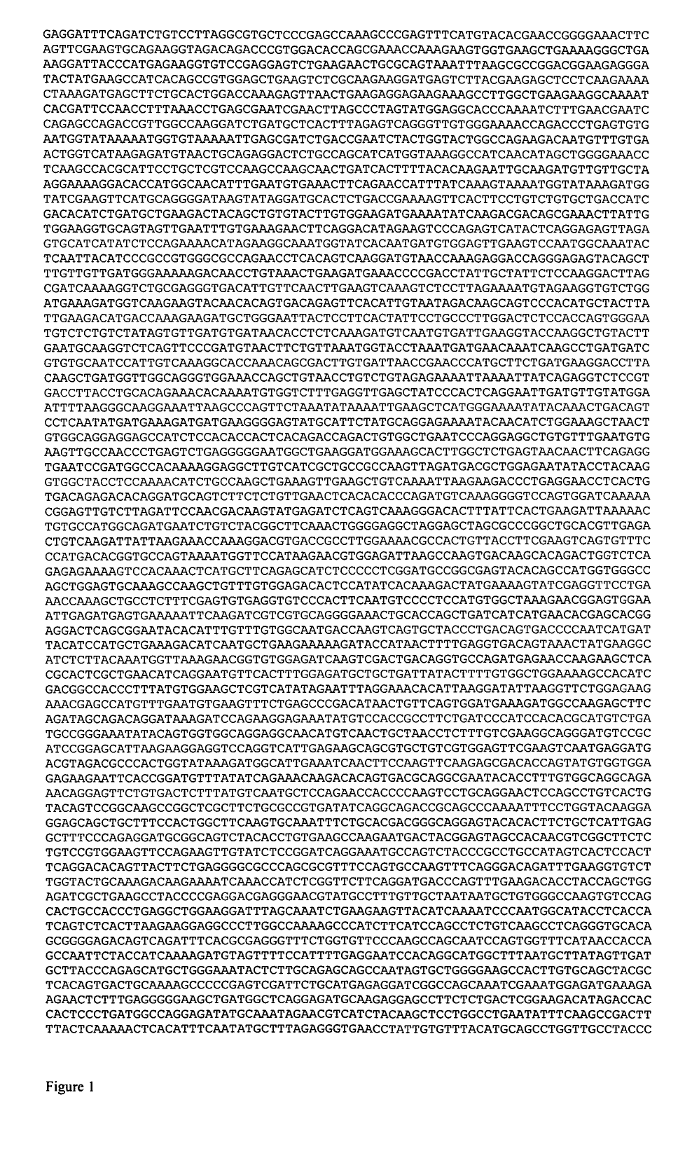

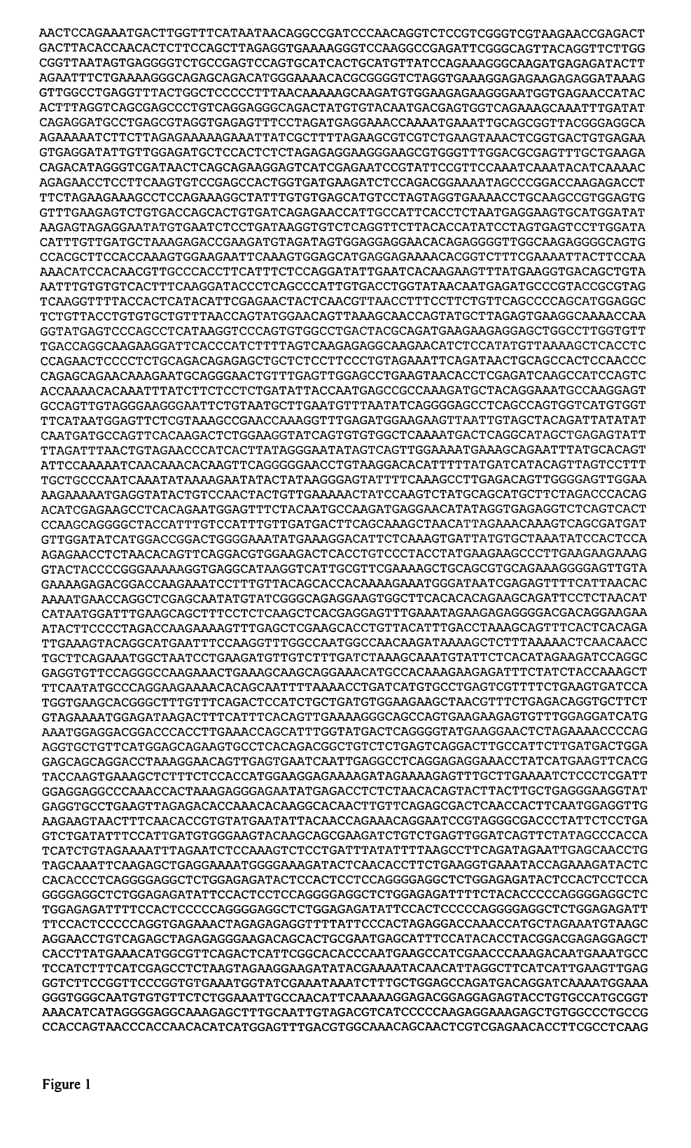

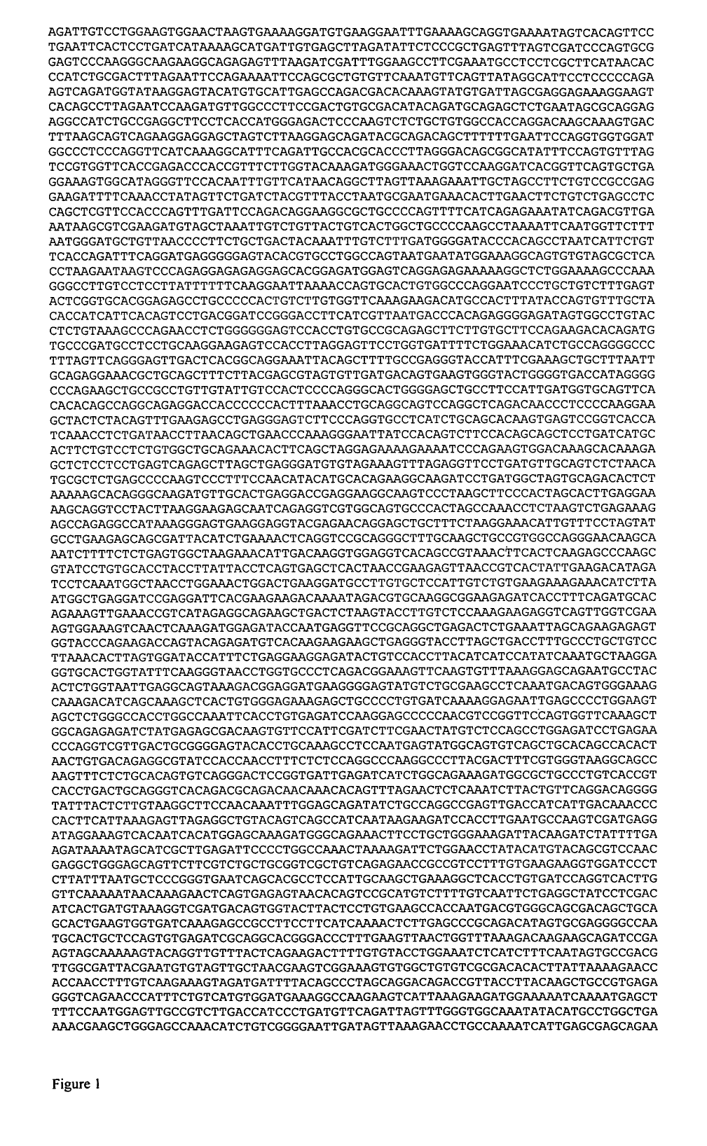

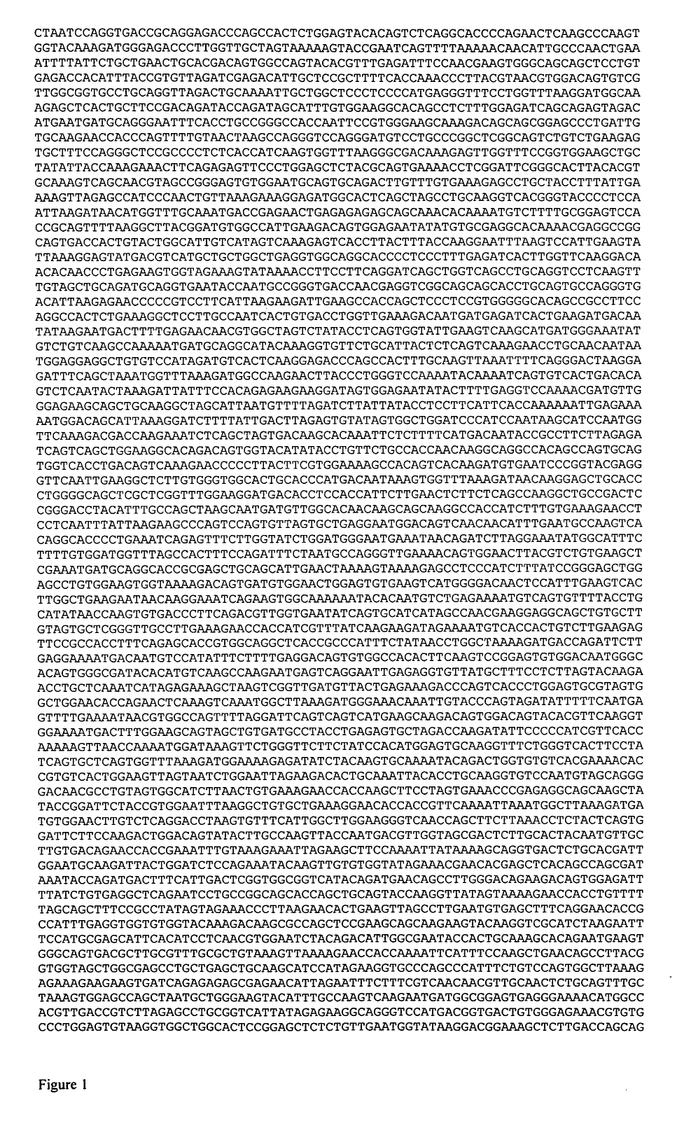

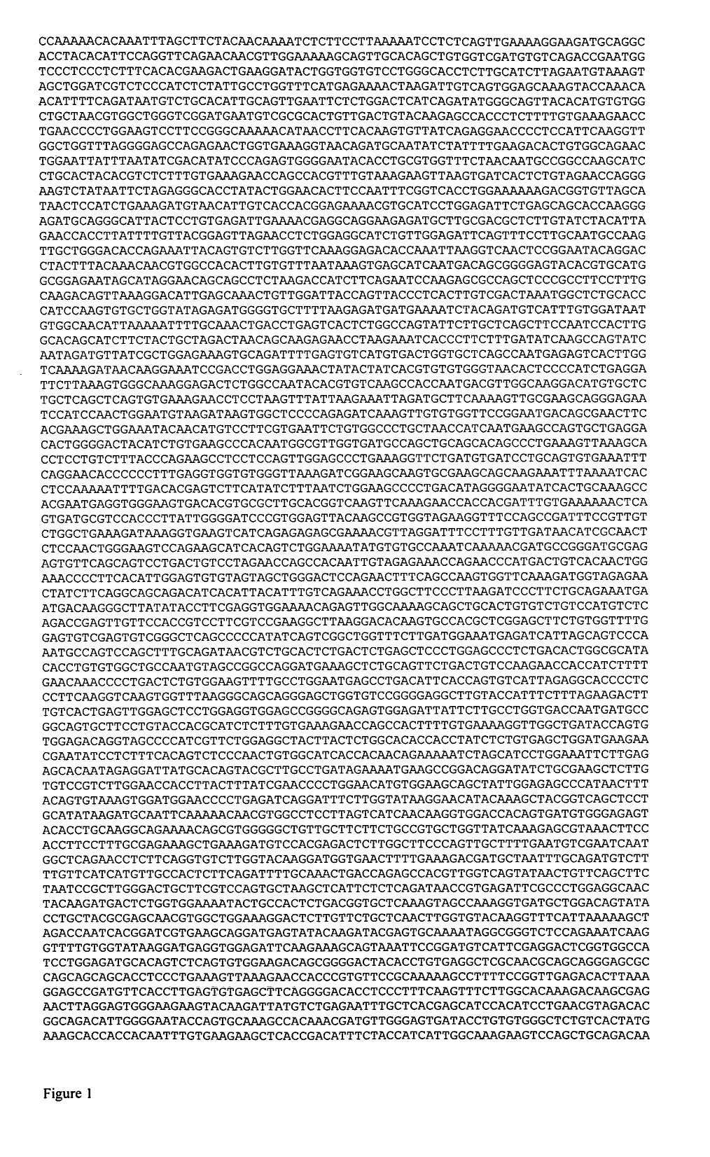

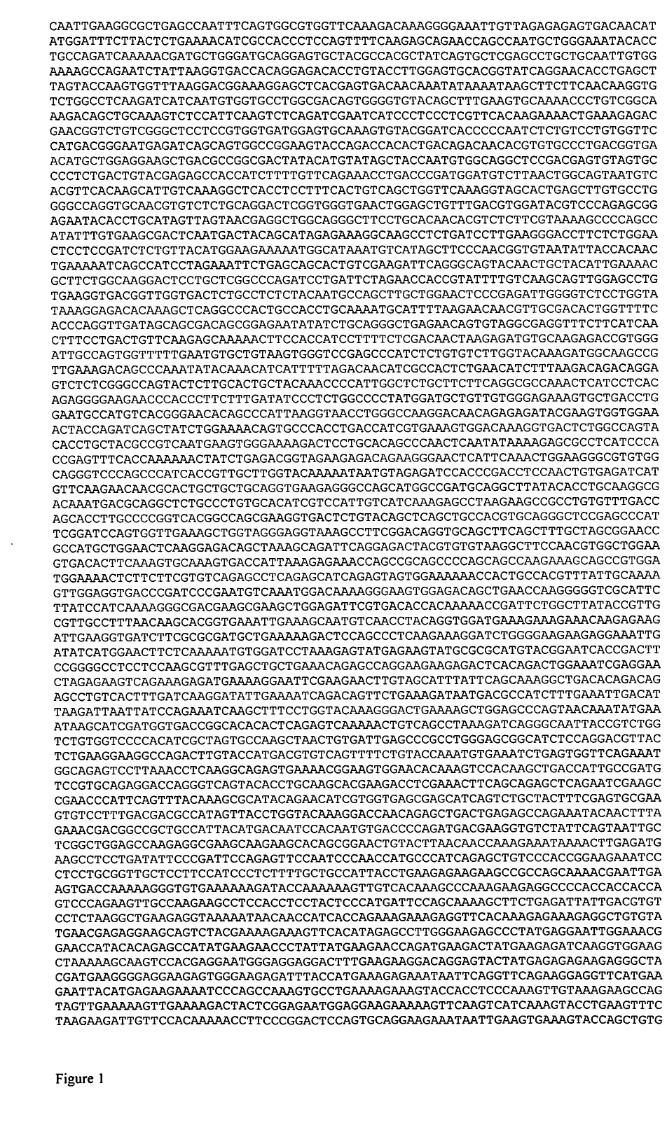

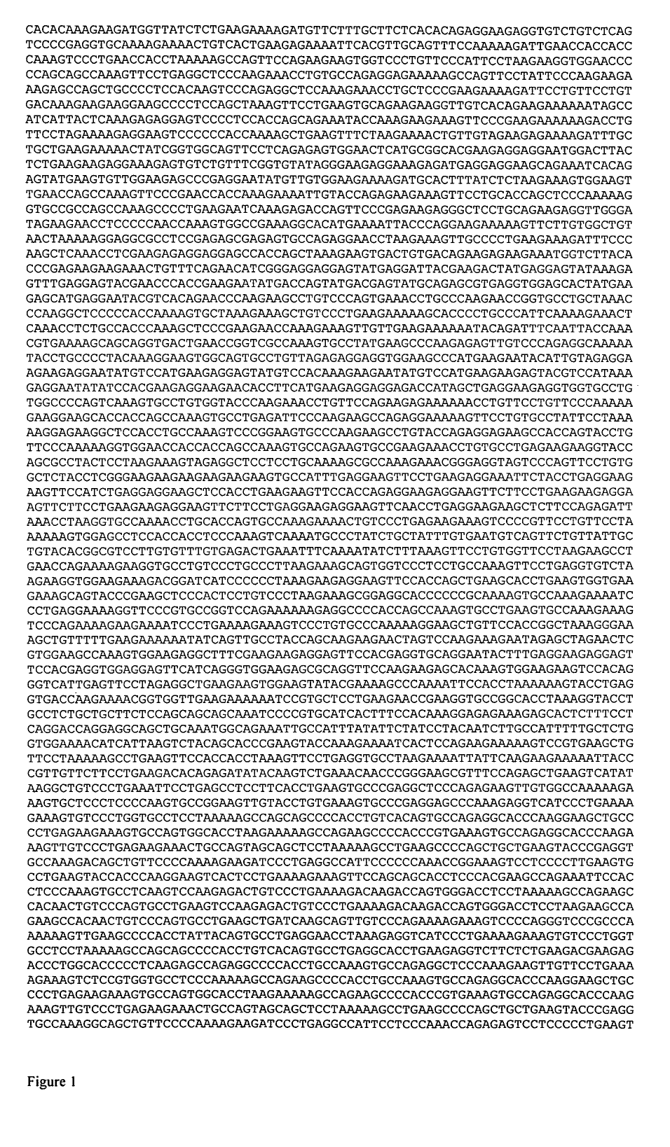

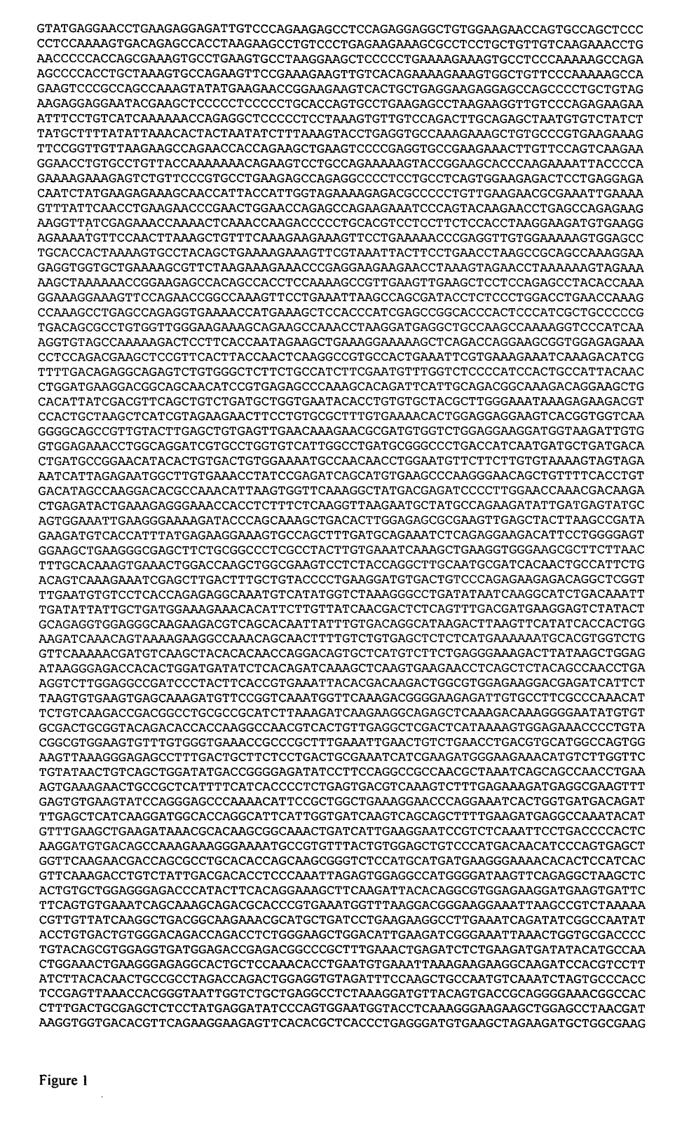

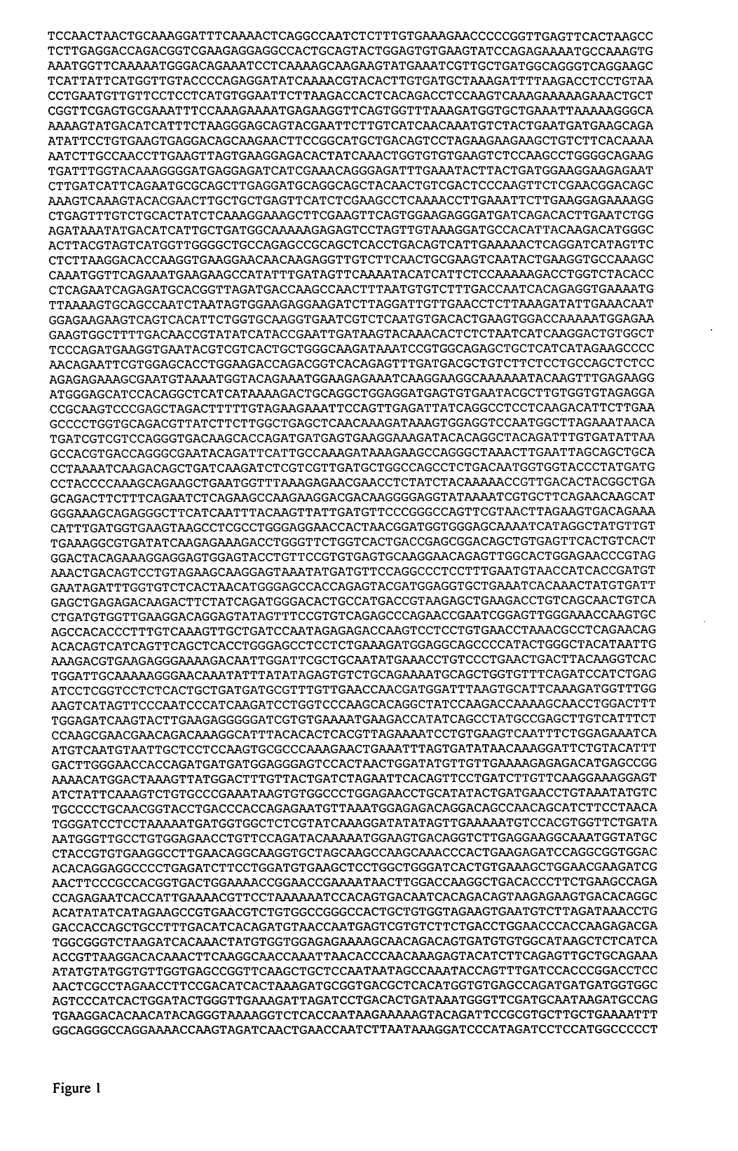

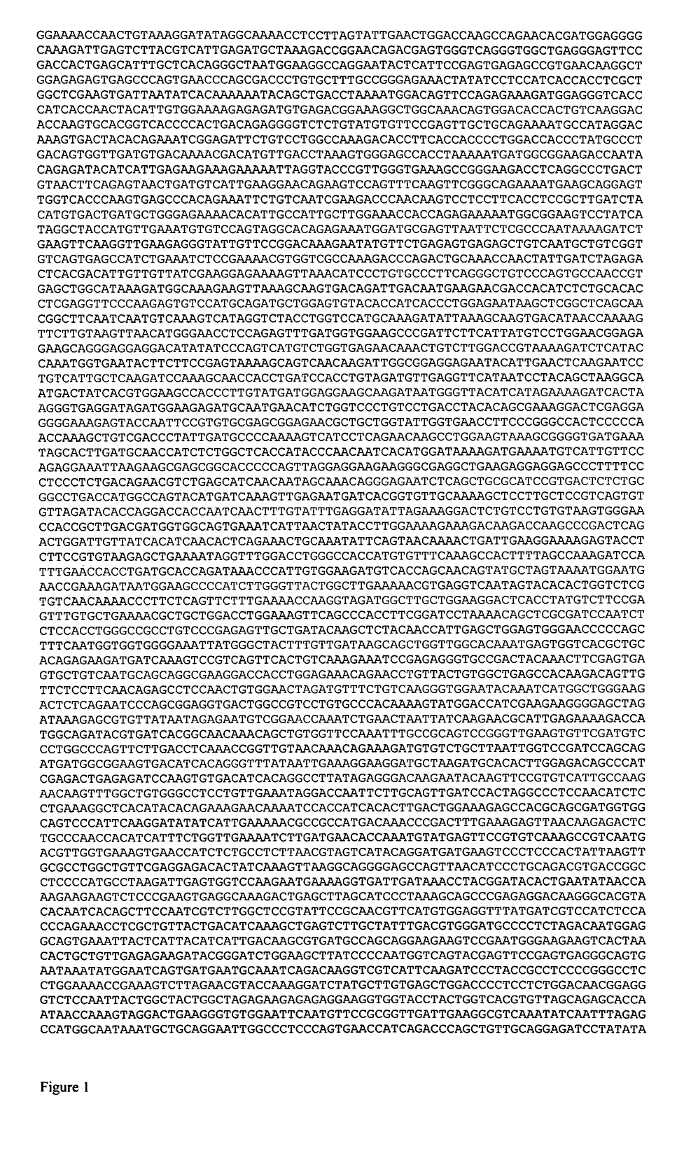

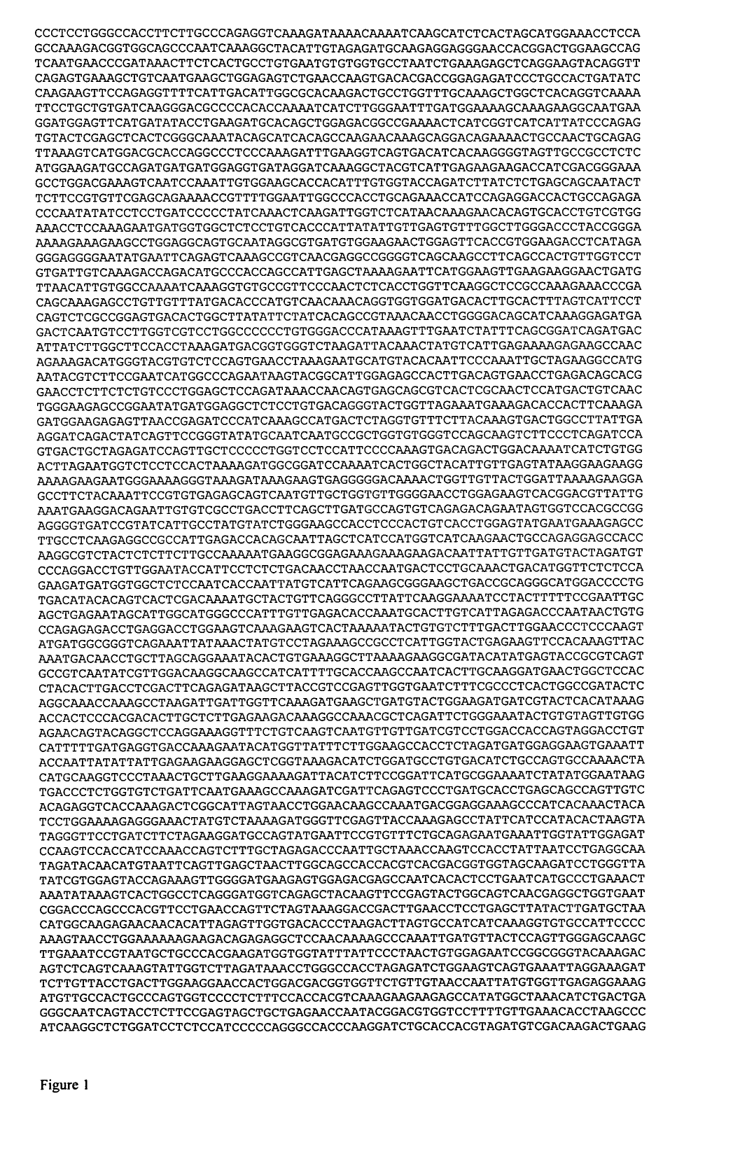

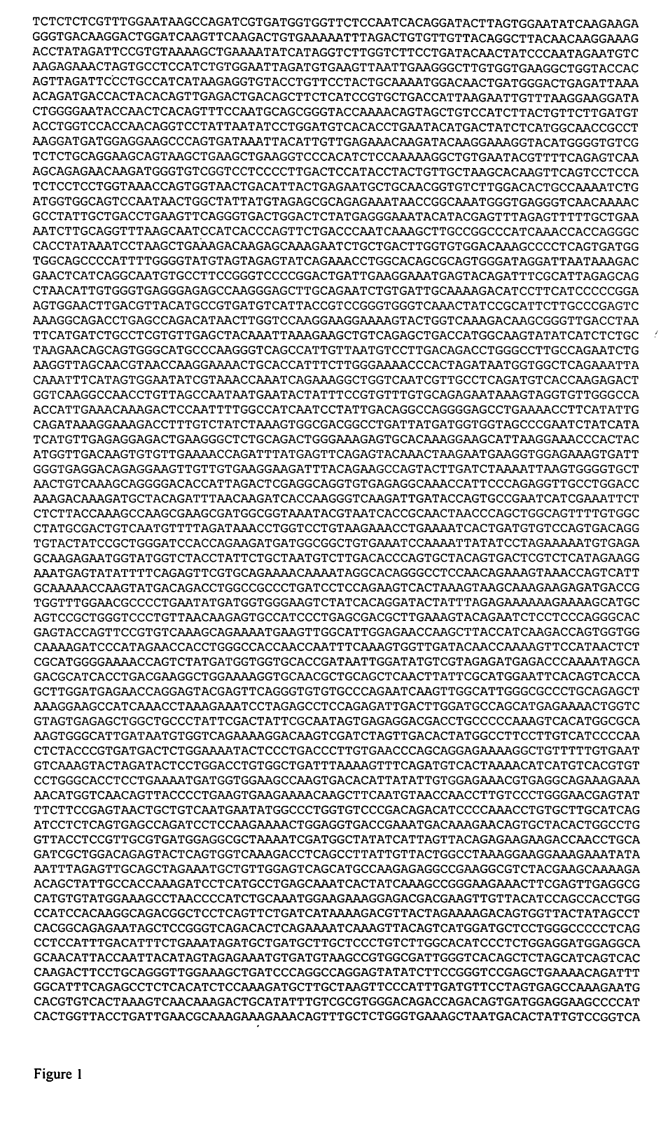

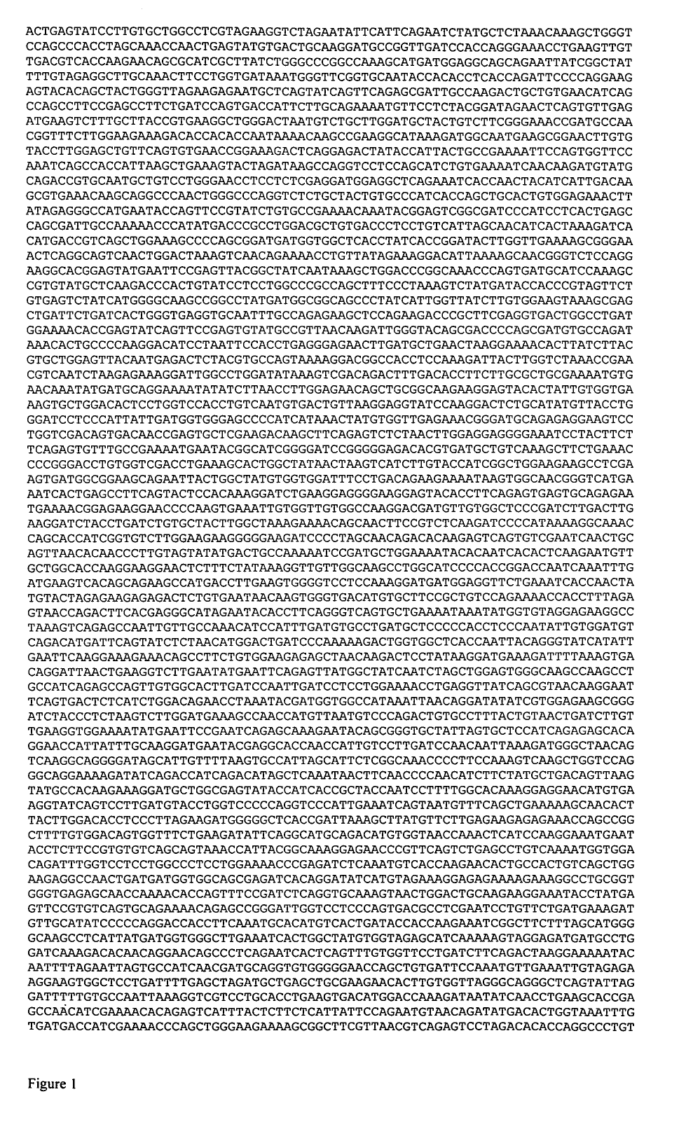

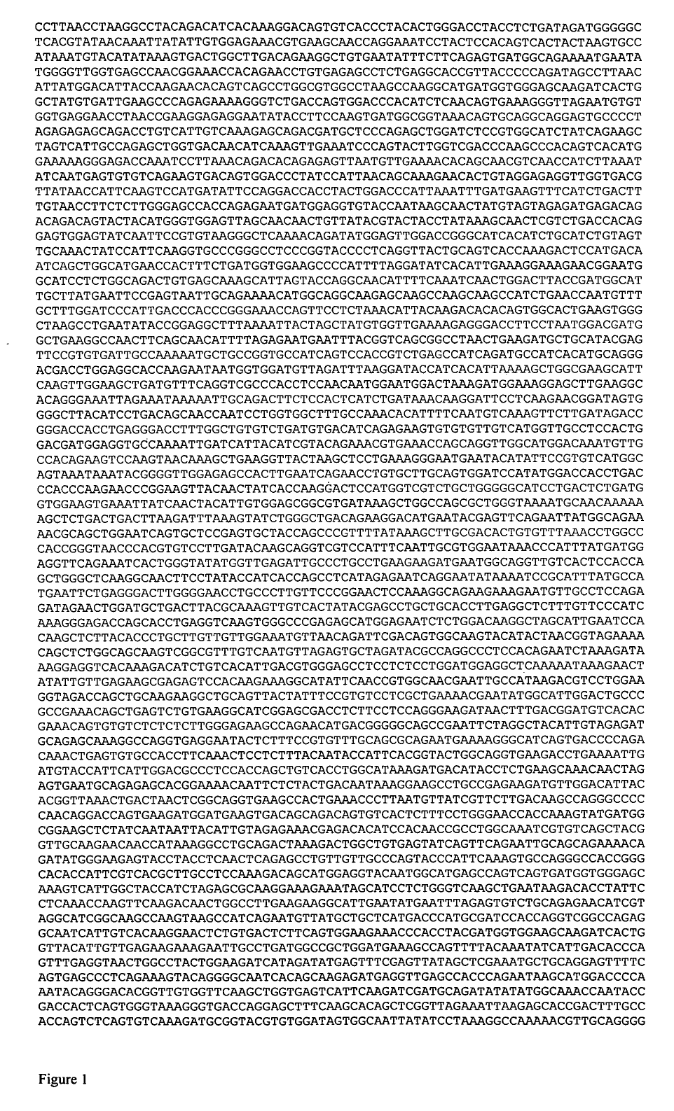

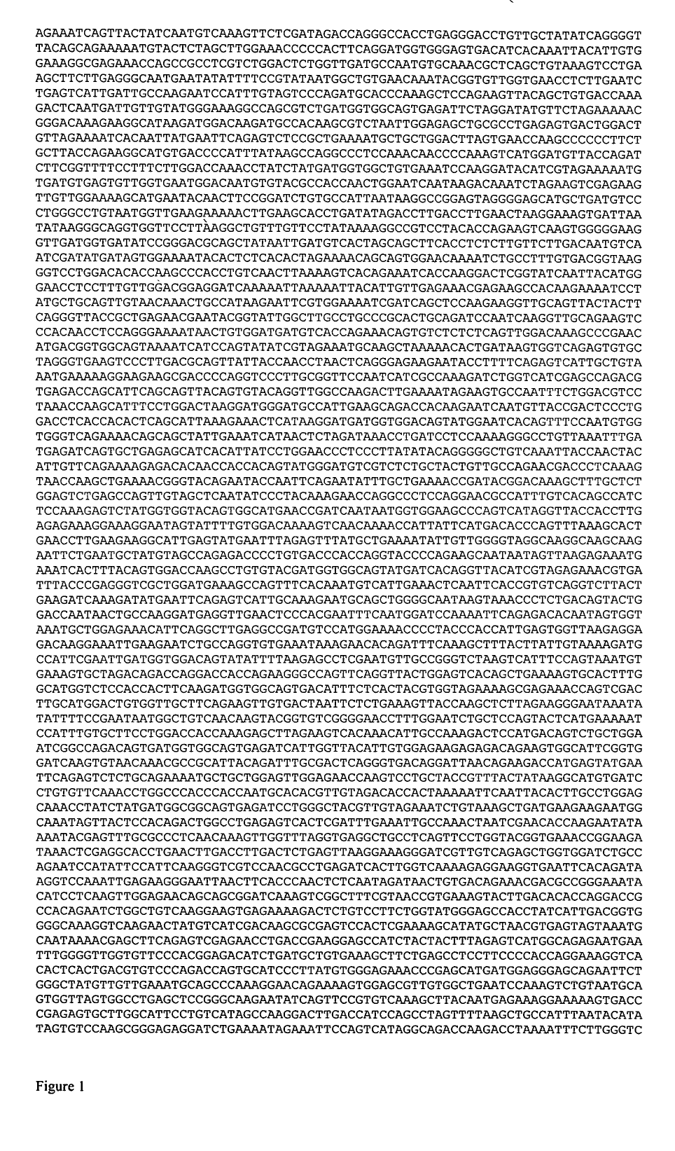

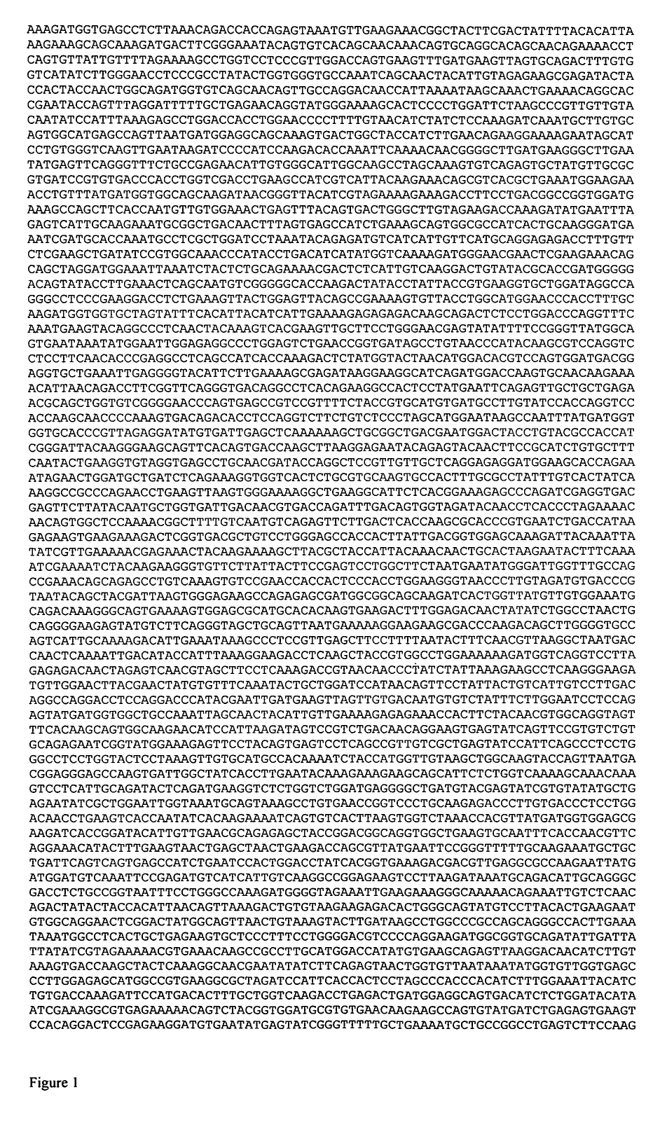

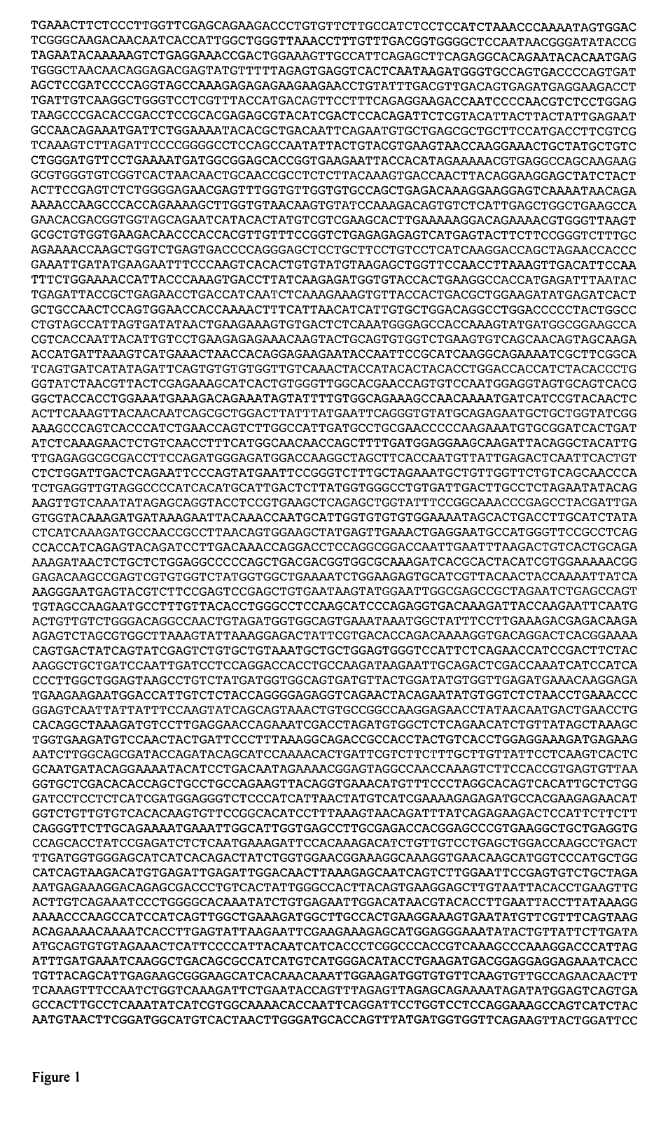

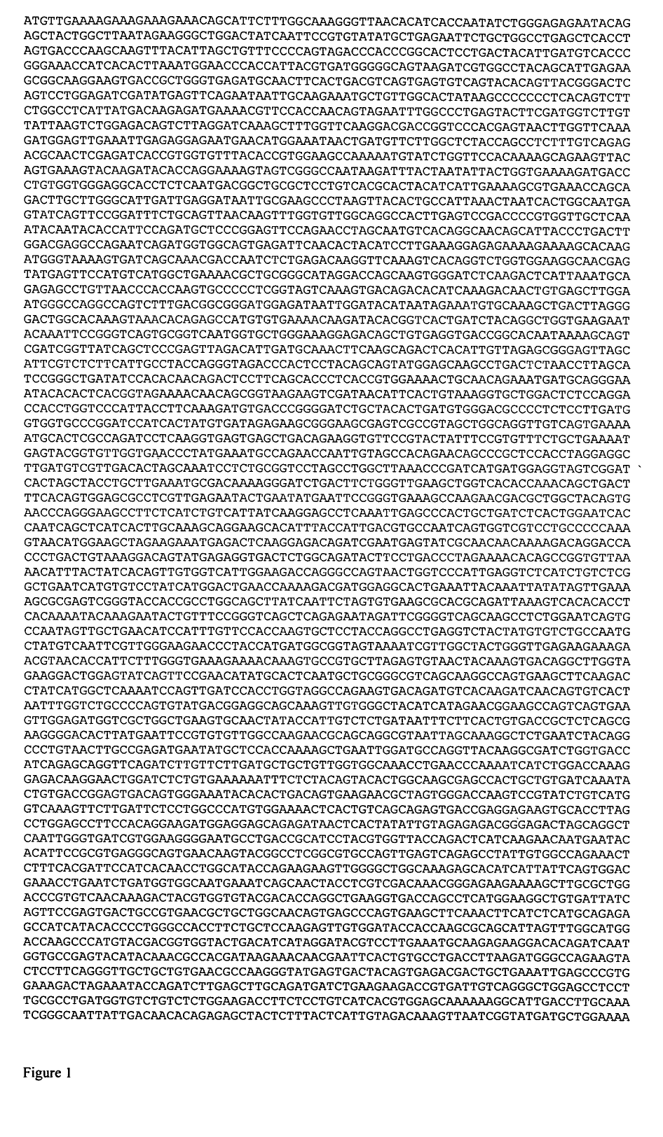

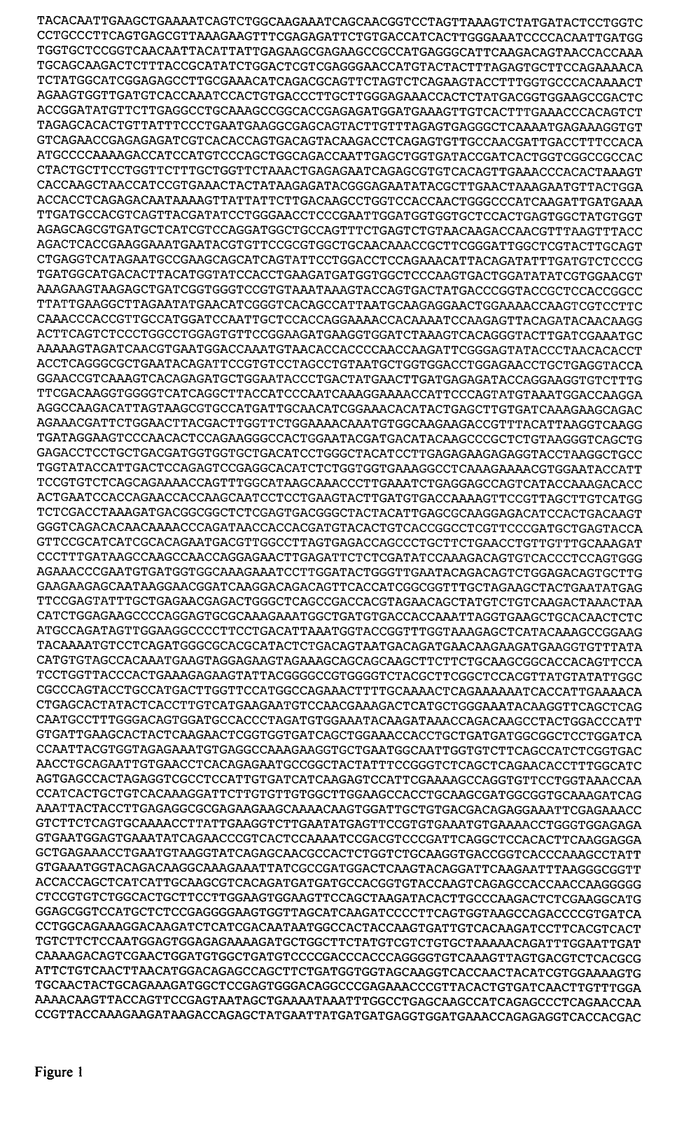

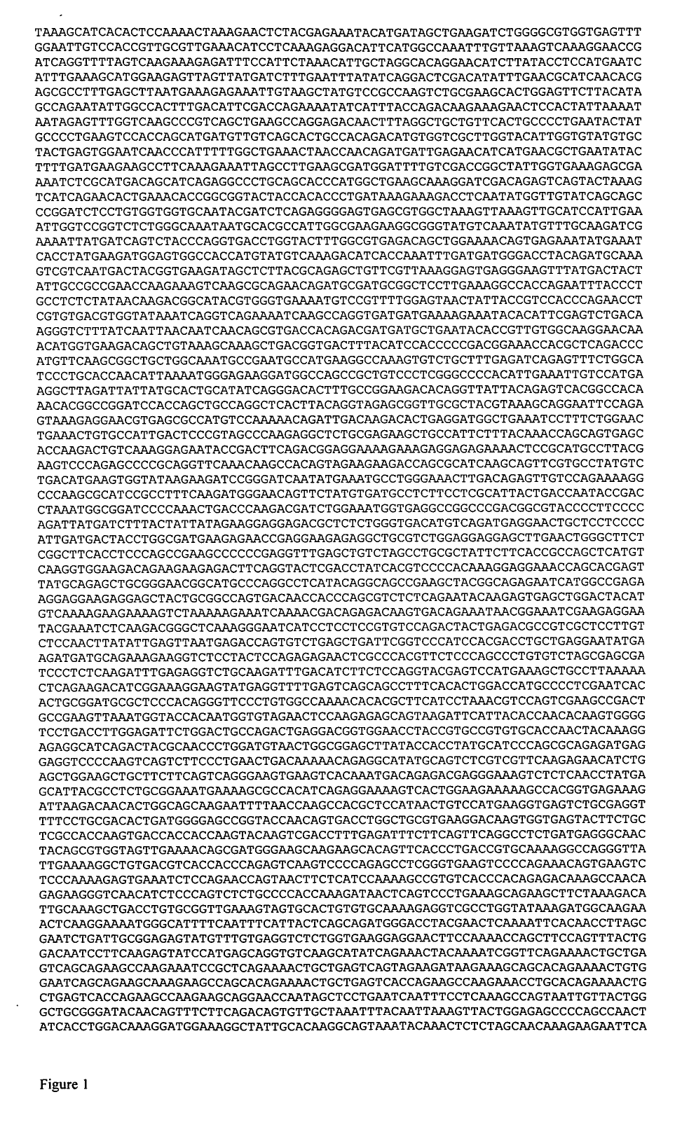

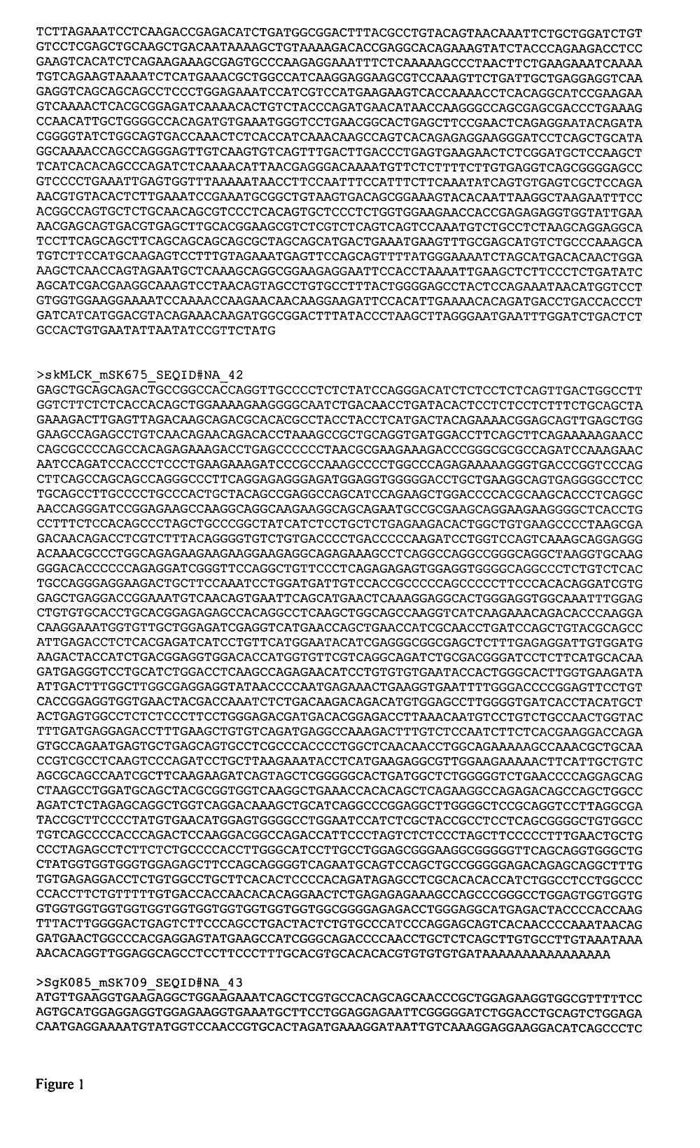

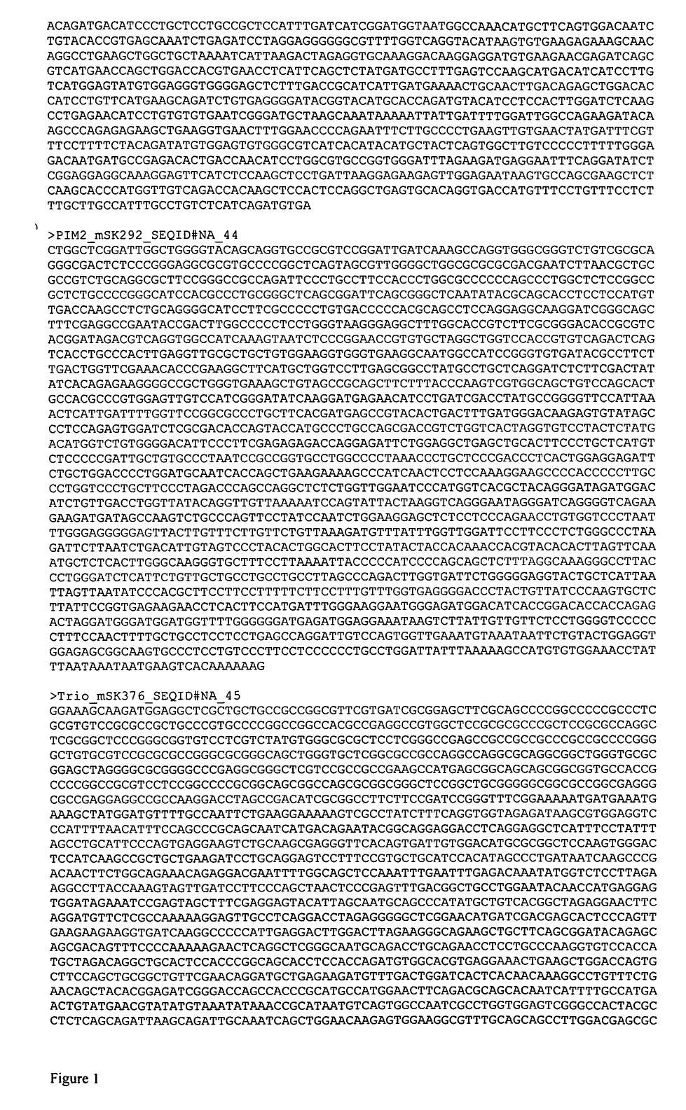

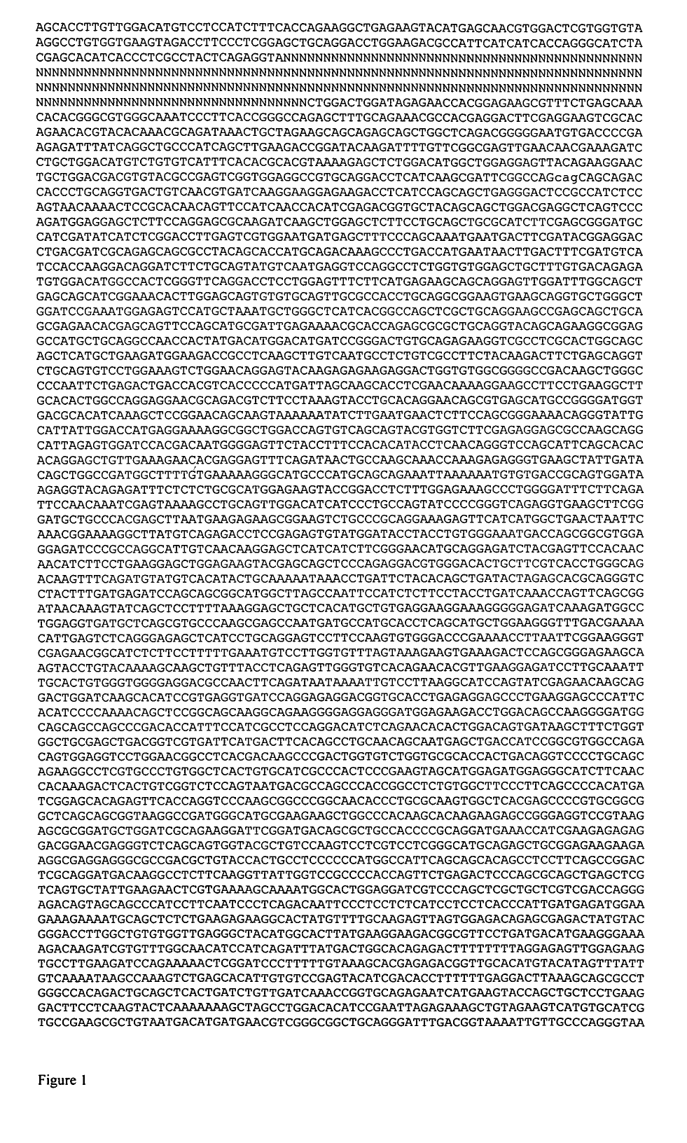

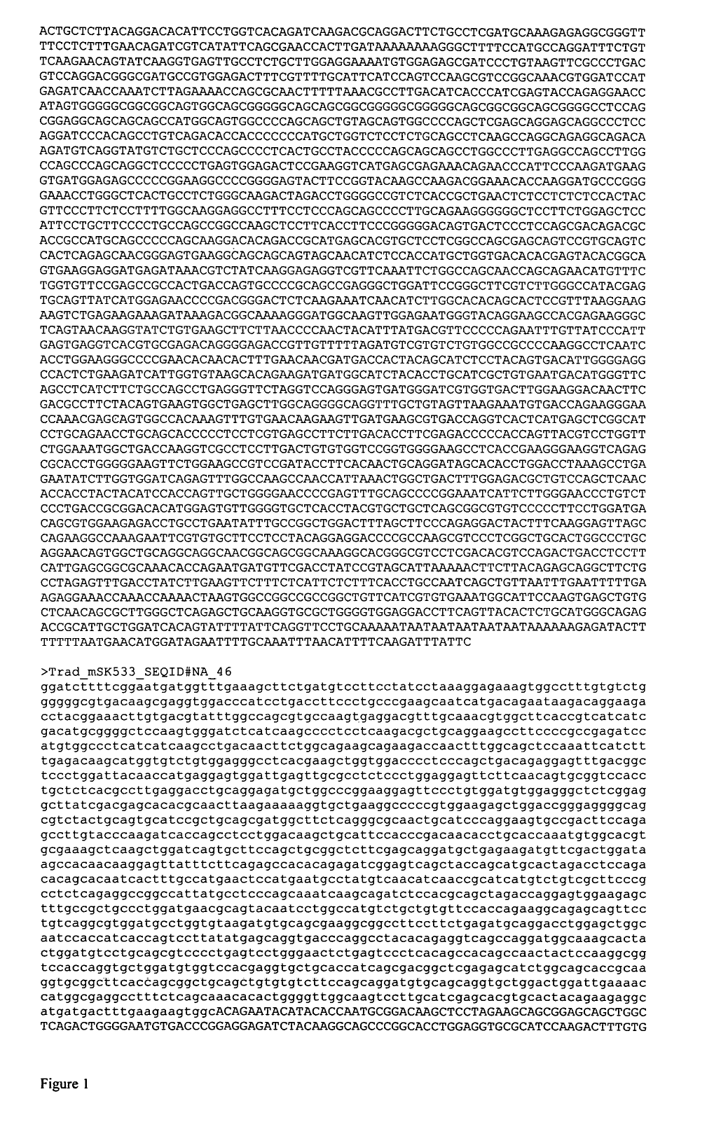

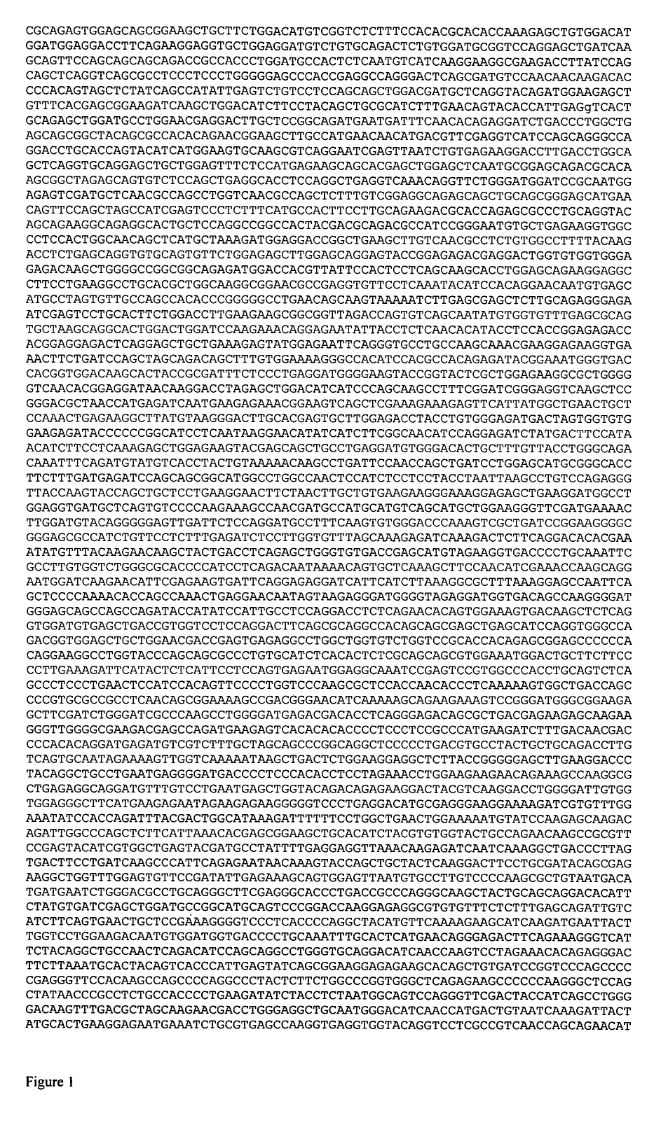

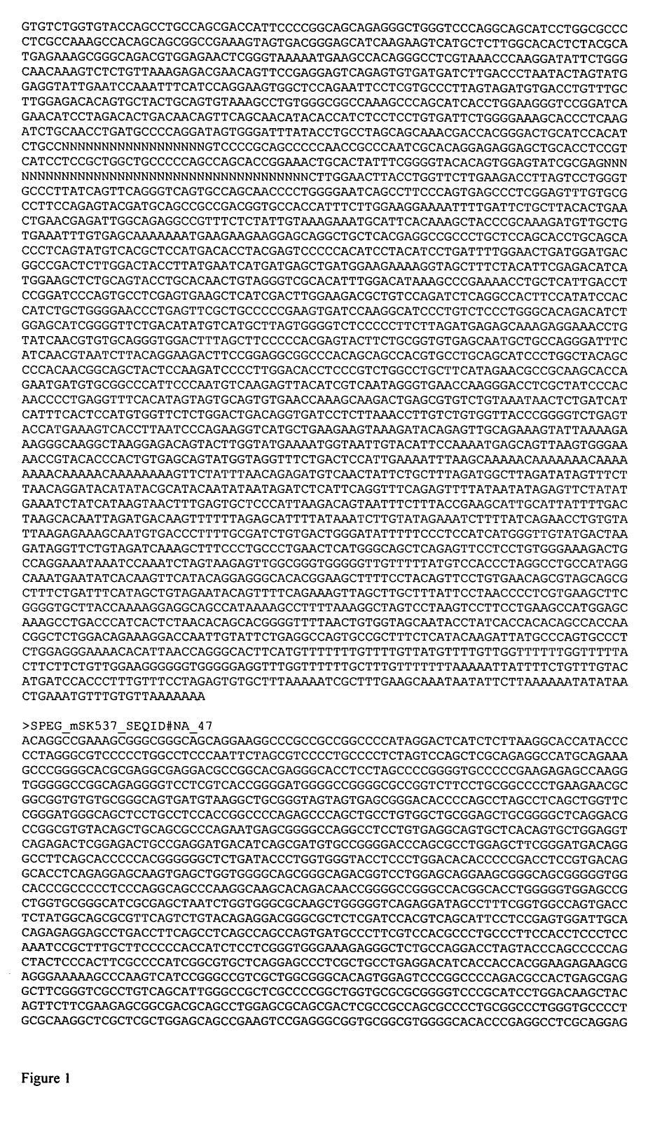

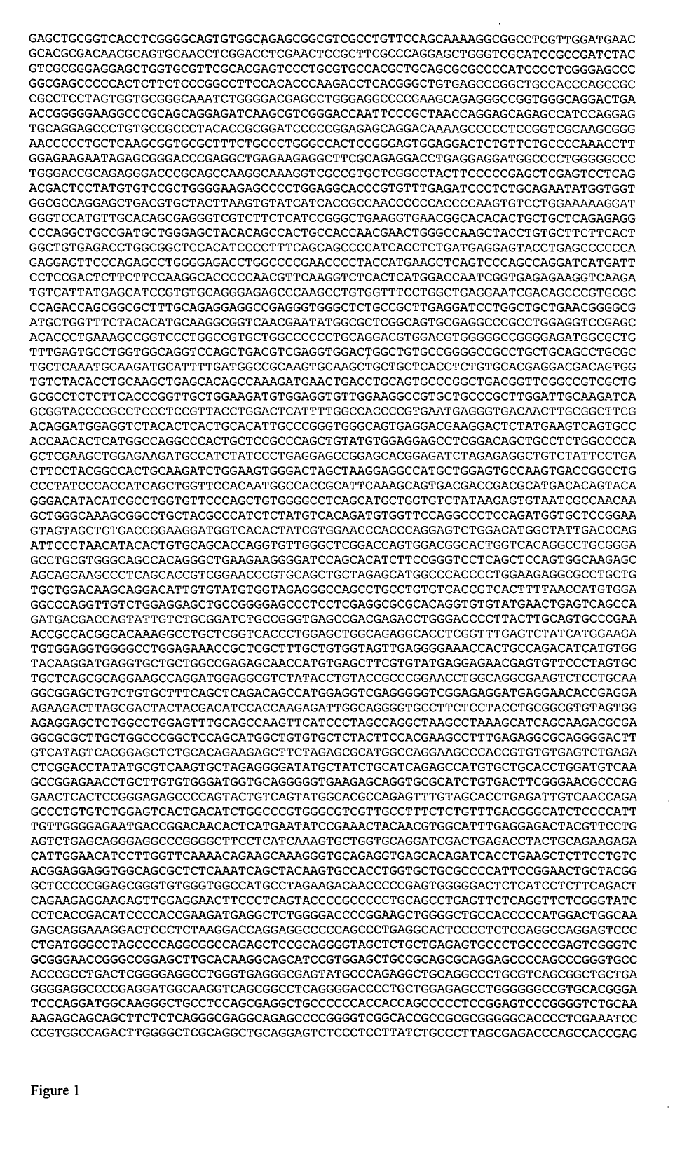

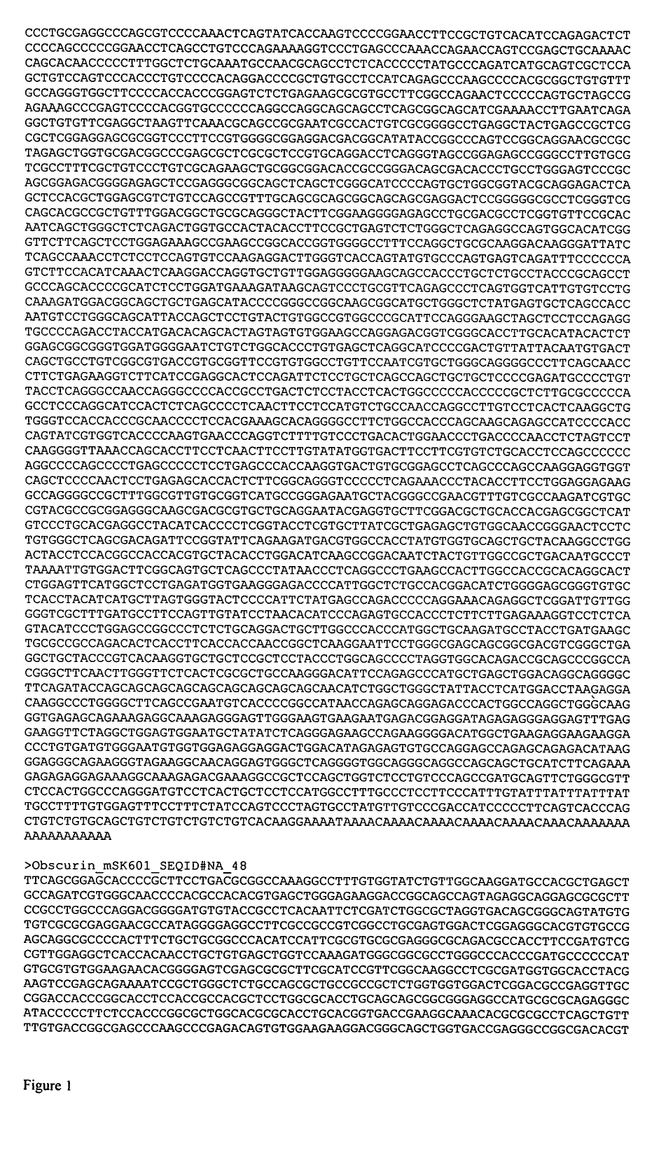

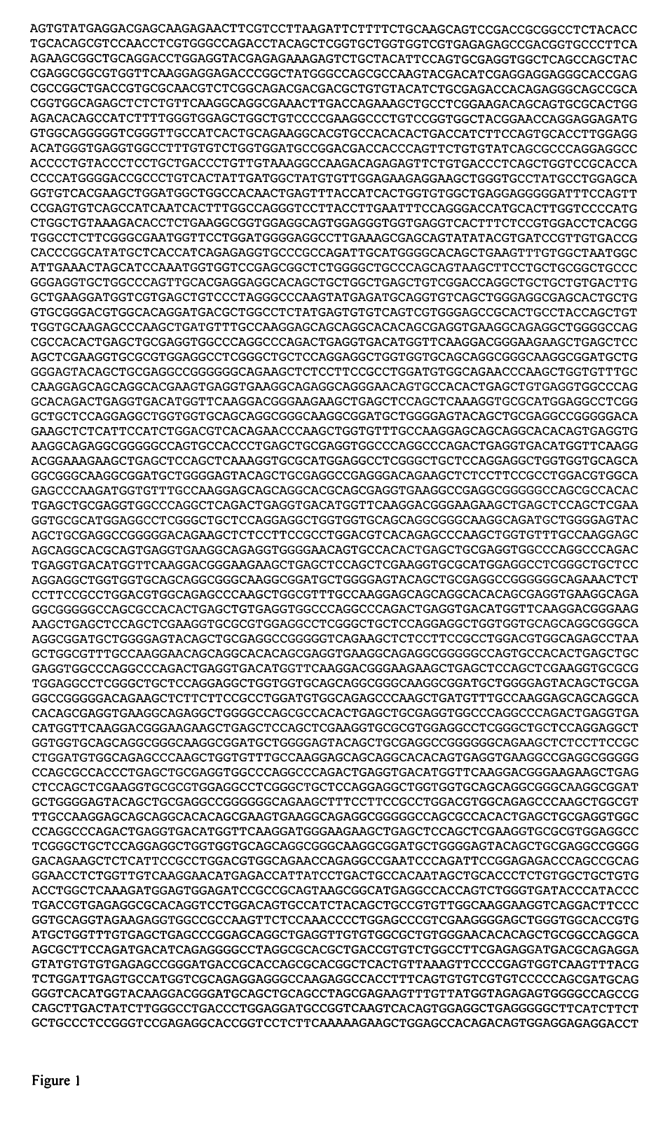

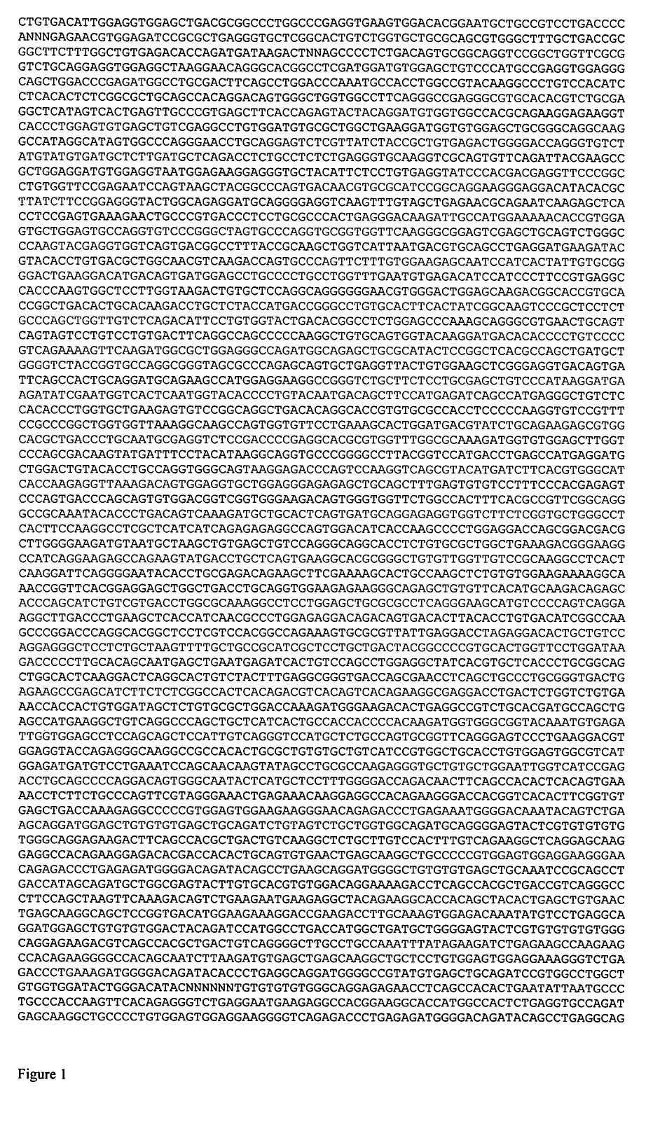

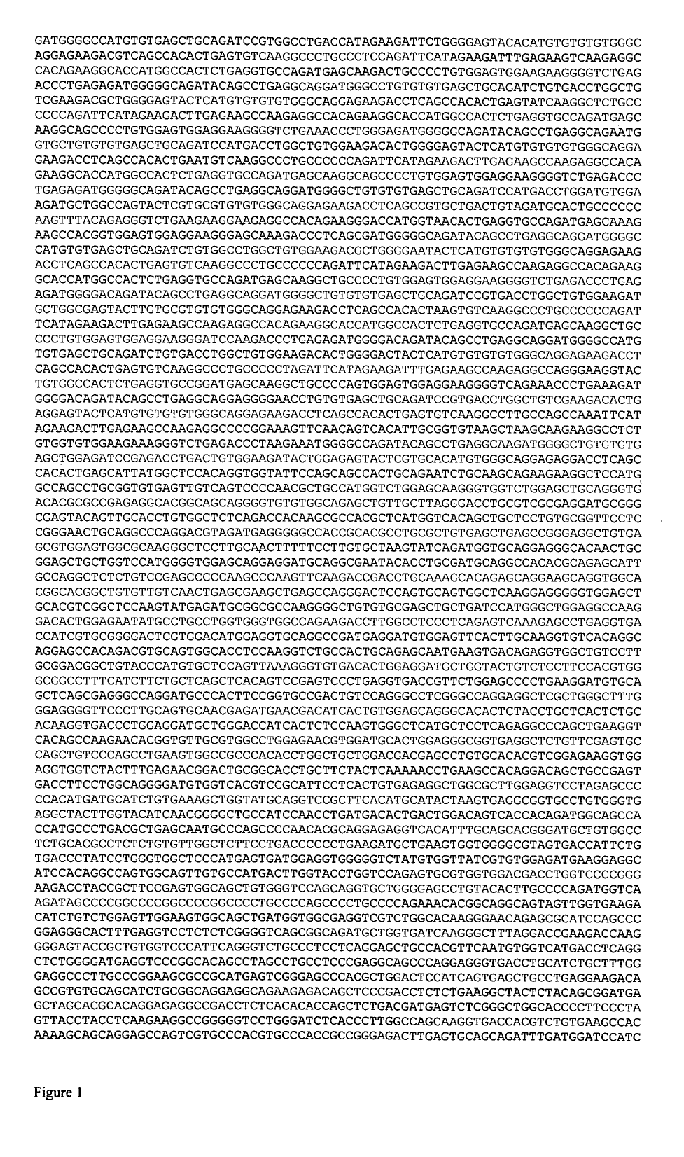

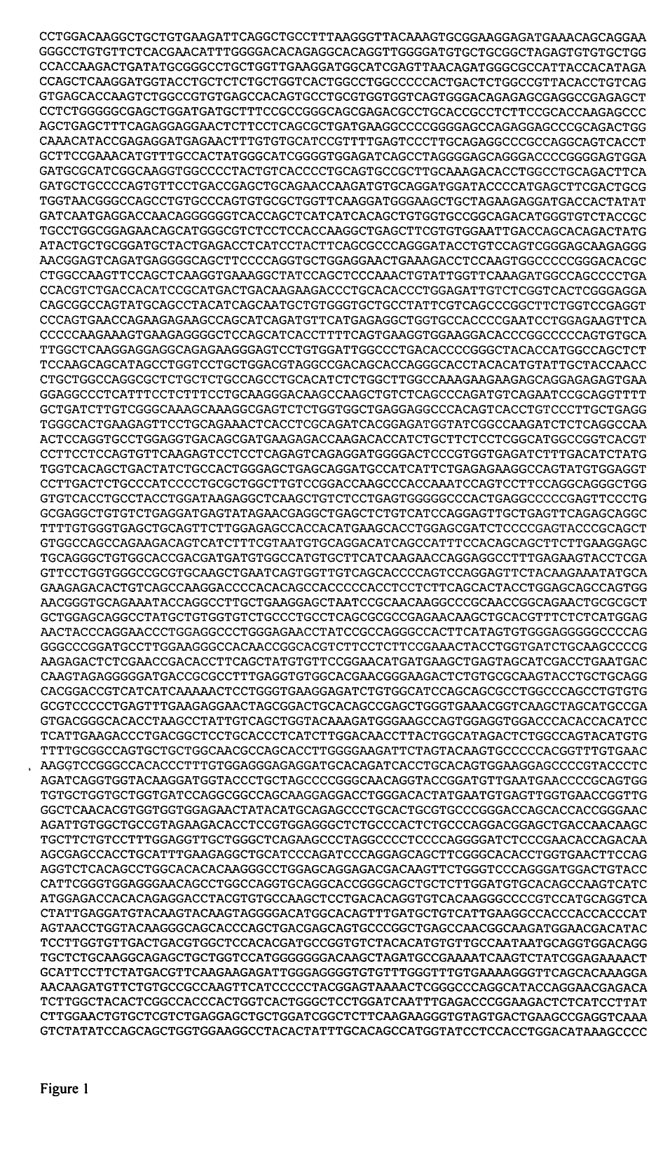

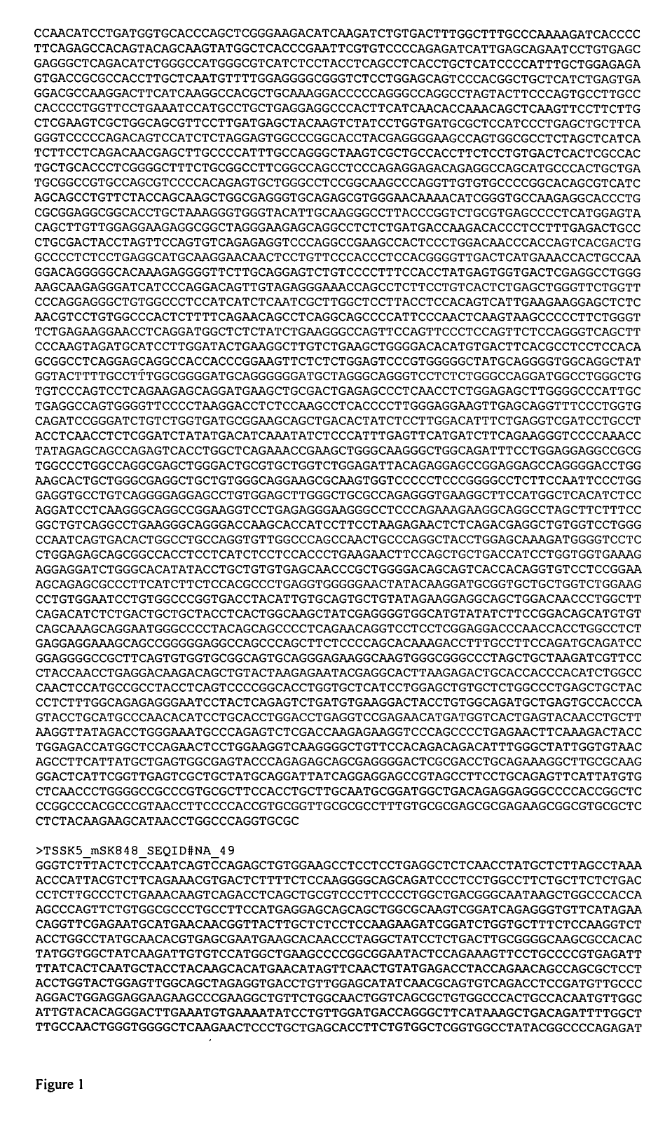

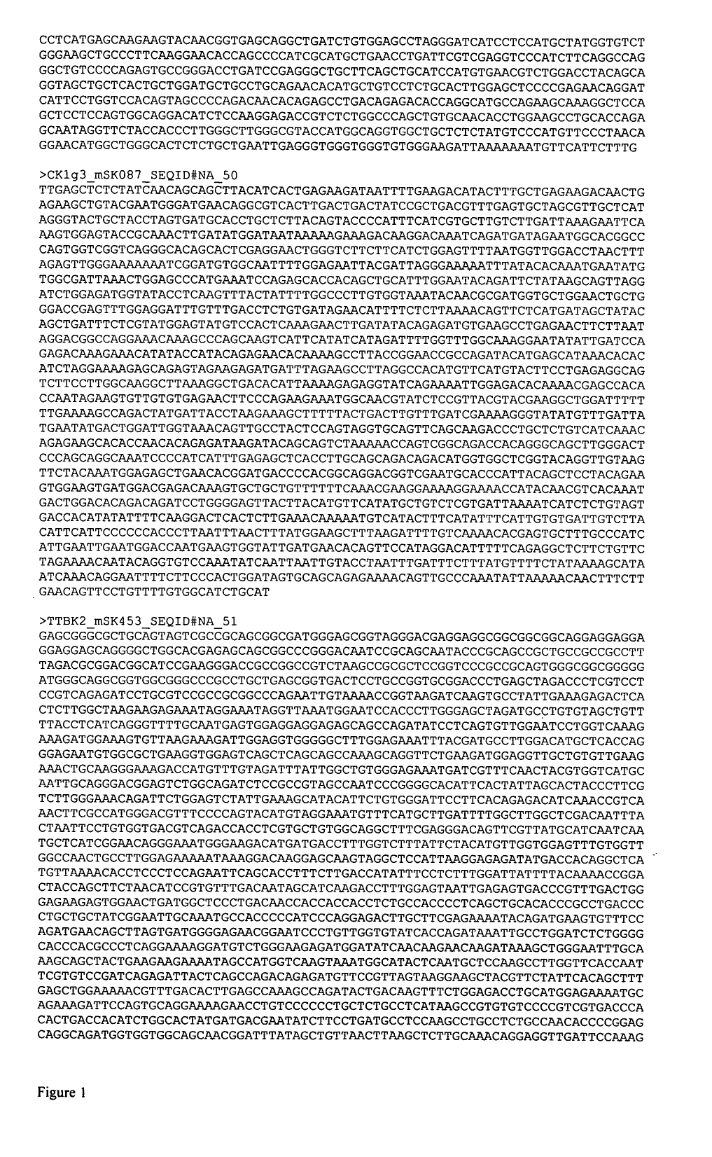

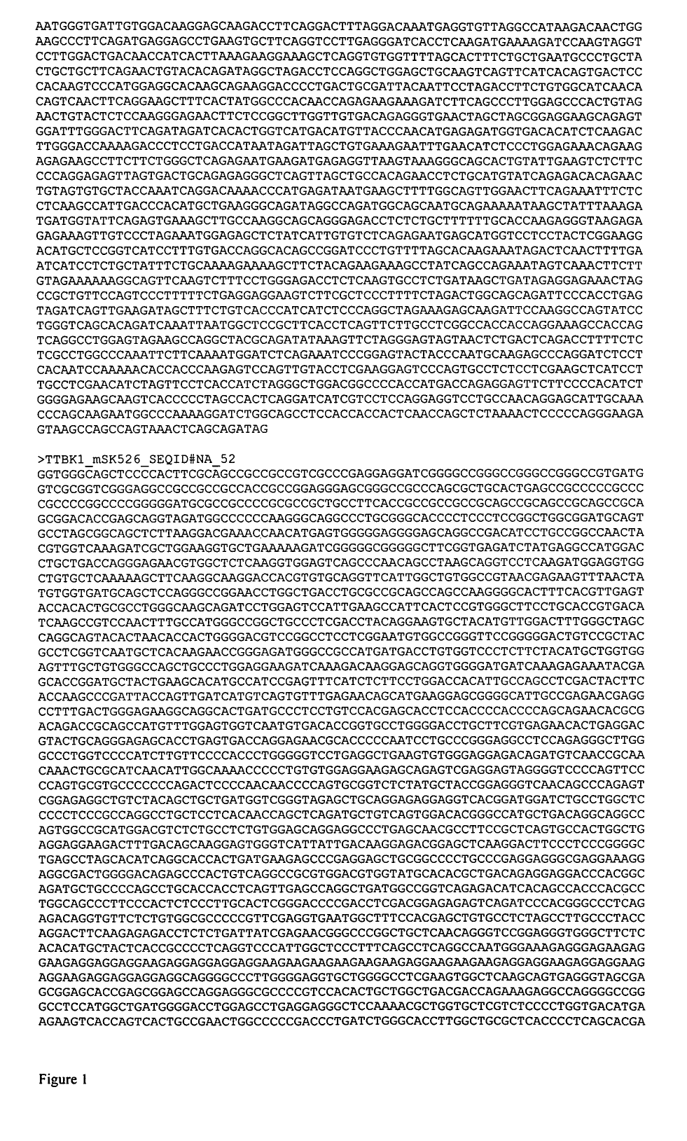

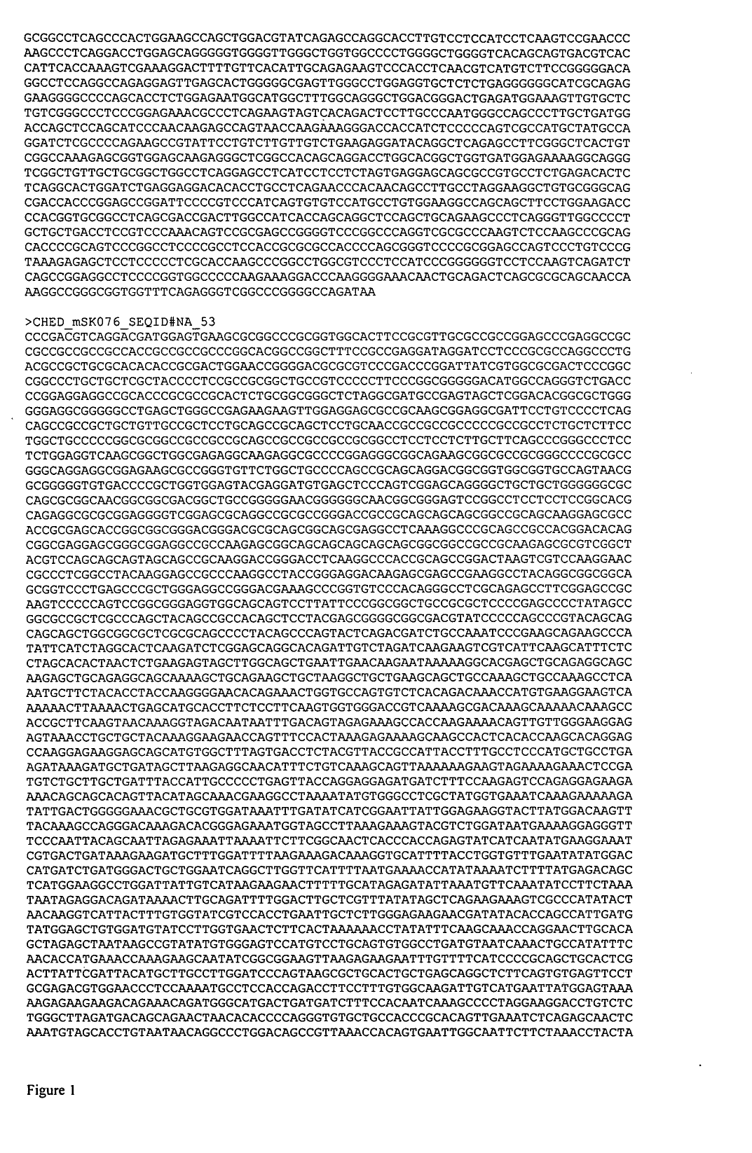

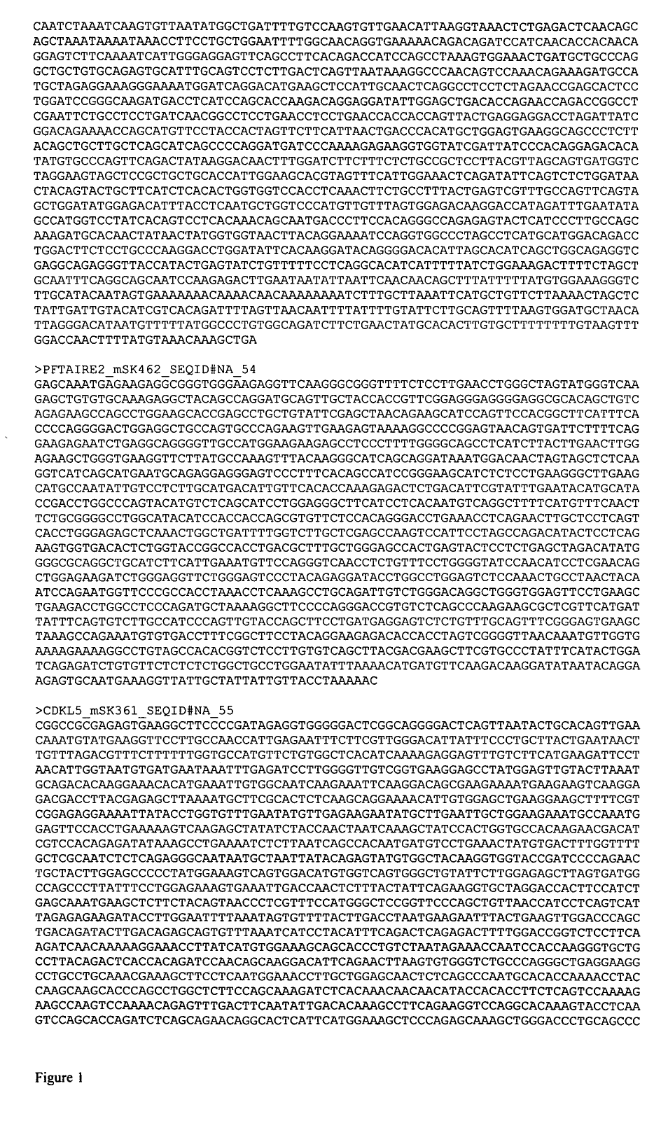

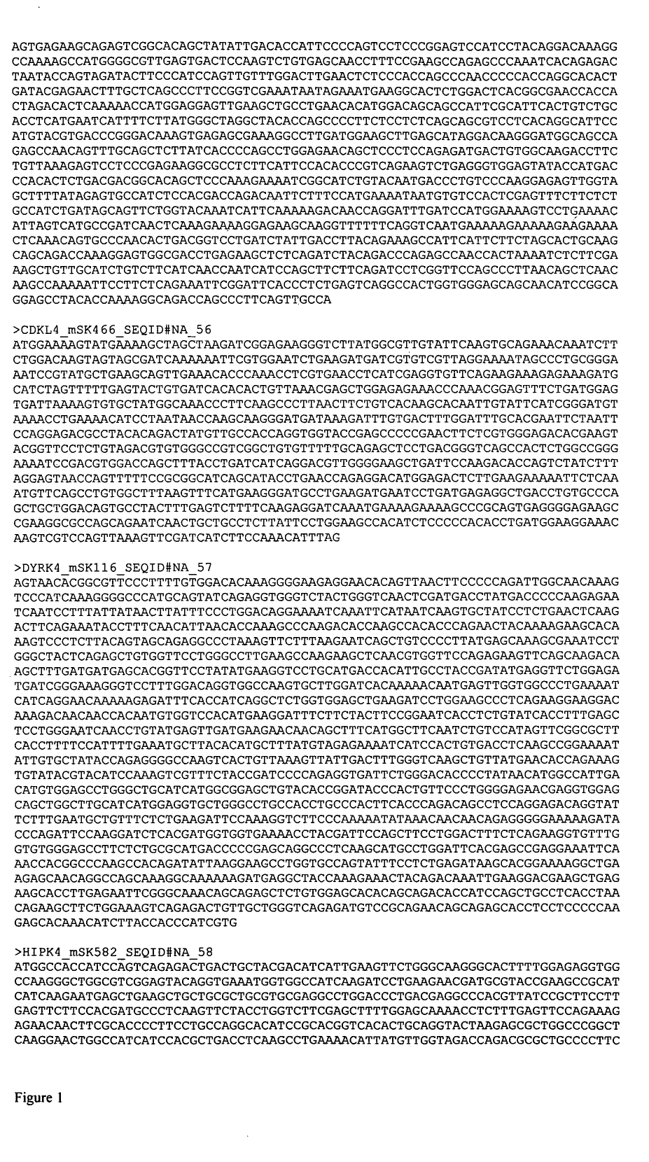

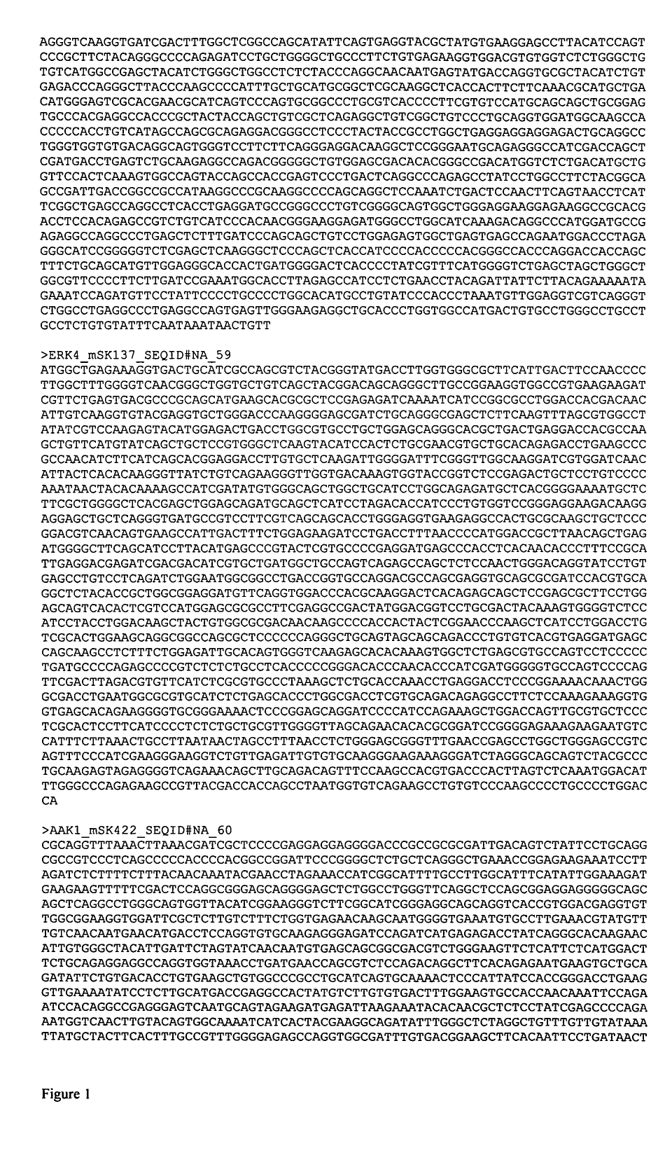

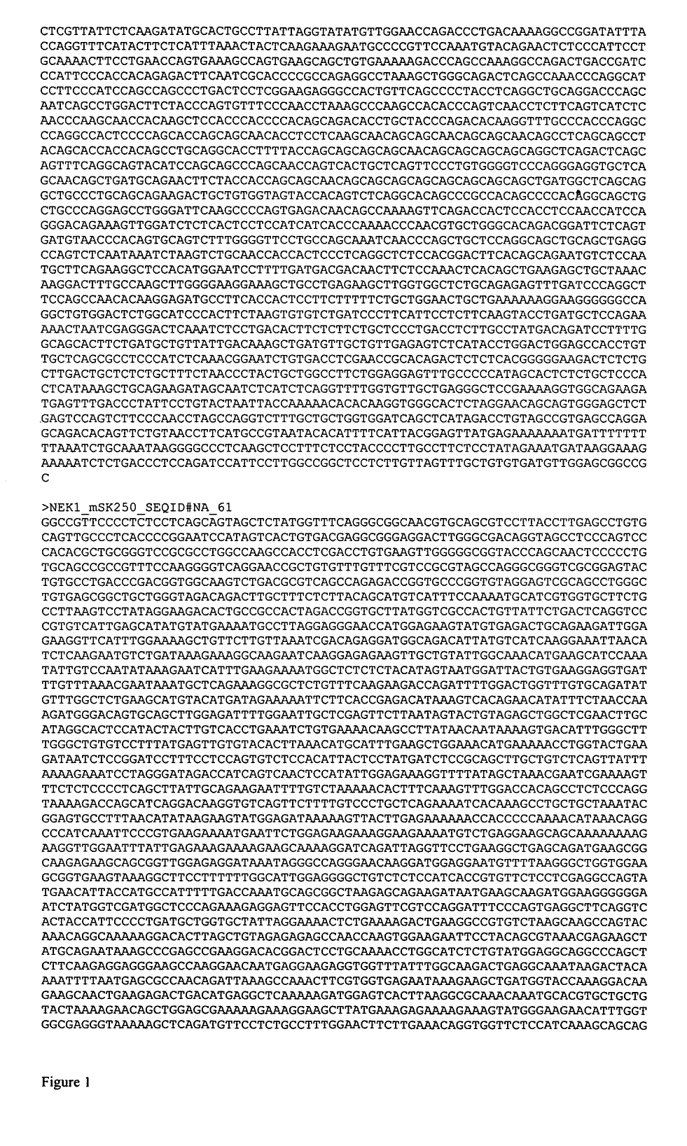

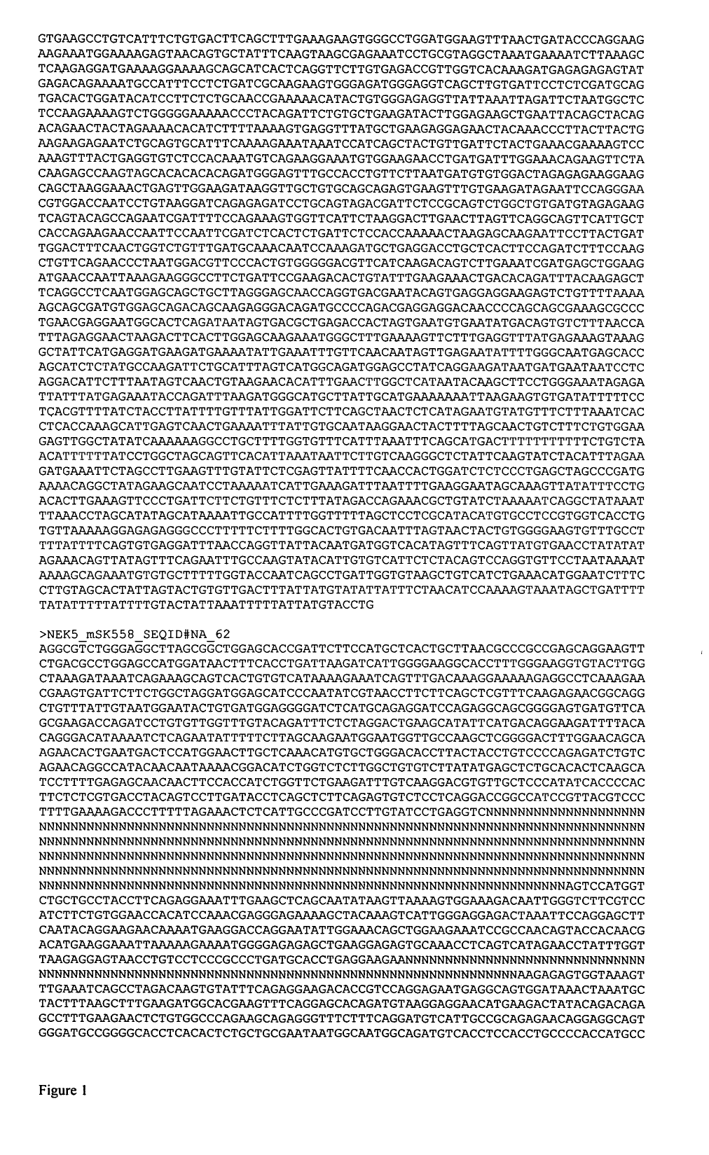

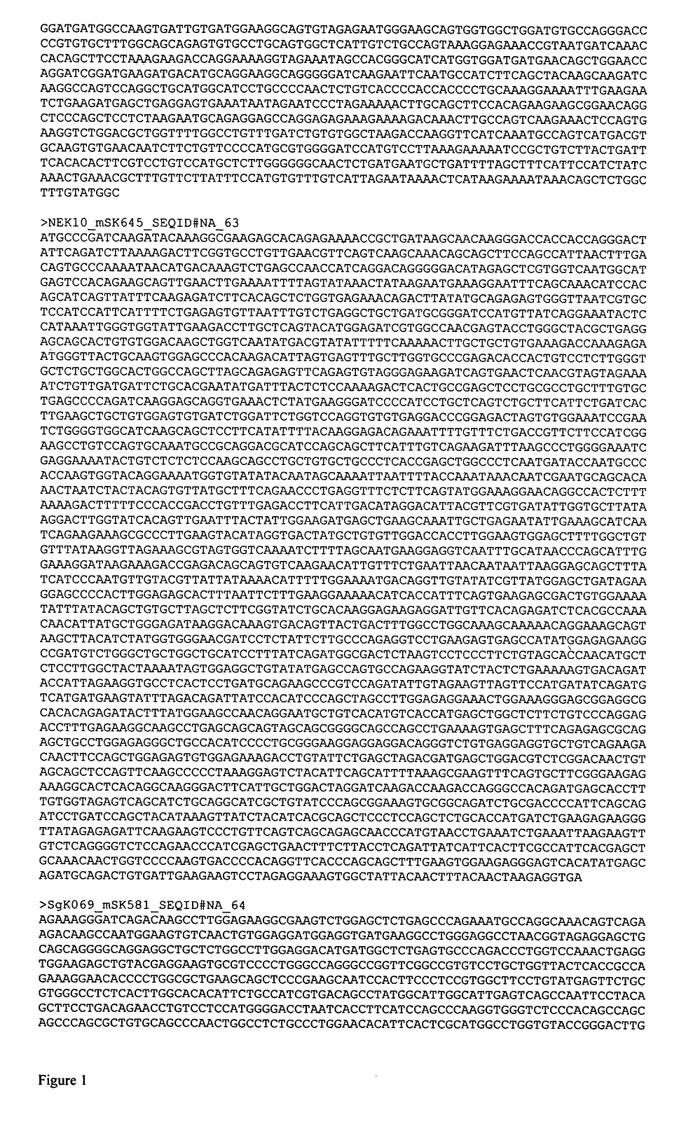

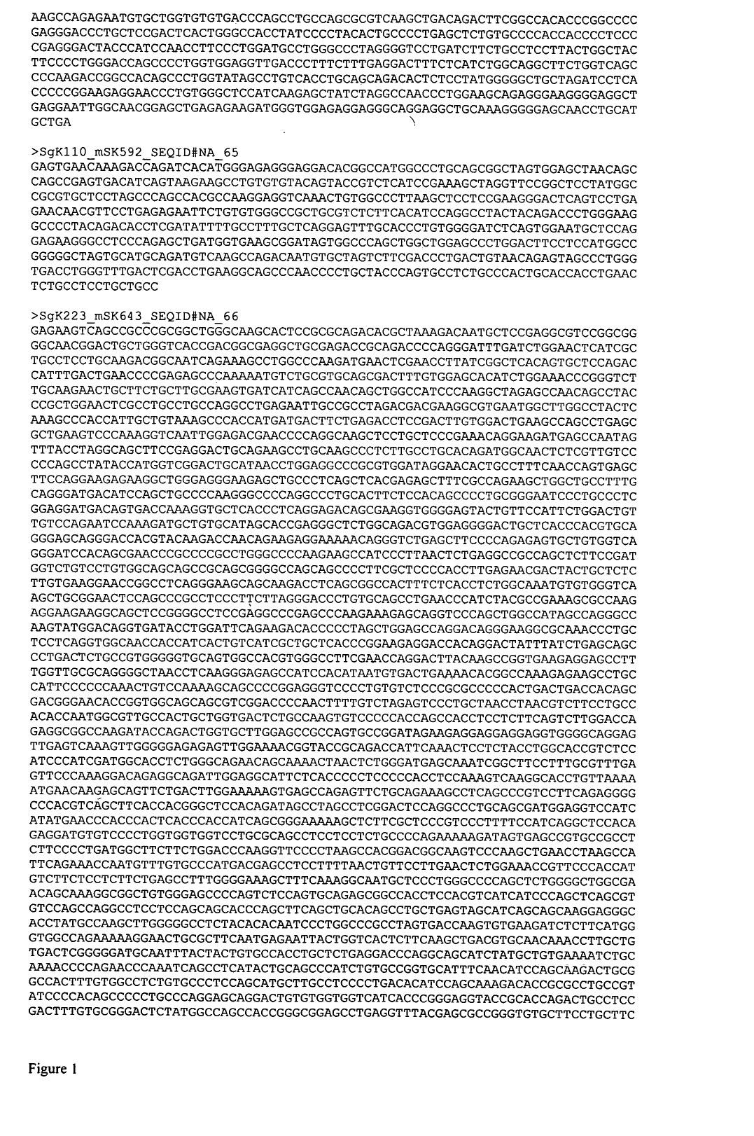

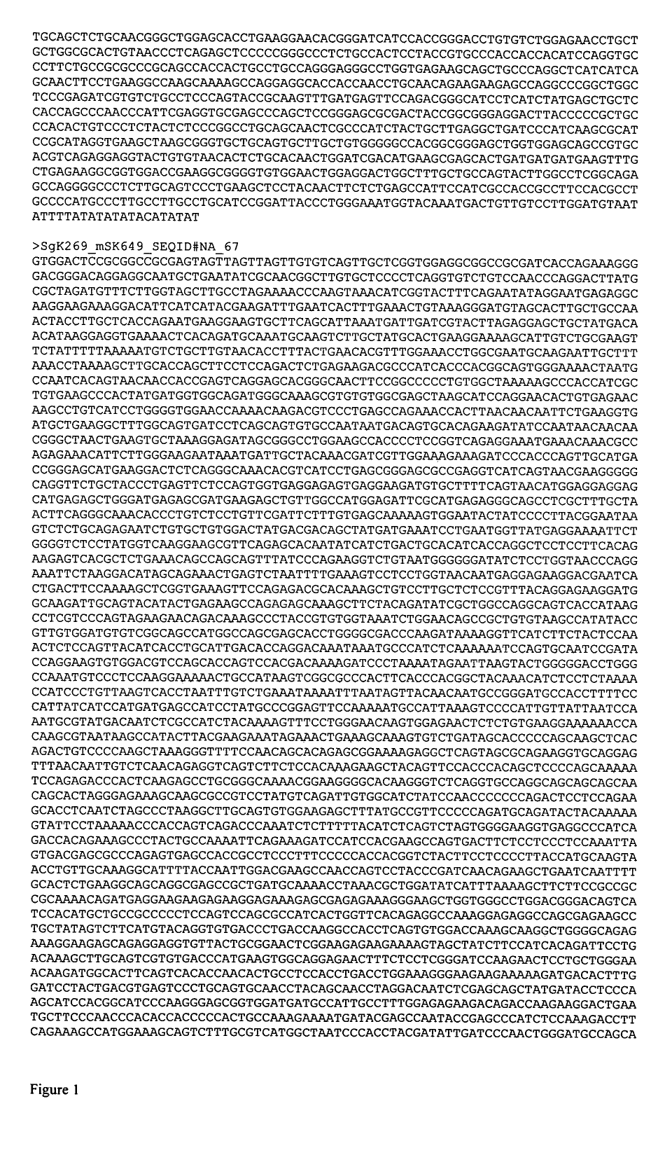

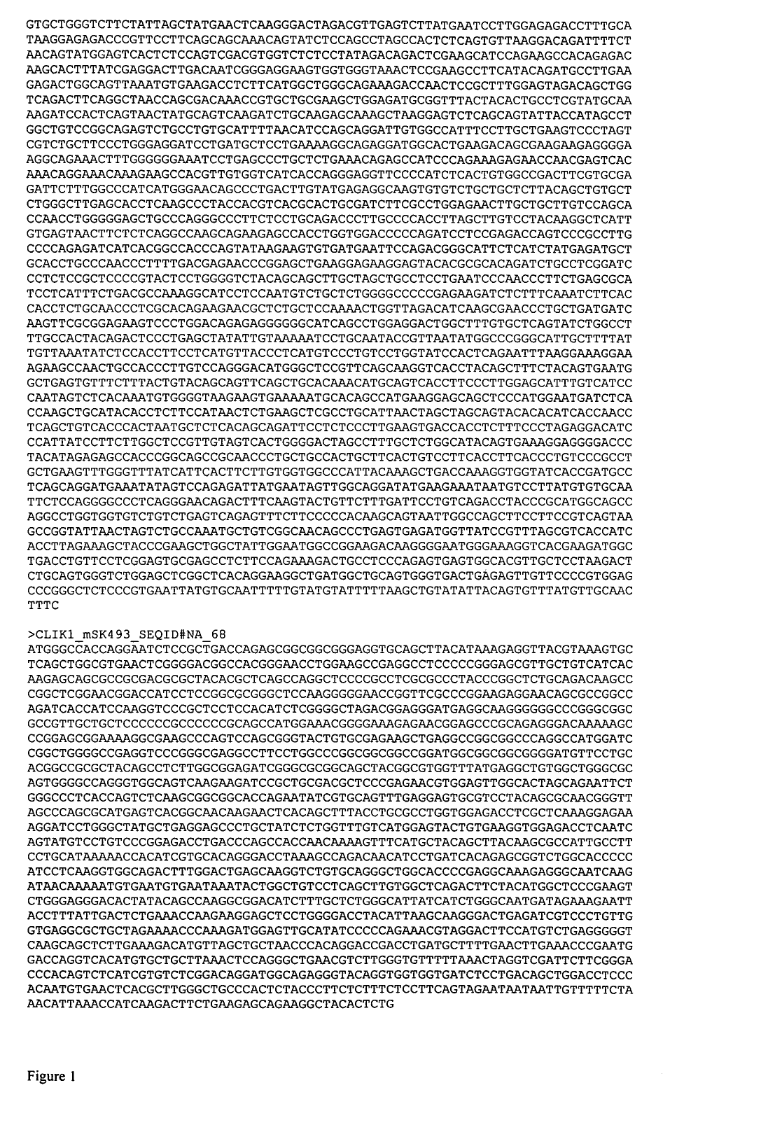

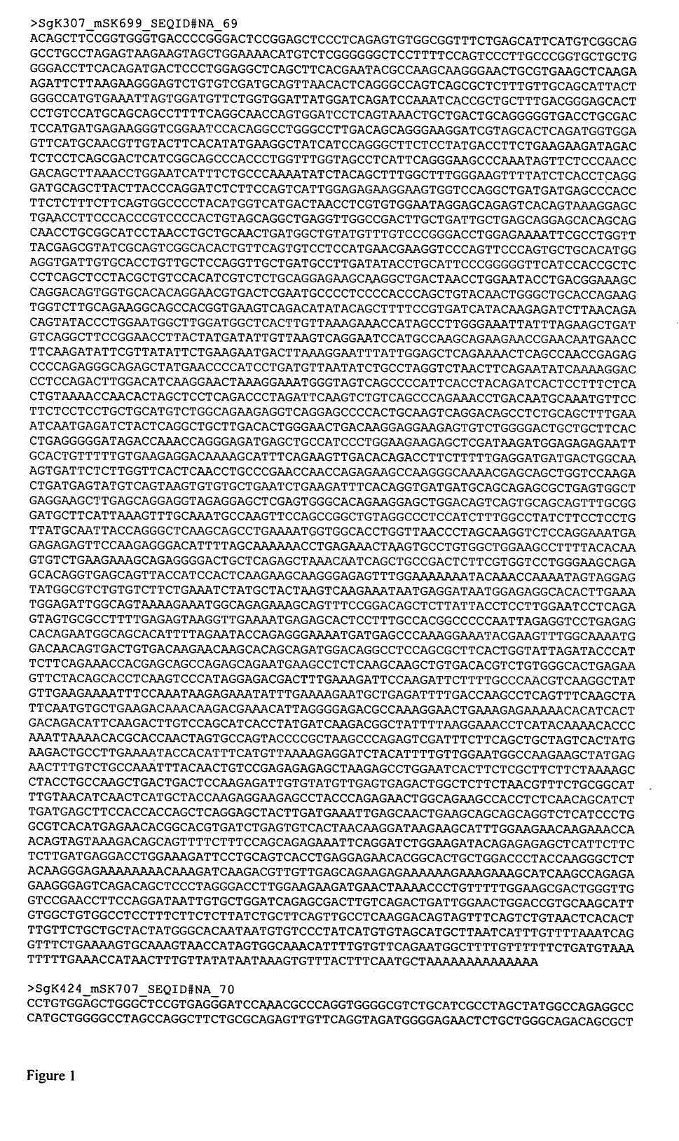

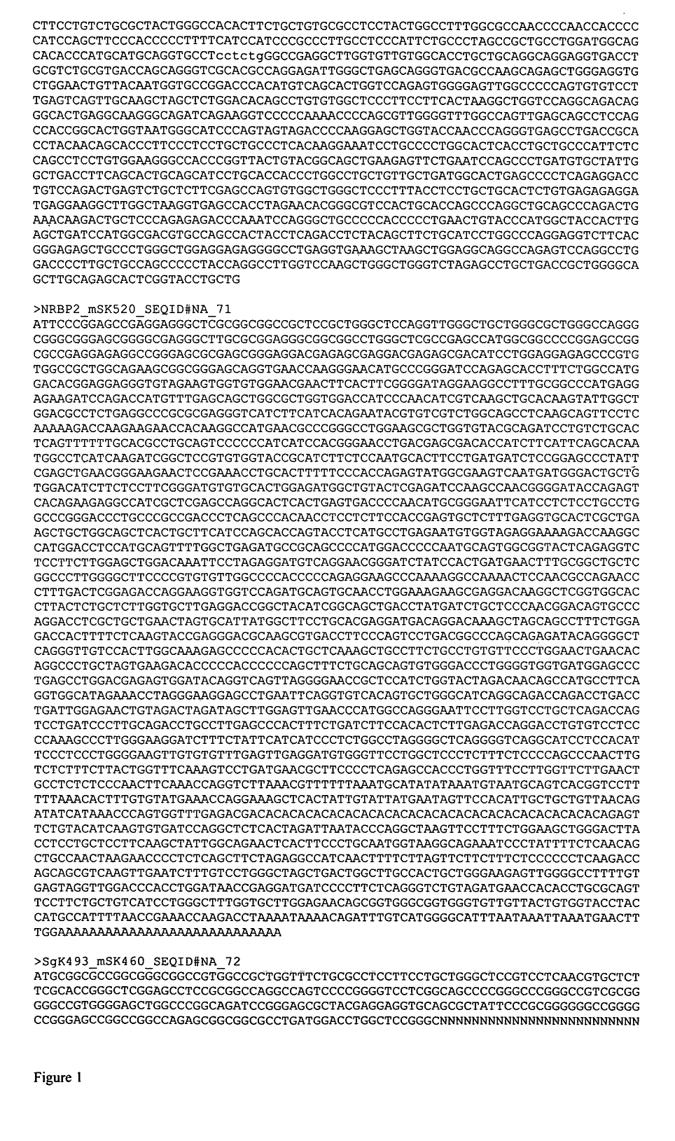

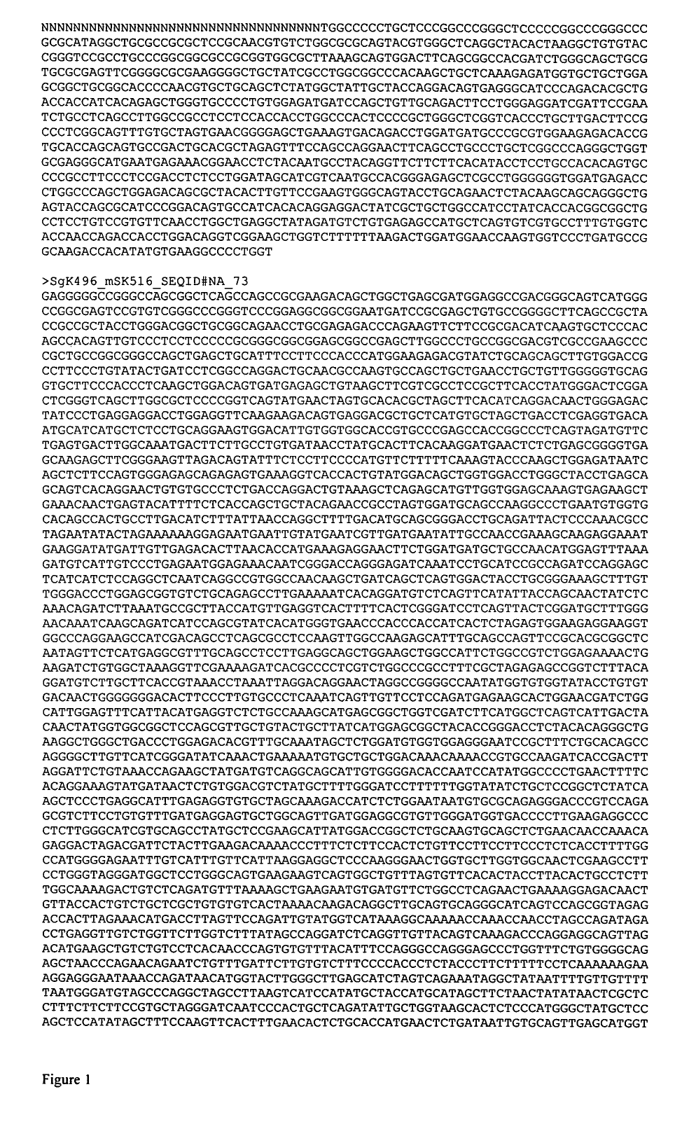

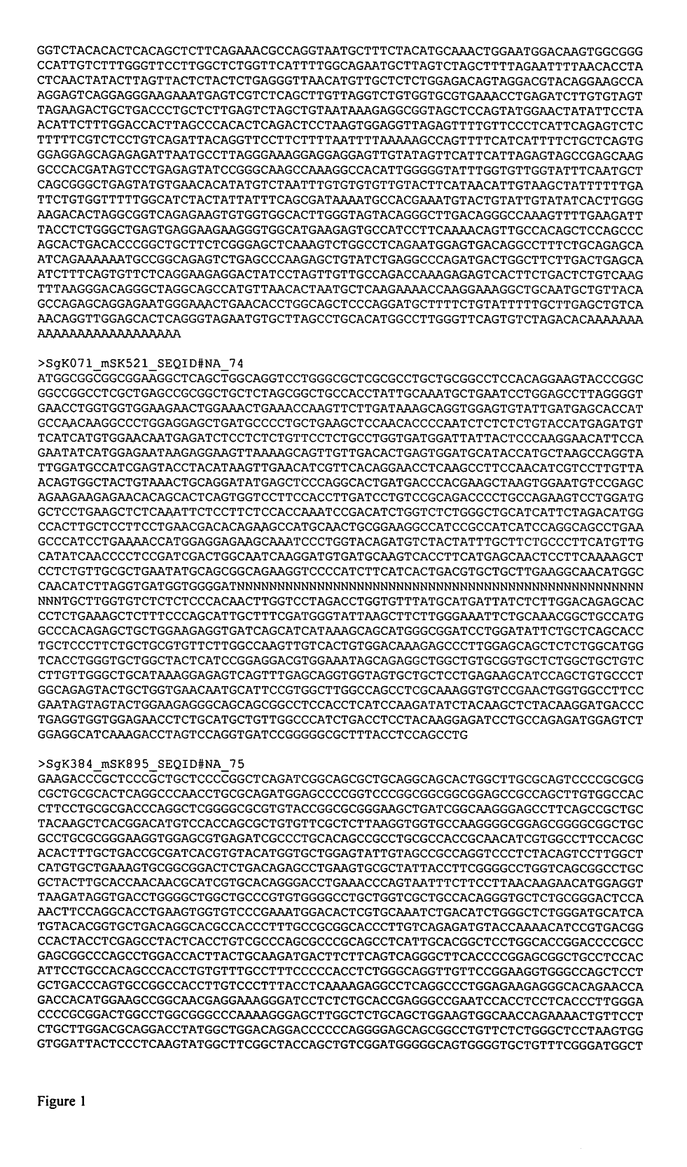

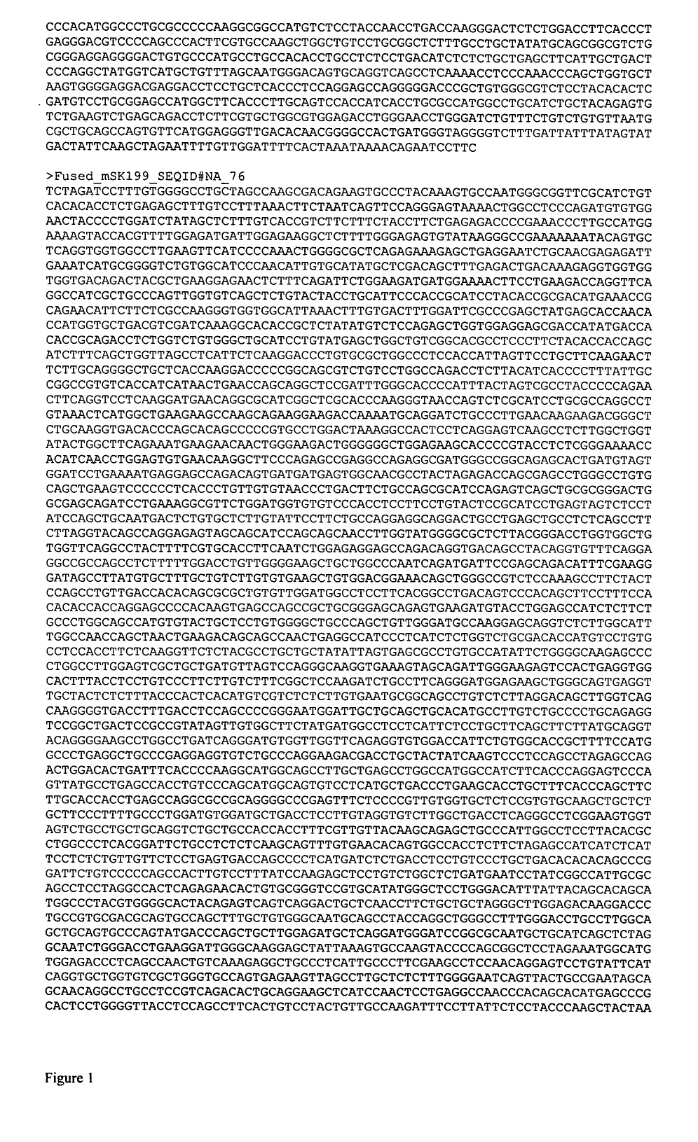

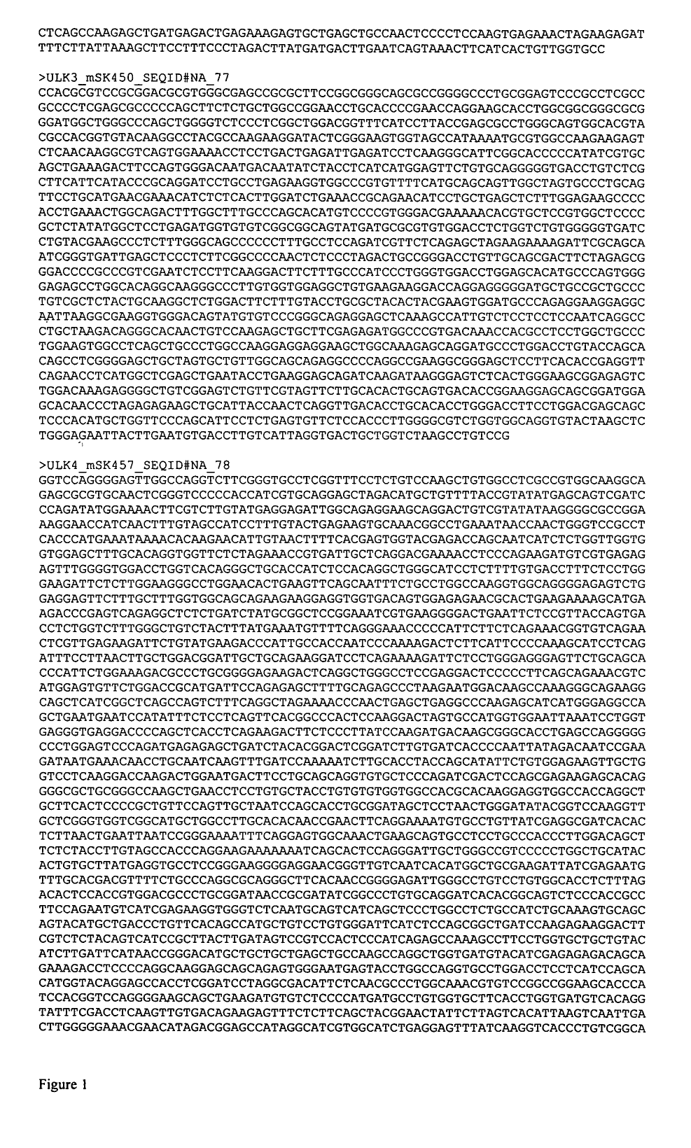

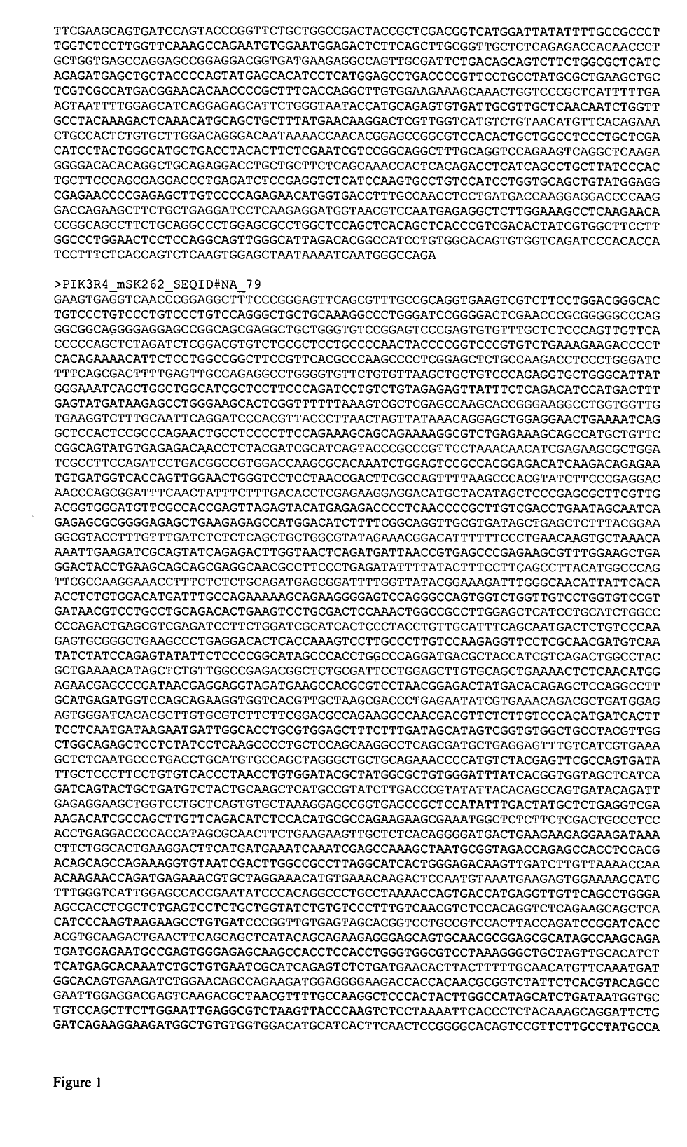

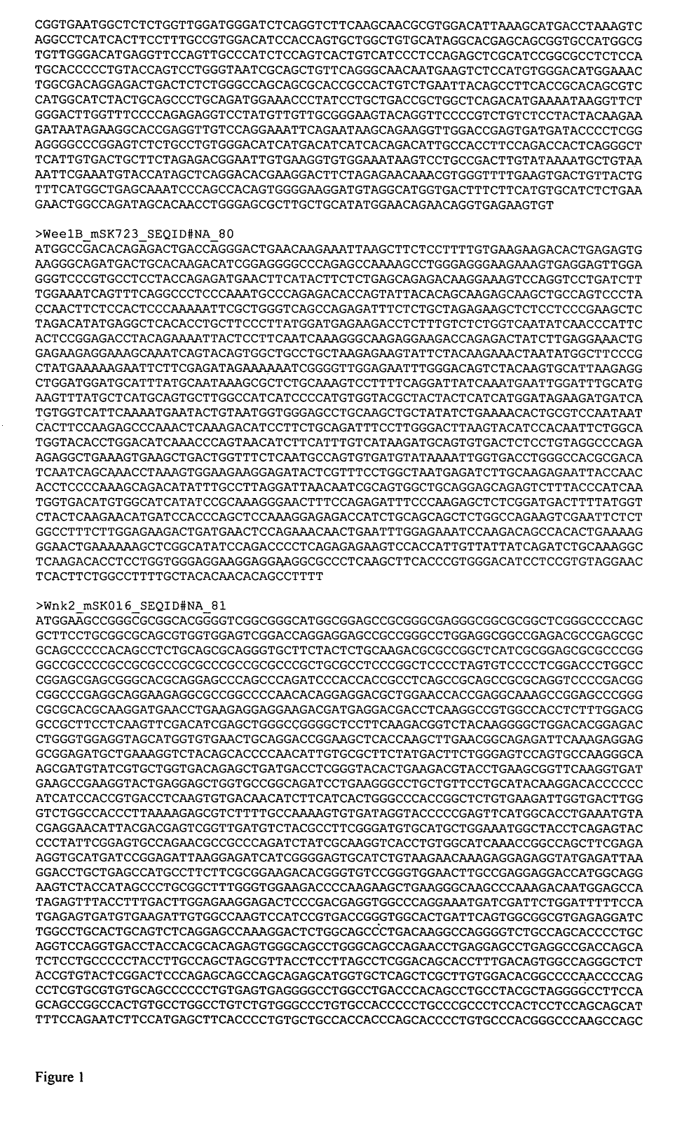

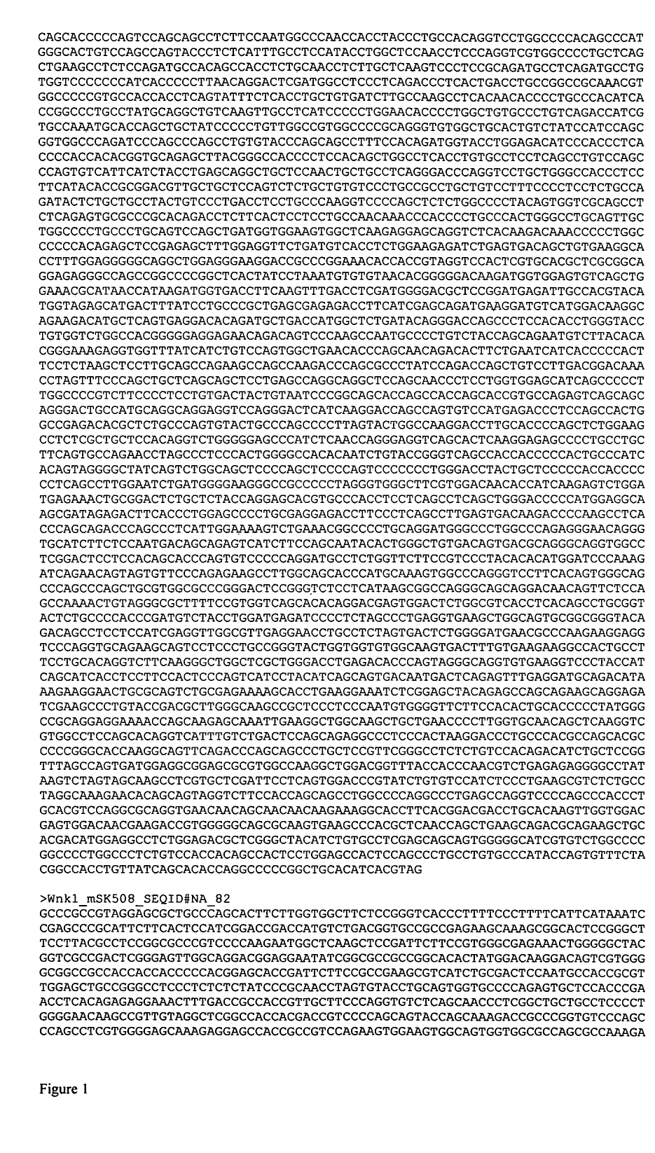

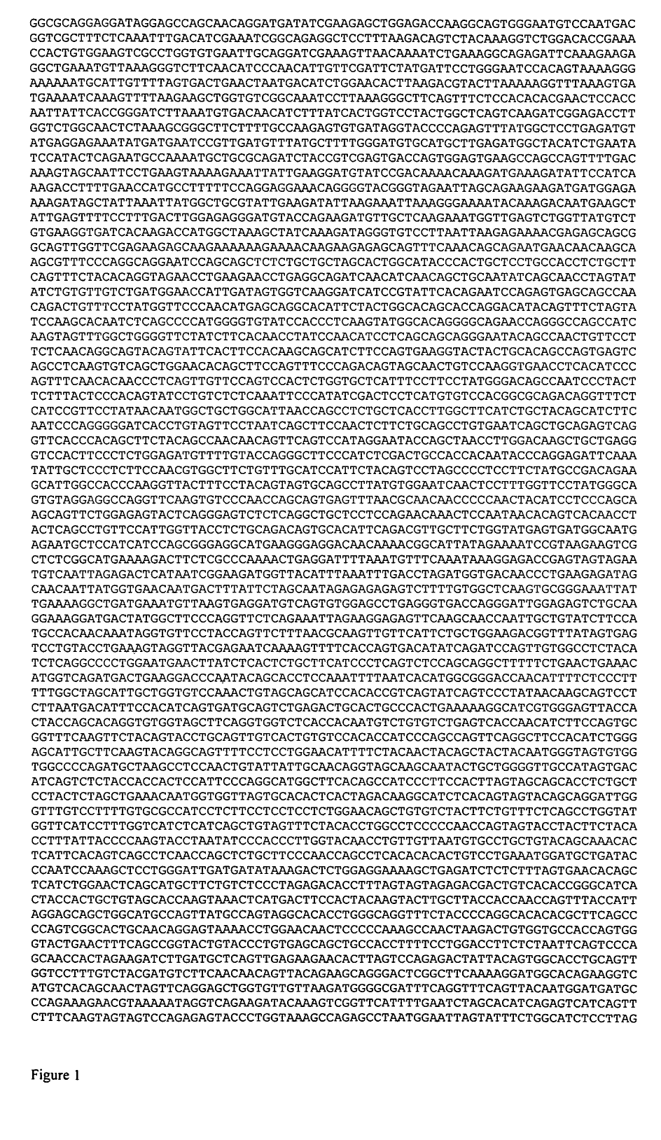

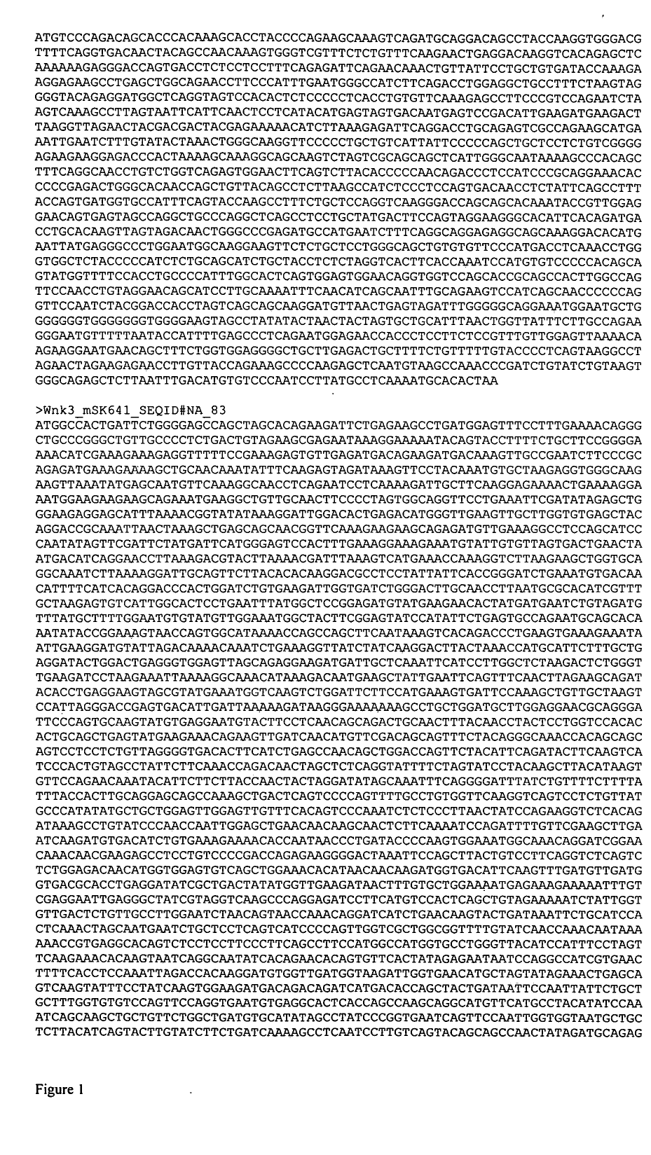

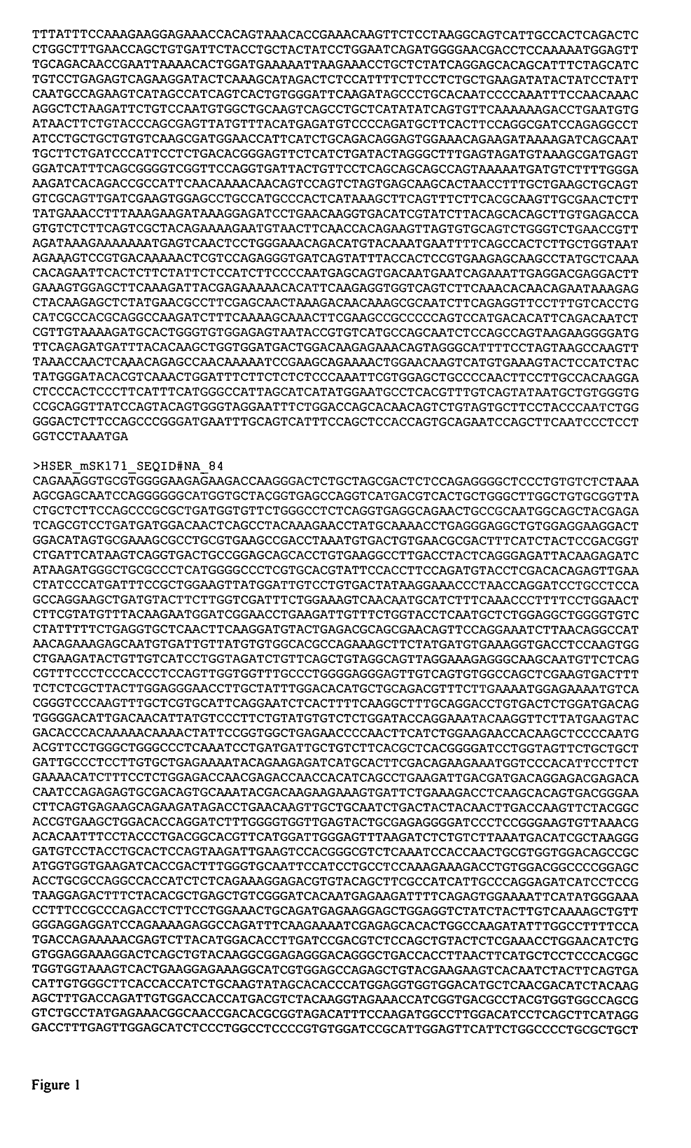

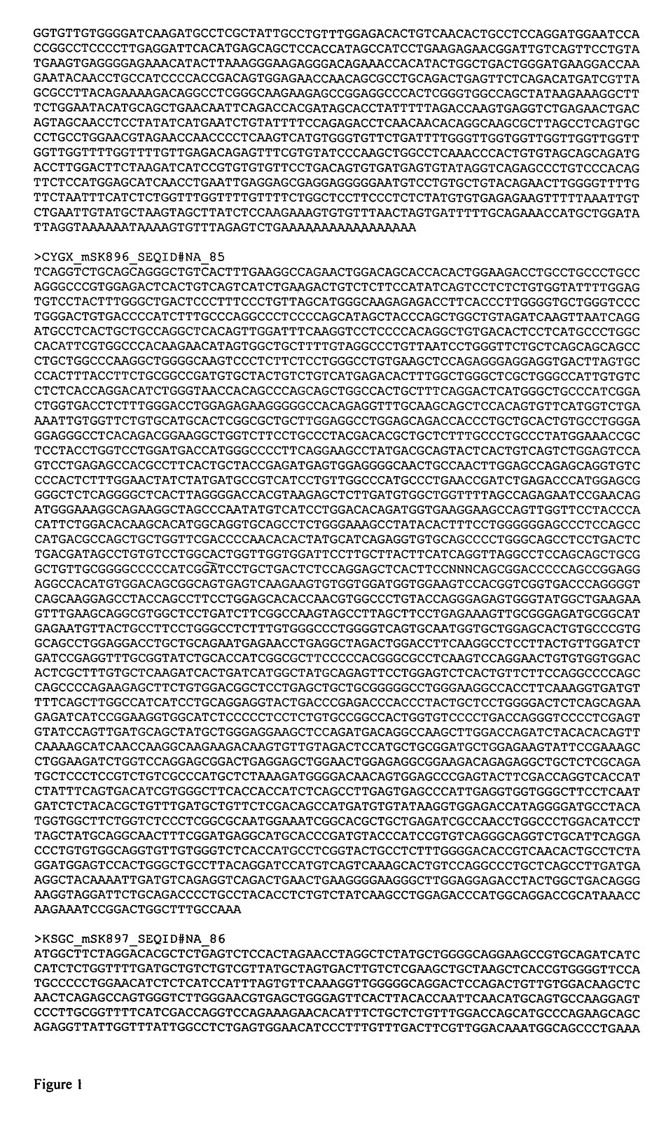

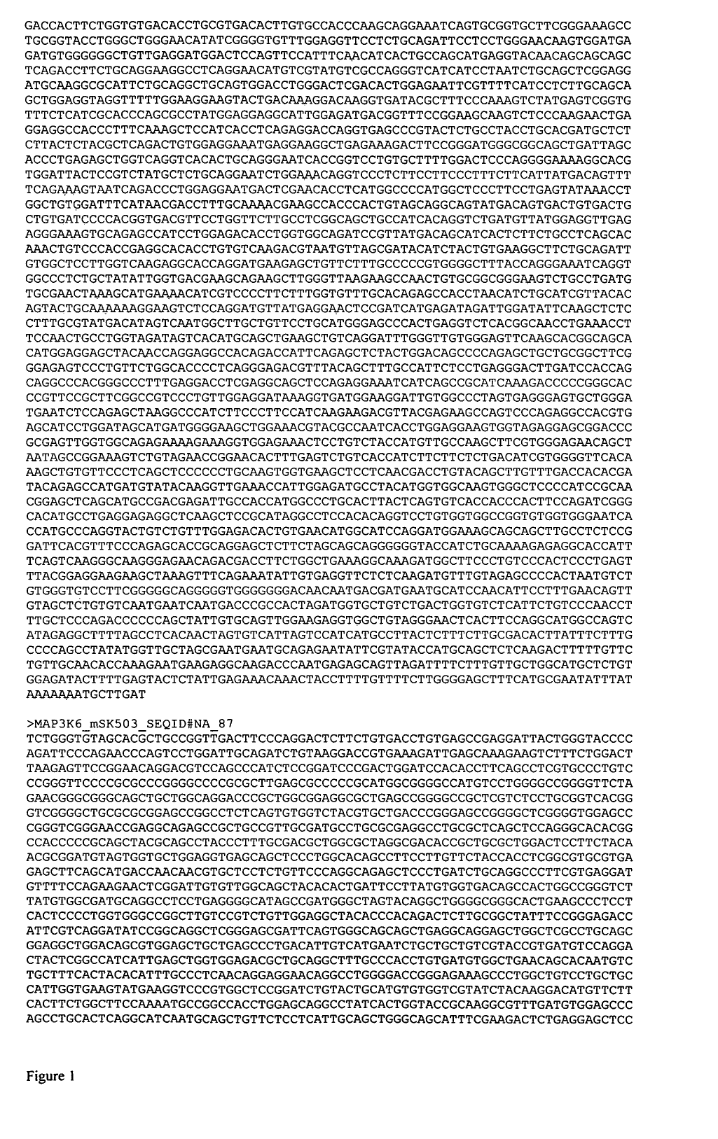

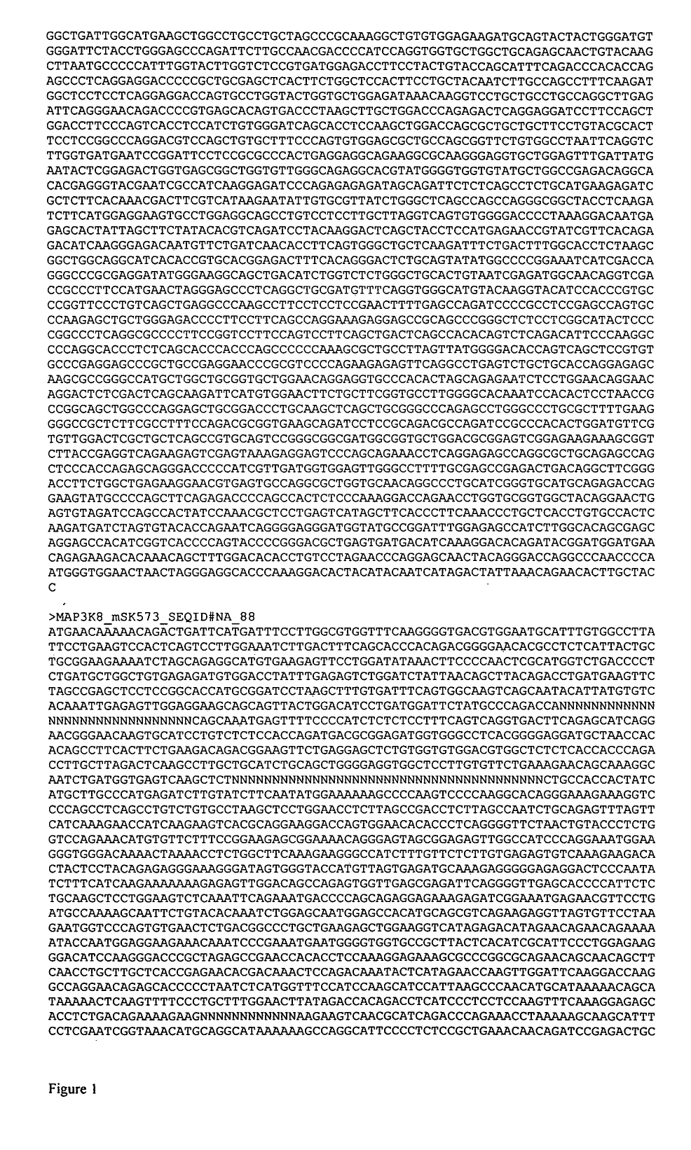

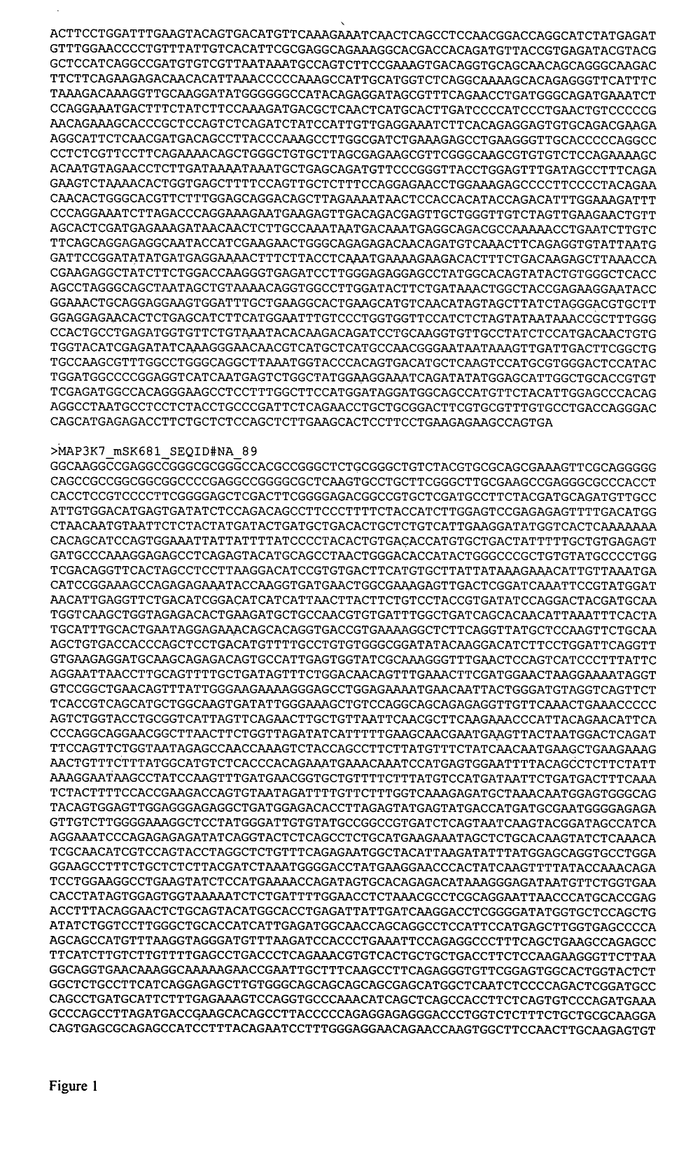

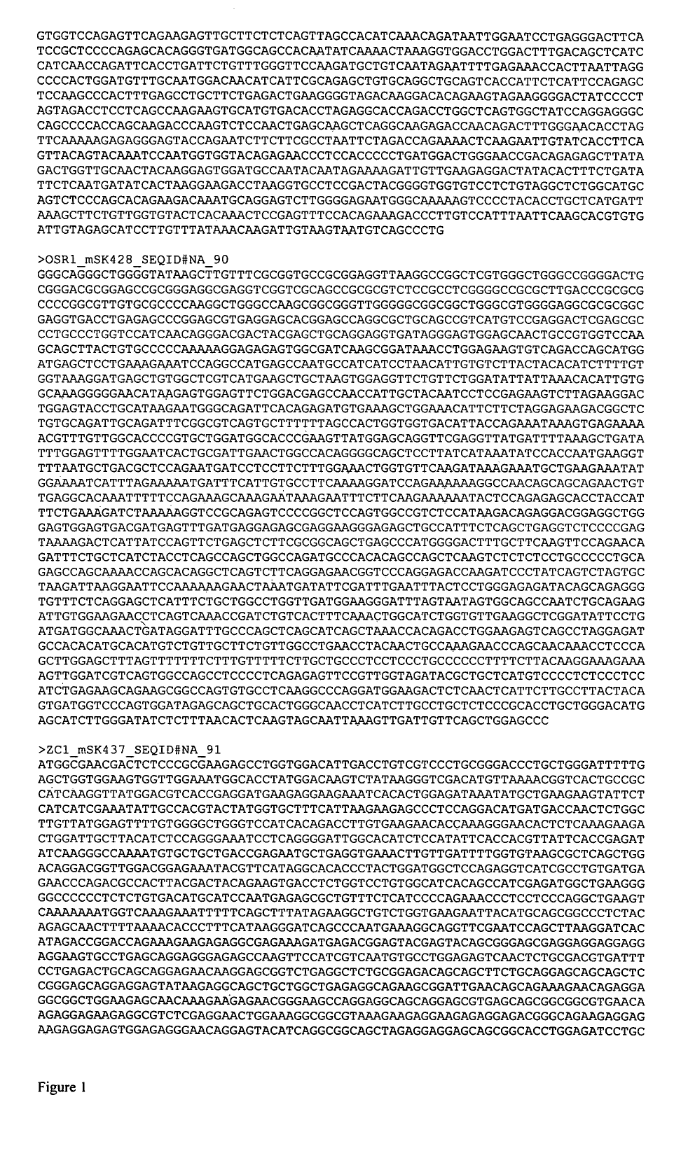

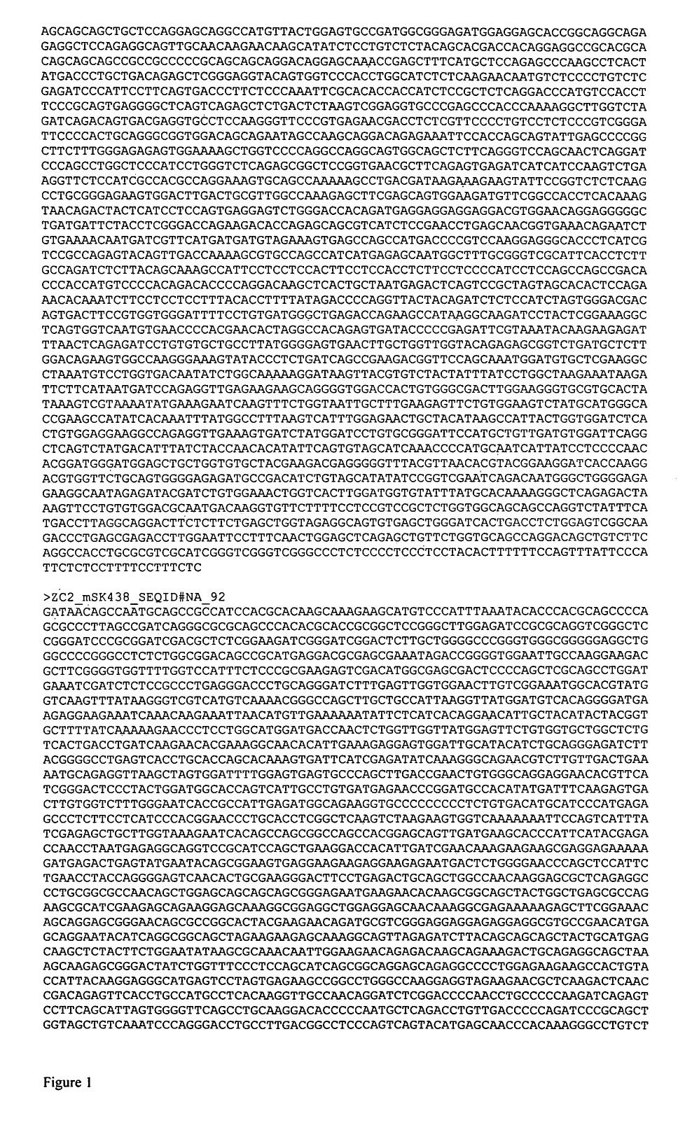

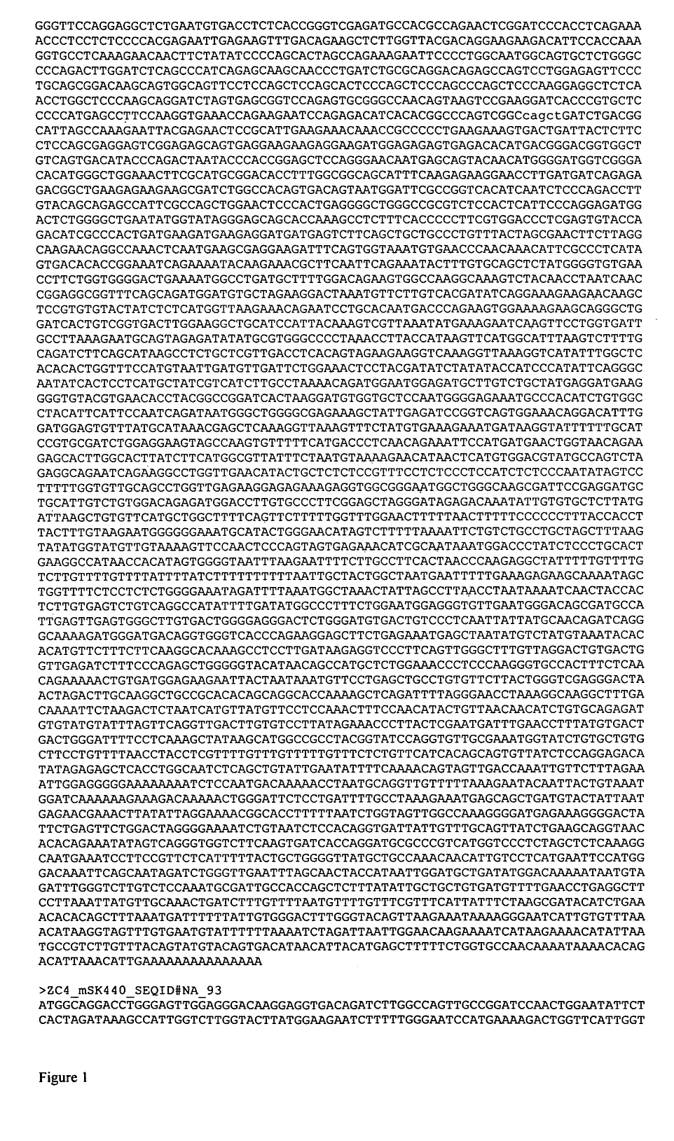

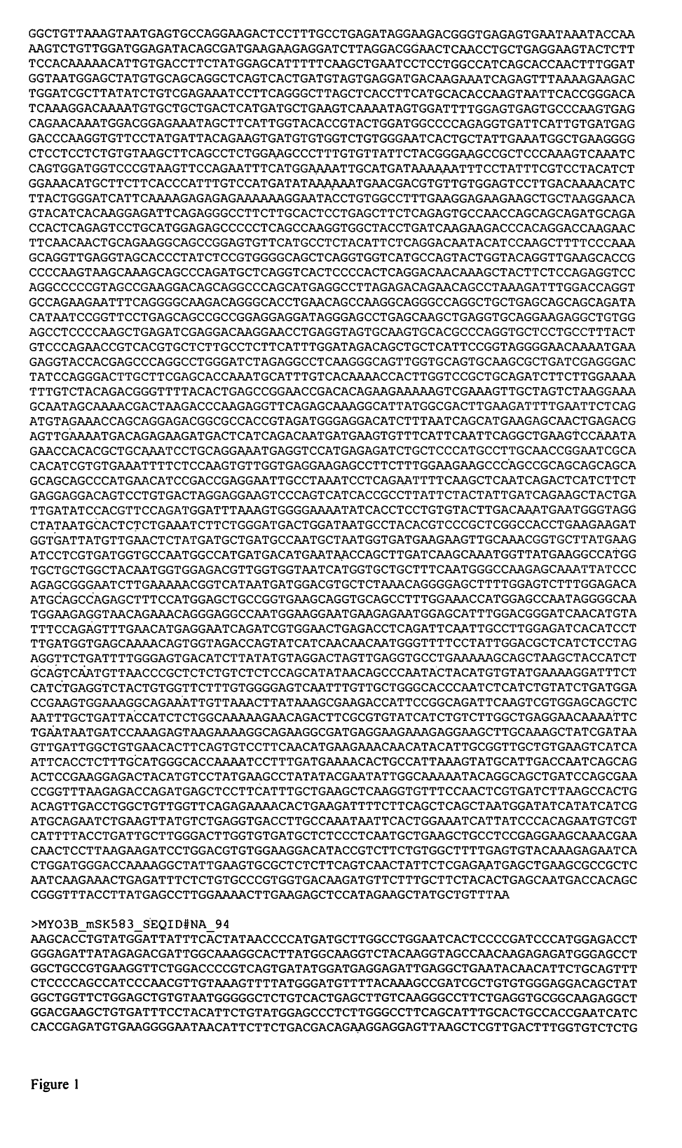

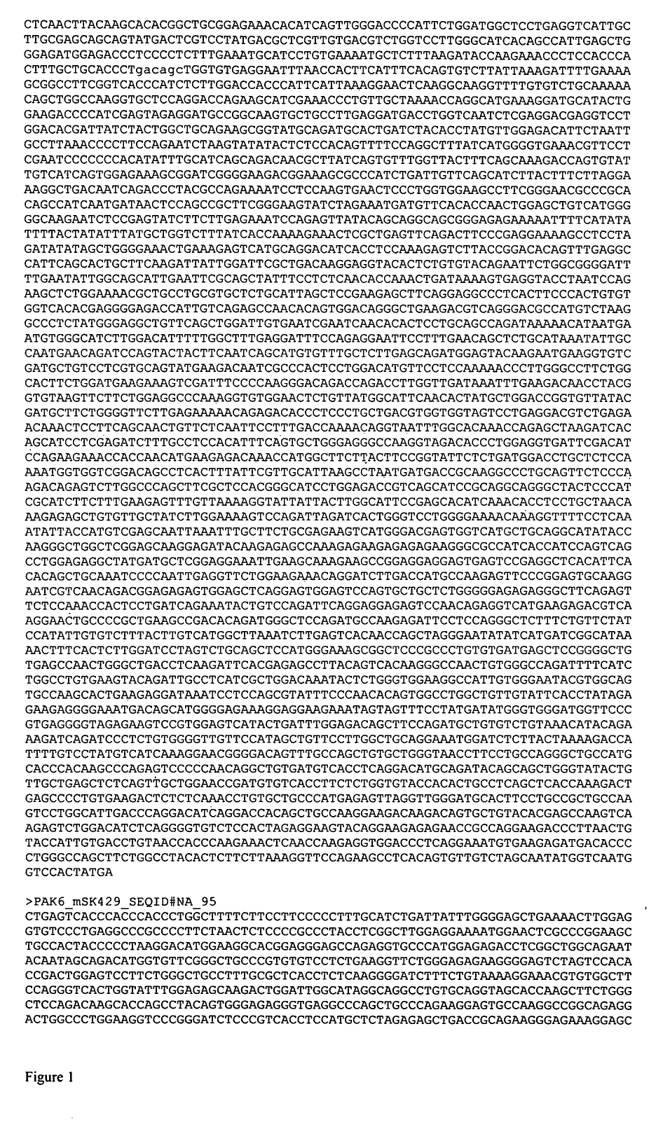

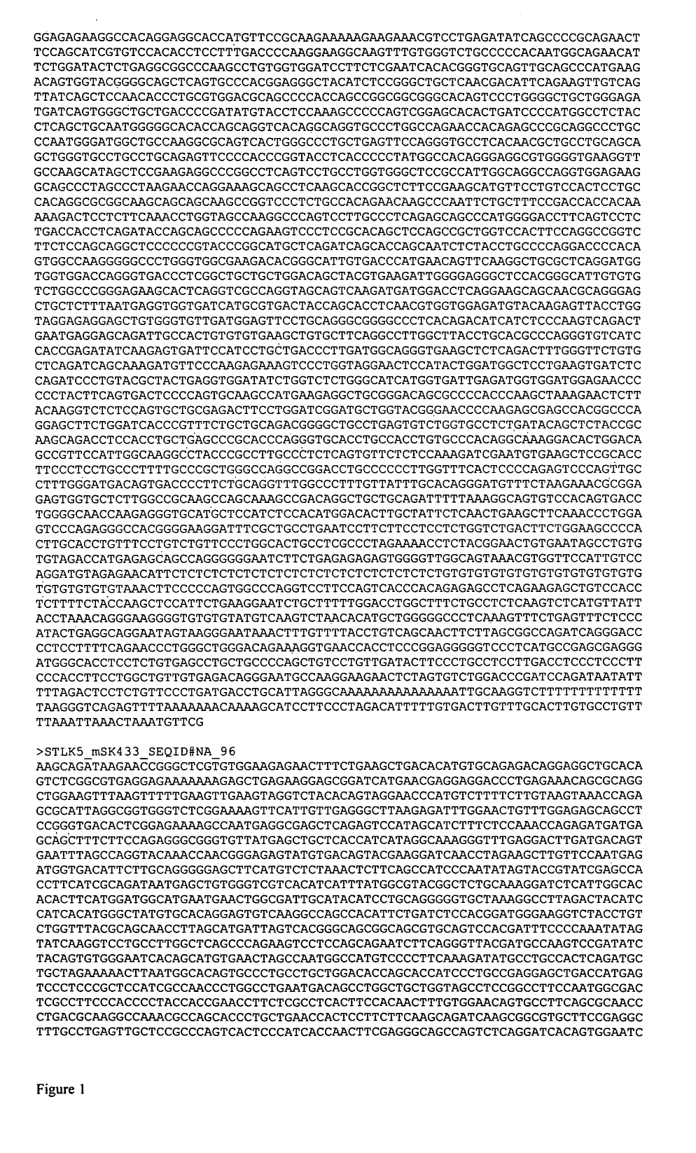

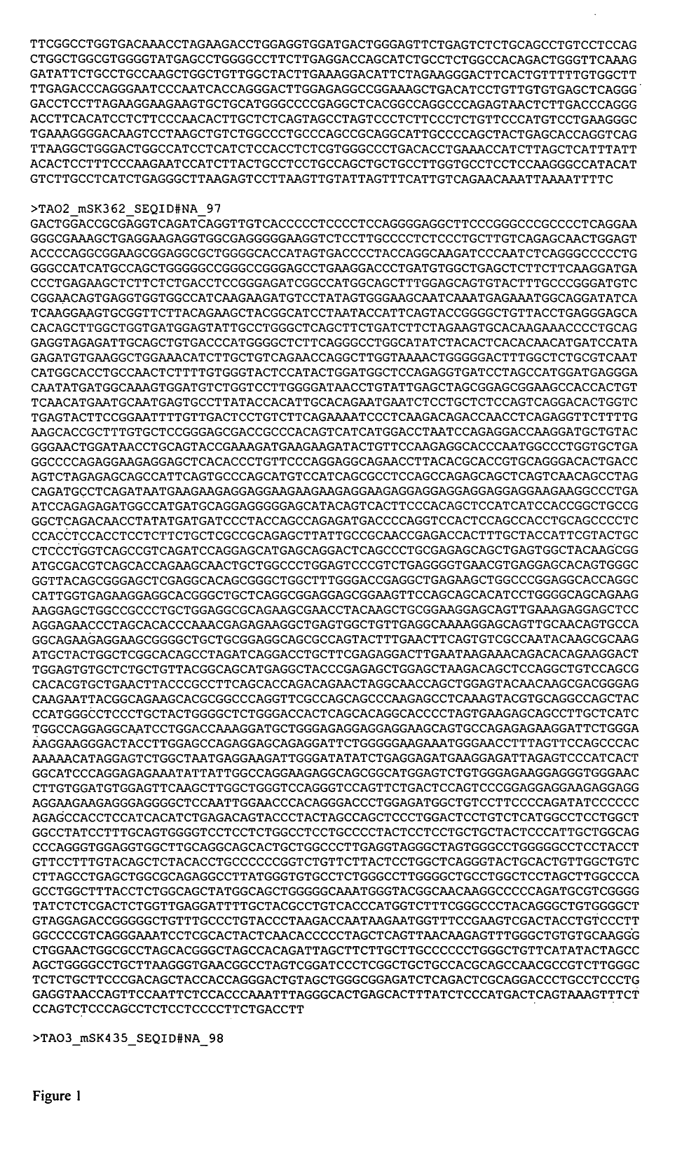

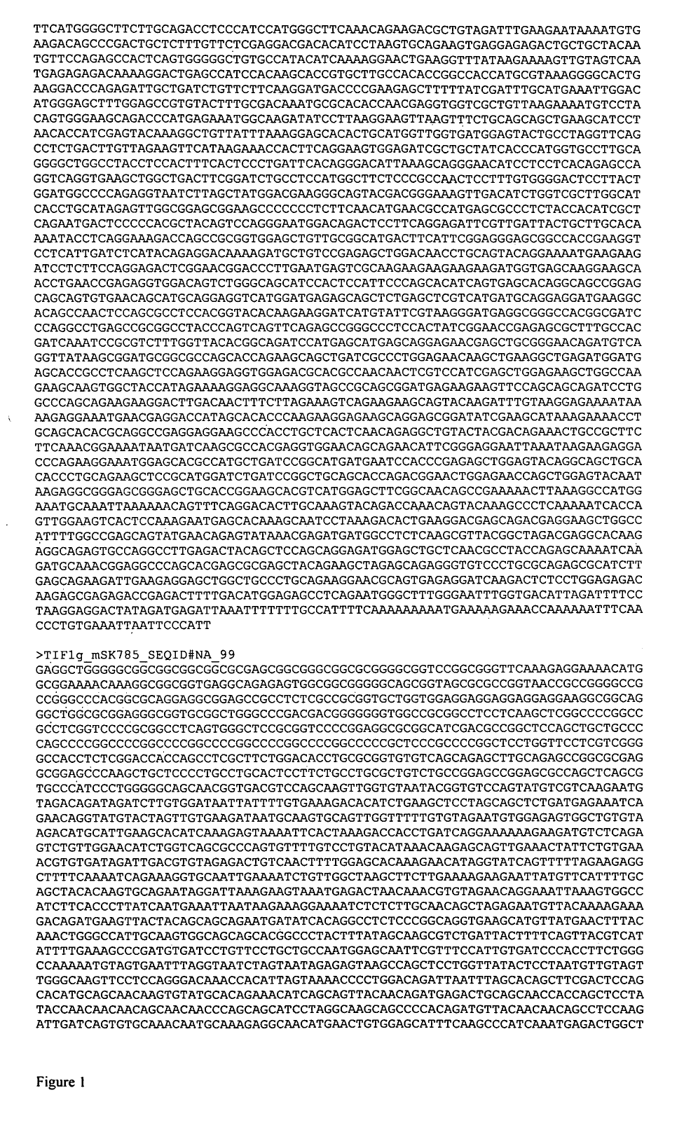

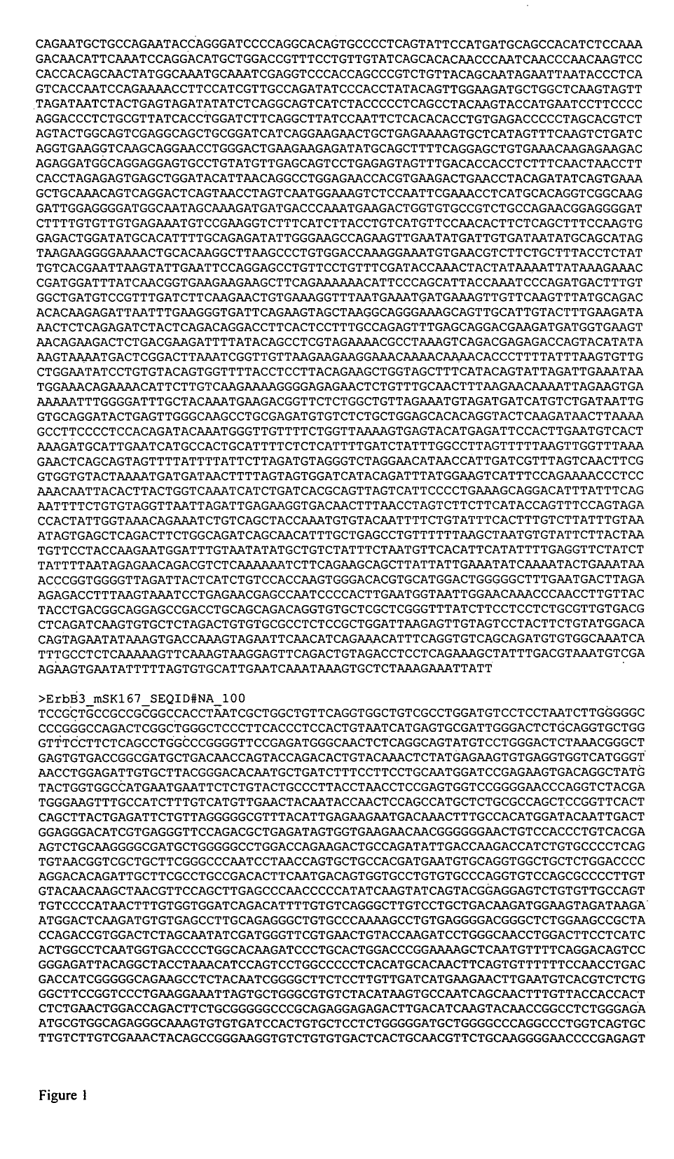

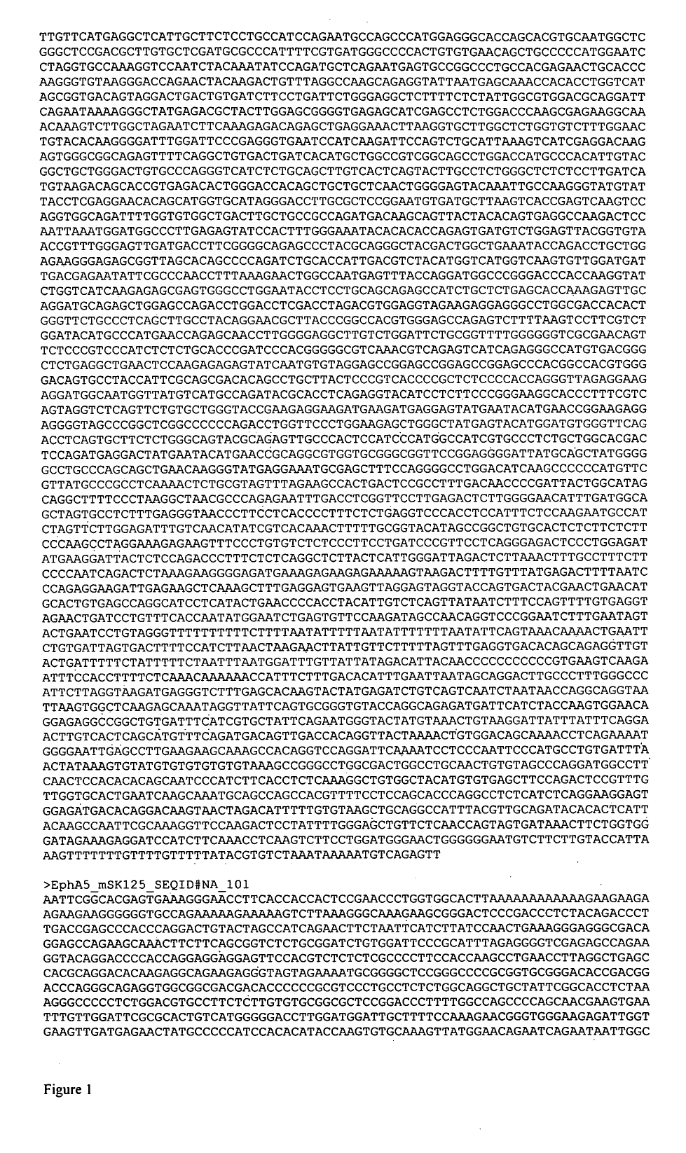

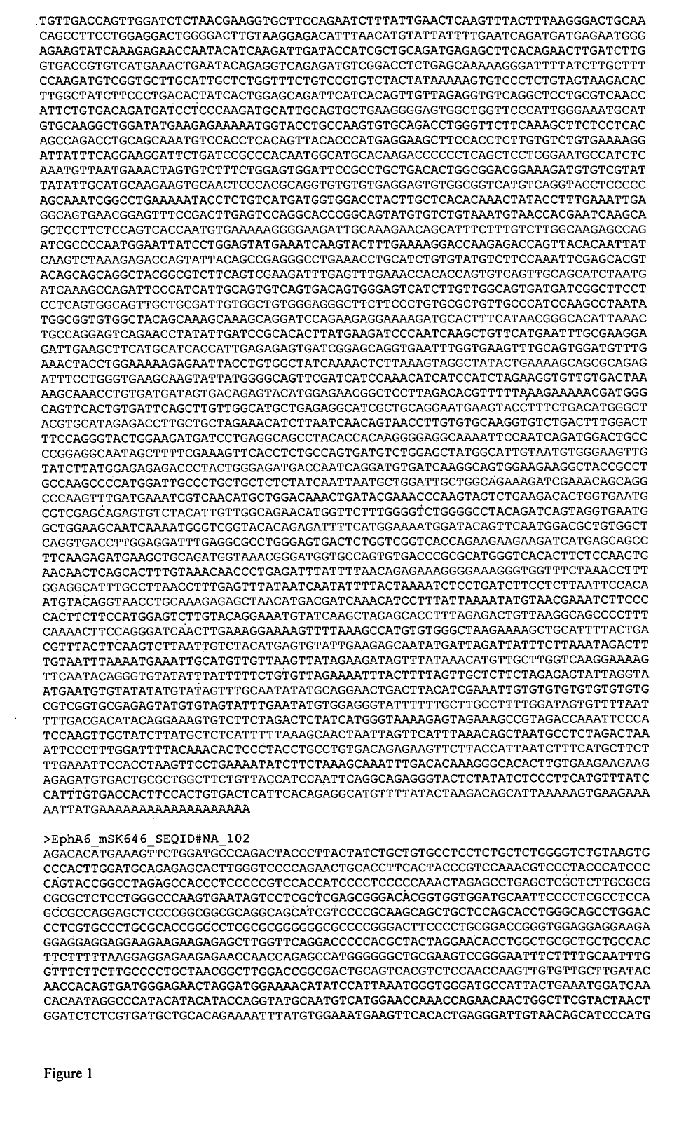

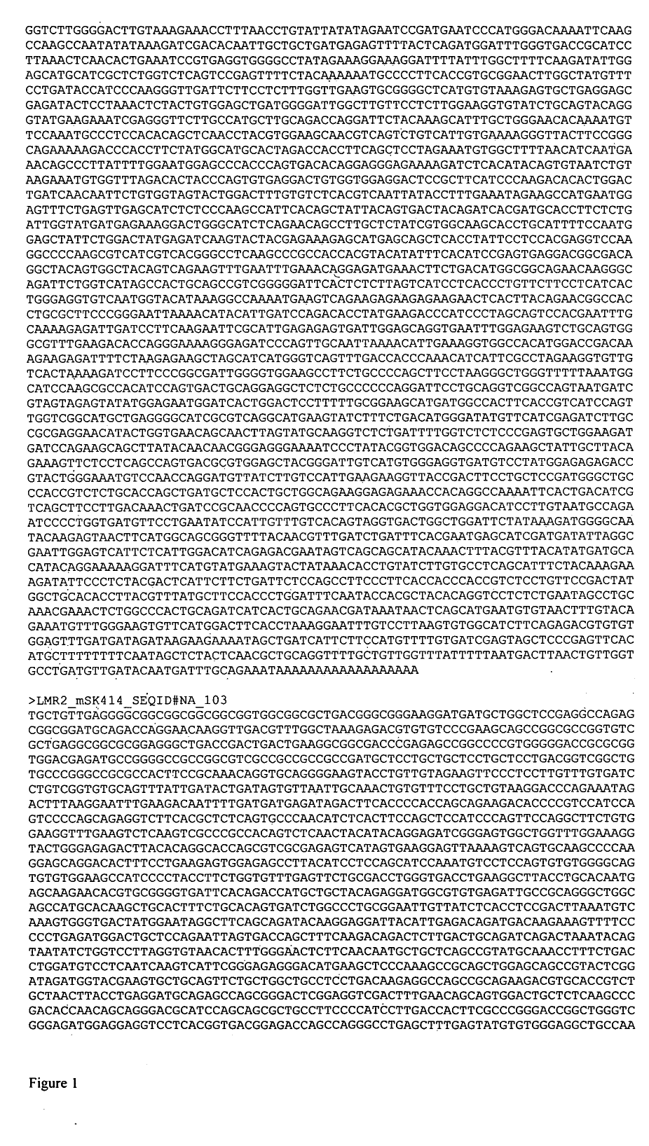

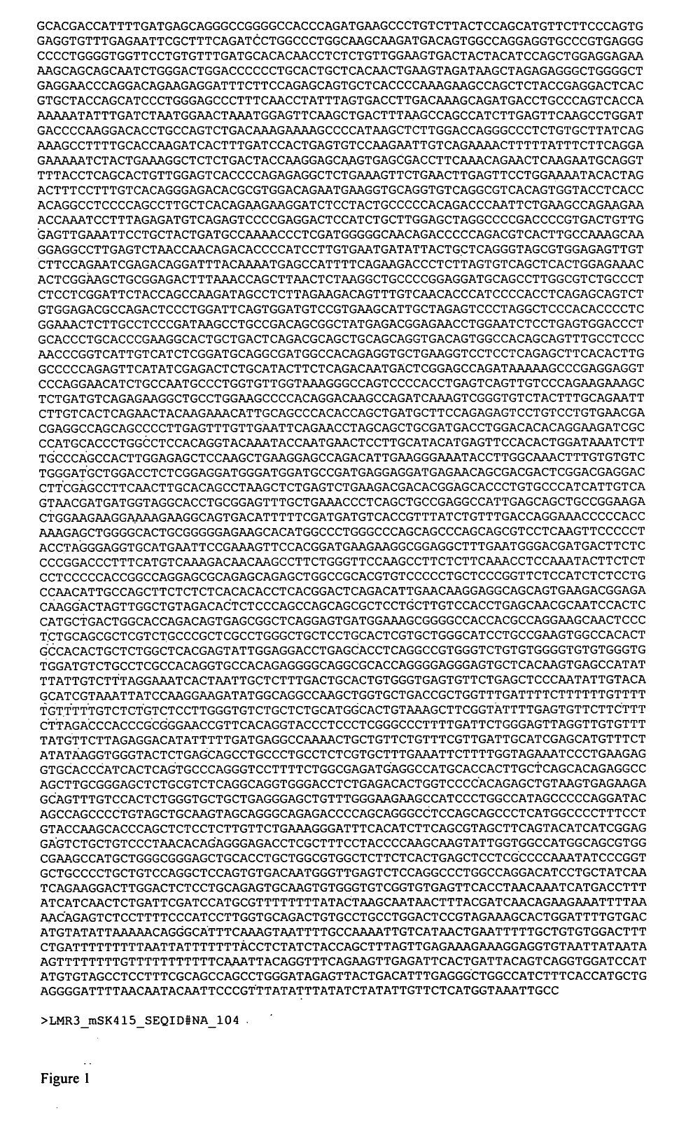

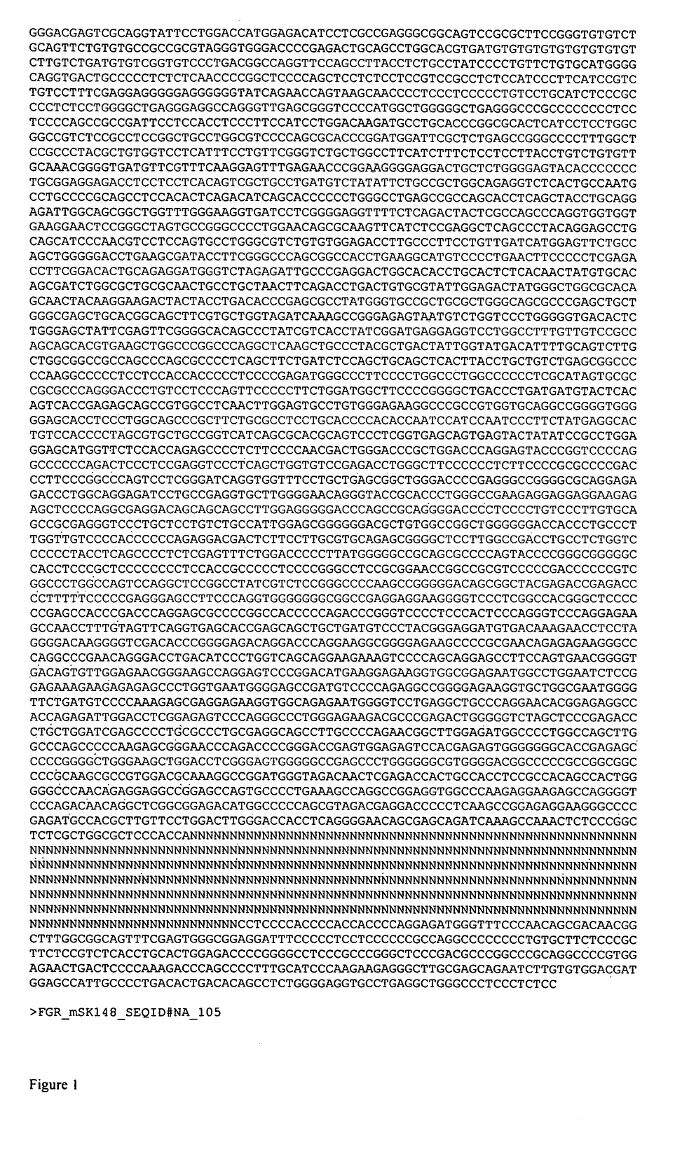

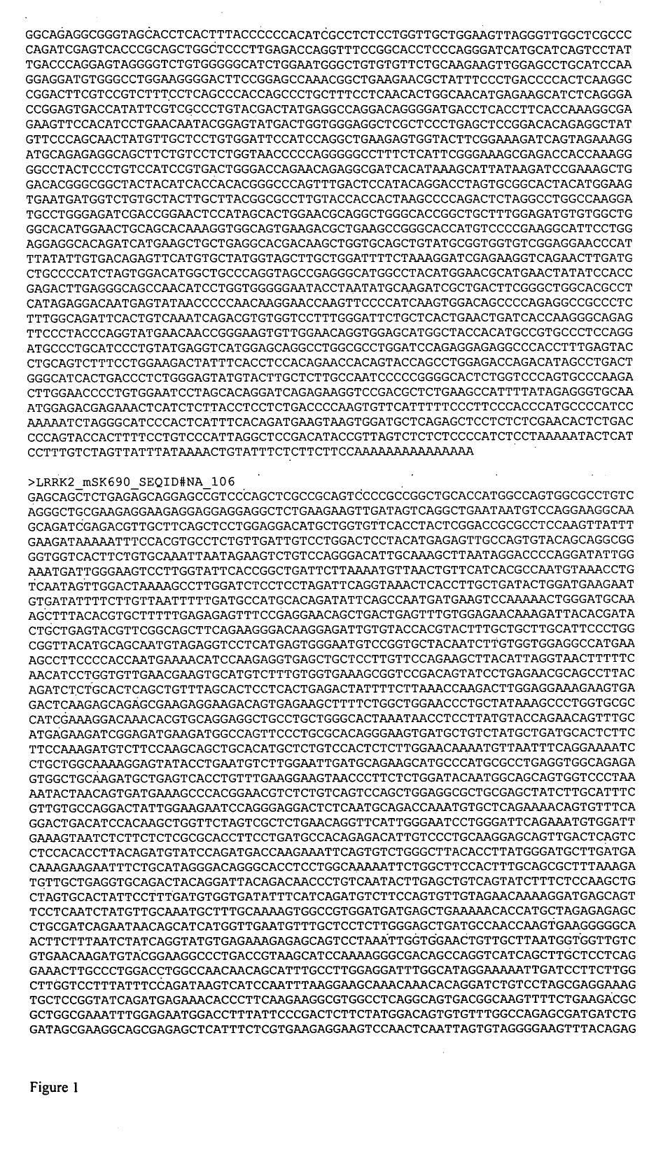

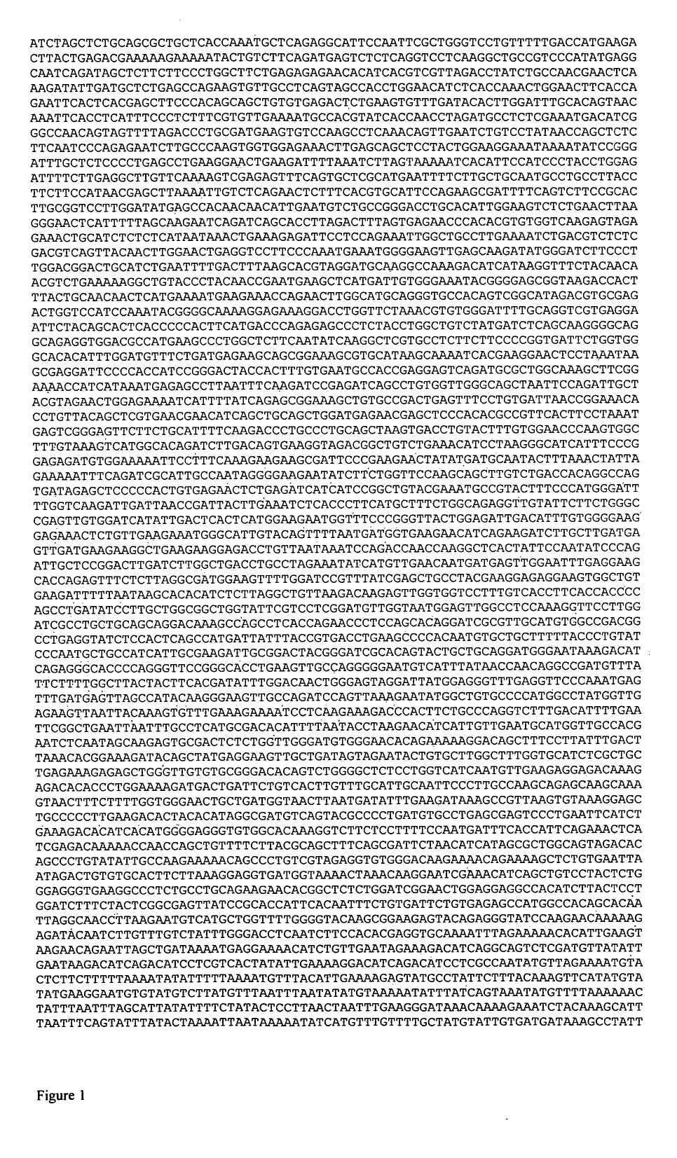

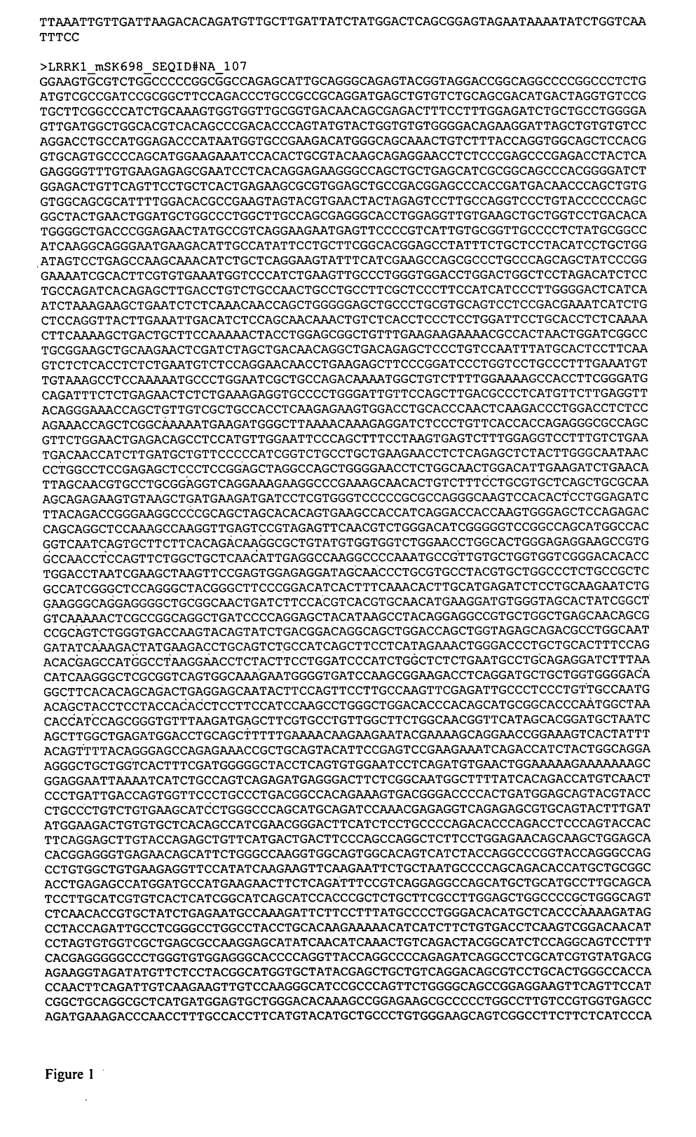

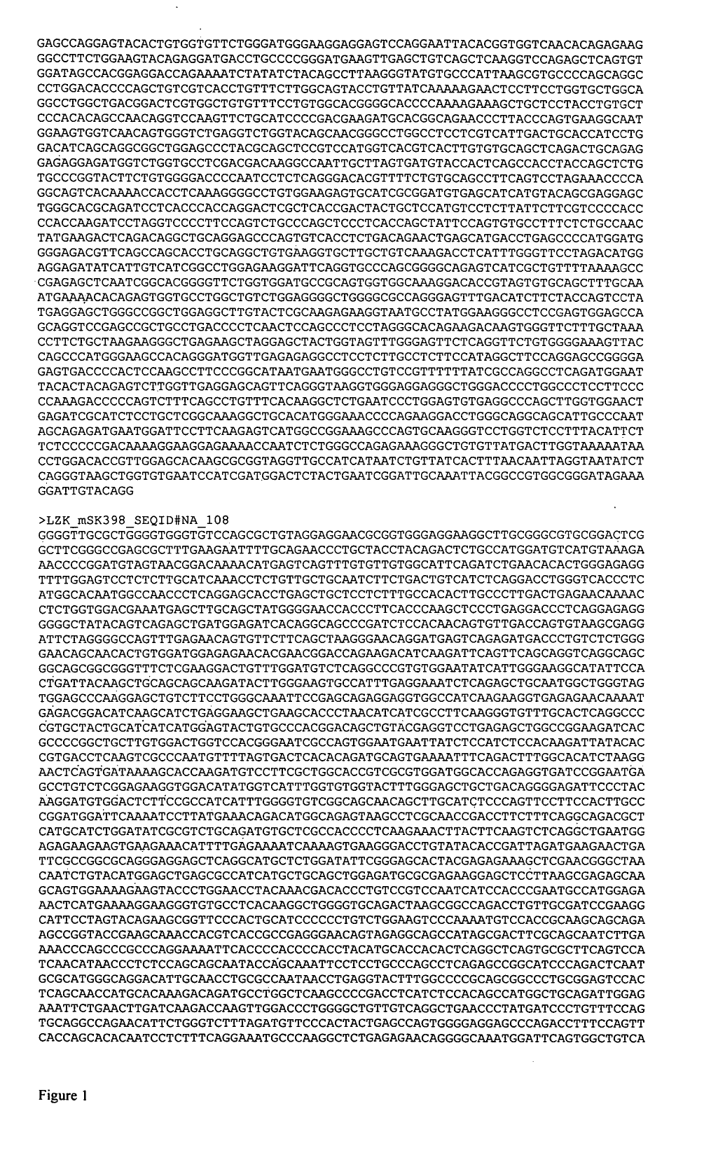

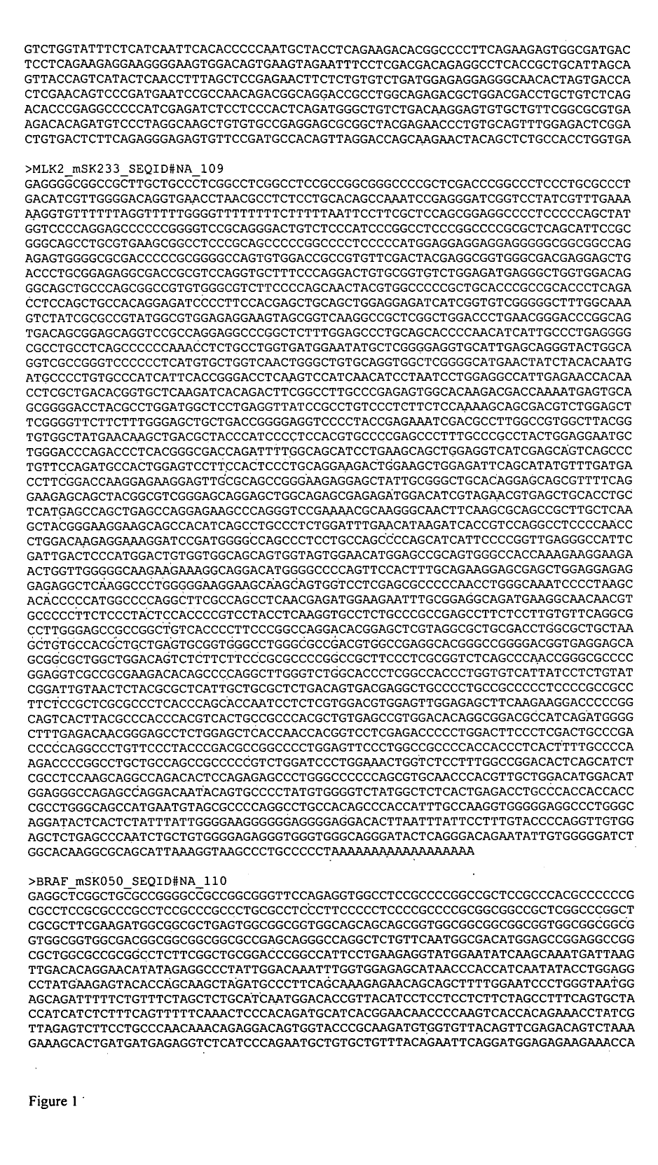

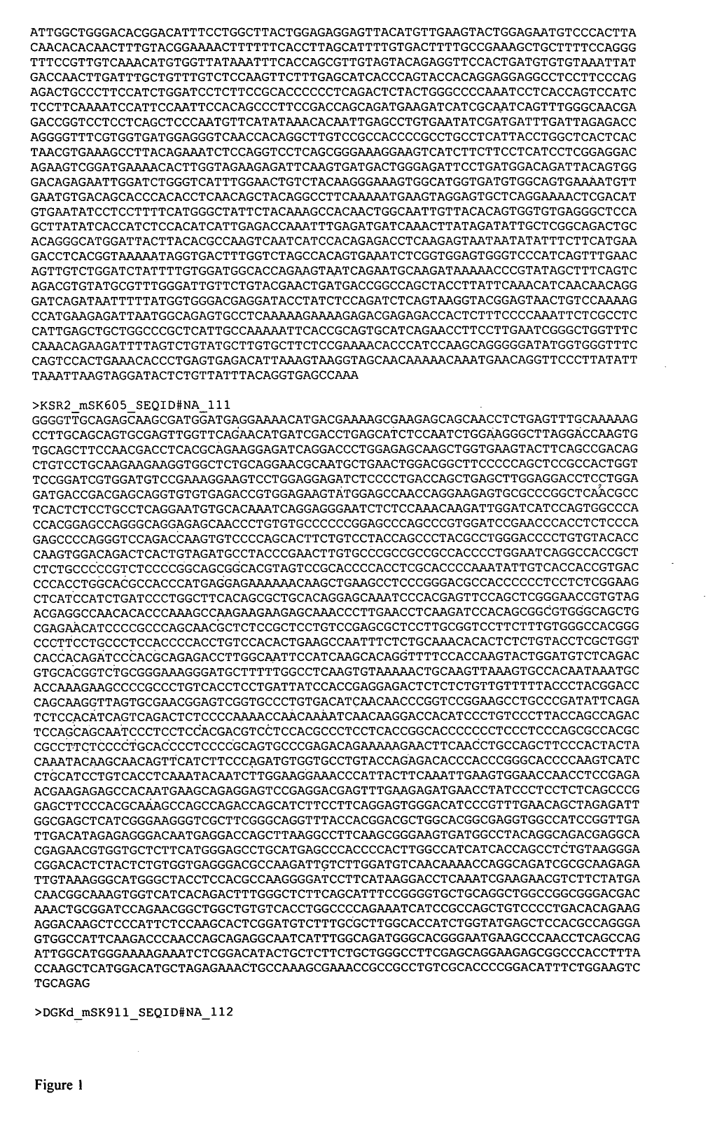

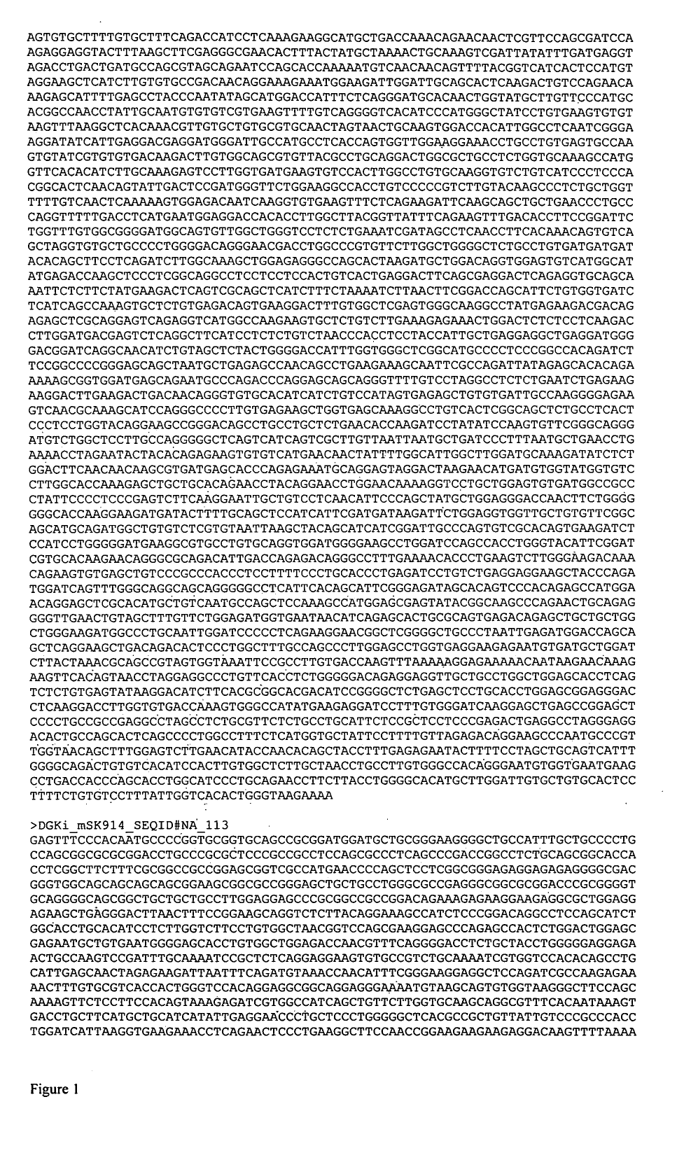

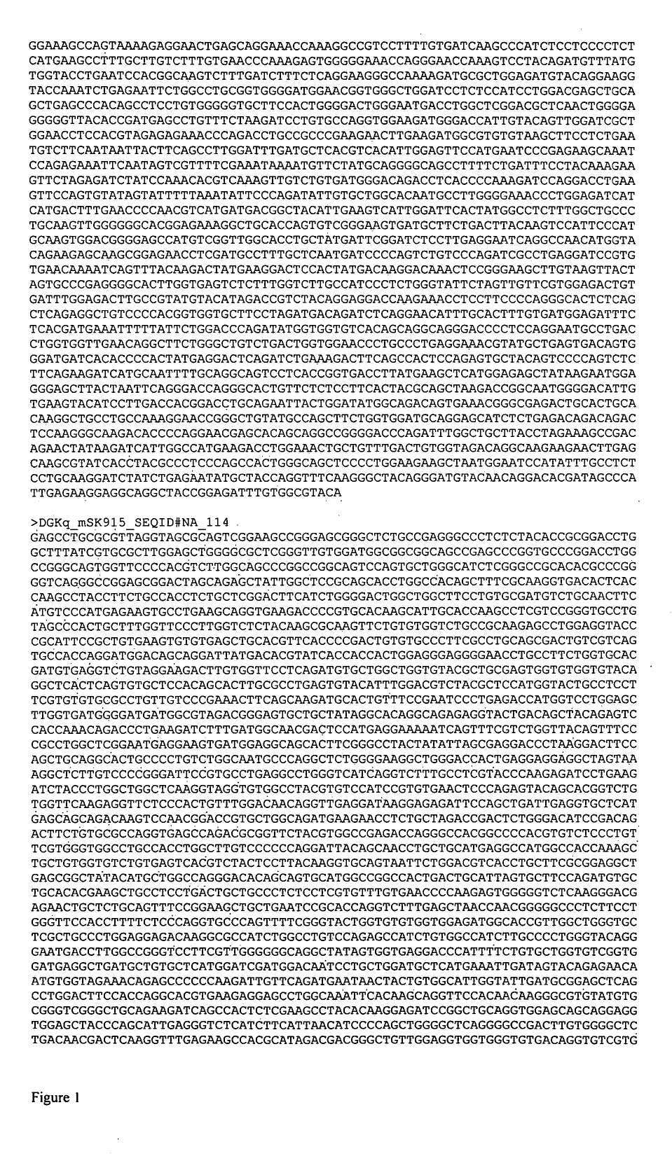

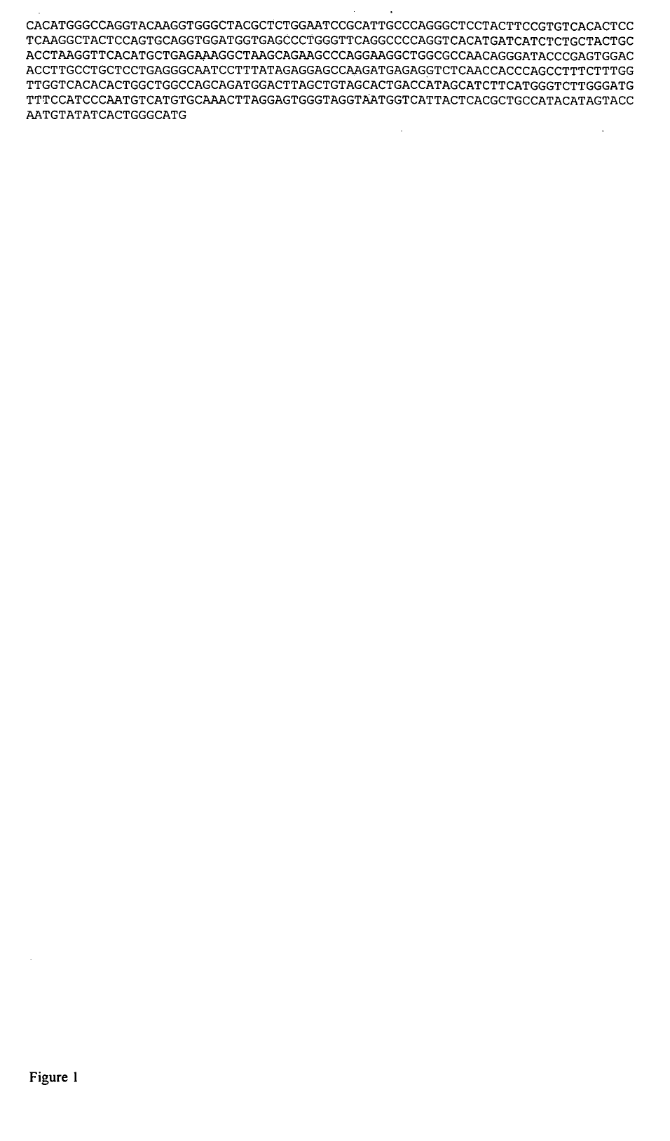

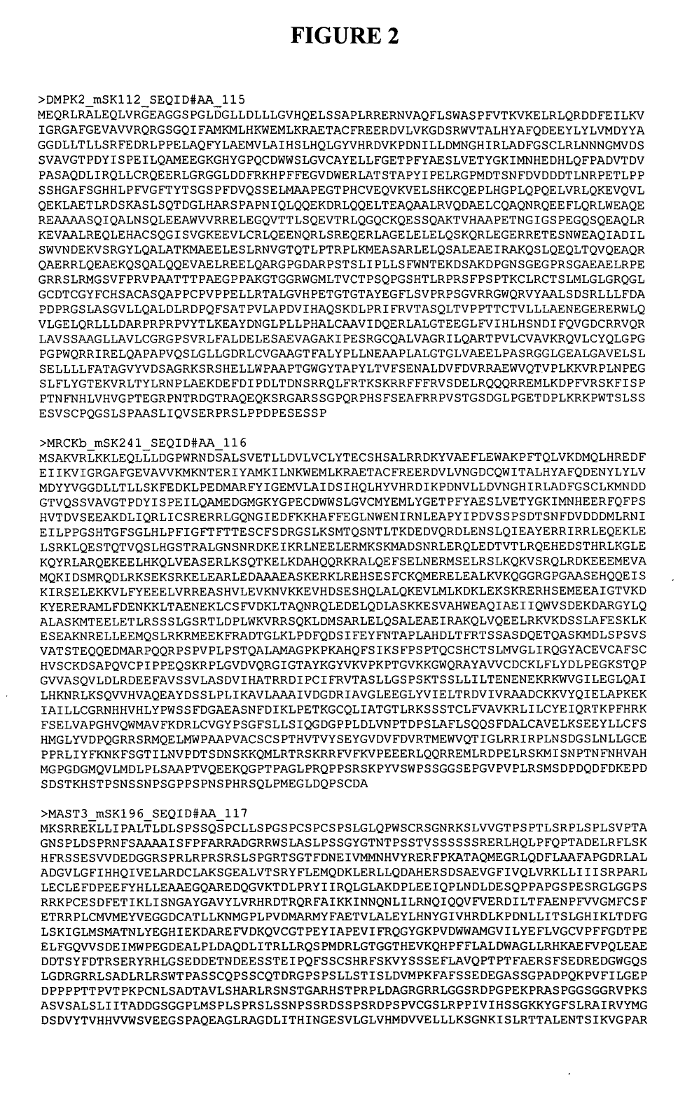

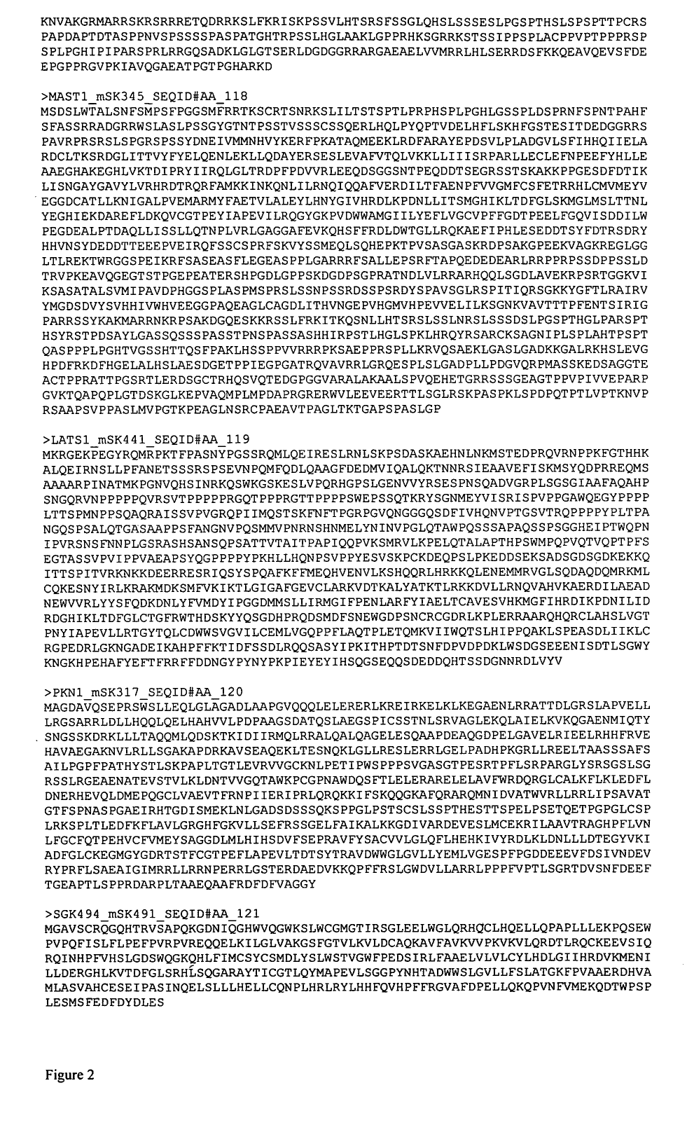

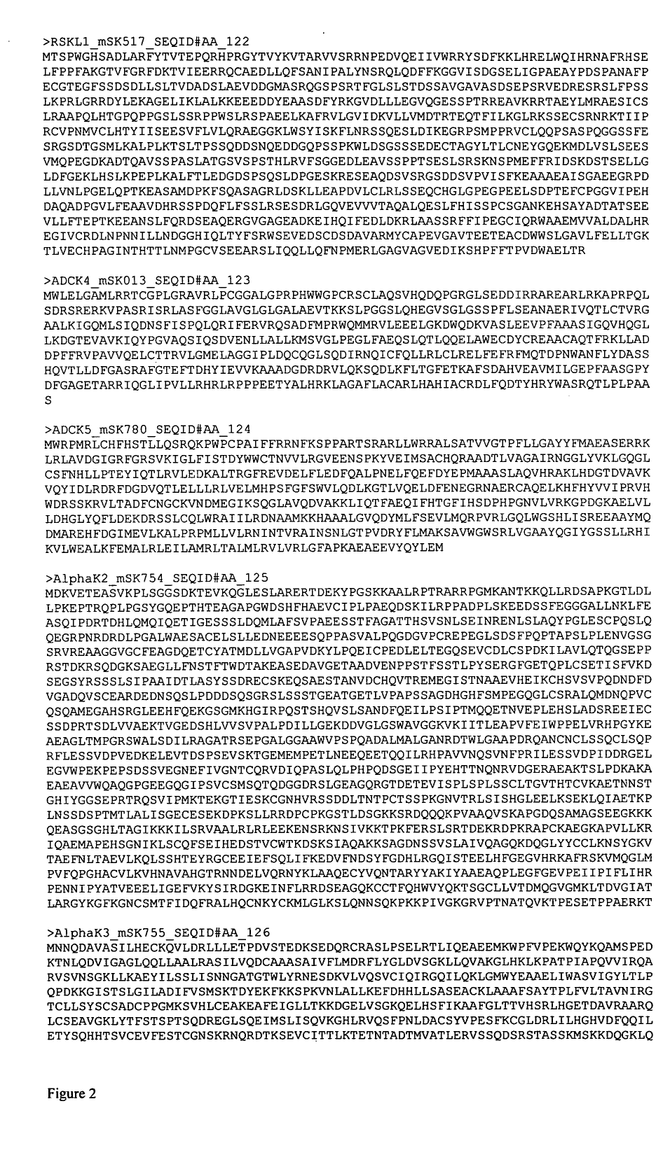

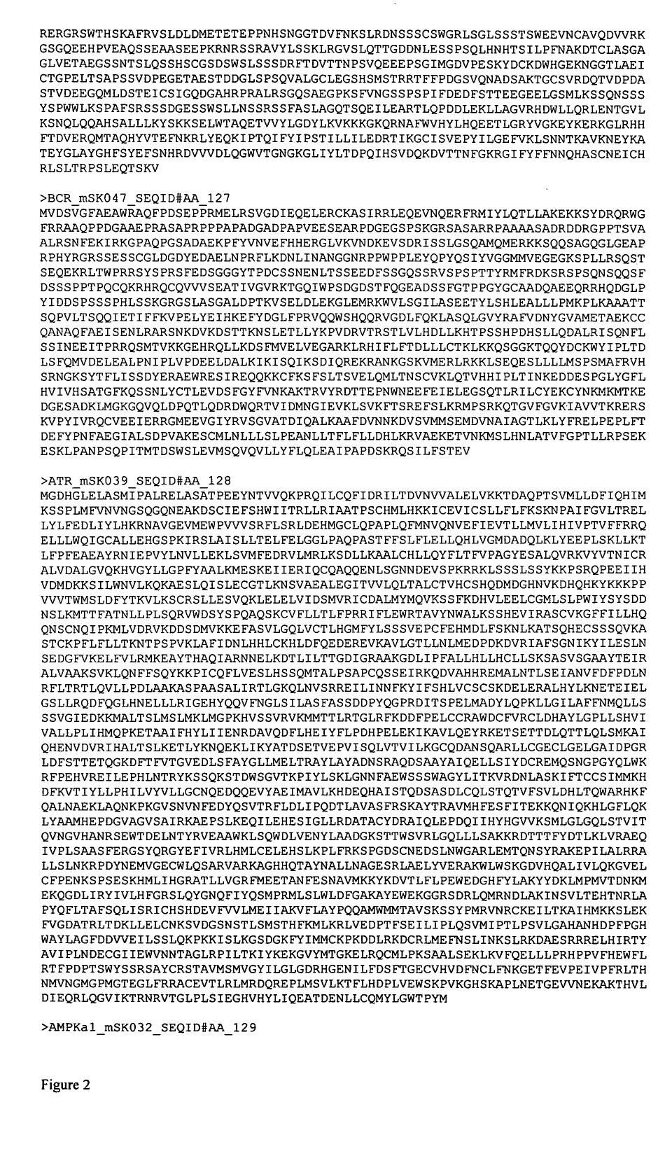

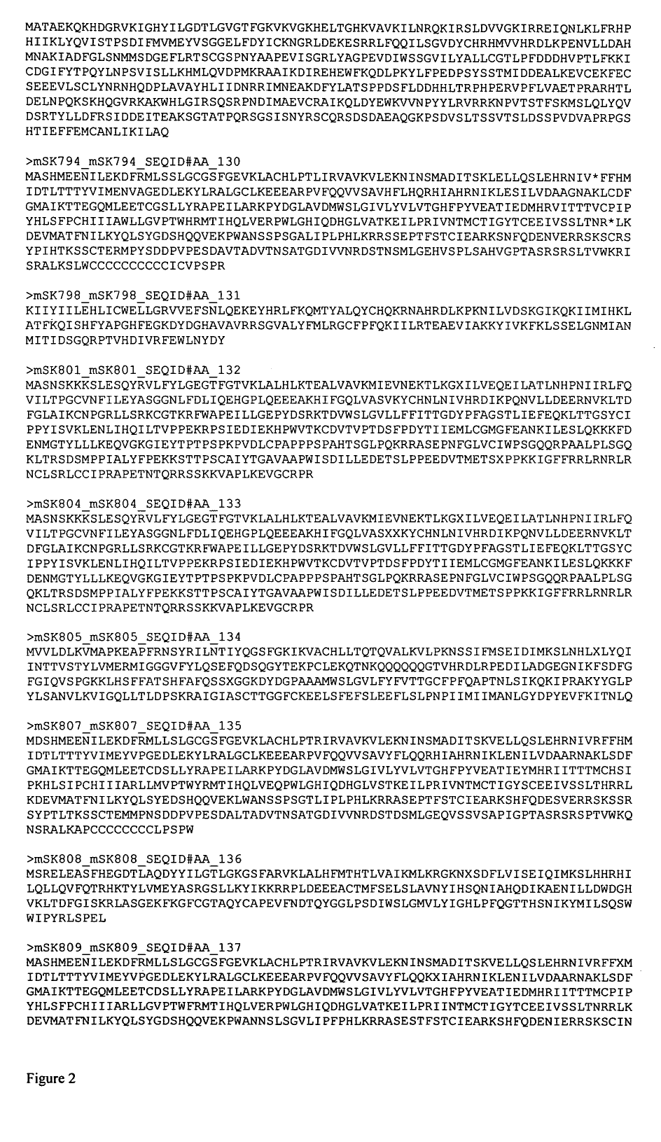

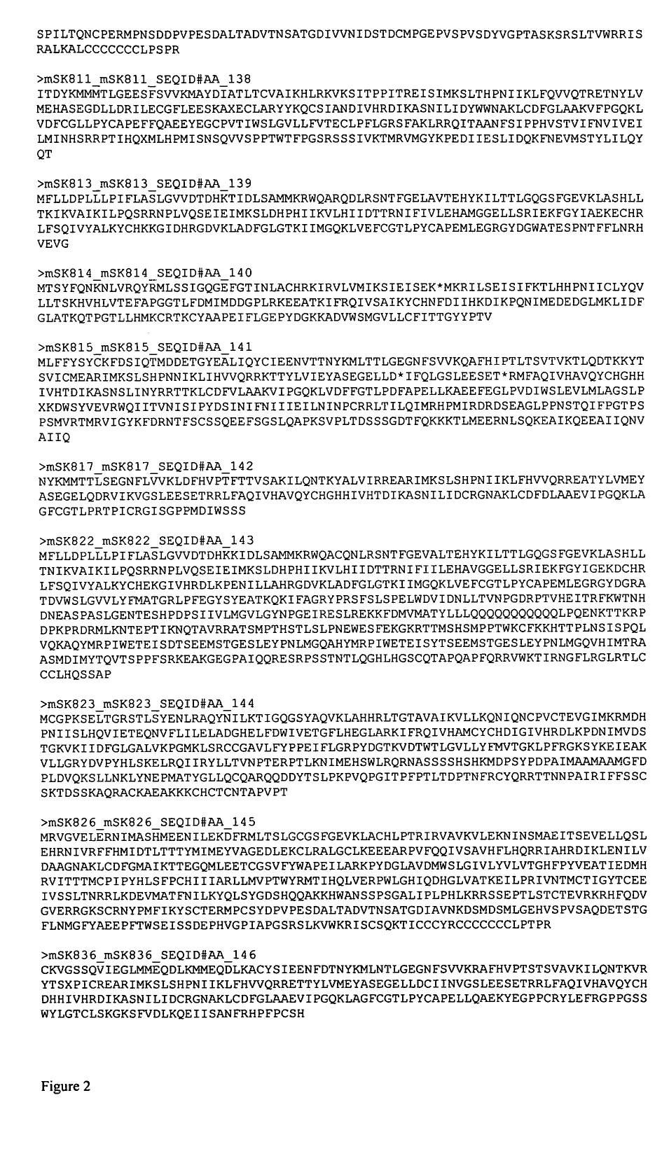

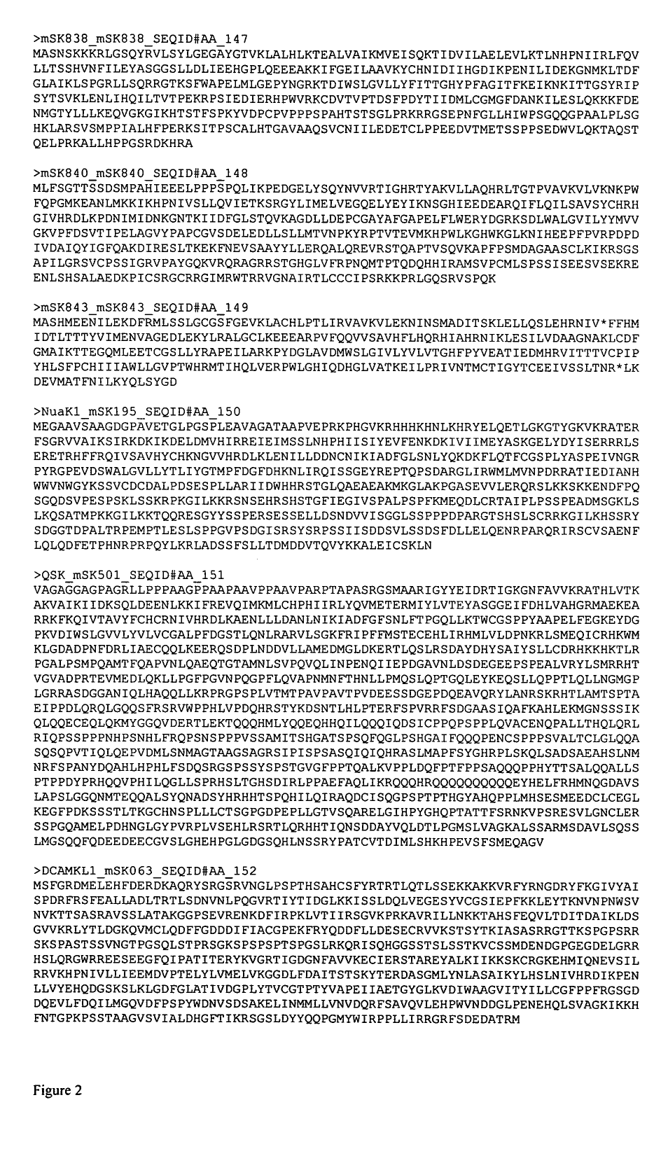

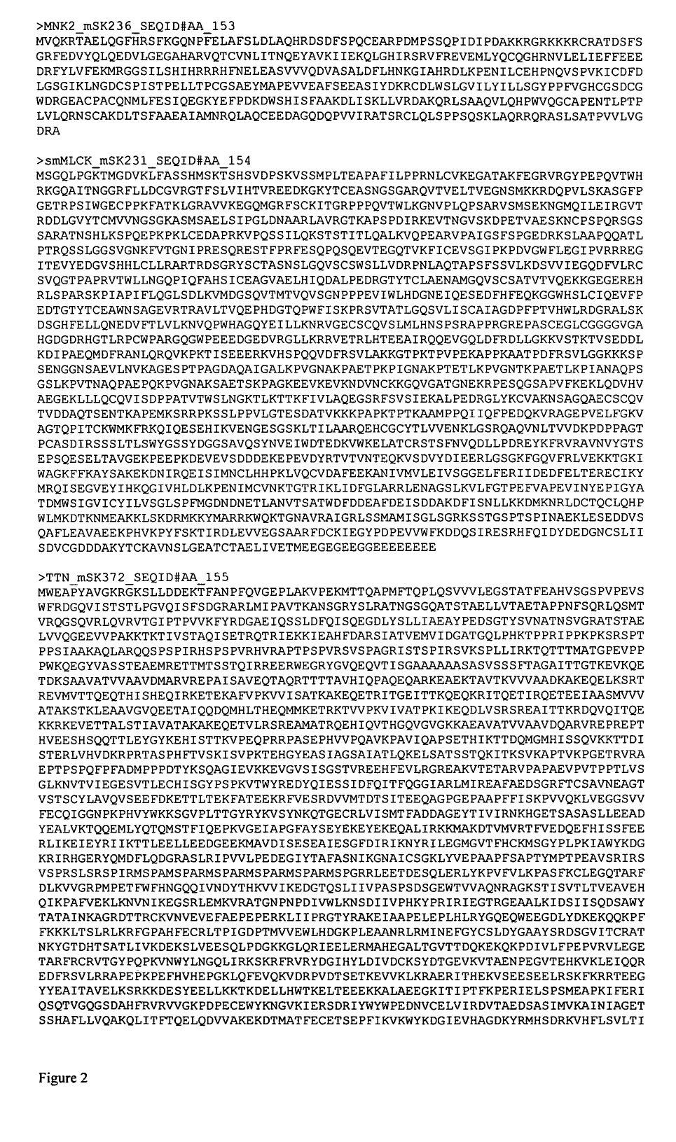

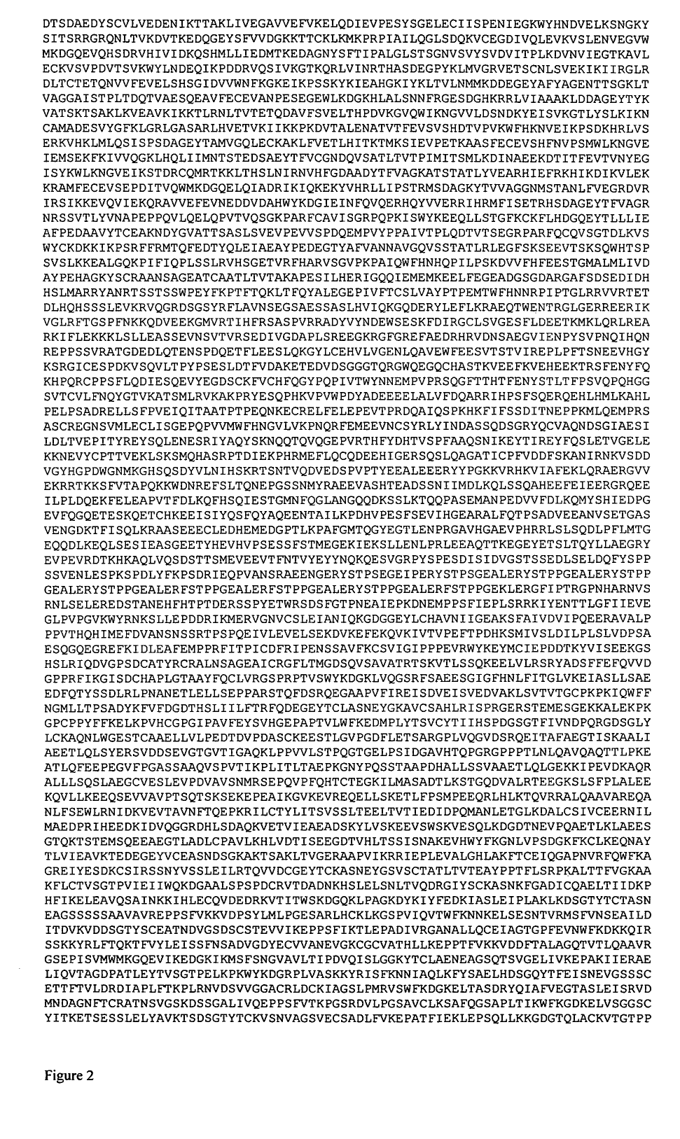

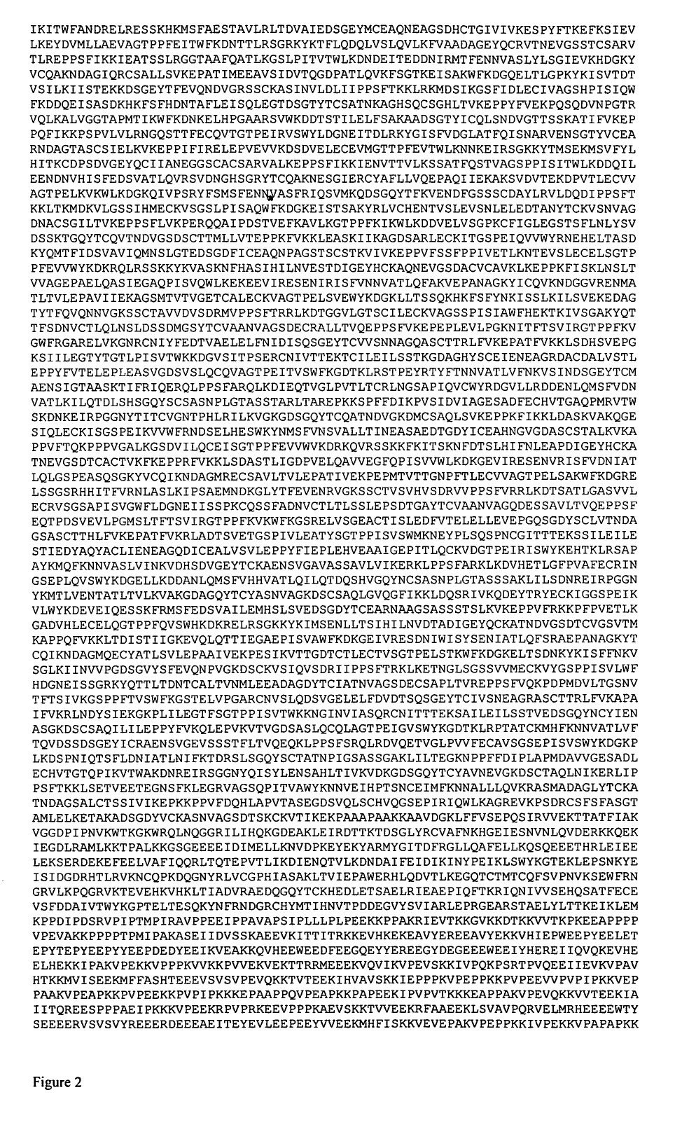

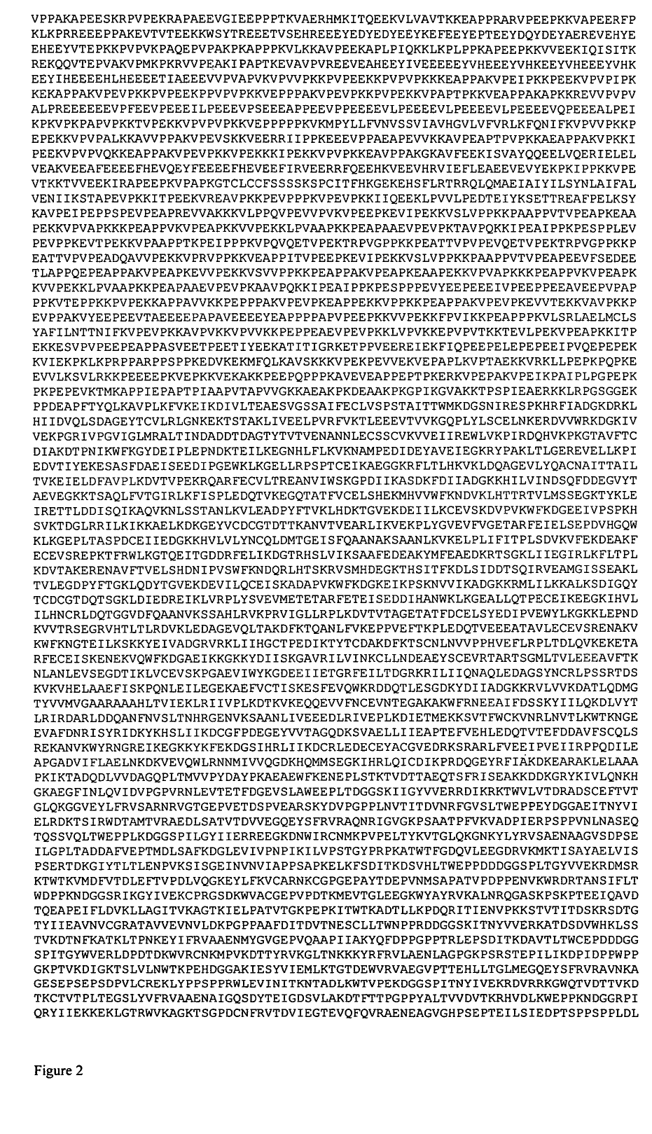

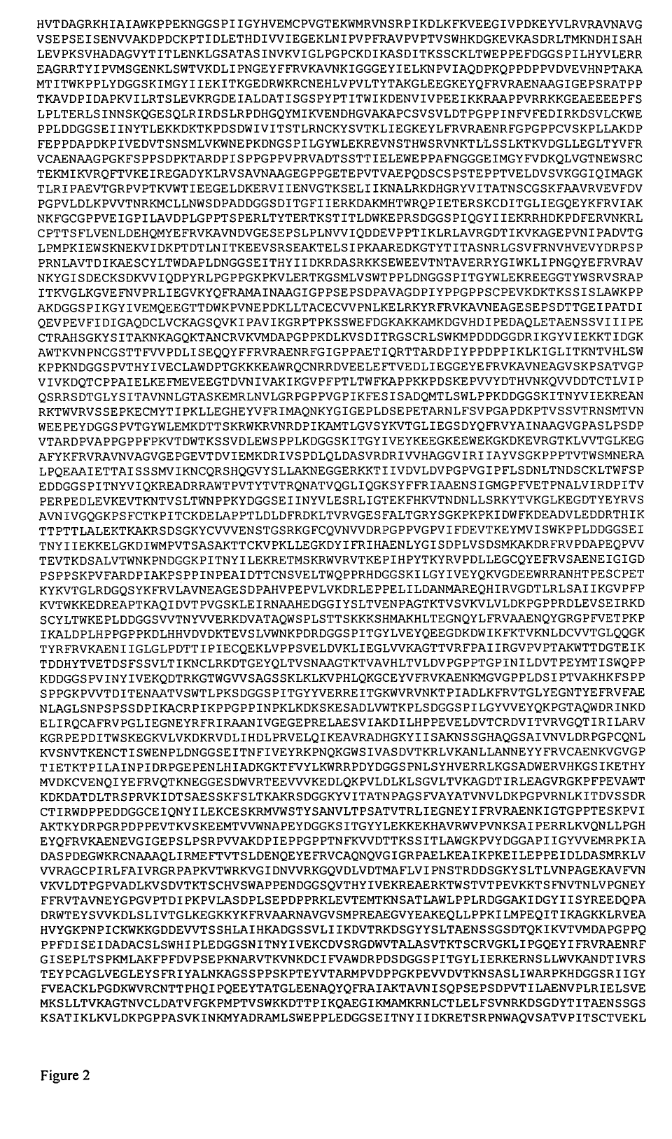

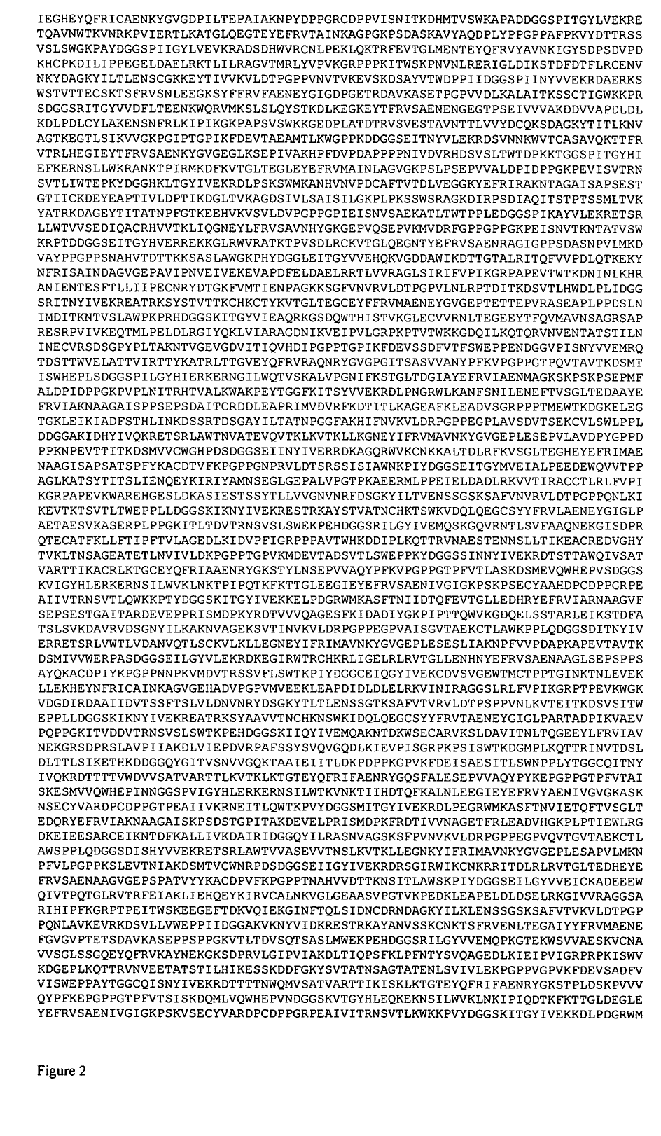

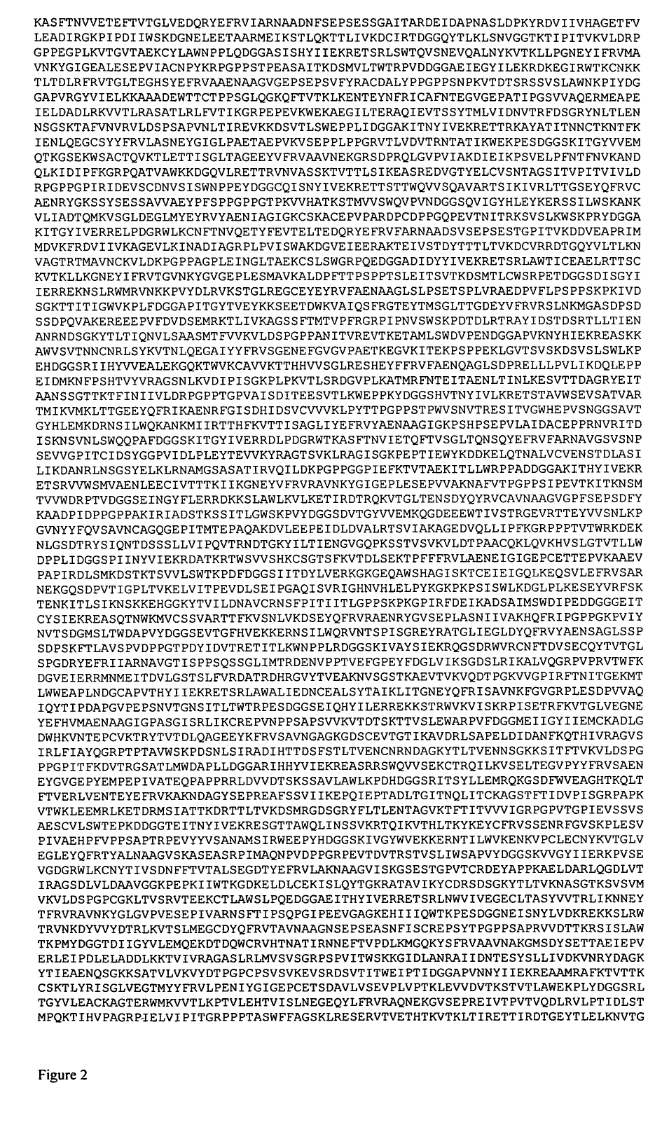

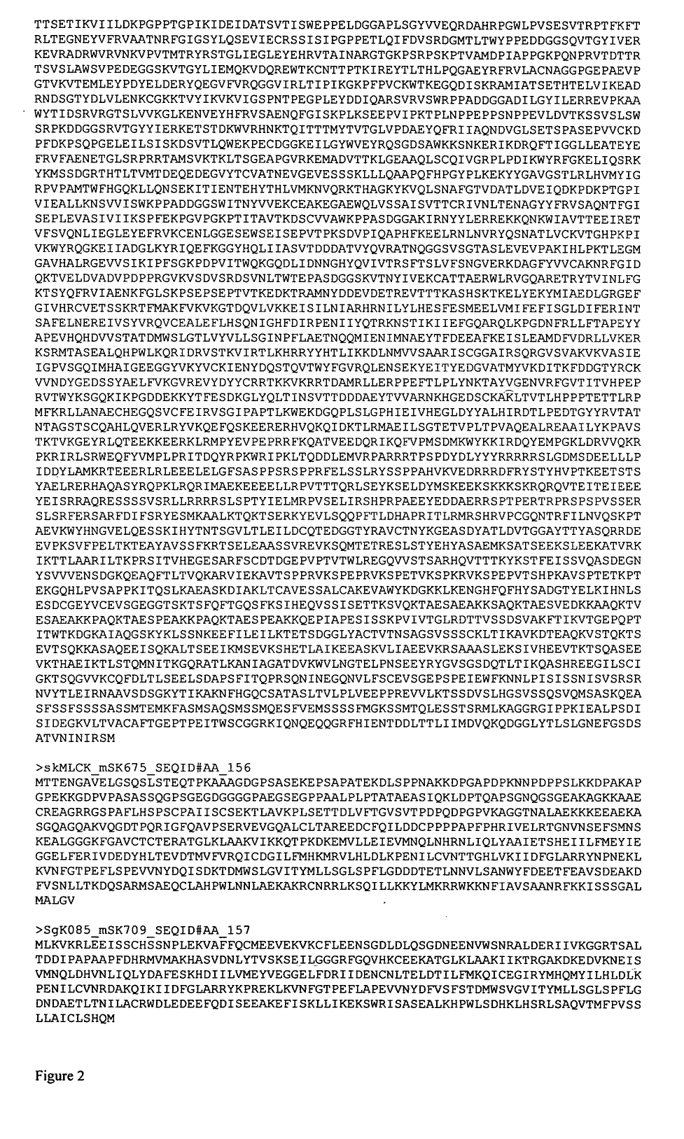

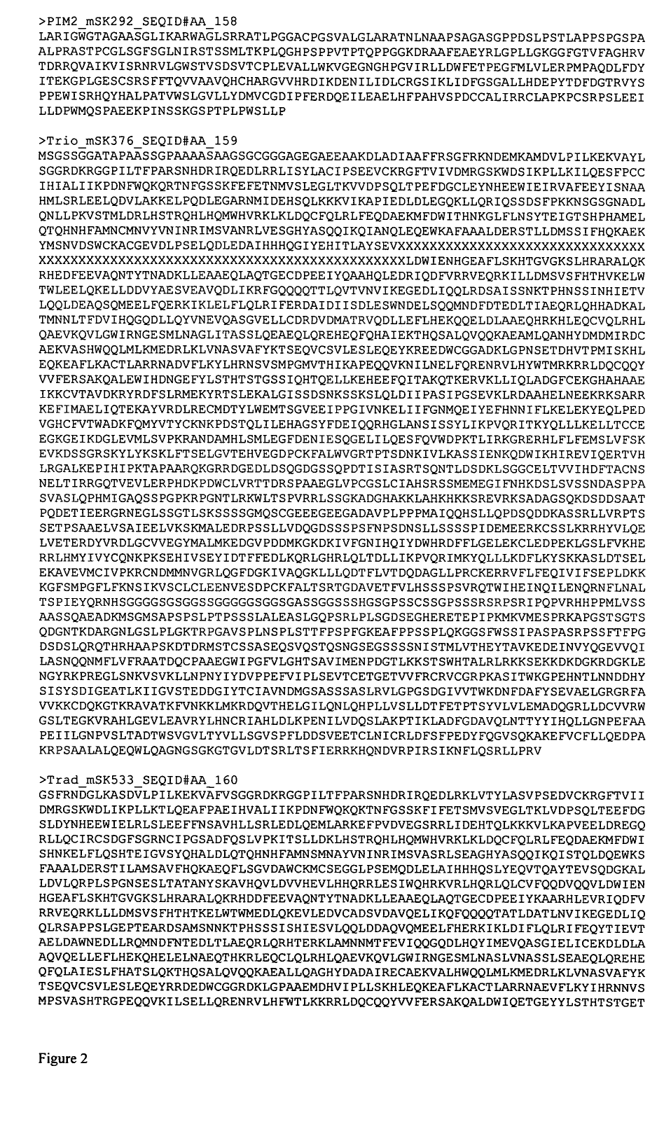

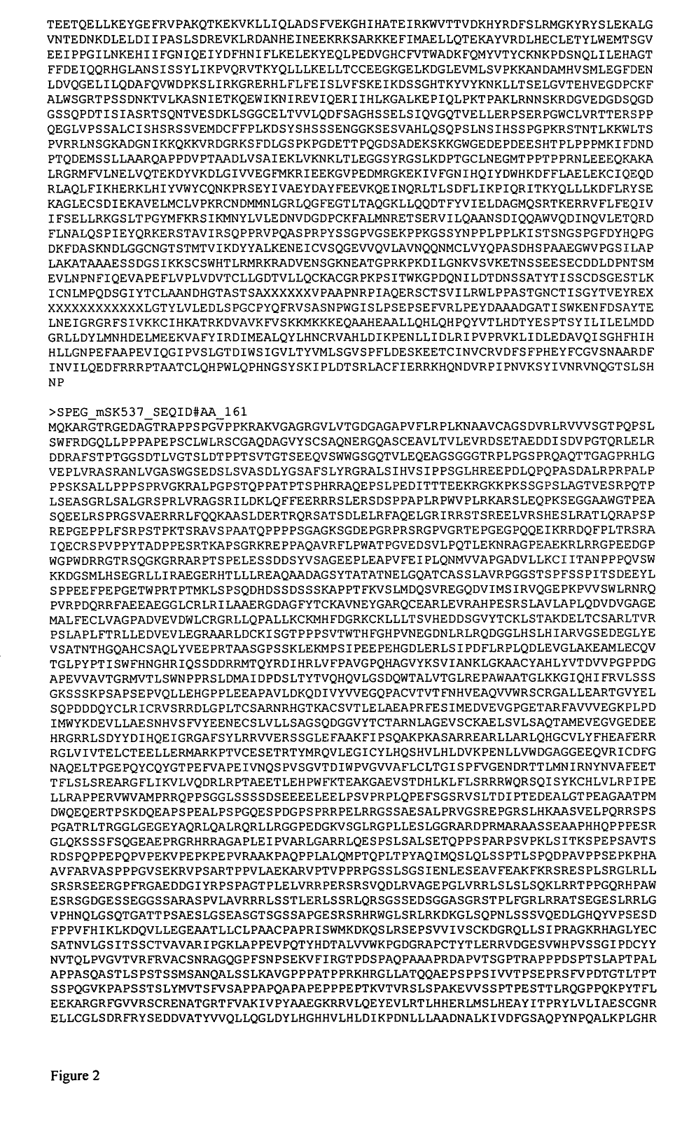

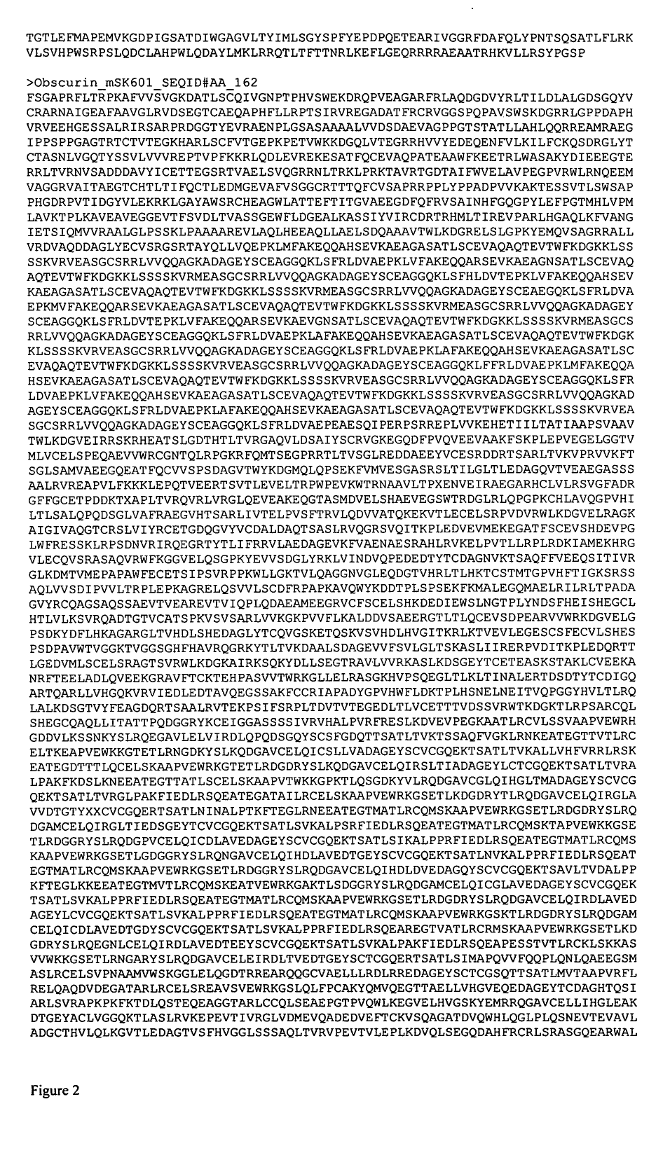

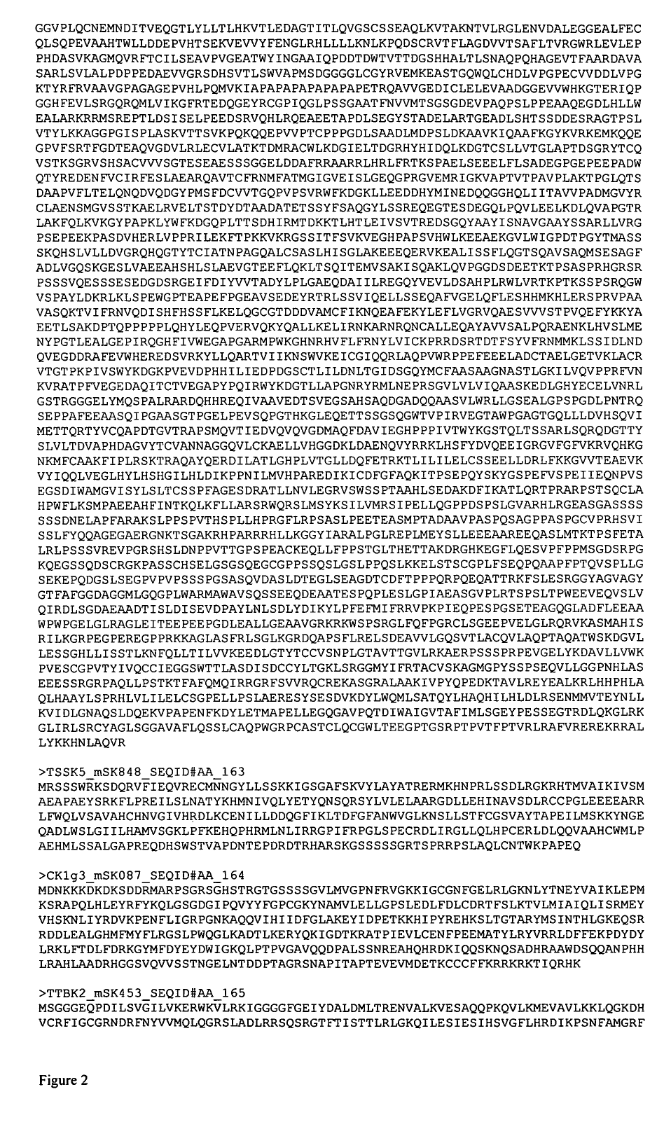

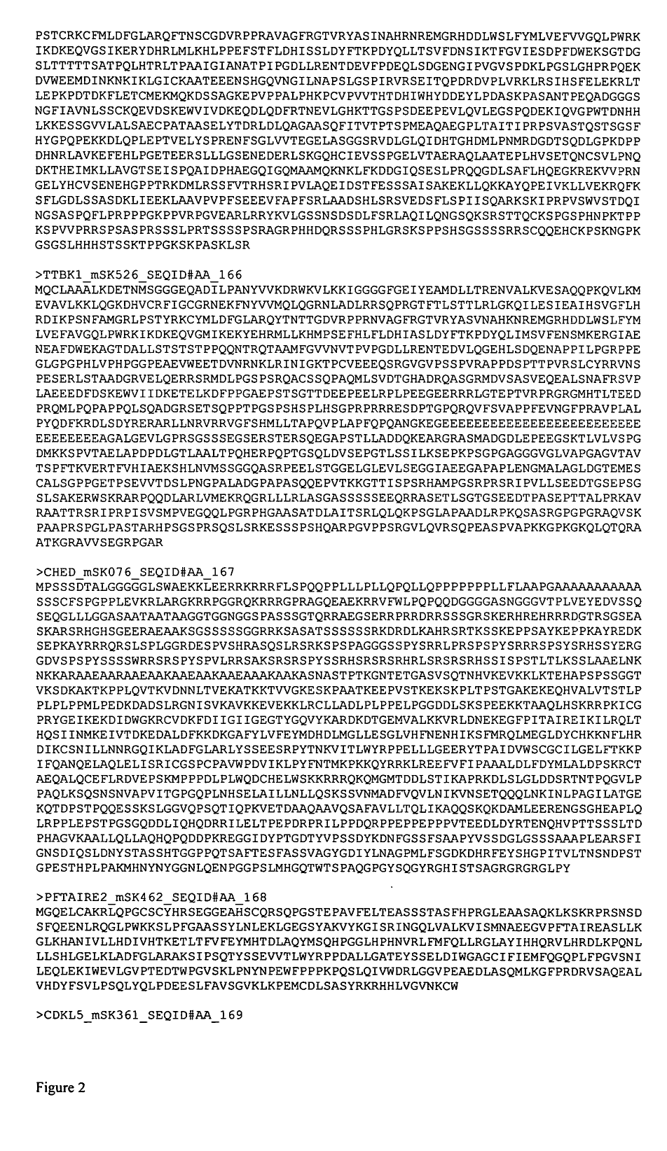

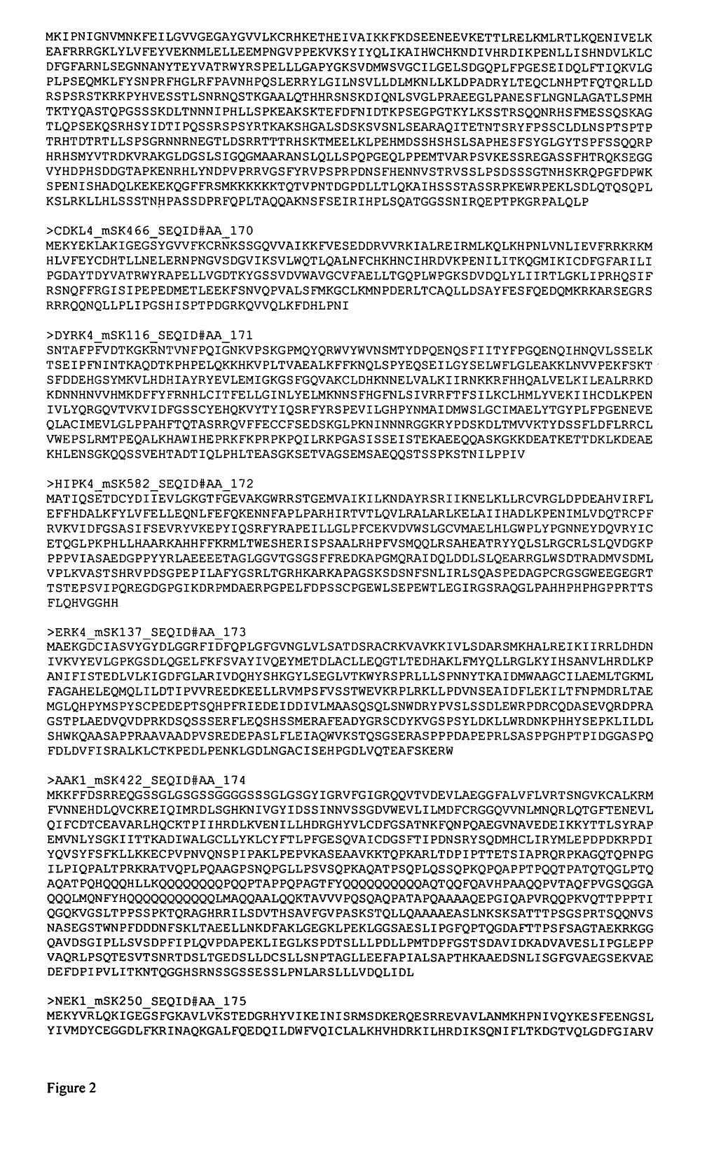

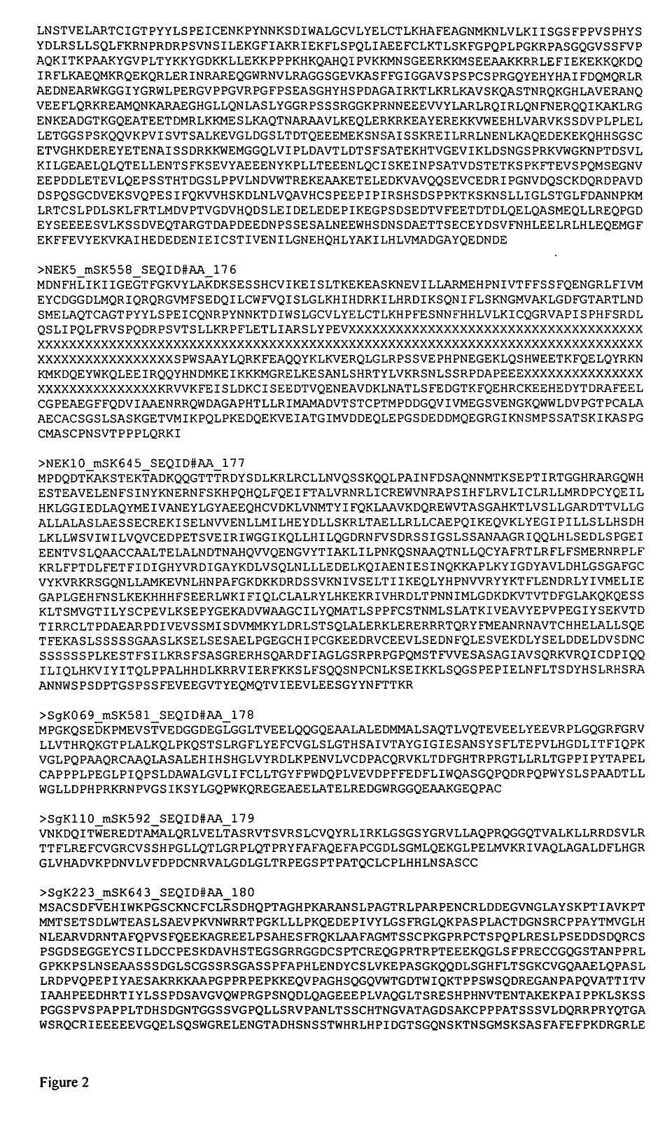

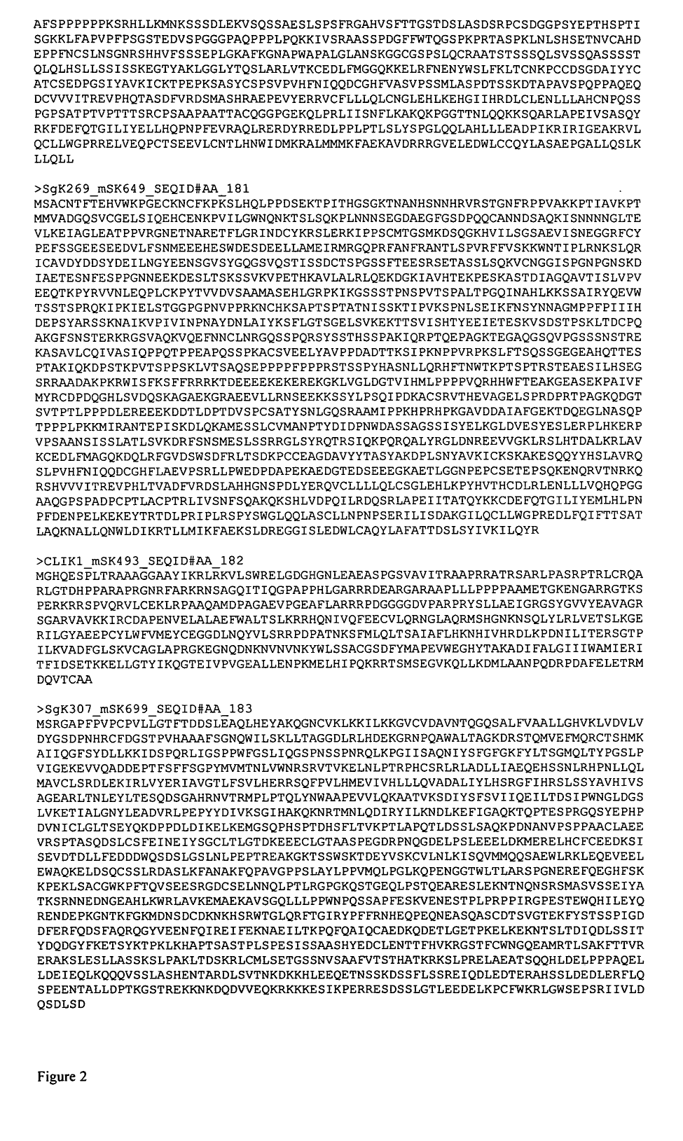

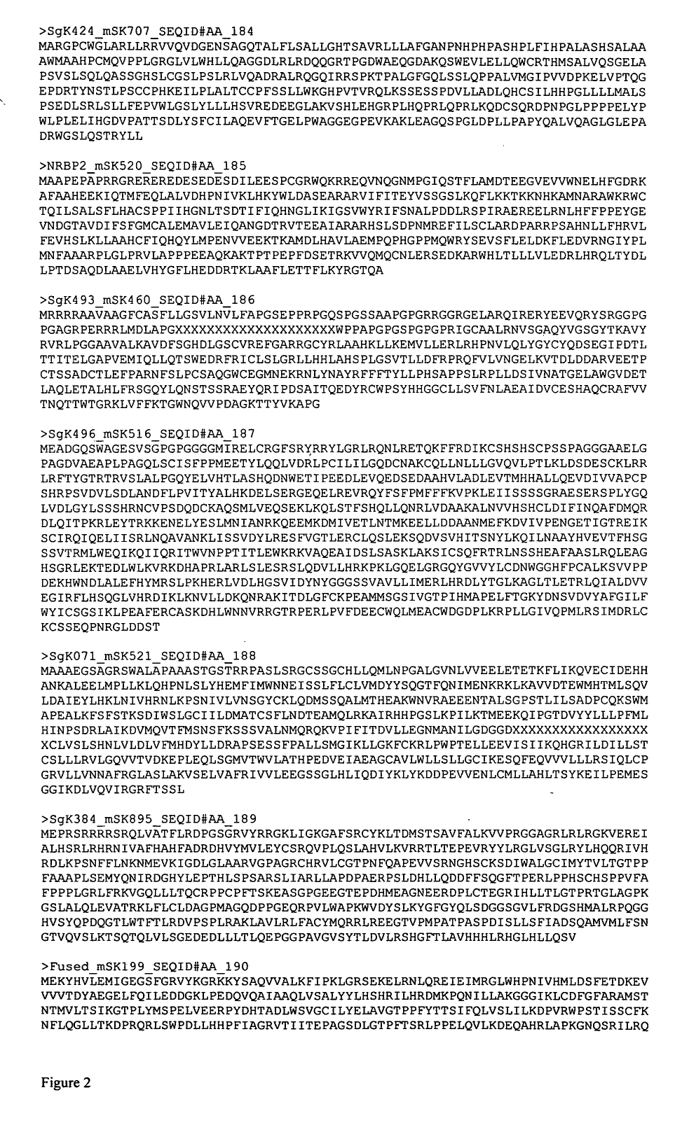

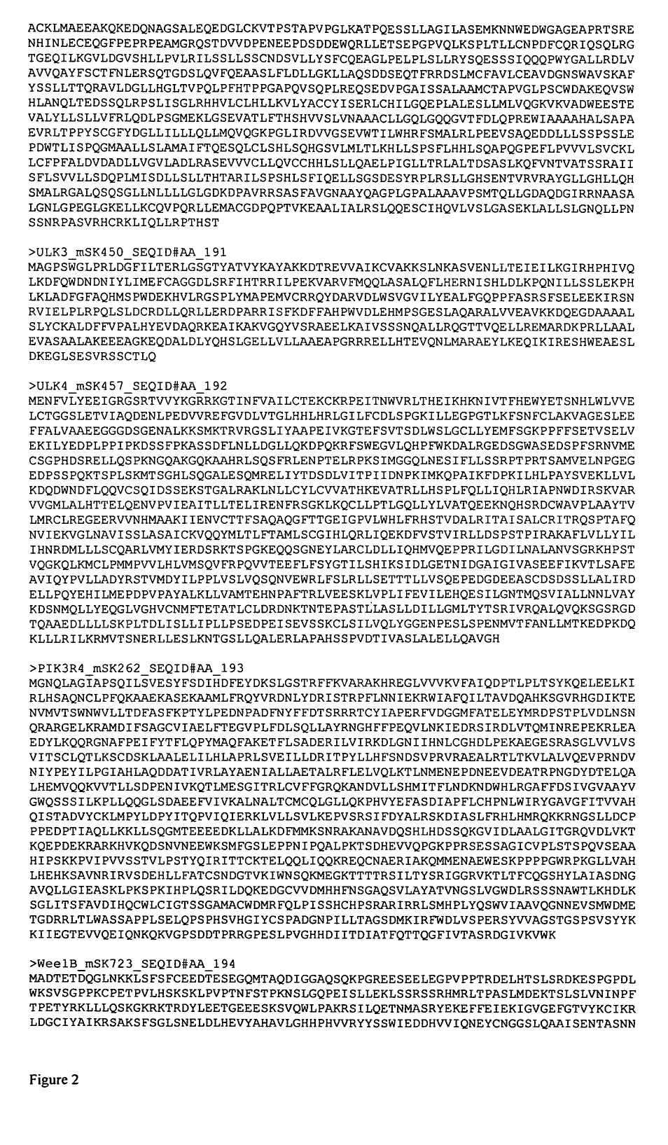

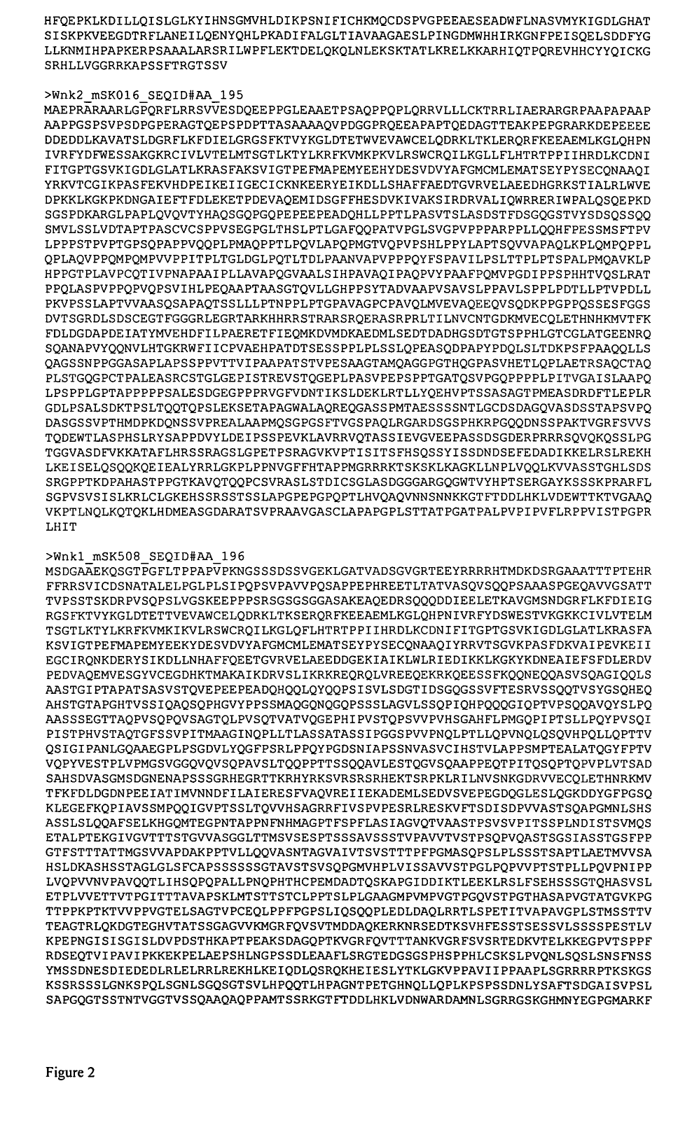

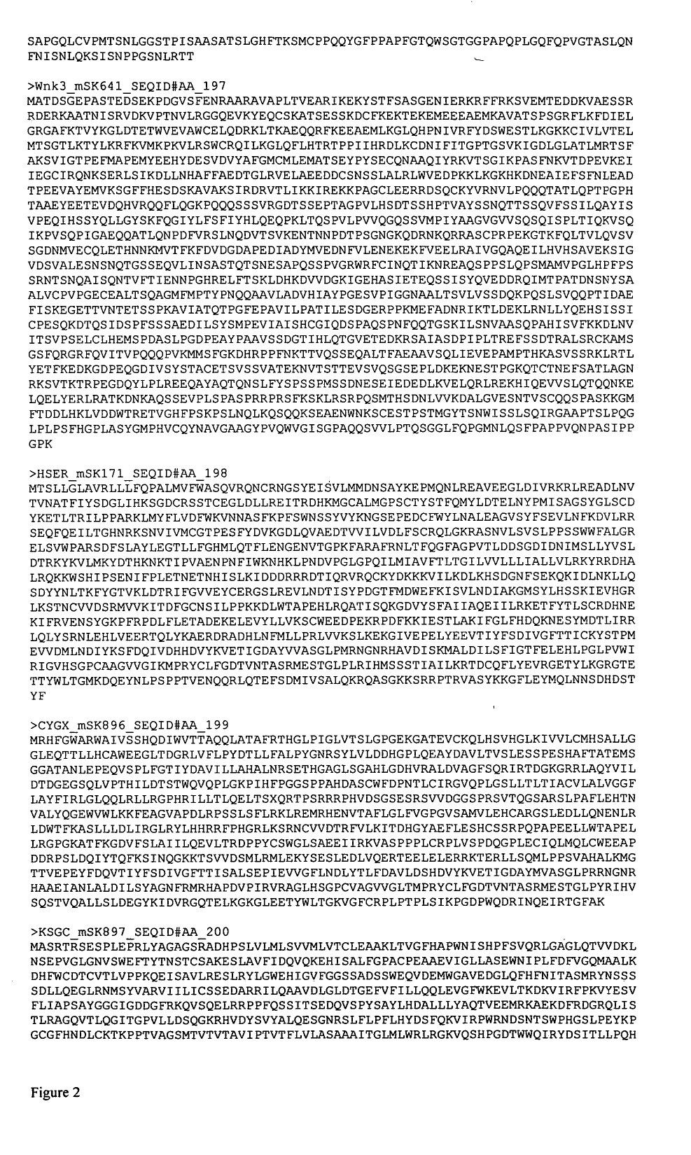

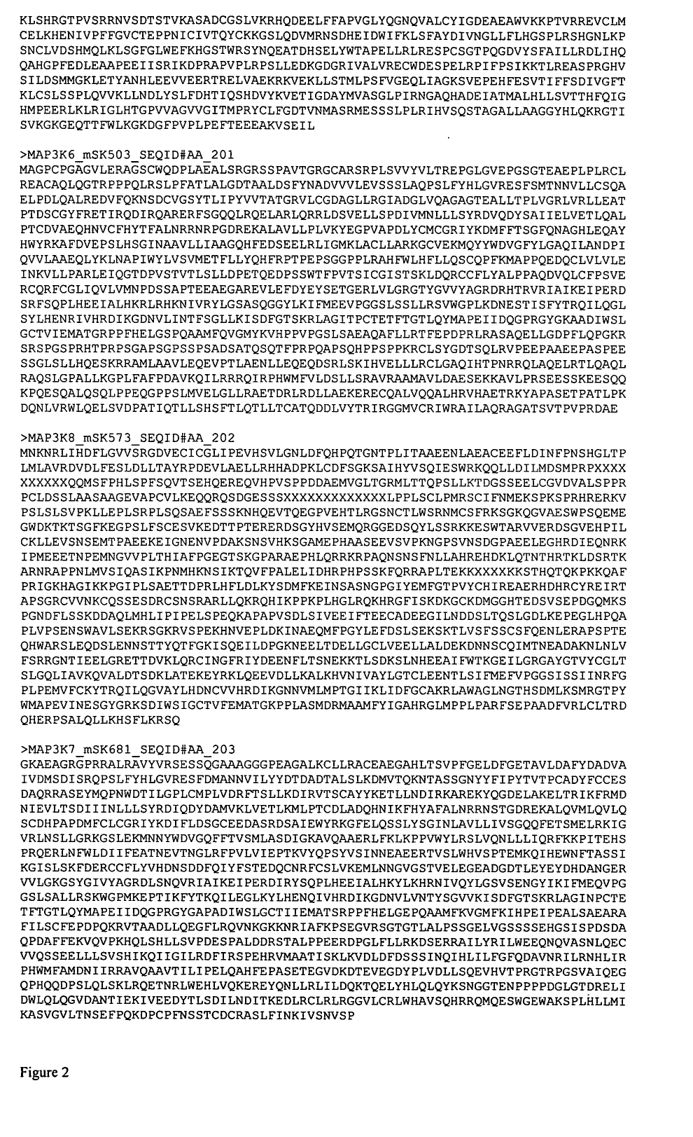

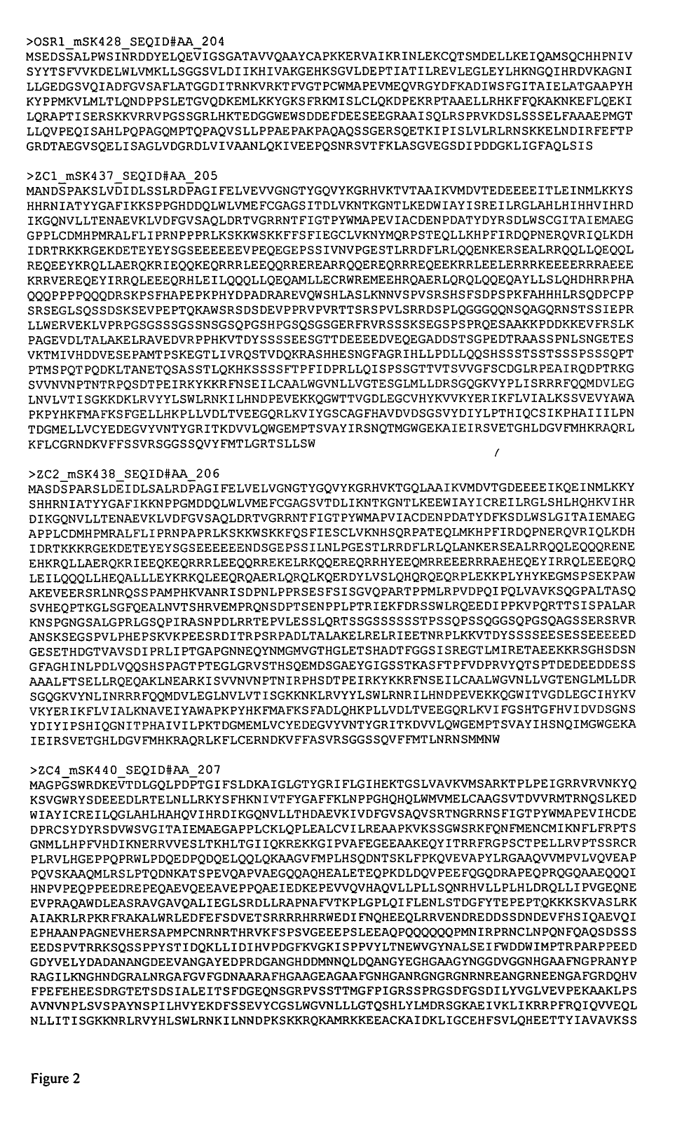

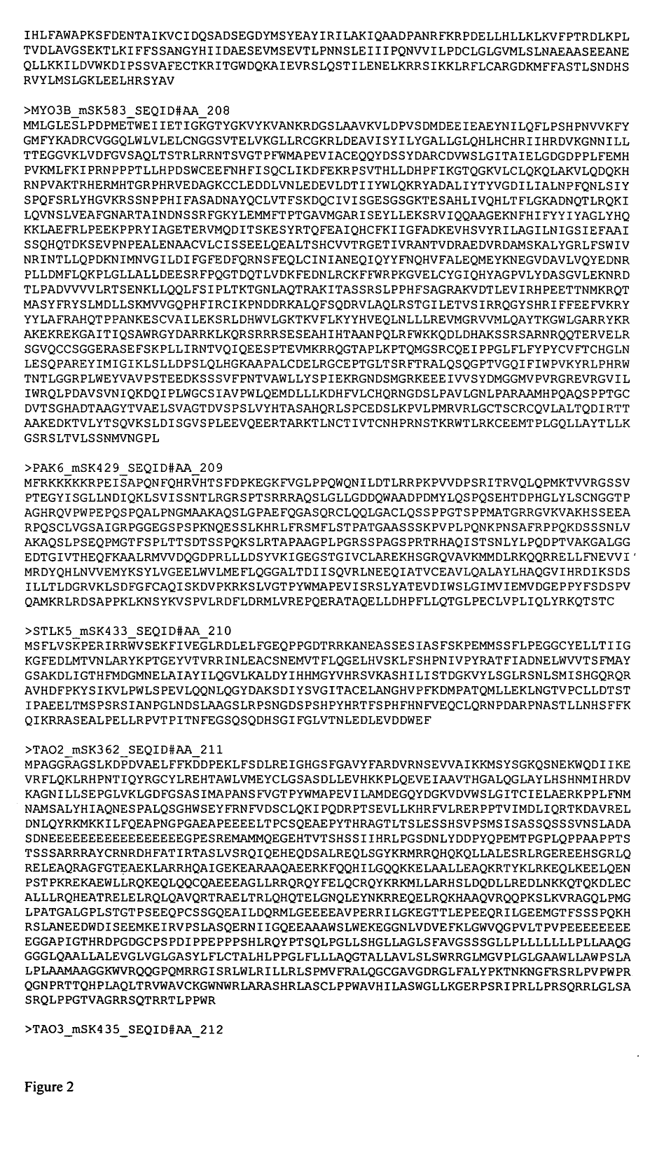

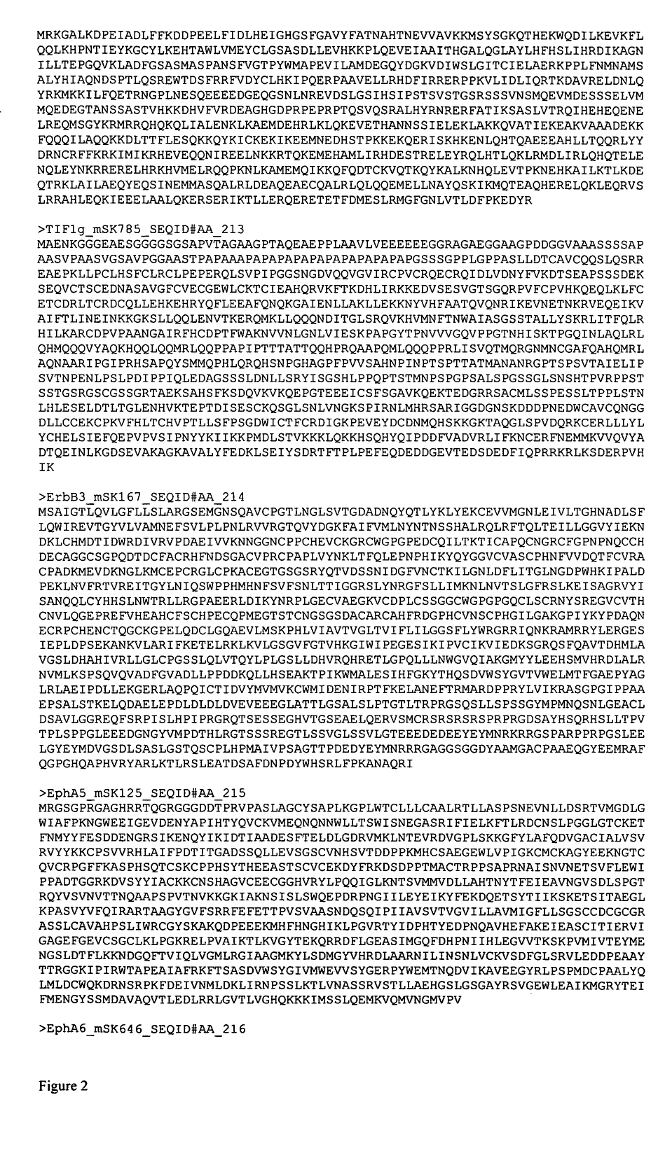

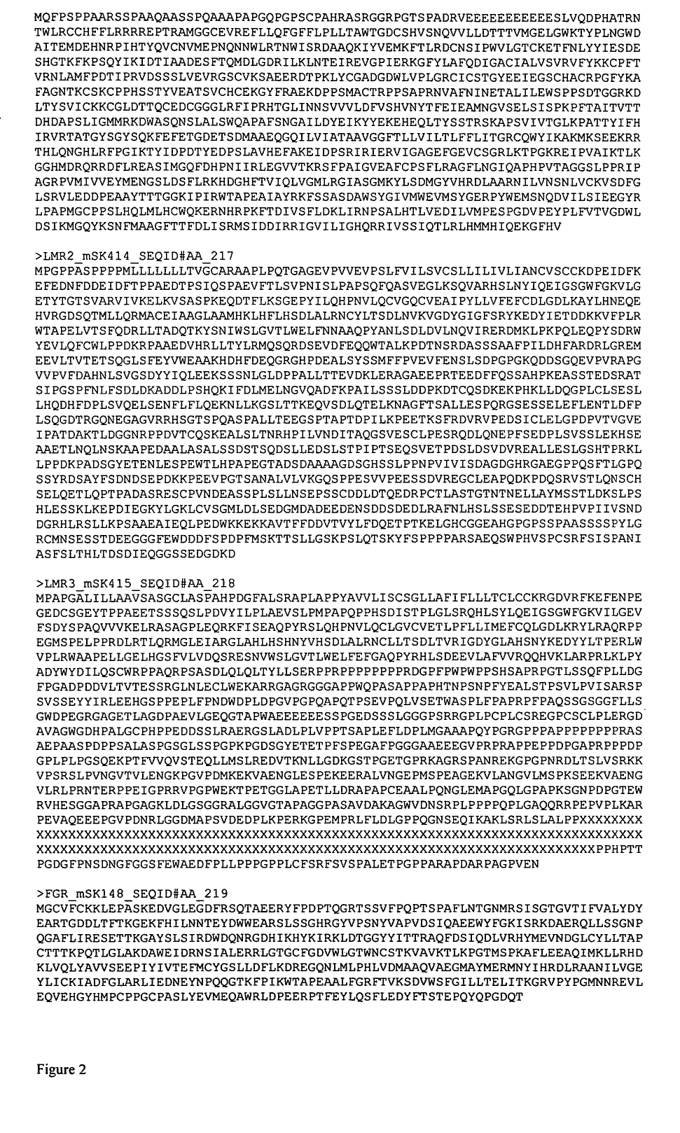

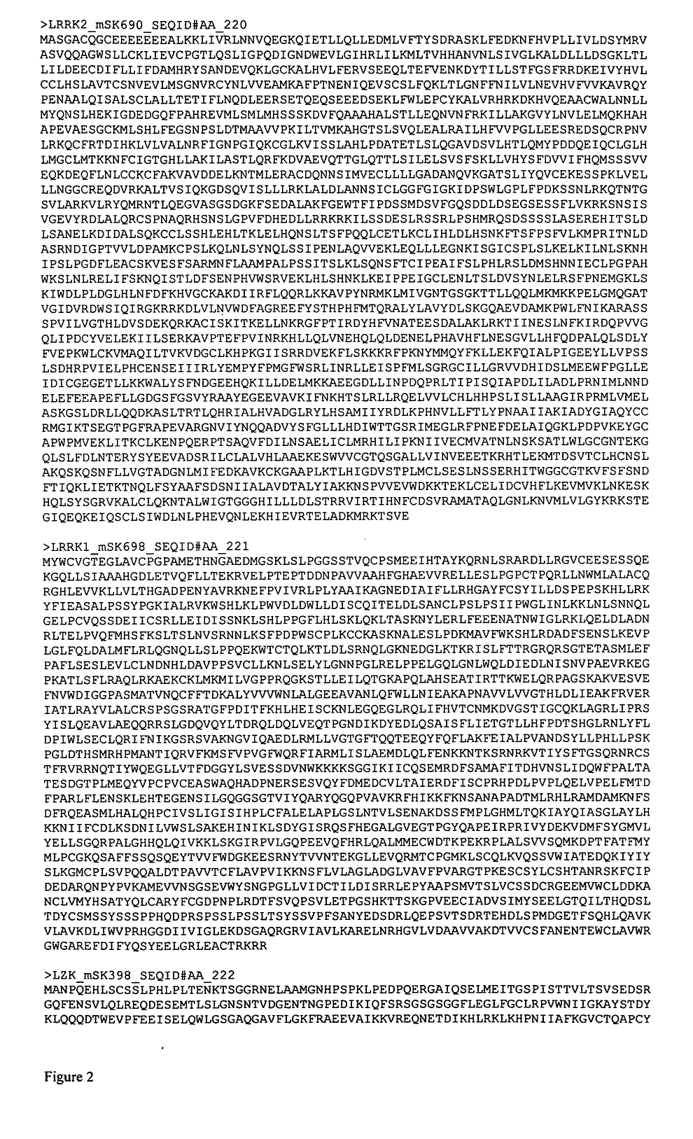

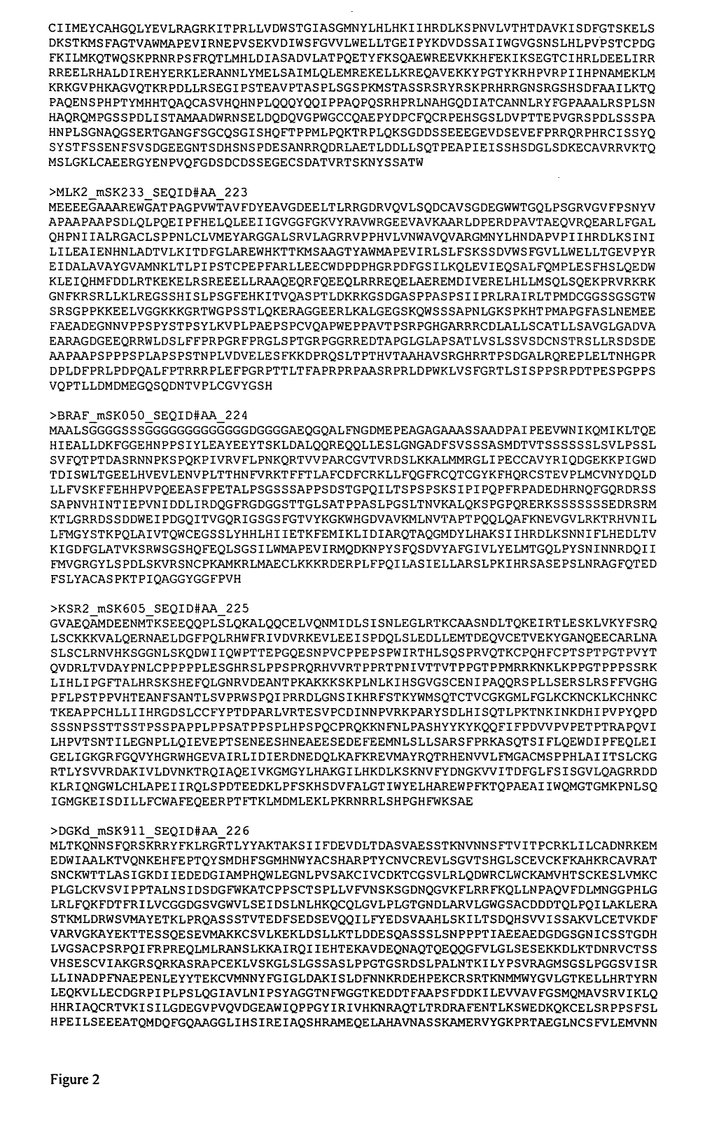

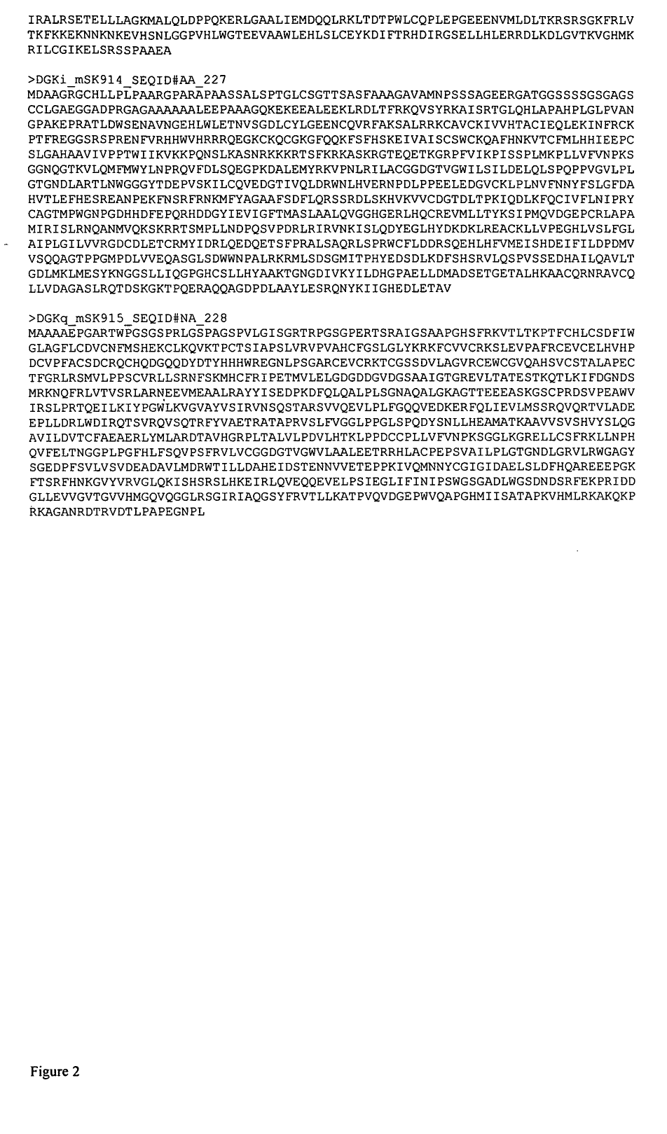

### DETAILED DESCRIPTION OF THE INVENTION

The invention provides, inter alia, protein and lipid kinases and kinase-like genes, as well as fragments thereof, which have been identified in genomic and expressed sequence databases. In part, the invention provides nucleic acid molecules that are capable of encoding polypeptides having a kinase or kinase-like activity. By reference to Tables and Figures provided herein, genes of the invention can be better understood. The invention additionally provides a number of different embodiments, such as those described below.

All of the sequences are derived from mouse genomic and expressed DNA.

Nucleic Acid Probes, Methods, and Kits for Detection of Kinases

The invention additionally provides nucleic acid probes and uses therefor. A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences which correspond to N-terminal and C-terminal portions of the amino acid sequence of the polypeptide of interest. The synthesized nucleic acid probes may be used as primers in a polymerase chain reaction (PCR) carried out in accordance with recognized PCR techniques, essentially according to PCR Protocols, “A Guide to Methods and Applications”, Academic Press, Michael, et al., eds., 1990, utilizing the appropriate chromosomal or cDNA library to obtain the fragment of the present invention.

One skilled in the art can readily design such probes based on the sequence disclosed herein using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin). Preferably, the kit further comprises instructions for use.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

Categorization of the Polypeptides According to the Invention

For a number of protein kinases of the invention, there is provided a classification of the protein class and family to which it belongs, a summary of non-catalytic protein motifs, as well as a chromosomal location, which provides information on function, regulation and/or therapeutic utility for each of the proteins. Amplification of chromosomal region can be associated with various cancers.

The kinase classification and protein domains often reflect pathways, cellular roles, or mechanisms of up- or down-stream regulation. Also disease-relevant genes often occur in families of related genes. For example, if one member of a kinase family functions as an oncogene, a tumor suppressor, or has been found to be disrupted in an immune, neurologic, cardiovascular, or metabolic disorder, frequently other family members may play a similar role.

Chromosomal location can identify candidate targets for a tumor amplicon or a tumor-suppressor locus. Summaries of prevalent tumor amplicons are available in the literature, and can identify tumor types to experimentally be confirmed to contain amplified copies of a kinase gene which localizes to an adjacent region.

As described herein, the polypeptides of the present invention can be classified. The salient features related to the biological and clinical implications of these different groups are described hereafter in more general terms.

A more specific characterization of the polypeptides of the invention, including potential biological and clinical implications, is provided, e.g., in EXAMPLES 2a and 2b.

Classification of Polypeptides Exhibiting Kinase Activity

The classification of the polypeptides described in this application is found in Tables 1 and 2. The present application describes members of the following superfamilies: protein kinase, lipid kinase, atypical protein kinase. The present application also describes members of the following groups: AGC group, CAMK Group, CKI (or CK1) Group, CMGC Group, OTHER Group, STE Group, TK Group, DAG (diacylglycerol) Group, BRD Group.

Potential biological and clinical implications of these novel kinases are described below.

Therapeutic Methods According to the Invention:

Diagnostics:

The invention provides methods for detecting a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide selected from the group consisting of SEQ ID NO:115 through SEQ ID NO:228, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic disorder including diabetes, reproductive disorders including infertility, and cancer.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

“Amplification” as it refers to RNA can be the detectable presence of RNA in cells, since in some normal cells there is no basal expression of RNA. In other normal cells, a basal level of expression exists, therefore in these cases amplification is the detection of at least 1-2-fold, and preferably more, compared to the basal level.

The diseases that could be diagnosed by detection of nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Antibodies, Hybridomas, Methods of Use and Kits for Detection of Kinases

The present invention relates to an antibody having binding affinity to a kinase of the invention. The polypeptide may have the amino acid sequence selected from the group consisting of those set forth in SEQ ID NO:115 through SEQ ID NO:228, or a functional derivative thereof, or at least 9 contiguous amino acids thereof (preferably, at least 20, 30, 35, or 40 contiguous amino acids thereof).

The present invention also relates to an antibody having specific binding affinity to a kinase of the invention. Such an antibody may be isolated by comparing its binding affinity to a kinase of the invention with its binding affinity to other polypeptides. Those which bind selectively to a kinase of the invention would be chosen for use in methods requiring a distinction between a kinase of the invention and other polypeptides. Such methods could include, but should not be limited to, the analysis of altered kinase expression in tissue containing other polypeptides.

The kinases of the present invention can be used in a variety of procedures and methods, such as for the generation of antibodies, for use in identifying pharmaceutical compositions, and for studying DNA/protein interaction.

The kinases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al., J. Histochem. Cytochem. 18:315, 1970; Bayer et al., Meth. Enzym. 62:308, 1979; Engval et al., Immunol. 109:129, 1972; Goding, J. Immunol. Meth. 13:215, 1976. The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilized antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, N.Y., pp. 289-307, 1992; Kaspczak et al., Biochemistry 28:9230-9238, 1989).

Anti-peptide peptides can be generated by replacing the basic amino acid residues found in the peptide sequences of the kinases of the invention with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

The present invention also encompasses a method of detecting a kinase polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of a kinase of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion-based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry,” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology,” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds Capable of Interacting with Kinases

The present invention also relates to a method of detecting a compound capable of binding to a kinase of the invention comprising incubating the compound with a kinase of the invention and detecting the presence of the compound bound to the kinase. The compound may be present within a complex mixture, for example, serum, body fluid, or cell extracts.

The present invention also relates to a method of detecting an agonist or antagonist of kinase activity or kinase binding partner activity comprising incubating cells that produce a kinase of the invention in the presence of a compound and detecting changes in the level of kinase activity or kinase binding partner activity. The compounds thus identified would produce a change in activity indicative of the presence of the compound.

The compound may be present within a complex mixture, for example, serum, body fluid, or cell extracts. Once the compound is identified it can be isolated using techniques well known in the art.

Modulating Polypeptide Activity:

The invention additionally provides methods for treating a disease or abnormal condition by administering to a patient in need of such treatment a substance that modulates the activity of a polypeptide selected from the group consisting of SEQ ID NO:115 through SEQ ID NO:228. Preferably, the disease is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic and reproductive disorders, and cancer.

Substances useful for treatment of disorders or diseases preferably show positive results in one or more assays for an activity corresponding to treatment of the disease or disorder in question Substances that modulate the activity of the polypeptides preferably include, but are not limited to, antisense oligonucleotides and inhibitors of protein kinases.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) a decrease in the proliferation, growth, and/or differentiation of cells; (b) inhibition (, slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation or cell survival. An abnormal condition may also include irregularities in cell cycle progression, i.e., irregularities in normal cell cycle progression through mitosis and meiosis.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to, neurodegenerative disorders, slow wound healing rates, and slow tissue grafting healing rates.

Abnormal cell survival conditions may also relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig or goat, more preferably a monkey or ape, and most preferably a human.

The present invention also encompasses a method of agonizing (stimulating) or antagonizing kinase associated activity in a mammal comprising administering to said mammal an agonist or antagonist to a kinase of the invention in an amount sufficient to effect said agonism or antagonism. A method of treating diseases in a mammal with an agonist or antagonist of the activity of one of the kinases of the invention comprising administering the agonist or antagonist to a mammal in an amount sufficient to agonize or antagonize kinase-associated functions is also encompassed in the present application.

In an effort to discover novel treatments for diseases, biomedical researchers and chemists have designed, synthesized, and tested molecules that inhibit the function of protein kinases. Some small organic molecules form a class of compounds that modulate the function of protein kinases. Examples of molecules that have been reported to inhibit the function of some protein kinases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein kinase inhibitors only weakly inhibit the function of protein kinases. In addition, many inhibit a variety of protein kinases and will therefore cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar moieties including hydroxylated alkyl, phosphate, and ether moieties. U.S. patent application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187) and 08/485,323, filed Jun. 7, 1995, entitled “Benzylidene-Z-Indoline Compounds for the Treatment of Disease” by Tang et al. (Lyon & Lyon Docket No. 223/298) and International Patent Publications WO 96/40116, published Dec. 19, 1996 by Tang, et al., and WO 96/22976, published Aug. 1, 1996 by Ballinari et al., all of which are incorporated herein by reference in their entirety, including any drawings, figures, or tables, describe indolinone chemical libraries of indolinone compounds harboring other bicyclic moieties as well as monocyclic moieties fused to the oxindole ring. Application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187), 08/485,323, filed Jun. 7, 1995, entitled “Benzylidene-Z-Indoline Compounds for the Treatment of Disease” by Tang et al. (Lyon & Lyon Docket No. 223/298), and WO 96/22976, published Aug. 1, 1996 by Ballinari et al. teach methods of indolinone synthesis, methods of testing the biological activity of indolinone compounds in cells, and inhibition patterns of indolinone derivatives.

Other examples of substances capable of modulating kinase activity include, but are not limited to, tyrphostins, quinazolines, quinoxolines, and quinolines. The quinazolines, tyrphostins, quinolines, and quinoxolines referred to above include well known compounds such as those described in the literature. For example, representative publications describing quinazolines include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5,316,553; Kreighbaum and Corner, U.S. Pat. No. 4,343,940; Pegg and Wardleworth, EPO Publication No. 0 562 734 A1; Barker et al., (1991) Proc. of Am. Assoc. for Cancer Research 32:327; Bertino, J. R., (1979) Cancer Research 3:293-304; Bertino, J. R., (1979) Cancer Research 9(2 part 1):293-304; Curtin et al., (1986) Br. J. Cancer 53:361-368; Fernandes et al., (1983) Cancer Research 43:1117-1123; Ferris et al. J. Org. Chem. 44(2):173-178; Fry et al., (1994) Science 265:1093-1095; Jackman et al., (1981) Cancer Research 51:5579-5586; Jones et al. J. Med. Chem. 29(6):1114-1118; Lee and Skibo, (1987) Biochemistry 26(23):7355-7362; Lemus et al., (1989) J. Org. Chem. 54:3511-3518; Ley and Seng, (1975) Synthesis 1975:415-522; Maxwell et al., (1991) Magnetic Resonance in Medicine 17:189-196; Mini et al., (1985) Cancer Research 45:325-330; Phillips and Castle, J. (1980) Heterocyclic Chem. 17(19):1489-1596; Reece et al., (1977) Cancer Research 47(11):2996-2999; Sculier et al., (1986) Cancer Immunol. and Immunother. 23, A65; Sikora et al., (1984) Cancer Letters 23:289-295; Sikora et al., (1988) Analytical Biochem. 172:344-355; all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., (1994) J. Med. Chem. 37:2627-2629; MaGuire, J. (1994) Med. Chem. 37:2129-2131; Burke et al., (1993) J. Med. Chem. 36:425-432; and Burke et al. (1992) BioOrganic Med. Chem. Letters 2:1771-1774, all of which are incorporated by reference in their entirety, including any drawings.

Tyrphostins are described in Allen et al., (1993) Clin. Exp. Immunol. 91:141-156; Anafi et al., (1993) Blood 82:12, 3524-3529; Baker et al., (1992) J Cell Sci. 102:543-555; Bilder et al., (1991) Amer. Physiol. Soc. pp. 6363-6143:C721-C730; Brunton et al., (1992) Proceedings of Amer. Assoc. Cancer Rsch. 33:558; Bryckaert et al., (1992) Exp. Cell Research 199:255-261; Dong et al., (1993) J Leukocyte Biology 53:53-60; Dong et al., (1993) J. Immunol. 151(5):2717-2724; Gazit et al., (1989) J. Med. Chem. 32, 2344-2352; Gazit et al., (1993) J. Med. Chem. 36:3556-3564; Kaur et al., (1994) Anti-Cancer Drugs 5:213-222; King et al., (1991) Biochem. J. 275:413-418; Kuo et al., (1993) Cancer Letters 74:197-202; Levitzki, A., (1992) The FASEB J. 6:3275-3282; Lyall et al., (1989) J. Biol. Chem. 264:14503-14509; Peterson et al., (1993) The Prostate 22:335-345; Pillemer et al., (1992) Int. J. Cancer 50:80-85; Posner et al., (1993) Molecular Pharmacology 45:673-683; Rendu et al., (1992) Biol. Pharmacology 44(5):881-888; Sauro and Thomas, (1993) Life Sciences 53:371-376; Sauro and Thomas, (1993) J. Pharm. and Experimental Therapeutics 267(3):119-1125; Wolbring et al., (1994) J. Biol. Chem. 269(36):22470-22472; and Yoneda et al., (1991) Cancer Research 51:4430-4435; all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

Recombinant DNA Technology:

DNA Constructs Comprising a Kinase Nucleic Acid Molecule and

Cells Containing These Constructs:

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a kinase of the invention may be obtained by the above-described methods. This region may be retained for its transcriptional termination regulatory sequences, such as termination and polyadenylation. Thus, by retaining the 3′-region naturally contiguous to the DNA sequence encoding a kinase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a kinase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a kinase of the invention, or (3) interfere with the ability of the gene sequence of a kinase of the invention to be transcribed by the promoter region sequence. Thus, a promoter region would be operably linked to a DNA sequence if the promoter were capable of effecting transcription of that DNA sequence. Thus, to express a gene encoding a kinase of the invention, transcriptional and translational signals recognized by an appropriate host are necessary.

The present invention encompasses the expression of a gene encoding a kinase of the invention (or a functional derivative thereof) in either prokaryotic or eukaryotic cells. Prokaryotic hosts are, generally, very efficient and convenient for the production of recombinant proteins and are, therefore, one type of preferred expression system for kinases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a kinase of the invention (or a functional derivative thereof) in a prokaryotic cell, it is necessary to operably link the sequence encoding the kinase of the invention to a functional prokaryotic promoter. Such promoters may be either constitutive or, more preferably, regulatable (i.e., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, λrecA, acZ, λacI, and gal promoters of E. coli, the α-amylase (Ulmanen et al., J. Bacteriol. 162:176-182, 1985) and the ζ-28-specific promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the kinase polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus ³⁵S and 19S, and nopaline synthase promoter and polyadenylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of kinases of the invention in insect cells (Jasny, Science 238:1653, 1987; Miller et al., in: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of kinases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of kinases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes a kinase of the invention (or a functional derivative thereof) does not contain any intervening codons which are capable of encoding a methionine (i.e., AUG). The presence of such codons results either in the formation of a fusion protein (if the AUG codon is in the same reading frame as the kinase of the invention coding sequence) or a frame-shift mutation (if the AUG codon is not in the same reading frame as the kinase of the invention coding sequence).

A nucleic acid molecule encoding a kinase of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColEl, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a kinase of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Transgenic Animals:

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 244:1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281-1288, 1989; and Simms et al., Bio/Technology 6:179-183, 1988).

Thus, the invention provides transgenic, nonhuman mammals containing a transgene encoding a kinase of the invention or a gene affecting the expression of the kinase. Such transgenic nonhuman mammals are particularly useful as an in vivo test system for studying the effects of introduction of a kinase, or regulating the expression of a kinase (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode mammalian kinases. Native expression in an animal may be reduced by providing an amount of antisense RNA or DNA effective to reduce expression of the receptor.

Knock-Out Animals:

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration may be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 5,616,491 describes knock-out mice having suppression of CD28 and CD45. Procedures for preparation and manipulation of cells and embryos are similar to those described above with respect to transgenic animals, and are well known to those of ordinary skill in the art.

A knock out construct refers to a uniquely configured fragment of nucleic acid which is introduced into a stem cell line and allowed to recombine with the genome at the chromosomal locus of the gene of interest to be mutated. Thus, a given knock out construct is specific for a given gene to be targeted for disruption. Nonetheless, many common elements exist among these constructs and these elements are well known in the art. A typical knock out construct contains nucleic acid fragments of about 0.5 kb to about 10.0 kb from both the 5′ and the 3′ ends of the genomic locus which encodes the gene to be mutated. These two fragments are typically separated by an intervening fragment of nucleic acid which encodes a positive selectable marker, such as the neomycin resistance gene. The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug.

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of the polypeptide product of the wild-type gene from which it was derived.

Each knockout construct to be inserted into the cell must first be in the linear form. Therefore, if the knockout construct has been inserted into a vector, linearization is accomplished by digesting the DNA with a suitable restriction endonuclease selected to cut only within the vector sequence and not within the knockout construct sequence. For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipette and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan. After the ES cell has been introduced into the embryo, the embryo may be implanted into the uterus of a pseudopregnant foster mother for gestation as described above.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

Uses of Transgenic and Knock-Out Animals:

The transgenic and knock-out animals of the present invention, or cells or cell lines obtained from such animals, can be used to identify substances that bind to and/or modulate the activity of a kinase polypeptide. A wide variety of assays may be used for this purpose, including screening assays, labeled in vitro protein-protein binding assays, protein-DNA binding assays, electrophoretic mobility shift assays, immunoassays for protein binding, kinase activity assays, and the like. Cells may be freshly isolated from an animal, or may be immortalized in culture as cell lines.

Test substances encompass numerous chemical classes, though typically they are organic molecules, preferably small compounds having a molecular weight of more than 50 and less than about 2,500 daltons. Test substances comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

In alternative embodiments, test substances may also include biomolecules including, but not limited to: peptides, polypeptides, proteins, saccharides, fatty acids, steroids, purines, pyrimidines, derivatives, structural analogs or combinations thereof.

Test substances may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

Antibodies disclosed herein may also be used in screening immunoassays, particularly to detect the binding of substrates to kinase polypeptides, or to confirm the presence and/or quantity of a kinase polypeptide in a cell or sample.

Samples obtained from transgenic mice or knock-out mice, as used herein, include biological fluids such as tracheal lavage, blood, cerebrospinal fluid, tears, saliva, lymph, dialysis fluid and the like; organ or tissue culture derived fluids; fluids extracted from physiological tissues; tissue and cells, or homogenates thereof. Also included in the term are derivatives and fractions of any of these types of samples.

Substances identified as modulators of kinase activity identified using invention transgenic or knock-out mice can be used for treating a disease or disorder by administering such as substance to a patient in need thereof. In addition, such substances identified in murine systems can be used to determine their effect on human orthologues of murine polypeptides. Human orthologues may be identified by hybridization of probes described herein obtained from nucleic acid sequences encoding the amino acid sequence of any of SEQ ID NOs:115 through 228.

Gene Therapy:

Kinases or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing a kinase coding sequence is inserted into cells, the cells are grown in vitro and then infused in large numbers into patients. In another preferred embodiment, a DNA segment containing a promoter of choice (for example a strong promoter) is transferred into cells containing an endogenous gene encoding kinases of the invention in such a manner that the promoter segment enhances expression of the endogenous kinase gene (for example, the promoter segment is transferred to the cell such that it becomes directly linked to the endogenous kinase gene).

The gene therapy may involve the use of an adenovirus containing kinase cDNA targeted to a tumor, systemic kinase increase by implantation of engineered cells, injection with kinase-encoding virus, or injection of naked kinase DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction, may be used to inhibit an abnormal, deleterious signal transduction event.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encod-ing recombinant kinase of the invention protein into the targeted cell population (e.g., tumor cells). Methods which are well known to those skilled in the art can be used to construct recombinant viral vectors containing coding sequences (Maniatis et al., Molecular Cloning: A Laboratory Manual, Cold Spring Harbor Laboratory, N.Y., 1989; Ausubel et al., Current Proto-cols in Molecular Biology, Greene Publishing Associates and Wiley Interscience, N.Y., 1989). Alter-natively, recombinant nucleic acid molecules encoding protein sequences can be used as naked DNA or in a recon-stituted system e.g., liposomes or other lipid systems for delivery to target cells (e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins (Miller, supra).

In its simplest form, gene transfer can be performed by simply injecting minute amounts of DNA into the nucleus of a cell, through a process of microinjection (Capecchi, Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with calcium phosphate and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophilic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad. Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expression of a particular product encoded by the gene. The product may include a protein, polypeptide, antisense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cyto-plasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a kinase polypeptide is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

Expression, including over-expression, of a kinase polypeptide of the invention can be inhibited by administration of an antisense molecule that binds to and inhibits expression of the mRNA encoding the polypeptide. Alternatively, expression can be inhibited in an analogous manner using a ribozyme that cleaves the mRNA. General methods of using antisense and ribozyme technology to control gene expression, or of gene therapy methods for expression of an exogenous gene in this manner are well known in the art. Each of these methods utilizes a system, such as a vector, encoding either an antisense or ribozyme transcript of a kinase polypeptide of the invention.

The term “ribozyme” refers to an RNA structure of one or more RNAs having catalytic properties. Ribozymes generally exhibit endonuclease, ligase or polymerase activity. Ribozymes are structural RNA molecules which mediate a number of RNA self-cleavage reactions. Various types of trans-acting ribozymes, including “hammerhead” and “hairpin” types, which have different secondary structures, have been identified. A variety of ribozymes have been characterized. See, for example, U.S. Pat. Nos. 5,246,921, 5,225,347, 5,225,337 and 5,149,796. Mixed ribozymes comprising deoxyribo and ribooligonucleotides with catalytic activity have been described. Perreault, et al., Nature, 344:565-567 (1990).

As used herein, “antisense” refers of nucleic acid molecules or their derivatives which specifically hybridize, e.g., bind, under cellular conditions, with the genomic DNA and/or cellular mRNA encoding a kinase polypeptide of the invention, so as to inhibit expression of that protein, for example, by inhibiting transcription and/or translation. The binding may be by conventional base pair complementarity, or, for example, in the case of binding to DNA duplexes, through specific interactions in the major groove of the double helix.

In one aspect, the antisense construct is an nucleic acid which is generated ex vivo and that, when introduced into the cell, can inhibit gene expression by, without limitation, hybridizing with the mRNA and/or genomic sequences of a kinase polynucleotide of the invention.

Antisense approaches can involve the design of oligonucleotides (either DNA or RNA) that are complementary to kinase polypeptide mRNA and are based on the kinase polynucleotides of the invention, including SEQ ID NO:1 through 66. The antisense oligonucleotides will bind to the kinase polypeptide mRNA transcripts and prevent translation.

Although absolute complementarity is preferred, it is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3:737 (2002). In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

In general, oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Antisense oligonucleotides complementary to mRNA coding regions are less efficient inhibitors of translation but could be used in accordance with the invention. Whether designed to hybridize to the 5′, 3′ or coding region of the kinase polypeptide mRNA, antisense nucleic acids should be at least six nucleotides in length, and are preferably less than about 100 and more preferably less than about 50 or 30 nucleotides in length. Typically they should be between 10 and 25 nucleotides in length. Such principles will inform the practitioner in selecting the appropriate oligonucleotides In preferred embodiments, the antisense sequence is selected from an oligonucleotide sequence that comprises, consists of, or consists essentially of about 10-30, and more preferably 15-25, contiguous nucleotide bases of a nucleic acid sequence selected from the group consisting of SEQ ID NO:1 through 114 or domains thereof.

In another preferred embodiment, the invention includes an isolated, enriched or purified nucleic acid molecule comprising, consisting of or consisting essentially of about 10-30, and more preferably 15-25 contiguous nucleotide bases of a nucleic acid sequence that encodes a polypeptide of SEQ ID NO:115 through SEQ ID NO:228.

Using the sequences of the present invention, antisense oligonucleotides can be designed. Such antisense oligonucleotides would be administered to cells expressing the target kinase and the levels of the target RNA or protein with that of an internal control RNA or protein would be compared. Results obtained using the antisense oligonucleotide would also be compared with those obtained using a suitable control oligonucleotide. A preferred control oligonucleotide is an oligonucleotide of approximately the same length as the test oligonucleotide. Those antisense oligonucleotides resulting in a reduction in levels of target RNA or protein would be selected.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988) or the blood-brain barrier (see, e.g., PCT Publication No. WO 89/10134, published Apr. 25, 1988), hybridization-triggered cleavage agents. (See, e.g., Krol et al. (1988) BioTechniques 6:958-976) or intercalating agents. (See, e.g, Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from moieties such as 5-fluorouracil, 5-bromouracil, 5-chlorouracil, 5-iodouracil, hypoxanthine, xanthine, 4-acetylcytosine, and 5-(carboxyhydroxyethyl) uracil. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. (see also U.S. Pat. Nos. 5,176,996; 5,264,564; and 5,256,775).

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Also suitable are peptidyl nucleic acids, which are polypeptides such as polyserine, polythreonine, etc. including copolymers containing various amino acids, which are substituted at side-chain positions with nucleic acids (T,A,G,C,U). Chains of such polymers are able to hybridize through complementary bases in the same manner as natural DNA/RNA. Alternatively, an antisense construct of the present invention can be delivered, for example, as an expression plasmid or vector that, when transcribed in the cell, produces RNA complementary to at least a unique portion of the cellular mRNA which encodes a kinase polypeptide of the invention.

While antisense nucleotides complementary to the kinase polypeptide coding region sequence can be used, those complementary to the transcribed untranslated region are most preferred.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

Pharmaceutical Formulations and Routes of Administration

The compounds described herein, including kinase polypeptides of the invention, antisense molecules, ribozymes, and any other compound that modulates the activity of a kinase polypeptide of the invention, can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

Routes Of Administration:

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Composition/Formulation:

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the tyrosine or serine/threonine kinase modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable Dosage Regimens:

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the tyrosine or serine/threonine kinase activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

In another example, toxicity studies can be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging:

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

Functional Derivatives

Also provided herein are functional derivatives of a polypeptide or nucleic acid of the invention. By “functional derivative” is meant a “chemical derivative,” “fragment,” or “variant,” of the polypeptide or nucleic acid of the invention, which terms are defined below. A functional derivative retains at least a portion of the function of the protein, for example reactivity with an antibody specific for the protein, enzymatic activity or binding activity mediated through noncatalytic domains, which permits its utility in accordance with the present invention. It is well known in the art that due to the degeneracy of the genetic code numerous different nucleic acid sequences can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from one selected from the group consisting of those set forth in SEQ ID NO:1 through SEQ ID NO:114. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula selected from the group consisting of those set forth in SEQ ID NO:1 through SEQ ID NO:114, or a derivative thereof. Any nucleotide or polynucleotide may be used in this regard, provided that its addition, deletion or substitution does not alter the amino acid sequence of selected from the group consisting of those set forth in SEQ ID NO:1 through 66, which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the kinase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenyl, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine alpha-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl) dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W. H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

The term “fragment” is used to indicate a polypeptide derived from the amino acid sequence of the proteins, of the complexes having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. Fragments of a protein are useful for screening for substances that act to modulate signal transduction, as described herein. It is understood that such fragments may retain one or more characterizing portions of the native complex. Examples of such retained characteristics include: catalytic activity; substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

### Tables and Description Thereof

This patent application describes 114 protein and lipid kinase polypeptides identified in genomic and cDNA sequence databases. The results are summarized in the Tables 1-4 described below.

Table 1 documents the name of each gene, the nucleic acid and amino acid sequence identification numbers, the classifications of each gene (superfamily, family and group), the lengths of the nucleic acid and protein sequences, the positions and lengths of the open reading frames within the sequence. From left to right the data presented is as follows: Gene name, ID#NA, ID#AA, Super-family, Group, Family, Subfamily NA_length, AA_length, ORF Start, ORF End, ORF Length, and Orthologous human gene. “Gene name” refers to name given the sequence encoding the kinase or kinase-like enzyme. The “ID#NA” and “ID#AA” refer to the SEQ ID NOS given each nucleic acid and amino acid sequence in this patent. “Superfamily” identifies whether the gene is a protein kinase, a lipid kinase, or protein-kinase-like. “Group”, “Family”, and “Subfamily” refer to the protein kinase classification defined by sequence homology and based on previously established phylogenetic analysis [Hardie, G. and Hanks S. The Protein Kinase Book, Academic Press (1995) and Hunter T. and Plowman, G. Trends in Biochemical Sciences (1977) 22:18-22 and Manning, G et al (2002) Science 298:1912-1934]. “NA_length” refers to the length in nucleotides of the corresponding nucleic acid sequence. “AA length” refers to the length in amino acids of the peptide encoded in the corresponding nuclei acid sequence. “ORF start” refers to the beginning nucleotide of the open reading frame. “ORF end” refers to the last nucleotide of the open reading frame, excluding the stop codon. “ORF length” refers to the length in nucleotides of the open reading frame (including the stop codon).

TABLE 1

| Gene_{—} | | ID# | ID# | Super- | | | | NA_{—} | AA_{—} | ORF | ORF | ORF | Orthologous |
|---|---|---|---|---|---|---|---|---|---|---|---|---|---|
| Name | mmSK | NA | AA | family | Group | Family | Subfamily | length | length | Start | End | Length | human gene |
| DMPK2 | mSK112 | 1 | 115 | Protein | AGC | DMPK | GEK | 5308 | 1556 | 260 | 4927 | 4668 | DMPK2 |
| | | | | Kinase | | | | | | | | | |
| MRCKb | mSK241 | 2 | 116 | Protein | AGC | DMPK | GEK | 5475 | 1713 | 89 | 5227 | 5139 | MRCKb |
| | | | | Kinase | | | | | | | | | |
| MAST3 | mSK196 | 3 | 117 | Protein | AGC | MAST | | 4149 | 1321 | 56 | 4018 | 3963 | MAST3 |
| | | | | Kinase | | | | | | | | | |
| MAST1 | mSK345 | 4 | 118 | Protein | AGC | MAST | | 4869 | 1570 | 60 | 4769 | 4710 | MAST1 |
| | | | | Kinase | | | | | | | | | |
| LATS1 | mSK441 | 5 | 119 | Protein | AGC | NDR | | 3714 | 1129 | 1 | 3387 | 3387 | LATS1 |
| | | | | Kinase | | | | | | | | | |
| PKN1 | mSK317 | 6 | 120 | Protein | AGC | PKN | | 3092 | 946 | 129 | 2966 | 2838 | PKN1 |
| | | | | Kinase | | | | | | | | | |
| SGK494 | mSK491 | 7 | 121 | Protein | AGC | RSK | | 1306 | 395 | 122 | 1306 | 1185 | SGK494 |
| | | | | Kinase | | | | | | | | | |
| RSKL1 | mSK517 | 8 | 122 | Protein | AGC | RSKL | | 3316 | 1056 | 88 | 3255 | 3168 | RSKL1 |
| | | | | Kinase | | | | | | | | | |
| ADCK4 | mSK013 | 9 | 123 | atyp PK | Atypical | ABC1 | ABC1-A | 2201 | 533 | 156 | 1754 | 1599 | ADCK4 |
| ADCK5 | mSK780 | 10 | 124 | atyp PK | Atypical | ABC1 | ABC1-B | 1953 | 582 | 62 | 1807 | 1746 | ADCK5 |
| AlphaK2 | mSK754 | 11 | 125 | atyp PK | Atypical | Alpha | | 5591 | 1672 | 17 | 5032 | 5016 | AlphaK2 |
| AlphaK3 | mSK755 | 12 | 126 | atyp PK | Atypical | Alpha | | 4398 | 1231 | 448 | 4140 | 3693 | AlphaK3 |
| BCR | mSK047 | 13 | 127 | atyp PK | Atypical | BCR | | 6478 | 1270 | 75 | 3884 | 3810 | BCR |
| ATR | mSK039 | 14 | 128 | atyp PK | Atypical | PIKK | ATR | 8184 | 2635 | 248 | 8152 | 7905 | ATR |
| AMPKa1 | mSK032 | 15 | 129 | Protein | CAMK | CAMKL | A MPK | 1735 | 550 | 25 | 1674 | 1650 | AMPKa1 |
| | | | | Kinase | | | | | | | | | |
| mSK794 | mSK794 | 16 | 130 | Protein | CAMK | CAMKL | M ARK | 1446 | 481 | 1 | 1443 | 1443 | <none> |
| | | | | Kinase | | | | | | | | | |
| mSK798 | mSK798 | 17 | 131 | Protein | CAMK | CAMKL | M ARK | 534 | 177 | 1 | 531 | 531 | <none> |
| | | | | Kinase | | | | | | | | | |
| mSK801 | mSK801 | 18 | 132 | Protein | CAMK | CAMKL | M ARK | 1479 | 492 | 1 | 1476 | 1476 | <none> |
| | | | | Kinase | | | | | | | | | |
| mSK804 | mSK804 | 19 | 133 | Protein | CAMK | CAMKL | M ARK | 1479 | 492 | 1 | 1476 | 1476 | <none> |
| | | | | Kinase | | | | | | | | | |
| mSK805 | mSK805 | 20 | 134 | Protein | CAMK | CAMKL | M ARK | 915 | 304 | 1 | 912 | 912 | <none> |
| | | | | Kinase | | | | | | | | | |
| mSK807 | mSK807 | 21 | 135 | Protein | CAMK | CAMKL | M ARK | 1434 | 477 | 1 | 1431 | 1431 | <none> |
| | | | | Kinase | | | | | | | | | |
| mSK808 | mSK808 | 22 | 136 | Protein | CAMK | CAMKL | M ARK | 717 | 238 | 1 | 714 | 714 | <none> |
| | | | | Kinase | | | | | | | | | |
| mSK809 | mSK809 | 23 | 137 | Protein | CAMK | CAMKL | M ARK | 1425 | 474 | 1 | 1422 | 1422 | <none> |
| | | | | Kinase | | | | | | | | | |
| mSK811 | mSK811 | 24 | 138 | Protein | CAMK | CAMKL | M ARK | 918 | 306 | 1 | 918 | 918 | <none> |
| | | | | Kinase | | | | | | | | | |
| mSK813 | mSK813 | 25 | 139 | Protein | CAMK | CAMKL | M ARK | 1039 | 232 | 387 | 1037 | 651 | <none> |
| | | | | Kinase | | | | | | | | | |
| mSK814 | mSK814 | 26 | 140 | Protein | CAMK | CAMKL | M ARK | 630 | 209 | 1 | 627 | 627 | <none> |
| | | | | Kinase | | | | | | | | | |
| mSK815 | mSK815 | 27 | 141 | Protein | CAMK | CAMKL | M ARK | 1155 | 384 | 1 | 1152 | 1152 | <none> |
| | | | | Kinase | | | | | | | | | |
| mSK817 | mSK817 | 28 | 142 | Protein | CAMK | CAMKL | M ARK | 537 | 178 | 1 | 534 | 534 | <none> |
| | | | | Kinase | | | | | | | | | |
| mSK822 | mSK822 | 29 | 143 | Protein | CAMK | CAMKL | M ARK | 1854 | 617 | 1 | 1851 | 1851 | <none> |
| | | | | Kinase | | | | | | | | | |
| mSK823 | mSK823 | 30 | 144 | Protein | CAMK | CAMKL | M ARK | 1236 | 411 | 1 | 1233 | 1233 | <none> |
| | | | | Kinase | | | | | | | | | |
| mSK826 | mSK826 | 31 | 145 | Protein | CAMK | CAMKL | M ARK | 1563 | 520 | 1 | 1560 | 1560 | <none> |
| | | | | Kinase | | | | | | | | | |
| mSK836 | mSK836 | 32 | 146 | Protein | CAMK | CAMKL | M ARK | 796 | 261 | 11 | 793 | 783 | <none> |
| | | | | Kinase | | | | | | | | | |
| mSK838 | mSK838 | 33 | 147 | Protein | CAMK | CAMKL | M ARK | 1431 | 476 | 1 | 1428 | 1428 | <none> |
| | | | | Kinase | | | | | | | | | |
| mSK840 | mSK840 | 34 | 148 | Protein | CAMK | CAMKL | M ARK | 1545 | 514 | 1 | 1542 | 1542 | <none> |
| | | | | Kinase | | | | | | | | | |
| mSK843 | mSK843 | 35 | 149 | Protein | CAMK | CAMKL | M ARK | 966 | 322 | 1 | 966 | 966 | <none> |
| | | | | Kinase | | | | | | | | | |
| NuaK1 | mSK195 | 36 | 150 | Protein | CAMK | CAMKL | NuaK | 4933 | 658 | 1 | 1974 | 1974 | NuaK1 |
| | | | | Kinase | | | | | | | | | |
| QSK | mSK501 | 37 | 151 | Protein | CAMK | CAMKL | QIK | 4094 | 1356 | 3 | 4070 | 4068 | QSK |
| | | | | Kinase | | | | | | | | | |
| DCAMKL1 | mSK063 | 38 | 152 | Protein | CAMK | DCAMKL | | 2382 | 745 | 145 | 2379 | 2235 | DCAMKL1 |
| | | | | Kinase | | | | | | | | | |
| MNK2 | mSK236 | 39 | 153 | Protein | CAMK | MAPKAPK | MNK | 2682 | 459 | 238 | 1614 | 1377 | MNK2 |
| | | | | Kinase | | | | | | | | | |
| smMLCK | mSK231 | 40 | 154 | Protein | CAMK | MLCK | | 5984 | 1950 | 55 | 5904 | 5850 | smMLCK |
| | | | | Kinase | | | | | | | | | |
| TTN | mSK372 | 41 | 155 | Protein | CAMK | MLCK | | 1 | 0 | 1 | 110838 | 110838 | TTN |
| | | | | Kinase | | | | | | | | | |
| skMLCK | mSK675 | 42 | 156 | Protein | CAMK | MLCK | | 2960 | 613 | 198 | 2036 | 1839 | skMLCK |
| | | | | Kinase | | | | | | | | | |
| SgK085 | mSK709 | 43 | 157 | Protein | CAMK | MLCK | | 1173 | 390 | 1 | 1170 | 1170 | SgK085 |
| | | | | Kinase | | | | | | | | | |
| PIM2 | mSK292 | 44 | 158 | Protein | CAMK | PIM | | 2155 | 411 | 1 | 1233 | 1233 | PIM2 |
| | | | | Kinase | | | | | | | | | |
| Trio | mSK376 | 45 | 159 | Protein | CAMK | Trio | | 10010 | 3103 | 353 | 9661 | 9309 | Trio |
| | | | | Kinase | | | | | | | | | |
| Trad | mSK533 | 46 | 160 | Protein | CAMK | Trio | | 10435 | 2966 | 1 | 8898 | 8898 | Trad |
| | | | | Kinase | | | | | | | | | |
| SPEG | mSK537 | 47 | 161 | Protein | CAMK | Trio | | 10803 | 3262 | 144 | 9929 | 9786 | SPEG |
| | | | | Kinase | | | | | | | | | |
| Obscurin | mSK601 | 48 | 162 | Protein | CAMK | Trio | | 25569 | 8523 | 1 | 25569 | 25569 | Obscurin |
| | | | | Kinase | | | | | | | | | |
| TSSK5 | mSK848 | 49 | 163 | Protein | CAMK | TSSK | | 1519 | 372 | 257 | 1372 | 1116 | <none> |
| | | | | Kinase | | | | | | | | | |
| CK1g3 | mSK087 | 50 | 164 | Protein | CK1 | CK1 | | 2081 | 448 | 252 | 1595 | 1344 | CK1g3 |
| | | | | Kinase | | | | | | | | | |
| TTBK2 | mSK453 | 51 | 165 | Protein | CK1 | TTBK | | 4209 | 1243 | 478 | 4206 | 3729 | TTBK2 |
| | | | | Kinase | | | | | | | | | |
| TTBK1 | mSK526 | 52 | 166 | Protein | CK1 | TTBK | | 4224 | 1308 | 298 | 4221 | 3924 | TTBK1 |
| | | | | Kinase | | | | | | | | | |
| CHED | mSK076 | 53 | 167 | Protein | CMGC | CDK | CRK7 | 5272 | 1511 | 352 | 4884 | 4533 | CHED |
| | | | | Kinase | | | | | | | | | |
| PFTAIRE2 | mSK462 | 54 | 168 | Protein | CMGC | CDK | TAIRE | 1563 | 433 | 68 | 1366 | 1299 | PFTAIRE2 |
| | | | | Kinase | | | | | | | | | |
| CDKL5 | mSK361 | 55 | 169 | Protein | CMGC | CDKL | | 2928 | 904 | 217 | 2928 | 2712 | CDKL5 |
| | | | | Kinase | | | | | | | | | |
| CDKL4 | mSK466 | 56 | 170 | Protein | CMGC | CDKL | | 1029 | 342 | 1 | 1026 | 1026 | CDKL4 |
| | | | | Kinase | | | | | | | | | |
| DYRK4 | mSK116 | 57 | 171 | Protein | CMGC | DYRK | DYRK2 | 1776 | 592 | 1 | 1776 | 1776 | DYRK4 |
| | | | | Kinase | | | | | | | | | |
| HIPK4 | mSK582 | 58 | 172 | Protein | CMGC | DYRK | HIPK | 1929 | 541 | 1 | 1623 | 1623 | HIPK4 |
| | | | | Kinase | | | | | | | | | |
| ERK4 | mSK137 | 59 | 173 | Protein | CMGC | MAPK | ERK | 2206 | 583 | 1 | 1749 | 1749 | ERK4 |
| | | | | Kinase | | | | | | | | | |
| AAK1 | mSK422 | 60 | 174 | Protein | Other | NAK | | 3345 | 958 | 227 | 3100 | 2874 | AAK1 |
| | | | | Kinase | | | | | | | | | |
| NEK1 | mSK250 | 61 | 175 | Protein | Other | NEK | | 5590 | 1275 | 576 | 4400 | 3825 | NEK1 |
| | | | | Kinase | | | | | | | | | |
| NEK5 | mSK558 | 62 | 176 | Protein | Other | NEK | | 2898 | 778 | 92 | 2425 | 2334 | NEK5 |
| | | | | Kinase | | | | | | | | | |
| NEK10 | mSK645 | 63 | 177 | Protein | Other | NEK | | 3336 | 1111 | 1 | 3333 | 3333 | NEK10 |
| | | | | Kinase | | | | | | | | | |
| SgK069 | mSK581 | 64 | 178 | Protein | Other | NKF1 | | 1145 | 362 | 57 | 1142 | 1086 | SgK069 |
| | | | | Kinase | | | | | | | | | |
| SgK110 | mSK592 | 65 | 179 | Protein | Other | NKF1 | | 624 | 207 | 3 | 623 | 621 | SgK110 |
| | | | | Kinase | | | | | | | | | |
| SgK223 | mSK643 | 66 | 180 | Protein | Other | NKF3 | | 4505 | 1373 | 257 | 4375 | 4119 | SgK223 |
| | | | | Kinase | | | | | | | | | |
| SgK269 | mSK649 | 67 | 181 | Protein | Other | NKF3 | | 6996 | 1735 | 470 | 5674 | 5205 | SgK269 |
| | | | | Kinase | | | | | | | | | |
| CLIK1 | mSK493 | 68 | 182 | Protein | Other | NKF4 | | 1870 | 539 | 1 | 1617 | 1617 | CLIK1 |
| | | | | Kinase | | | | | | | | | |
| SgK307 | mSK699 | 69 | 183 | Protein | Other | NKF5 | | 4776 | 1450 | 108 | 4457 | 4350 | SgK307 |
| | | | | Kinase | | | | | | | | | |
| SgK424 | mSK707 | 70 | 184 | Protein | Other | NKF5 | | 1470 | 469 | 64 | 1470 | 1407 | SgK424 |
| | | | | Kinase | | | | | | | | | |
| NRBP2 | mSK520 | 71 | 185 | Protein | Other | NRBP | | 3147 | 499 | 134 | 1630 | 1497 | NRBP2 |
| | | | | Kinase | | | | | | | | | |
| SgK493 | mSK460 | 72 | 186 | Protein | Other | Other-Unique | | 1473 | 491 | 1 | 1473 | 1473 | SgK493 |
| | | | | Kinase | | | | | | | | | |
| SgK496 | mSK516 | 73 | 187 | Protein | Other | Other-Unique | | 5262 | 927 | 52 | 2832 | 2781 | SgK496 |
| | | | | Kinase | | | | | | | | | |
| SgK071 | mSK521 | 74 | 188 | Protein | Other | Other-Unique | | 1878 | 626 | 1 | 1878 | 1878 | SgK071 |
| | | | | Kinase | | | | | | | | | |
| SgK384 | mSK895 | 75 | 189 | Protein | Other | PLK | | 2106 | 599 | 106 | 1902 | 1797 | SgK384 |
| | | | | Kinase | | | | | | | | | |
| Fused | mSK199 | 76 | 190 | Protein | Other | ULK | | 4403 | 1316 | 225 | 4172 | 3948 | Fused |
| | | | | Kinase | | | | | | | | | |
| ULK3 | mSK450 | 77 | 191 | Protein | Other | ULK | | 1807 | 472 | 155 | 1570 | 1416 | ULK3 |
| | | | | Kinase | | | | | | | | | |
| ULK4 | mSK457 | 78 | 192 | Protein | Other | ULK | | 4076 | 1275 | 159 | 3983 | 3825 | ULK4 |
| | | | | Kinase | | | | | | | | | |
| PIK3R4 | mSK262 | 79 | 193 | Protein | Other | VPS15 | | 4777 | 1358 | 455 | 4528 | 4074 | PIK3R4 |
| | | | | Kinase | | | | | | | | | |
| Wee1B | mSK723 | 80 | 194 | Protein | Other | WEE | | 1708 | 555 | 1 | 1665 | 1665 | Wee1B |
| | | | | Kinase | | | | | | | | | |
| Wnk2 | mSK016 | 81 | 195 | Protein | Other | Wnk | | 6432 | 2132 | 34 | 6429 | 6396 | Wnk2 |
| | | | | Kinase | | | | | | | | | |
| Wnk1 | mSK508 | 82 | 196 | Protein | Other | Wnk | | 7658 | 2377 | 110 | 7240 | 7131 | Wnk1 |
| | | | | Kinase | | | | | | | | | |
| Wnk3 | mSK641 | 83 | 197 | Protein | Other | Wnk | | 5256 | 1751 | 1 | 5253 | 5253 | Wnk3 |
| | | | | Kinase | | | | | | | | | |
| HSER | mSK171 | 84 | 198 | Protein | RGC | RGC | | 3924 | 1066 | 120 | 3317 | 3198 | HSER |
| | | | | Kinase | | | | | | | | | |
| CYGX | mSK896 | 85 | 199 | Protein | RGC | RGC | | 3294 | 908 | 571 | 3294 | 2724 | <none> |
| | | | | Kinase | | | | | | | | | |
| KSGC | mSK897 | 86 | 200 | Protein | RGC | RGC | | 3891 | 1101 | 1 | 3303 | 3303 | <none> |
| | | | | Kinase | | | | | | | | | |
| MAP3K6 | mSK503 | 87 | 201 | Protein | STE | STE11 | | 4333 | 1291 | 272 | 4144 | 3873 | MAP3K6 |
| | | | | Kinase | | | | | | | | | |
| MAP3K8 | mSK573 | 88 | 202 | Protein | STE | STE11 | | 4167 | 1388 | 1 | 4164 | 4164 | MAP3K8 |
| | | | | Kinase | | | | | | | | | |
| MAP3K7 | mSK681 | 89 | 203 | Protein | STE | STE11 | | 4003 | 1334 | 1 | 4002 | 4002 | MAP3K7 |
| | | | | Kinase | | | | | | | | | |
| OSR1 | mSK428 | 90 | 204 | Protein | STE | STE20 | FRAY | 2344 | 527 | 285 | 1865 | 1581 | OSR1 |
| | | | | Kinase | | | | | | | | | |
| ZC1 | mSK437 | 91 | 205 | Protein | STE | STE20 | MSN | 4200 | 1328 | 1 | 3984 | 3984 | ZC1 |
| | | | | Kinase | | | | | | | | | |
| ZC2 | mSK438 | 92 | 206 | Protein | STE | STE20 | MSN | 7020 | 1351 | 348 | 4400 | 4053 | ZC2 |
| | | | | Kinase | | | | | | | | | |
| ZC4 | mSK440 | 93 | 207 | Protein | STE | STE20 | MSN | 4620 | 1539 | 1 | 4617 | 4617 | ZC4 |
| | | | | Kinase | | | | | | | | | |
| MYO3B | mSK583 | 94 | 208 | Protein | STE | STE20 | NinaC | 4875 | 1613 | 34 | 4872 | 4839 | MYO3B |
| | | | | Kinase | | | | | | | | | |
| PAK6 | mSK429 | 95 | 209 | Protein | STE | STE20 | PAKB | 4125 | 682 | 633 | 2678 | 2046 | PAK6 |
| | | | | Kinase | | | | | | | | | |
| STLK5 | mSK433 | 96 | 210 | Protein | STE | STE20 | STLK | 2201 | 431 | 203 | 1495 | 1293 | STLK5 |
| | | | | Kinase | | | | | | | | | |
| TAO2 | mSK362 | 97 | 211 | Protein | STE | STE20 | TAO | 4062 | 1240 | 237 | 3956 | 3720 | TAO2 |
| | | | | Kinase | | | | | | | | | |
| TAO3 | mSK435 | 98 | 212 | Protein | STE | STE20 | TAO | 3062 | 898 | 287 | 2980 | 2694 | TAO3 |
| | | | | Kinase | | | | | | | | | |
| TIF1g | mSK785 | 99 | 213 | atyp PK | TIF1 | | | 5149 | 1142 | 74 | 3499 | 3426 | TIF1g |
| ErbB3 | mSK167 | 100 | 214 | Protein | TK | EGFR | | 5978 | 1339 | 122 | 4138 | 4017 | ErbB3 |
| | | | | Kinase | | | | | | | | | |
| EphA5 | mSK125 | 101 | 215 | Protein | TK | Eph | | 4814 | 1041 | 418 | 3540 | 3123 | EphA5 |
| | | | | Kinase | | | | | | | | | |
| EphA6 | mSK646 | 102 | 216 | Protein | TK | Eph | | 4229 | 1130 | 284 | 3673 | 3390 | EphA6 |
| | | | | Kinase | | | | | | | | | |
| LMR2 | mSK414 | 103 | 217 | Protein | TK | Lmr | | 6677 | 1469 | 237 | 4643 | 4407 | LMR2 |
| | | | | Kinase | | | | | | | | | |
| LMR3 | mSK415 | 104 | 218 | Protein | TK | Lmr | | 4930 | 1431 | 501 | 4793 | 4293 | LMR3 |
| | | | | Kinase | | | | | | | | | |
| FGR | mSK148 | 105 | 219 | Protein | TK | Src | | 2184 | 517 | 188 | 1738 | 1551 | FGR |
| | | | | Kinase | | | | | | | | | |
| LRRK2 | mSK690 | 106 | 220 | Protein | TKL | LRRK | | 7985 | 2478 | 58 | 7491 | 7434 | LRRK2 |
| | | | | Kinase | | | | | | | | | |
| LRRK1 | mSK698 | 107 | 221 | Protein | TKL | LRRK | | 7156 | 2004 | 264 | 6275 | 6012 | LRRK1 |
| | | | | Kinase | | | | | | | | | |
| LZK | mSK398 | 108 | 222 | Protein | TKL | MLK | LZK | 3192 | 959 | 313 | 3189 | 2877 | LZK |
| | | | | Kinase | | | | | | | | | |
| MLK2 | mSK233 | 109 | 223 | Protein | TKL | MLK | MLK | 3476 | 940 | 352 | 3171 | 2820 | MLK2 |
| | | | | Kinase | | | | | | | | | |
| BRAF | mSK050 | 110 | 224 | Protein | TKL | RAF | | 2625 | 784 | 165 | 2516 | 2352 | BRAF |
| | | | | Kinase | | | | | | | | | |
| KSR2 | mSK605 | 111 | 225 | Protein | TKL | RAF | | 2895 | 965 | 1 | 2895 | 2895 | KSR2 |
| | | | | Kinase | | | | | | | | | |

| | | | | | | | | | | |
|---|---|---|---|---|---|---|---|---|---|---|
| DGKd | mSK911 | 112 | 226 | Lipid Kinase | 3914 | 1158 | 40 | 3513 | 3474 | DGKd |
| DGKi | mSK914 | 113 | 227 | Lipid Kinase | 3383 | 1041 | 38 | 3160 | 3123 | DGKi |
| DGKq | mSK915 | 114 | 228 | Lipid Kinase | 3136 | 934 | 117 | 2918 | 2802 | DGKq |

Table 2 describes the results of Smith Waterman similarity searches (Matrix: Pam 100; gap open/extension penalties 12/2) of the amino acid sequences against the NCBI database of non-redundant protein sequences (www.ncbi.nlm.nih.gov/Entrez/protein.html). It is broken into three sections, Tables 2a, 2b and 2c. For Table 2a: from left to right the data presented is as follows: Gene_NAME, ID#na, ID#aa, Super-family, Group, Family, Subfamily, AA length, PSCORE, MATCHES, % Identity; Table 2b: from left to right ID#na, ID#aa, % Similarity, ACCESSION, and DESCRIPTION. The first columns (Gene_NAME, ID#na, ID#aa, Super-family, Group, Family, Subfamily, AA length) are the same as in Table 1. “PSCORE” refers to the Smith Waterman probability score. This number approximates the chance that the alignment occurred by chance. Thus, a very low number, such as 2.10E-64, indicates that there is a very significant match between the query and the database target. “Matches” indicates the number of amino acids that were identical in the alignment. “% Identity” lists the percent of amino acids that were identical over the alignment. “% Similarity” lists the percent of amino acids that were similar over the alignment. ACCESSION refers to the accession number of the most similar protein in the NCBI database of non-redundant proteins. “Description” contains the name and species of origin of the most similar protein in the NCBI database of non-redundant proteins. Table 2c continues the tabulation of the Smith Waterman results. The headings are: Gene_NAME, ID#na, ID#aa, Super-family, Group, Family, Subfamily, QUERYSTART, QUERYEND, TARGETSTART, TARGETEND, % QUERY, % TARGET. The “QUERY” is the patent sequence, and the “TARGET” is the best hit within the NCBI protein database. “QUERYSTART” refers to the amino acid number at which the Query (the patent protein sequence) begins to align with the TARGET (database) sequence. “QUERYEND” refers to the amino acid position within the patent protein sequence (the QUERY) at which the alignment with the database protein (the TARGET) ends. “TARGETSTART” refers to the amino acid position of the database protein (the TARGET) at which the alignment with the patent sequence (the QUERY) begins. “TARGETEND” refers to the amino acid position within the database sequence (the TARGET) at which alignment with the QUERY ends. % QUERY gives the percent of the patent amino acid sequence which is aligned with the database hit (the TARGET). % TARGET gives the percent of the database hit which aligns with the patent sequence.

TABLE 2a

| Gene_{—} | ID# | ID# | Super- | | | | AA | | | |
|---|---|---|---|---|---|---|---|---|---|---|
| Name | na | aa | family | Group | Family | Subfamily | length | PSCORE | MATCHES | % Identity |
| DMPK2 | 1 | 115 | Protein Kinase | AGC | DMPK | GEK | 1556 | 0 | 1424 | 92 |
| MRCKb | 2 | 116 | Protein Kinase | AGC | DMPK | GEK | 1713 | 0 | 1618 | 94 |
| MAST3 | 3 | 117 | Protein Kinase | AGC | MAST | | 1321 | 0 | 1162 | 88 |
| MAST1 | 4 | 118 | Protein Kinase | AGC | MAST | | 1570 | 0 | 1548 | 99 |
| LATS1 | 5 | 119 | Protein Kinase | AGC | NDR | | 1129 | 0 | 1082 | 96 |
| PKN1 | 6 | 120 | Protein Kinase | AGC | PKN | | 946 | 0 | 919 | 97 |
| SGK494 | 7 | 121 | Protein Kinase | AGC | RSK | | 395 | 9.70E−118 | 224 | 57 |
| RSKL1 | 8 | 122 | Protein Kinase | AGC | RSKL | | 1056 | 0 | 878 | 83 |
| ADCK4 | 9 | 123 | atyp PK | Atypical | ABC1 | ABC1-A | 533 | 4.30E−290 | 459 | 86 |
| ADCK5 | 10 | 124 | atyp PK | Atypical | ABC1 | ABC1-B | 582 | 3.00E−303 | 460 | 79 |
| AlphaK2 | 11 | 125 | atyp PK | Atypical | Alpha | | 1672 | 0 | 1469 | 88 |
| AlphaK3 | 12 | 126 | atyp PK | Atypical | Alpha | | 1231 | 0 | 1071 | 87 |
| BCR | 13 | 127 | atyp PK | Atypical | BCR | | 1270 | 0 | 1194 | 94 |
| ATR | 14 | 128 | atyp PK | Atypical | PIKK | ATR | 2635 | 0 | 2389 | 91 |
| AMPKa1 | 15 | 129 | Protein Kinase | CAMK | CAMKL | AMPK | 550 | 4.90E−300 | 545 | 99 |
| mSK794 | 16 | 130 | Protein Kinase | CAMK | CAMKL | MARK | 481 | 3.90E−214 | 393 | 82 |
| mSK798 | 17 | 131 | Protein Kinase | CAMK | CAMKL | MARK | 177 | 1.30E−37 | 89 | 50 |
| mSK801 | 18 | 132 | Protein Kinase | CAMK | CAMKL | MARK | 492 | 4.00E−133 | 302 | 61 |
| mSK804 | 19 | 133 | Protein Kinase | CAMK | CAMKL | MARK | 492 | 9.90E−131 | 301 | 61 |
| mSK805 | 20 | 134 | Protein Kinase | CAMK | CAMKL | MARK | 304 | 4.80E−102 | 224 | 74 |
| mSK807 | 21 | 135 | Protein Kinase | CAMK | CAMKL | MARK | 477 | 1.10E−203 | 384 | 81 |
| mSK808 | 22 | 136 | Protein Kinase | CAMK | CAMKL | MARK | 238 | 2.90E−82 | 166 | 70 |
| mSK809 | 23 | 137 | Protein Kinase | CAMK | CAMKL | MARK | 474 | 7.10E−225 | 397 | 84 |
| mSK811 | 24 | 138 | Protein Kinase | CAMK | CAMKL | MARK | 306 | 1.70E−103 | 198 | 65 |
| mSK813 | 25 | 139 | Protein Kinase | CAMK | CAMKL | MARK | 232 | 4.10E−89 | 155 | 67 |
| mSK814 | 26 | 140 | Protein Kinase | CAMK | CAMKL | MARK | 209 | 2.30E−76 | 147 | 70 |
| mSK815 | 27 | 141 | Protein Kinase | CAMK | CAMKL | MARK | 384 | 2.00E−137 | 276 | 72 |
| mSK817 | 28 | 142 | Protein Kinase | CAMK | CAMKL | MARK | 178 | 1.90E−74 | 140 | 79 |
| mSK822 | 29 | 143 | Protein Kinase | CAMK | CAMKL | MARK | 617 | 5.10E−140 | 451 | 73 |
| mSK823 | 30 | 144 | Protein Kinase | CAMK | CAMKL | MARK | 411 | 5.20E−78 | 180 | 44 |
| mSK826 | 31 | 145 | Protein Kinase | CAMK | CAMKL | MARK | 520 | 1.90E−266 | 480 | 92 |
| mSK836 | 32 | 146 | Protein Kinase | CAMK | CAMKL | MARK | 261 | 2.00E−105 | 214 | 82 |
| mSK838 | 33 | 147 | Protein Kinase | CAMK | CAMKL | MARK | 476 | 2.70E−133 | 287 | 60 |
| mSK840 | 34 | 148 | Protein Kinase | CAMK | CAMKL | MARK | 514 | 1.90E−236 | 457 | 89 |
| mSK843 | 35 | 149 | Protein Kinase | CAMK | CAMKL | MARK | 322 | 8.40E−165 | 301 | 93 |
| NuaK1 | 36 | 150 | Protein Kinase | CAMK | CAMKL | NuaK | 658 | 7.3e−312 | 640 | 97 |
| QSK | 37 | 151 | Protein Kinase | CAMK | CAMKL | QIK | 1356 | 0 | 1194 | 88 |
| DCAMKL1 | 38 | 152 | Protein Kinase | CAMK | DCAMKL | | 745 | 0 | 718 | 96 |
| MNK2 | 39 | 153 | Protein Kinase | CAMK | MAPKA | MNK | 459 | 3.40E−231 | 431 | 94 |
| | | | | | PK | | | | | |
| smMLCK | 40 | 154 | Protein Kinase | CAMK | MLCK | | 1950 | 0 | 1922 | 99 |
| TTN | 41 | 155 | Protein Kinase | CAMK | MLCK | | 36946 | 0 | | 0 |
| skMLCK | 42 | 156 | Protein Kinase | CAMK | MLCK | | 613 | 8.00E−205 | 562 | 92 |
| SgK085 | 43 | 157 | Protein Kinase | CAMK | MLCK | | 390 | 3.00E−158 | 295 | 76 |
| PIM2 | 44 | 158 | Protein Kinase | CAMK | PIM | | 411 | 1.00E−167 | 369 | 90 |
| Trio | 45 | 159 | Protein Kinase | CAMK | Trio | | 3103 | 0 | 2854 | 92 |
| Trad | 46 | 160 | Protein Kinase | CAMK | Trio | | 2966 | 0 | 2901 | 98 |
| SPEC | 47 | 161 | Protein Kinase | CAMK | Trio | | 3262 | 0 | 3231 | 99 |
| Obscurin | 48 | 162 | Protein Kinase | CAMK | Trio | | 8523 | 0 | 5062 | 59 |
| TSSK5 | 49 | 163 | Protein Kinase | CAMK | TSSK | | 372 | 4.80E−180 | 337 | 91 |
| CK1g3 | 50 | 164 | Protein Kinase | CK1 | CK1 | | 448 | 4.70E−267 | 448 | 100 |
| TTBK2 | 51 | 165 | Protein Kinase | CK1 | TTBK | | 1243 | 0 | 1044 | 84 |
| TTBK1 | 52 | 166 | Protein Kinase | CK1 | TTBK | | 1308 | 0 | 1133 | 87 |
| CHED | 53 | 167 | Protein Kinase | CMGC | CDK | CRK7 | 1511 | 0 | 1436 | 95 |
| PFTAIRE2 | 54 | 168 | Protein Kinase | CMGC | CDK | TAIRE | 433 | 3.70E−194 | 352 | 81 |
| CDKL5 | 55 | 169 | Protein Kinase | CMGC | CDKL | | 904 | 0 | 866 | 96 |
| CDKL4 | 56 | 170 | Protein Kinase | CMGC | CDKL | | 342 | 1.80E−137 | 263 | 77 |
| DYRK4 | 57 | 171 | Protein Kinase | CMGC | DYRK | DYRK2 | 592 | 5.6e−320 | 559 | 94 |
| HIPK4 | 58 | 172 | Protein Kinase | CMGC | DYRK | HIPK | 541 | 5.00E−291 | 530 | 98 |
| ERK4 | 59 | 173 | Protein Kinase | CMGC | MAPK | ERK | 583 | 4.20E−224 | 479 | 82 |
| AAK1 | 60 | 174 | Protein Kinase | Other | NAK | | 958 | 1.50E−230 | 757 | 79 |
| NEK1 | 61 | 175 | Protein Kinase | Other | NEK | | 1275 | 0 | 1030 | 81 |
| NEK5 | 62 | 176 | Protein Kinase | Other | NEK | | 778 | 4.00E−274 | 590 | 76 |
| NEK10 | 63 | 177 | Protein Kinase | Other | NEK | | 1111 | 0 | 857 | 77 |
| SgK069 | 64 | 178 | Protein Kinase | Other | NKF1 | | 362 | 3.40E−178 | 304 | 84 |
| SgK110 | 65 | 179 | Protein Kinase | Other | NKF1 | | 207 | 1.30E−88 | 162 | 78 |
| SgK223 | 66 | 180 | Protein Kinase | Other | NKF3 | | 1373 | 0 | 1178 | 86 |
| SgK269 | 67 | 181 | Protein Kinase | Other | NKF3 | | 1735 | 0 | 1042 | 60 |
| CLIK1 | 68 | 182 | Protein Kinase | Other | NKF4 | | 539 | 1.00E−202 | 457 | 85 |
| SgK307 | 69 | 183 | Protein Kinase | Other | NKF5 | | 1450 | 0 | 1232 | 85 |
| SgK424 | 70 | 184 | Protein Kinase | Other | NKF5 | | 469 | 6.40E−163 | 312 | 67 |
| NRBP2 | 71 | 185 | Protein Kinase | Other | NRBP | | 499 | 9.20E−289 | 497 | 100 |
| SgK493 | 72 | 186 | Protein Kinase | Other | Other-Unique | | 491 | 3.80E−213 | 354 | 72 |
| SgK496 | 73 | 187 | Protein Kinase | Other | Other-Unique | | 927 | 0 | 817 | 88 |
| SgK071 | 74 | 188 | Protein Kinase | Other | Other-Unique | | 626 | 1.90E−209 | 435 | 69 |
| SgK384 | 75 | 189 | Protein Kinase | Other | PLK | | 599 | 9.90E−201 | 380 | 63 |
| Fused | 76 | 190 | Protein Kinase | Other | ULK | | 1316 | 0 | 1113 | 85 |
| ULK3 | 77 | 191 | Protein Kinase | Other | ULK | | 472 | 1.80E−226 | 444 | 94 |
| ULK4 | 78 | 192 | Protein Kinase | Other | ULK | | 1275 | 0 | 878 | 69 |
| PIK3R4 | 79 | 193 | Protein Kinase | Other | VPS15 | | 1358 | 0 | 1302 | 96 |
| Wee1B | 80 | 194 | Protein Kinase | Other | WEE | | 555 | 1.40E−194 | 330 | 59 |
| Wnk2 | 81 | 195 | Protein Kinase | Other | Wnk | | 2132 | 0 | 1897 | 89 |
| Wnk1 | 82 | 196 | Protein Kinase | Other | Wnk | | 2377 | 0 | 2045 | 86 |
| Wnk3 | 83 | 197 | Protein Kinase | Other | Wnk | | 1751 | 0 | 1401 | 80 |
| HSER | 84 | 198 | Protein Kinase | RGC | RGC | | 1066 | 0 | 1001 | 94 |
| CYGX | 85 | 199 | Protein Kinase | RGC | RGC | | 908 | 0 | 852 | 94 |
| KSGC | 86 | 200 | Protein Kinase | RGC | RGC | | 1101 | 0 | 970 | 88 |
| MAP3K6 | 87 | 201 | Protein Kinase | STE | STE11 | | 1291 | 0 | 1248 | 97 |
| MAP3K8 | 88 | 202 | Protein Kinase | STE | STE11 | | 1388 | 0 | 1327 | 96 |
| MAP3K7 | 89 | 203 | Protein Kinase | STE | STE11 | | 1334 | 0 | 1157 | 87 |
| OSR1 | 90 | 204 | Protein Kinase | STE | STE20 | FRAY | 527 | 1.80E−241 | 505 | 96 |
| ZC1 | 91 | 205 | Protein Kinase | STE | STE20 | MSN | 1328 | 1.20E−300 | 1233 | 93 |
| ZC2 | 92 | 206 | Protein Kinase | STE | STE20 | MSN | 1351 | 0 | 1335 | 99 |
| ZC4 | 93 | 207 | Protein Kinase | STE | STE20 | MSN | 1539 | 0 | 1454 | 94 |
| MYO3B | 94 | 208 | Protein Kinase | STE | STE20 | NinaC | 1613 | 0 | 1007 | 62 |
| PAK6 | 95 | 209 | Protein Kinase | STE | STE20 | PAKB | 682 | 8.66e−320 | 668 | 98 |
| STLK5 | 96 | 210 | Protein Kinase | STE | STE20 | STLK | 431 | 5.30E−244 | 408 | 95 |
| TAO2 | 97 | 211 | Protein Kinase | STE | STE20 | TAO | 1240 | 0 | 1211 | 98 |
| TAO3 | 98 | 212 | Protein Kinase | STE | STE20 | TAO | 898 | 0 | 859 | 96 |
| TIF1g | 99 | 213 | atyp PK | TIF1 | | | 1142 | 4.40E−267 | 1084 | 95 |
| ErbB3 | 100 | 214 | Protein Kinase | TK | EGFR | | 1339 | 0 | 1294 | 97 |
| EphA5 | 101 | 215 | Protein Kinase | TK | Eph | | 1041 | 0 | 997 | 96 |
| EphA6 | 102 | 216 | Protein Kinase | TK | Eph | | 1130 | 0 | 1035 | 92 |
| LMR2 | 103 | 217 | Protein Kinase | TK | Lmr | | 1469 | 0 | 1144 | 78 |
| LMR3 | 104 | 218 | Protein Kinase | TK | Lmr | | 1431 | 0 | 1145 | 80 |
| FGR | 105 | 219 | Protein Kinase | TK | Src | | 517 | 2.70E−276 | 517 | 100 |
| LRRK2 | 106 | 220 | Protein Kinase | TKL | LRRK | | 2478 | 0 | 1269 | 51 |
| LRRK1 | 107 | 221 | Protein Kinase | TKL | LRRK | | 2004 | 0 | 1901 | 95 |
| LZK | 108 | 222 | Protein Kinase | TKL | MLK | LZK | 959 | 0 | 860 | 90 |
| MLK2 | 109 | 223 | Protein Kinase | TKL | MLK | MLK | 940 | 0 | 919 | 98 |
| BRAF | 110 | 224 | Protein Kinase | TKL | RAF | | 784 | 1.50E−235 | 742 | 95 |
| KSR2 | 111 | 225 | Protein Kinase | TKL | RAF | | 965 | 7.00E−145 | 400 | 41 |

| | | | | | | | |
|---|---|---|---|---|---|---|---|
| DGKd | 112 | 226 | Lipid Kinase | 1158 | 0 | 1086 | 94 |
| DGKi | 113 | 227 | Lipid Kinase | 1041 | 0 | 1010 | 97 |
| DGKq | 114 | 228 | Lipid Kinase | 934 | 0 | 900 | 96 |

TABLE 2b

| ID# | ID# | % | | |
|---|---|---|---|---|
| na | aa | Similarity | ACCESSION | DESCRIPTION |
| 1 | 115 | 94 | gi\|27661270\|ref\| | similar to Ser-Thr protein kinase related to the myotonic dystrophy protein |
| | | | XP_219530.1\| | kinase [Rattus norvegicus] |
| 2 | 116 | 97 | gi\|16758420\|ref\| | Cdc42-binding protein kinase beta [Rattus norvegicus] |
| | | | NP_446072.1\| | |
| 3 | 117 | 92 | gi\|3043646\|dbj\| | KIAA0561 protein [Homo sapiens] |
| | | | BAA25487.1\| | |
| 4 | 118 | 99 | gi\|29373057\|gb\| | syntrophin-associated serine/threonine kinase SAST170 [Rattus norvegicus] |
| | | | AAO72536.1\| | |
| 5 | 119 | 98 | gi\|27730757\|ref\| | similar to LATS homolog 1 [Homo sapiens] [Rattus norvegicus] |
| | | | XP_218062.1\| | |
| 6 | 120 | 99 | gi\|16905491\|gb\| | cardiolipin/protease-activated protein kinase-1 [Rattus norvegicus] |
| | | | AAL31374.1\|L35634_1 | |
| 7 | 121 | 61 | gi\|21389411\|ref\| | hypothetical protein FLJ25006 [Homo sapiens] |
| | | | NP_653211.1\| | |
| 8 | 122 | 83 | gi\|28483931\|ref\| | RIKEN cDNA B130003F20 gene [Mus musculus] |
| | | | XP_129675.2\| | |
| 9 | 123 | 91 | gi\|27363457\|ref\| | hypothetical protein FLJ12229 [Homo sapiens] |
| | | | NP_079152.3\| | |
| 10 | 124 | 79 | gi\|27370472\|ref\| | hypothetical protein A230108P17 [Mus musculus] |
| | | | NP_766548.1\| | |
| 11 | 125 | 88 | gi\|15430296\|gb\| | heart alpha-kinase [Mus musculus] |
| | | | AAK95953.1\| | |
| 12 | 126 | 87 | gi\|20875939\|ref\| | similar to lymphocyte alpha-kinase [Homo sapiens] [Mus musculus] |
| | | | XP_143521.1\| | |
| 13 | 127 | 97 | gi\|11038639\|ref\| | breakpoint cluster region isoform 1 [Homo sapiens] |
| | | | NP_004318.2\| | |
| 14 | 128 | 96 | gi\|1235902\|gb\| | FRAP-related protein |
| | | | AAC50405.1\| | |
| 15 | 129 | 99 | gi\|11862980\|ref\| | protein kinase, AMP-activated, alpha 1 catalytic subunit; 5′-AMP-activated |
| | | | NP_062015.1\| | protein kinase alpha-1 catalytic subunit [Rattus norvegicus] |
| 16 | 130 | 85 | gi\|20900474\|ref\| | similar to MAP/microtubule affinity-regulating kinase 2 isoform a; ELKL motif |
| | | | XP_140045.1\| | kinase 1; ELKL motif kinase [Homo sapiens] [Mus musculus] |
| 17 | 131 | 64 | gi\|26325454\|dbj\| | unnamed protein product [Mus musculus] |
| | | | BAC26481.1\| | |
| 18 | 132 | 76 | gi\|27369690\|ref\| | hypothetical protein 4930509O22 [Mus musculus] |
| | | | NP_766092.1\| | |
| 19 | 133 | 76 | gi\|27369690\|ref\| | hypothetical protein 4930509O22 [Mus musculus] |
| | | | NP_766092.1\| | |
| 20 | 134 | 74 | gi\|25050195\|ref\| | similar to MAP/microtubule affinity-regulating kinase like 1; MARK4 |
| | | | XP_195585.1\| | serine/threonine protein kinase [Homo sapiens] [Mus musculus] |
| 21 | 135 | 84 | gi\|20900474\|ref\| | similar to MAP/microtubule affinity-regulating kinase 2 isoform a; ELKL motif |
| | | | XP_140045.1\| | kinase 1; ELKL motif kinase [Homo sapiens] [Mus musculus] |
| 22 | 136 | 83 | gi\|27703602\|ref\| | similar to serine/threonine kinase [Rattus norvegicus] |
| | | | XP_230703.1\| | |
| 23 | 137 | 84 | gi\|29243962\|ref\| | hypothetical protein 4932415M13 [Mus musculus] |
| | | | NP_808267.1\| | |
| 24 | 138 | 78 | gi\|27679020\|ref\| | similar to serine/threonine kinase [Rattus norvegicus] |
| | | | XP_222975.1] | |
| 25 | 139 | 72 | gi\|26325454\|dbj\| | unnamed protein product [Mus musculus] |
| | | | BAC26481.1\| | |
| 26 | 140 | 84 | gi\|27687803\|ref\| | similar to Ser/Thr protein kinase PAR-1alpha [Drosophila melanogaster] |
| | | | XP_237567.1\| | [Rattus norvegicus] |
| 27 | 141 | 72 | gi\|20822134\|ref\| | similar to Ser/Thr protein kinase PAR-1alpha [Drosophila melanogaster] |
| | | | XP_145432.1\| | [Mus musculus] |
| 28 | 142 | 79 | gi\|20822164\|ref\| | similar to KP78a gene product [Drosophila melanogaster] [Mus musculus] |
| | | | XP_145444.1\| | |
| 29 | 143 | 82 | gi\|26325454\|dbj\| | unnamed protein product [Mus musculus] |
| | | | BAC26481.1\| | |
| 30 | 144 | 60 | gi\|27731641\|ref\| | similar to MAP/microtubule affinity-regulating kinase like 1; MARK4 |
| | | | XP_218667.1\| | serine/threonine protein kinase [Homo sapiens] [Rattus norvegicus] |
| 31 | 145 | 92 | gi\|20900474\|ref\| | similar to MAP/microtubule affinity-regulating kinase 2 isoform a; ELKL motif |
| | | | XP 140045.1\| | kinase 1; ELKL motif kinase [Homo sapiens] [Mus musculus] |
| 32 | 146 | 84 | gi\|28503636\|ref\| | similar to serine/threonine kinase [Rattus norvegicus] [Mus musculus] |
| | | | XP_195367.2\| | |
| 33 | 147 | 72 | gi\|27369690\|ref\| | hypothetical protein 4930509O22 [Mus musculus] |
| | | | NP_766092.1\| | |
| 34 | 148 | 92 | gi\|20956294\|ref\| | similar to putative protein kinase [Mus musculus] |
| | | | XP_142616.1\| | |
| 35 | 149 | 97 | gi\|20900474\|ref\| | similar to MAP/microtubule affinity-regulating kinase 2 isoform a; ELKL motif |
| | | | XP_140045.1\| | kinase 1; ELKL motif kinase [Homo sapiens] [Mus musculus] |
| 36 | 150 | 98 | gi\|27717823\|ref\| | similar to Probable serine/threonine-protein kinase KIAA0537 [Rattus norvegicus] |
| | | | XP_234998.1\| | |
| 37 | 151 | 93 | gi\|14133229\|dbj\| | KIAA0999 protein [Homo sapiens] |
| | | | BAA76843.2\| | |
| 38 | 152 | 97 | gi\|4758128\|ref\| | doublecortin and CaM kinase-like 1; doublecortin-like kinase [Homo sapiens] |
| | | | NP_004725.1\| | |
| 39 | 153 | 97 | gi\|4464284\|gb\| | Putative map kinase interacting kinase [Homo sapiens] |
| | | | AAD21217.1\| | |
| 40 | 154 | 99 | gi\|29650205\|gb\| | 220 kDa myosin light chain kinase [Mus musculus] |
| | | | AAO85807.1\| | |
| 41 | 155 | 0 | gi\|17066105\|emb\| | Titin [Homo sapiens] |
| | | | CAD12456.1\| | |
| 42 | 156 | 95 | gi\|125494\|sp\| | Myosin light chain kinase 2, skeletal/cardiac muscle (MLCK2) |
| | | | P20689\|KML2_RAT | |
| 43 | 157 | 76 | gi\|20345411\|ref\| | similar to myosin light chain kinase (MLCK) [Homo sapiens] [Mus musculus] |
| | | | XP_111421.1\| | |
| 44 | 158 | 90 | gi\|20070430\|ref\| | serine-threonine protein kinase pim-2 isoform 1; DNA segment, Chr X, |
| | | | NP_613072.1\| | Celltech Chiroscience 3 [Mus musculus] |
| 45 | 159 | 94 | gi\|8928460\|sp\| | Triple functional domain protein (PTPRF interacting protein) |
| | | | O75962\|TRIO_HUMAN | |
| 46 | 160 | 99 | gi\|14091744\|ref\| | huntingtin-associated protein interacting protein (duo) [Rattus norvegicus] |
| | | | NP_114451.1\| | |
| 47 | 161 | 99 | gi\|11385416\|gb\| | striated muscle-specific serine/threonine protein kinase [Mus musculus] |
| | | | AAG34791.1\|AF215896_1 | |
| 48 | 162 | 66 | gi\|15026974\|emb\| | obscurin [Homo sapiens] |
| | | | \|CAC44768.1\| | |
| 49 | 163 | 95 | gi\|27662252\|ref\| | similar to serine/threonine kinase 22B (spermiogenesis associated); testis specific |
| | | | XP_235450.1\| | serine threonine kinase 2; spermiogenesis associated 2 [Homo sapiens] [Rattus norvegicus] |
| 50 | 164 | 100 | gi\|12408306\|ref\| | casein kinase 1 gamma 3 isoform [Rattus norvegicus] |
| | | | NP_074046.1\| | |
| 51 | 165 | 89 | gi\|28466991\|ref\| | tau-tubulin kinase [Homo sapiens] |
| | | | NP_775771.2\| | |
| 52 | 166 | 91 | gi\|20555151\|ref\| | similar to tau tubulin kinase 1; tau-tubulin kinase [Mus musculus] |
| | | | XP_166453.1\| | [Homo sapiens] |
| 53 | 167 | 98 | gi\|14110387\|ref\| | cell division cycle 2-like 5 isoform 1; CDC2-related protein kinase |
| | | | NP_003709.2\| | 5 [Homo sapiens] |
| 54 | 168 | 85 | gi\|21040235\|ref\| | amyotrophic lateral sclerosis 2 (juvenile) chromosome region, candidate 7 |
| | | | NP_631897.1\| | [Homo sapiens] |
| 55 | 169 | 99 | gi\|4507281\|ref\| | cyclin-dependent kinase-like 5; serine/threonine kinase 9 [Homo sapiens] |
| | | | NP_003150.1\| | |
| 56 | 170 | 84 | gi\|29731492\|ref\| | similar to cyclin-dependent kinase-like 1 (CDC2-related kinase) [Mus musculus] |
| | | | XP_293029.1\| | [Homo sapiens] |
| 57 | 171 | 94 | gi\|28526538\|ref\| | expressed sequence AW049118 [Mus musculus] |
| | | | XP_132896.3\| | |
| 58 | 172 | 99 | gi\|27676688\|ref\| | similar to hypothetical protein [Macaca fascicularis] [Rattus norvegicus] |
| | | | XP_218355.1\| | |
| 59 | 173 | 85 | gi\|4506089\|ref\| | mitogen-activated protein kinase 4; Erk3-related; protein kinase, mitogen- |
| | | | NP_002738.1\| | activated 4 (MAP kinase 4; p63) [Homo sapiens] |
| 60 | 174 | 83 | gi\|5689433\|dbj\| | KIAA1048 protein [Homo sapiens] |
| | | | BAA83000.1\| | |
| 61 | 175 | 88 | gi\|15620861\|dbj\| | KIAA1901 protein [Homo sapiens] |
| | | | BAB67794.1\| | |
| 62 | 176 | 76 | gi\|28483717\|ref\| | similar to Serine/threonine-protein kinase NEK1 (NimA-related protein |
| | | | XP_284399.1\| | kinase 1) (NY-REN-55 antigen) [Mus musculus] |
| 63 | 177 | 80 | gi\|27674063\|ref\| | similar to hypothetical protein FLJ32685 [Homo sapiens] [Rattus norvegicus] |
| | | | XP_223815.1\| | |
| 64 | 178 | 86 | gi\|27675620\|ref\| | similar to protein kinase Bsk146 [Danio rerio] [Rattus norvegicus] |
| | | | XP_218202.1\| | |
| 65 | 179 | 86 | gi\|27485033\|ref\| | similar to protein kinase Bsk146 [Danio rerio] [Homo sapiens] |
| | | | XP_210370.1\| | |
| 66 | 180 | 86 | gi\|27370398\|ref\| | DNA segment, Chr 8, ERATO Doi 82, expressed [Mus musculus] |
| | | | NP_766499.1\| | |
| 67 | 181 | 62 | gi\|27720351\|ref\| | hypothetical protein XP_236266 [Rattus norvegicus] |
| | | | XP_236266.1\| | |
| 68 | 182 | 89 | gi\|12830335\|emb\| | bA550O8.2 (novel protein kinase) [Homo sapiens |
| | | | CAC10518.2\| | ] |
| 69 | 183 | 85 | gi\|13878215\|ref\| | testis expressed gene 14 [Mus musculus] |
| | | | NP_113563.1\| | |
| 70 | 184 | 68 | gi\|28477970\|ref\| | similar to testis protein TEX14 [Mus musculus] |
| | | | XP_145510.2\| | |
| 71 | 185 | 100 | gi\|27662242\|ref\| | similar to nuclear receptor binding protein; multiple domain putative nuclear |
| | | | XP_235443.1\| | protein [Homo sapiens] [Rattus norvegicus] |
| 72 | 186 | 74 | gi\|27716501\|ref\| | hypothetical protein XP_233838 [Rattus norvegicus] |
| | | | XP_233838.1\| | |
| 73 | 187 | 90 | gi\|27712032\|ref\| | similar to Retinoblastoma-binding protein 5 (RBBP-5) (Retinoblastoma- |
| | | | XP_222647.1\| | binding protein RBQ-3) [Rattus norvegicus] |
| 74 | 188 | 76 | gi\|27706574\|ref\| | similar to Protein kinase [Caenorhabditis elegans] [Rattus norvegicus] |
| | | | XP_231122.1\| | |
| 75 | 189 | 64 | gi\|28497763\|ref\| | similar to Cytokine-inducible serine/threonine-protein kinase (FGF-inducible |
| | | | XP_125726.3\| | kinase) [Mus musculus] |
| 76 | 190 | 92 | gi\|24308123\|ref\| | serine/threonine kinase 36 (fused homolog, Drosophila); serine/threonine |
| | | | NP_056505.1\| | kinase 36, fused homolog (Drosophila) [Homo sapiens] |
| 77 | 191 | 98 | gi\|27483725\|ref\| | DKFZP434C131 protein [Homo sapiens] |
| | | | XP_044630.2\| | |
| 78 | 192 | 69 | gi\|12855303\|dbj\| | unnamed protein product [Mus musculus] |
| | | | BAB30285.1\| | |
| 79 | 193 | 98 | gi\|23943912\|ref\| | phosphoinositide-3-kinase, regulatory subunit 4, p150; phosphatidylinositol |
| | | | NP_055417.1\| | 3-kinase-associated p150 [Homo sapiens] |
| 80 | 194 | 63 | gi\|27709826\|ref\| | similar to Wee1-like protein kinase (WEE1hu) [Rattus norvegicus] |
| | | | XP_231708.1\| | |
| 81 | 195 | 92 | gi\|27683635\|ref\| | similar to KIAA1760 protein [Homo sapiens] [Rattus norvegicus] |
| | | | XP_225204.1\| | |
| 82 | 196 | 92 | gi\|12711660\|ref\| | protein kinase, lysine deficient 1; kinase deficient protein [Homo sapiens] |
| | | | NP_061852.1\| | |
| 83 | 197 | 89 | gi\|19032238\|emb\| | protein kinase WNK3 [Homo sapiens] |
| | | | CAC32455.2\| | |
| 84 | 198 | 97 | gi\|6981000\|ref\| | guanylate cyclase 2C (heat stable enterotoxin receptor) [Rattus norvegicus] |
| | | | NP_037302.1\| | |
| 85 | 199 | 97 | gi\|18543337\|ref\| | guanylate cyclase 2d [Rattus norvegicus] |
| | | | NP_570093.1\| | |
| 86 | 200 | 93 | gi\|20514776\|ref\| | guanylyl cyclase with kinase-like domain, soluble [Rattus norvegicus] |
| | | | NP_620611.1\| | |
| 87 | 201 | 97 | gi\|7709976\|ref\| | mitogen-activated protein kinase kinase kinase 6; apoptosis signal- |
| | | | NP_057902.1\| | regulating kinase 2 [Mus musculus] |
| 88 | 202 | 96 | gi\|28482297\|ref\| | similar to hypothetical protein FLJ23074 |
| | | | XP_136210.3\| | [Homo sapiens] [Mus musculus] |
| 89 | 203 | 87 | gi\|25056550\|ref\| | similar to MAP/ERK kinase kinase 5; apoptosis signal regulating kinase |
| | | | XP_194648.1\| | [Homo sapiens] [Mus musculus] |
| 90 | 204 | 98 | gi\|4826878\|ref\| | oxidative-stress responsive 1 [Homo sapiens] |
| | | | NP_005100.1\| | |
| 91 | 205 | 93 | gi\|6679060\|ref\| | mitogen-activated protein kinase kinase kinase kinase 4; NCK interacting |
| | | | NP_032722.1\| | kinase; HPK/GCK-like kinase [Mus musculus] |
| 92 | 206 | 100 | gi\|6110355\|gb\| | Traf2 and NCK interacting kinase, splice variant 4 [Homo sapiens] |
| | | | AAF03785.1\|AF172267_1 | |
| 93 | 207 | 95 | gi\|6472874\|dbj\| | Nek-interacting kinase-like embryo specific kinase [Mus musculus] |
| | | | BAA87066.1\| | |
| 94 | 208 | 66 | gi\|27448205\|gb\| | myosin IIIB variant MYO3B.2 [Homo sapiens] |
| | | | AAO13800.1\| | |
| 95 | 209 | 99 | gi\|27731989\|ref\| | similar to Serine/threonine-protein kinase PAK 6 (p21-activated kinase 6) |
| | | | XP_230519.1\| | (PAK-6) (PAK-5) [Rattus norvegicus] |
| 96 | 210 | 98 | gi\|12060855\|gb\| | serologically defined breast cancer antigen NY-BR-96 [Homo sapiens] |
| | | | AAG48269.1\|AF308302_1 | |
| 97 | 211 | 98 | gi\|12083665\|ref\| | serine/threonine protein kinase TA02 [Rattus norvegicus] |
| | | | NP_073193.1\| | |
| 98 | 212 | 98 | gi\|19923464\|ref\| | STE20-like kinase; STE2-like kinase [Homo sapiens] |
| | | | NP_057365.2\| | |
| 99 | 213 | 97 | gi\|5689563\|dbj\| | KIAA1113 protein [Homo sapiens] |
| | | | BAA83065.1\| | |
| 100 | 214 | 99 | gi\|17432904\|ref\| | avian erythroblastosis oncogene B 3; v-erb-b2 erythroblastic leukemia viral |
| | | | NP_058914.2\| | oncogene homolog 3 (avian) [Rattus norvegicus] |
| 101 | 215 | 98 | gi\|24307885\|ref\| | EphA5; Hek7; ephrin receptor EphA5; TYRO4 protein tyrosine |
| | | | NP_004430.1\| | kinase [Homo sapiens] |
| 102 | 216 | 92 | gi\|20893175\|ref\| | Eph receptor A6 [Mus musculus] |
| | | | XP_147261.1\| | |
| 103 | 217 | 87 | gi\|27356940\|gb\| | KPI-2 protein [Homo sapiens] |
| | | | AAN08717.1\| | |
| 104 | 218 | 84 | gi\|20546870\|ref\| | similar to KIAA1883 protein [Homo sapiens] |
| | | | XP_055866.4\| | |
| 105 | 219 | 100 | gi\|26331398\|dbj\| | unnamed protein product [Mus musculus] |
| | | | BAC29429.1\| | |
| 106 | 220 | 54 | gi\|29745105\|ref\| | similar to RIKEN cDNA 4921513O20 [Mus musculus] [Homo sapiens] |
| | | | XP_058513.5\| | |
| 107 | 221 | 97 | gi\|27676850\|ref\| | similar to KIAA1790 protein [Homo sapiens] [Rattus norvegicus] |
| | | | XP_218760.1\| | |
| 108 | 222 | 95 | gi\|4758696\|ref\| | mitogen-activated protein kinase kinase kinase 13; leucine zipper-bearing |
| | | | NP_004712.1\| | kinase [Homo sapiens] |
| 109 | 223 | 99 | gi\|27676716\|ref\| | similar to mitogen-activated protein kinase kinase kinase 10; mixed lineage |
| | | | XP_218368.1\| | kinase 2; MKN28 kinase; MKN28 derived nonreceptor_type serine/threonine |
| | | | | kinase [Homo sapiens] [Rattus norvegicus] |
| 110 | 224 | 96 | gi\|4757868\|ref\| | v-raf murine sarcoma viral oncogene homolog B1; Murine sarcoma viral (v-raf) |
| | | | NP_004324.1\| | oncogene homolog B1 [Homo sapiens] |
| 111 | 225 | 42 | gi\|27499671\|ref\| | similar to protein kinase related to Raf protein kinases; Method: conceptual |
| | | | XP_208683.1\| | translation supplied by author [Homo sapiens] |
| 112 | 226 | 97 | gi\|12644420\|sp\| | Diacylglycerol kinase, delta (Diglyceride kinase) (DGK-delta) (DAG kinase |
| | | | Q16760\|KDGD_HUMAN | delta) (130 kDa diacylglycerol kinase) |
| 113 | 227 | 98 | gi\|29466777\|dbj\| | diacylglycerol kinase iota-1 [Rattus norvegicus] |
| | | | BAC66854.1\| | |
| 114 | 228 | 98 | gi\|27677206\|ref\| | similar to Diacylglycerol kinase, theta (Diglyceride kinase) (DGK-theta) (DAG |
| | | | XP_223739.1\| | kinase theta) [Rattus norvegicus] |

TABLE 2c

| Gene_{—} | ID# | ID# | Super- | | | | QUERY | QUERY | TARGET | TARGET | % | % |
|---|---|---|---|---|---|---|---|---|---|---|---|---|
| Name | na | aa | family | Group | Family | Subfamily | START | END | START | END | QUERY | TARGET |
| DMPK2 | 1 | 115 | Protein | AGC | DMPK | GEK | 1 | 1509 | 1 | 1544 | 92 | 85 |
| | | | Kinase | | | | | | | | | |
| MRCKb | 2 | 116 | Protein | AGC | DMPK | GEK | 1 | 1693 | 1 | 1693 | 94 | 95 |
| | | | Kinase | | | | | | | | | |
| MAST3 | 3 | 117 | Protein | AGC | MAST | | 39 | 1321 | 16 | 1308 | 88 | 89 |
| | | | Kinase | | | | | | | | | |
| MAST1 | 4 | 118 | Protein | AGC | MAST | | 1 | 1570 | 1 | 1570 | 99 | 99 |
| | | | Kinase | | | | | | | | | |
| LATS1 | 5 | 119 | Protein | AGC | NDR | | 1 | 1127 | 1 | 1128 | 96 | 88 |
| | | | Kinase | | | | | | | | | |
| PKN1 | 6 | 120 | Protein | AGC | PKN | | 1 | 946 | 1 | 946 | 97 | 97 |
| | | | Kinase | | | | | | | | | |
| SGK494 | 7 | 121 | Protein | AGC | RSK | | 1 | 264 | 1 | 271 | 57 | 82 |
| | | | Kinase | | | | | | | | | |
| RSKL1 | 8 | 122 | Protein | AGC | RSKL | | 1 | 960 | 1 | 878 | 83 | 100 |
| | | | Kinase | | | | | | | | | |
| ADCK4 | 9 | 123 | atyp PK | Atypical | ABC1 | ABC1-A | 1 | 525 | 1 | 524 | 86 | 84 |
| ADCK5 | 10 | 124 | atyp PK | Atypical | ABC1 | ABC1-B | 123 | 582 | 1 | 460 | 79 | 100 |
| AlphaK2 | 11 | 125 | atyp PK | Atypical | Alpha | | 200 | 1672 | 3 | 1475 | 88 | 100 |
| AlphaK3 | 12 | 126 | atyp PK | Atypical | Alpha | | 158 | 1228 | 16 | 1134 | 87 | 94 |
| BCR | 13 | 127 | atyp PK | Atypical | BCR | | 1 | 1270 | 1 | 1271 | 94 | 94 |
| ATR | 14 | 128 | atyp PK | Atypical | PIKK | ATR | 1 | 2635 | 1 | 2644 | 91 | 90 |
| AMPKa1 | 15 | 129 | Protein | CAMK | CAMKL | A MPK | 4 | 550 | 2 | 548 | 99 | 99 |
| | | | Kinase | | | | | | | | | |
| mSK794 | 16 | 130 | Protein | CAMK | CAMKL | M ARK | 1 | 439 | 1 | 439 | 82 | 73 |
| | | | Kinase | | | | | | | | | |
| mSK798 | 17 | 131 | Protein | CAMK | CAMKL | M ARK | 3 | 174 | 92 | 272 | 50 | 14 |
| | | | Kinase | | | | | | | | | |
| mSK801 | 18 | 132 | Protein | CAMK | CAMKL | M ARK | 1 | 483 | 1 | 499 | 61 | 61 |
| | | | Kinase | | | | | | | | | |
| mSK804 | 19 | 133 | Protein | CAMK | CAMKL | M ARK | 1 | 483 | 1 | 499 | 61 | 60 |
| | | | Kinase | | | | | | | | | |
| mSK805 | 20 | 134 | Protein | CAMK | CAMKL | M ARK | 1 | 304 | 1 | 231 | 74 | 97 |
| | | | Kinase | | | | | | | | | |
| mSK807 | 21 | 135 | Protein | CAMK | CAMKL | M ARK | 1 | 439 | 1 | 438 | 81 | 72 |
| | | | Kinase | | | | | | | | | |
| mSK808 | 22 | 136 | Protein | CAMK | CAMKL | M ARK | 4 | 238 | 179 | 415 | 70 | 28 |
| | | | Kinase | | | | | | | | | |
| mSK809 | 23 | 137 | Protein | CAMK | CAMKL | M ARK | 76 | 474 | 1 | 399 | 84 | 98 |
| | | | Kinase | | | | | | | | | |
| mSK811 | 24 | 138 | Protein | CAMK | CAMKL | M ARK | 2 | 303 | 72 | 379 | 65 | 28 |
| | | | Kinase | | | | | | | | | |
| mSK813 | 25 | 139 | Protein | CAMK | CAMKL | M ARK | 32 | 218 | 1 | 199 | 67 | 24 |
| | | | Kinase | | | | | | | | | |
| mSK814 | 26 | 140 | Protein | CAMK | CAMKL | M ARK | 1 | 207 | 1 | 207 | 70 | 42 |
| | | | Kinase | | | | | | | | | |
| mSK815 | 27 | 141 | Protein | CAMK | CAMKL | M ARK | 16 | 384 | 1 | 277 | 72 | 100 |
| | | | Kinase | | | | | | | | | |
| mSK817 | 28 | 142 | Protein | CAMK | CAMKL | M ARK | 5 | 144 | 1 | 140 | 79 | 67 |
| | | | Kinase | | | | | | | | | |
| mSK822 | 29 | 143 | Protein | CAMK | CAMKL | M ARK | 32 | 611 | 1 | 602 | 73 | 70 |
| | | | Kinase | | | | | | | | | |
| mSK823 | 30 | 144 | Protein | CAMK | CAMKL | M ARK | 22 | 375 | 92 | 444 | 44 | 28 |
| | | | Kinase | | | | | | | | | |
| mSK826 | 31 | 145 | Protein | CAMK | CAMKL | M ARK | 12 | 491 | 1 | 480 | 92 | 90 |
| | | | Kinase | | | | | | | | | |
| mSK836 | 32 | 146 | Protein | CAMK | CAMKL | M ARK | 13 | 256 | 7 | 247 | 82 | 69 |
| | | | Kinase | | | | | | | | | |
| mSK838 | 33 | 147 | Protein | CAMK | CAMKL | M ARK | 1 | 437 | 1 | 438 | 60 | 58 |
| | | | Kinase | | | | | | | | | |
| mSK840 | 34 | 148 | Protein | CAMK | CAMKL | M ARK | 12 | 514 | 1 | 508 | 89 | 90 |
| | | | Kinase | | | | | | | | | |
| mSK843 | 35 | 149 | Protein | CAMK | CAMKL | M ARK | 1 | 322 | 1 | 322 | 93 | 56 |
| | | | Kinase | | | | | | | | | |
| NuaK1 | 36 | 150 | Protein | CAMK | CAMKL | NuaK | 1 | 658 | 1 | 754 | 97 | 85 |
| | | | Kinase | | | | | | | | | |
| QSK | 37 | 151 | Protein | CAMK | CAMKL | QIK | 2 | 1356 | 65 | 1371 | 88 | 87 |
| | | | Kinase | | | | | | | | | |
| DCAMKL1 | 38 | 152 | Protein | CAMK | DCAMKL | | 1 | 745 | 1 | 729 | 96 | 98 |
| | | | Kinase | | | | | | | | | |
| MNK2 | 39 | 153 | Protein | CAMK | MAPKAPK | MNK | 1 | 459 | 8 | 472 | 94 | 91 |
| | | | Kinase | | | | | | | | | |
| smMLCK | 40 | 154 | Protein | CAMK | MLCK | | 1 | 1950 | 1 | 1950 | 99 | 99 |
| | | | Kinase | | | | | | | | | |
| TTN | 41 | 155 | Protein | CAMK | MLCK | | | | | | 0 | 0 |
| | | | Kinase | | | | | | | | | |
| skMLCK | 42 | 156 | Protein | CAMK | MLCK | | 1 | 613 | 1 | 610 | 92 | 92 |
| | | | Kinase | | | | | | | | | |
| SgK085 | 43 | 157 | Protein | CAMK | MLCK | | 78 | 372 | 366 | 660 | 76 | 39 |
| | | | Kinase | | | | | | | | | |
| PIM2 | 44 | 158 | Protein | CAMK | PIM | | 42 | 411 | 1 | 370 | 90 | 100 |
| | | | Kinase | | | | | | | | | |
| Trio | 45 | 159 | Protein | CAMK | Trio | | 60 | 3103 | 1 | 3038 | 92 | 94 |
| | | | Kinase | | | | | | | | | |
| Trad | 46 | 160 | Protein | CAMK | Trio | | 1 | 2960 | 5 | 2959 | 98 | 98 |
| | | | Kinase | | | | | | | | | |
| SPEG | 47 | 161 | Protein | CAMK | Trio | | 1 | 3262 | 1 | 3262 | 99 | 99 |
| | | | Kinase | | | | | | | | | |
| Obscurin | 48 | 162 | Protein | CAMK | Trio | | 1 | 6801 | 6 | 6225 | 59 | 76 |
| | | | Kinase | | | | | | | | | |
| TSSK5 | 49 | 163 | Protein | CAMK | TSSK | | 1 | 372 | 95 | 457 | 91 | 74 |
| | | | Kinase | | | | | | | | | |
| CK1g3 | 50 | 164 | Protein | CK1 | CK1 | | 1 | 448 | 1 | 448 | 100 | 100 |
| | | | Kinase | | | | | | | | | |
| TTBK2 | 51 | 165 | Protein | CK1 | TTBK | | 74 | 1243 | 54 | 1649 | 84 | 63 |
| | | | Kinase | | | | | | | | | |
| TTBK1 | 52 | 166 | Protein | CK1 | TTBK | | 52 | 1308 | 1 | 1270 | 87 | 89 |
| | | | Kinase | | | | | | | | | |
| CHED | 53 | 167 | Protein | CMGC | CDK | CRK7 | 1 | 1511 | 1 | 1512 | 95 | 95 |
| | | | Kinase | | | | | | | | | |
| PFTAIRE2 | 54 | 168 | Protein | CMGC | CDK | TAIRE | 51 | 433 | 2 | 384 | 81 | 92 |
| | | | Kinase | | | | | | | | | |
| CDKL5 | 55 | 169 | Protein | CMGC | CDKL | | 1 | 904 | 1 | 904 | 96 | 84 |
| | | | Kinase | | | | | | | | | |
| CDKL4 | 56 | 170 | Protein | CMGC | CDKL | | 1 | 309 | 1 | 315 | 77 | 68 |
| | | | Kinase | | | | | | | | | |
| DYRK4 | 57 | 171 | Protein | CMGC | DYRK | DYRK2 | 1 | 592 | 74 | 632 | 94 | 88 |
| | | | Kinase | | | | | | | | | |
| HIPK4 | 58 | 172 | Protein | CMGC | DYRK | HIPK | 1 | 541 | 1 | 616 | 98 | 86 |
| | | | Kinase | | | | | | | | | |
| ERK4 | 59 | 173 | Protein | CMGC | MAPK | ERK | 1 | 522 | 1 | 526 | 82 | 86 |
| | | | Kinase | | | | | | | | | |
| AAK1 | 60 | 174 | Protein | Other | NAK | | 1 | 833 | 1 | 836 | 79 | 88 |
| | | | Kinase | | | | | | | | | |
| NEK1 | 61 | 175 | Protein | Other | NEK | | 1 | 1275 | 8 | 1265 | 81 | 81 |
| | | | Kinase | | | | | | | | | |
| NEK5 | 62 | 176 | Protein | Other | NEK | | 1 | 778 | 1 | 614 | 76 | 96 |
| | | | Kinase | | | | | | | | | |
| NEK10 | 63 | 177 | Protein | Other | NEK | | 45 | 1001 | 781 | 1887 | 77 | 45 |
| | | | Kinase | | | | | | | | | |
| SgK069 | 64 | 178 | Protein | Other | NKF1 | | 1 | 317 | 174 | 490 | 84 | 51 |
| | | | Kinase | | | | | | | | | |
| SgK110 | 65 | 179 | Protein | Other | NKF1 | | 5 | 191 | 35 | 221 | 78 | 19 |
| | | | Kinase | | | | | | | | | |
| SgK223 | 66 | 180 | Protein | Other | NKF3 | | 196 | 1373 | 1 | 1178 | 86 | 100 |
| | | | Kinase | | | | | | | | | |
| SgK269 | 67 | 181 | Protein | Other | NKF3 | | 1 | 1102 | 1 | 1100 | 60 | 83 |
| | | | Kinase | | | | | | | | | |
| CLIK1 | 68 | 182 | Protein | Other | NKF4 | | 17 | 539 | 1 | 517 | 85 | 88 |
| | | | Kinase | | | | | | | | | |
| SgK307 | 69 | 183 | Protein | Other | NKF5 | | 219 | 1450 | 1 | 1232 | 85 | 100 |
| | | | Kinase | | | | | | | | | |
| SgK424 | 70 | 184 | Protein | Other | NKF5 | | 91 | 469 | 8 | 375 | 67 | 78 |
| | | | Kinase | | | | | | | | | |
| NRBP2 | 71 | 185 | Protein | Other | NRBP | | 1 | 499 | 1 | 535 | 100 | 93 |
| | | | Kinase | | | | | | | | | |
| SgK493 | 72 | 186 | Protein | Other | Other- | | 114 | 491 | 299 | 677 | 72 | 52 |
| | | | Kinase | | Unique | | | | | | | |
| SgK496 | 73 | 187 | Protein | Other | Other- | | 23 | 913 | 1 | 851 | 88 | 66 |
| | | | Kinase | | Unique | | | | | | | |
| SgK071 | 74 | 188 | Protein | Other | Other- | | 69 | 626 | 6 | 682 | 69 | 60 |
| | | | Kinase | | Unique | | | | | | | |
| SgK384 | 75 | 189 | Protein | Other | PLK | | 219 | 599 | 1 | 381 | 63 | 100 |
| | | | Kinase | | | | | | | | | |
| Fused | 76 | 190 | Protein | Other | ULK | | 1 | 1315 | 1 | 1314 | 85 | 85 |
| | | | Kinase | | | | | | | | | |
| ULK3 | 77 | 191 | Protein | Other | ULK | | 1 | 472 | 44 | 515 | 94 | 86 |
| | | | Kinase | | | | | | | | | |
| ULK4 | 78 | 192 | Protein | Other | ULK | | 1 | 878 | 1 | 878 | 69 | 96 |
| | | | Kinase | | | | | | | | | |
| PIK3R4 | 79 | 193 | Protein | Other | VPS15 | | 1 | 1358 | 1 | 1358 | 96 | 96 |
| | | | Kinase | | | | | | | | | |
| Wee1B | 80 | 194 | Protein | Other | WEE | | 192 | 555 | 10 | 376 | 59 | 88 |
| | | | Kinase | | | | | | | | | |
| Wnk2 | 81 | 195 | Protein | Other | Wnk | | 1 | 2114 | 12 | 2275 | 89 | 83 |
| | | | Kinase | | | | | | | | | |
| Wnk1 | 82 | 196 | Protein | Other | Wnk | | 1 | 2377 | 1 | 2382 | 86 | 86 |
| | | | Kinase | | | | | | | | | |
| Wnk3 | 83 | 197 | Protein | Other | Wnk | | 1 | 1751 | 1 | 1743 | 80 | 80 |
| | | | Kinase | | | | | | | | | |
| HSER | 84 | 198 | Protein | RGC | RGC | | 1 | 1066 | 1 | 1072 | 94 | 93 |
| | | | Kinase | | | | | | | | | |
| CYGX | 85 | 199 | Protein | RGC | RGC | | 1 | 908 | 192 | 1105 | 94 | 77 |
| | | | Kinase | | | | | | | | | |
| KSGC | 86 | 200 | Protein | RGC | RGC | | 1 | 1101 | 1 | 1100 | 88 | 88 |
| | | | Kinase | | | | | | | | | |
| MAP3K6 | 87 | 201 | Protein | STE | STE11 | | 1 | 1291 | 1 | 1289 | 97 | 97 |
| | | | Kinase | | | | | | | | | |
| MAP3K8 | 88 | 202 | Protein | STE | STE11 | | 1 | 1367 | 1 | 1454 | 96 | 91 |
| | | | Kinase | | | | | | | | | |
| MAP3K7 | 89 | 203 | Protein | STE | STE11 | | 122 | 1278 | 1 | 1157 | 87 | 100 |
| | | | Kinase | | | | | | | | | |
| OSR1 | 90 | 204 | Protein | STE | STE20 | FRAY | 1 | 527 | 1 | 527 | 96 | 96 |
| | | | Kinase | | | | | | | | | |
| ZC1 | 91 | 205 | Protein | STE | STE20 | MSN | 1 | 1328 | 1 | 1233 | 93 | 100 |
| | | | Kinase | | | | | | | | | |
| ZC2 | 92 | 206 | Protein | STE | STE20 | MSN | 1 | 1351 | 1 | 1352 | 99 | 99 |
| | | | Kinase | | | | | | | | | |
| ZC4 | 93 | 207 | Protein | STE | STE20 | MSN | 1 | 1539 | 1 | 1455 | 94 | 100 |
| | | | Kinase | | | | | | | | | |
| MYO3B | 94 | 208 | Protein | STE | STE20 | NinaC | 1 | 1106 | 13 | 1124 | 62 | 75 |
| | | | Kinase | | | | | | | | | |
| PAK6 | 95 | 209 | Protein | STE | STE20 | PAKB | 1 | 682 | 1 | 681 | 98 | 98 |
| | | | Kinase | | | | | | | | | |
| STLK5 | 96 | 210 | Protein | STE | STE20 | STLK | 1 | 431 | 1 | 431 | 95 | 95 |
| | | | Kinase | | | | | | | | | |
| TAO2 | 97 | 211 | Protein | STE | STE20 | TAO | 1 | 1240 | 1 | 1235 | 98 | 98 |
| | | | Kinase | | | | | | | | | |
| TAO3 | 98 | 212 | Protein | STE | STE20 | TAO | 1 | 898 | 1 | 898 | 96 | 96 |
| | | | Kinase | | | | | | | | | |
| TIF1g | 99 | 213 | atyp PK | TIF1 | | | 1 | 1142 | 5 | 1131 | 95 | 96 |
| ErbB3 | 100 | 214 | Protein | TK | EGFR | | 1 | 1338 | 1 | 1338 | 97 | 97 |
| | | | Kinase | | | | | | | | | |
| EphA5 | 101 | 215 | Protein | TK | Eph | | 1 | 1040 | 1 | 1036 | 96 | 96 |
| | | | Kinase | | | | | | | | | |
| EphA6 | 102 | 216 | Protein | TK | Eph | | 96 | 1130 | 1 | 1035 | 92 | 100 |
| | | | Kinase | | | | | | | | | |
| LMR2 | 103 | 217 | Protein | TK | Lmr | | 1 | 1469 | 1 | 1503 | 78 | 76 |
| | | | Kinase | | | | | | | | | |
| LMR3 | 104 | 218 | Protein | TK | Lmr | | 1 | 1431 | 1 | 1460 | 80 | 78 |
| | | | Kinase | | | | | | | | | |
| FGR | 105 | 219 | Protein | TK | Src | | 1 | 517 | 1 | 517 | 100 | 100 |
| | | | Kinase | | | | | | | | | |
| LRRK2 | 106 | 220 | Protein | TKL | LRRK | | 1057 | 2478 | 1 | 1471 | 51 | 86 |
| | | | Kinase | | | | | | | | | |
| LRRK1 | 107 | 221 | Protein | TKL | LRRK | | 22 | 2004 | 104 | 2108 | 95 | 87 |
| | | | Kinase | | | | | | | | | |
| LZK | 108 | 222 | Protein | TKL | MLK | LZK | 1 | 959 | 1 | 966 | 90 | 89 |
| | | | Kinase | | | | | | | | | |
| MLK2 | 109 | 223 | Protein | TKL | MLK | MLK | 1 | 940 | 1 | 1018 | 98 | 90 |
| | | | Kinase | | | | | | | | | |
| BRAF | 110 | 224 | Protein | TKL | RAF | | 25 | 784 | 6 | 765 | 95 | 97 |
| | | | Kinase | | | | | | | | | |
| KSR2 | 111 | 225 | Protein | TKL | RAF | | 335 | 755 | 25 | 436 | 41 | 91 |
| | | | Kinase | | | | | | | | | |

| | | | | | | | | | |
|---|---|---|---|---|---|---|---|---|---|
| DGKd | 112 | 226 | Lipid Kinase | 1 | 1158 | 40 | 1195 | 94 | 91 |
| DGKi | 113 | 227 | Lipid Kinase | 1 | 1041 | 1 | 1050 | 97 | 96 |
| DGKq | 114 | 228 | Lipid Kinase | 1 | 934 | 1 | 994 | 96 | 91 |

Table 3 describes the extent and the boundaries of the kinase catalytic domains, and other protein domains. These domains were identified using PFAM (pfam.wustl.edu/hmmsearch.shtml) models, a large collection of multiple sequence alignments and hidden Markov models covering many common protein domains. Version Pfam 7.4 (October 2002) contains alignments and models for 4463 protein families. The PFAM alignments were downloaded from pfam.wustl.edu/hmmsearch.shtml and the HMMr searches were run locally on a Timelogic computer (TimeLogic Corporation, Incline Village, Nev.). The column headings are: “Gene”, “ID#na”, “ID#aa ”, “Profile Description”, “Profile Accession”, “Pscore”, “Domain Start”, “Domain End”, “Prof Start”, “Prof End”, “Profile Length”, and “Query Length”. The “Profile Description” column contains the name of the protein domain; “Profile Accession” refers to the PFAM accession number for the domain; “Pscore” lists the probability score, or E-value, and is the number of hits that would be expected to have a score equal or better by chance alone. A good E-value is much less than 1. Around 1 is what is expected just by chance; “Domain Start” lists the amino acid number within the protein sequence at which the domain begins; “Domain End” lists the amino acid number within the protein sequence at which the domain ends; “Prof Start” (Profile Start) refers to the position within the profile at which it begins alignment with the patent sequence; “Prof End” (Profile End) lists the position within the profile at which it the alignment with the patent sequence ends; “Profile Length” lists the length in amino acid residues of the PFAM profile; and “Query Length” lists the amino acid length of the patent protein.

TABLE 3

| | ID# | ID# | | Profile | | Domain | Domain | Prof | Prof | Profile | Query |
|---|---|---|---|---|---|---|---|---|---|---|---|
| Gene | na | aa | Profile Description | Accession | Pscore | Start | End | Start | End | Length | Length |
| DMPK2 | 1 | 115 | Protein kinase domain | PF00069 | 2.50E−64 | 71 | 337 | 1 | 294 | 294 | 1556 |
| DMPK2 | 1 | 115 | Phorbol esters/diacylglycerol | PF00130 | 9.20E−13 | 884 | 932 | 1 | 51 | 51 | 1556 |
| | | | binding domain (C1 domain) | | | | | | | | |
| DMPK2 | 1 | 115 | PH domain | PF00169 | 9.40E−11 | 953 | 1071 | 1 | 85 | 85 | 1556 |
| DMPK2 | 1 | 115 | CNH domain | PF00780 | 3.00E−48 | 1098 | 1371 | 1 | 362 | 362 | 1556 |
| MRCKb | 2 | 116 | Protein kinase domain | PF00069 | 3.60E−64 | 76 | 342 | 1 | 294 | 294 | 1713 |
| MRCKb | 2 | 116 | Phorbol esters/diacylglycerol | PF00130 | 1.50E−14 | 1027 | 1076 | 1 | 51 | 51 | 1713 |
| | | | binding domain (C1 domain) | | | | | | | | |
| MRCKb | 2 | 116 | PH domain | PF00169 | 0.000003 | 1097 | 1215 | 1 | 85 | 85 | 1713 |
| MRCKb | 2 | 116 | Protein kinase C terminal | PF00433 | 0.000031 | 343 | 371 | 1 | 31 | 70 | 1713 |
| | | | domain | | | | | | | | |
| MRCKb | 2 | 116 | CNH domain | PF00780 | 1.90E−96 | 1242 | 1515 | 1 | 362 | 362 | 1713 |
| MAST3 | 3 | 117 | Protein kinase domain | PF00069 | 6.20E−72 | 389 | 662 | 1 | 294 | 294 | 1321 |
| MAST3 | 3 | 117 | PDZ domain | PF00595 | 1.50E−09 | 966 | 1048 | 1 | 79 | 84 | 1321 |
| MAST1 | 4 | 118 | Protein kinase domain | PF00069 | 4.10E−67 | 376 | 649 | 1 | 294 | 294 | 1570 |
| MAST1 | 4 | 118 | PDZ domain | PF00595 | 7.50E−10 | 969 | 1052 | 1 | 80 | 84 | 1570 |
| LATS1 | 5 | 119 | Protein kinase domain | PF00069 | 1.20E−67 | 704 | 850 | 1 | 149 | 294 | 1129 |
| LATS1 | 5 | 119 | Protein kinase domain | PF00069 | 1.20E−67 | 906 | 1009 | 157 | 294 | 294 | 1129 |
| LATS1 | 5 | 119 | UBA domain | PF00627 | 3.40E−08 | 101 | 141 | 1 | 45 | 45 | 1129 |
| PKN1 | 6 | 120 | Protein kinase domain | PF00069 | 1.40E−81 | 619 | 878 | 1 | 294 | 294 | 946 |
| PKN1 | 6 | 120 | Protein kinase C terminal | PF00433 | 5.80E−17 | 879 | 943 | 1 | 64 | 70 | 946 |
| | | | domain | | | | | | | | |
| PKN1 | 6 | 120 | HR1 repeat | PF02185 | 6.30E−56 | 37 | 110 | 1 | 87 | 87 | 946 |
| PKN1 | 6 | 120 | HR1 repeat | PF02185 | 6.30E−56 | 126 | 186 | 1 | 70 | 87 | 946 |
| PKN1 | 6 | 120 | HR1 repeat | PF02185 | 6.30E−56 | 216 | 294 | 1 | 87 | 87 | 946 |
| SGK494 | 7 | 121 | Protein kinase domain | PF00069 | 6.70E−60 | 100 | 352 | 1 | 294 | 294 | 395 |
| RSKL1 | 8 | 122 | Protein kinase domain | PF00069 | 1.30E−18 | 878 | 990 | 85 | 201 | 294 | 1056 |
| RSKL1 | 8 | 122 | Protein kinase domain | PF00069 | 1.30E−18 | 1010 | 1046 | 260 | 294 | 294 | 1056 |
| RSKL1 | 8 | 122 | MIT domain | PF04212 | 3.10E−18 | 235 | 304 | 1 | 74 | 74 | 1056 |
| RSKL1 | 8 | 122 | PX domain | PF00787 | 3.50E−13 | 9 | 128 | 1 | 134 | 134 | 1056 |
| ADCK4 | 9 | 123 | ABC1 Family | PF03109 | 1.40E−42 | 198 | 314 | 1 | 124 | 124 | 533 |
| ADCK5 | 10 | 124 | ABC1 Family | PF03109 | 1.00E−45 | 188 | 304 | 1 | 124 | 124 | 582 |
| AlphaK2 | 11 | 125 | Immunoglobulin domain | PF00047 | 0.000166 | 1302 | 1361 | 1 | 50 | 50 | 1672 |
| AlphaK2 | 11 | 125 | Alpha Kinase | PF02816 | 1.70E−08 | 1508 | 1626 | 116 | 251 | 251 | 1672 |
| AlphaK3 | 12 | 126 | Alpha Kinase | PF02816 | 7.00E−136 | 1000 | 1215 | 1 | 251 | 251 | 1231 |
| BCR | 13 | 127 | PH domain | PF00169 | 0.000035 | 708 | 865 | 1 | 85 | 85 | 1270 |
| BCR | 13 | 127 | C2 domain | PF00168 | 6.60E−12 | 912 | 1001 | 1 | 88 | 88 | 1270 |
| BCR | 13 | 127 | RhoGAP domain | PF00620 | 4.40E−78 | 1067 | 1220 | 1 | 170 | 170 | 1270 |
| BCR | 13 | 127 | RhoGEF domain | PF00621 | 5.80E−84 | 501 | 689 | 1 | 207 | 207 | 1270 |
| ATR | 14 | 128 | FAT domain | PF02259 | 1.20E−188 | 1634 | 2179 | 1 | 722 | 722 | 2635 |
| ATR | 14 | 128 | FATC domain | PF02260 | 2.10E−20 | 2603 | 2635 | 1 | 33 | 33 | 2635 |
| ATR | 14 | 128 | Phosphatidylinositol 3- and | PF00454 | 3.10E−118 | 2312 | 2558 | 1 | 286 | 286 | 2635 |
| | | | 4-kinase | | | | | | | | |
| AMPKa1 | 15 | 129 | Protein kinase domain | PF00069 | 1.20E−93 | 18 | 270 | 1 | 294 | 294 | 550 |
| mSK794 | 16 | 130 | Protein kinase domain | PF00069 | 1.80E−56 | 14 | 262 | 1 | 294 | 294 | 481 |
| mSK798 | 17 | 131 | Protein kinase domain | PF00069 | 7.10E−09 | 3 | 65 | 74 | 138 | 294 | 177 |
| mSK798 | 17 | 131 | Protein kinase domain | PF00069 | 7.10E−09 | 142 | 173 | 260 | 294 | 294 | 177 |
| mSK801 | 18 | 132 | Protein kinase domain | PF00069 | 6.90E−83 | 14 | 263 | 1 | 294 | 294 | 492 |
| mSK804 | 19 | 133 | Protein kinase domain | PF00069 | 3.40E−77 | 14 | 264 | 1 | 294 | 294 | 492 |
| mSK805 | 20 | 134 | Protein kinase domain | PF00069 | 8.60E−20 | 20 | 93 | 1 | 84 | 294 | 304 |
| mSK805 | 20 | 134 | Protein kinase domain | PF00069 | 8.60E−20 | 129 | 249 | 122 | 279 | 294 | 304 |
| mSK807 | 21 | 135 | Protein kinase domain | PF00069 | 4.90E−61 | 14 | 263 | 1 | 294 | 294 | 477 |
| mSK808 | 22 | 136 | Protein kinase domain | PF00069 | 1.10E−64 | 19 | 238 | 1 | 264 | 294 | 238 |
| mSK809 | 23 | 137 | Protein kinase domain | PF00069 | 4.40E−57 | 14 | 262 | 1 | 294 | 294 | 474 |
| mSK811 | 24 | 138 | Protein kinase domain | PF00069 | 5.90E−43 | 4 | 97 | 1 | 99 | 294 | 306 |
| mSK811 | 24 | 138 | Protein kinase domain | PF00069 | 5.90E−43 | 115 | 248 | 119 | 294 | 294 | 306 |
| mSK813 | 25 | 139 | Protein kinase domain | PF00069 | 6.90E−43 | 55 | 172 | 1 | 124 | 294 | 232 |
| mSK813 | 25 | 139 | Protein kinase domain | PF00069 | 6.90E−43 | 173 | 213 | 138 | 180 | 294 | 232 |
| mSK814 | 26 | 140 | Protein kinase domain | PF00069 | 5.90E−61 | 14 | 198 | 1 | 192 | 294 | 209 |
| mSK815 | 27 | 141 | Protein kinase domain | PF00069 | 1.80E−45 | 38 | 218 | 1 | 188 | 294 | 384 |
| mSK815 | 27 | 141 | Protein kinase domain | PF00069 | 1.80E−45 | 252 | 282 | 261 | 294 | 294 | 384 |
| mSK817 | 28 | 142 | Protein kinase domain | PF00069 | 7.10E−40 | 2 | 160 | 1 | 166 | 294 | 178 |
| mSK822 | 29 | 143 | Protein kinase domain | PF00069 | 5.10E−85 | 55 | 302 | 1 | 294 | 294 | 617 |
| mSK823 | 30 | 144 | Protein kinase domain | PF00069 | 1.50E−84 | 23 | 271 | 1 | 294 | 294 | 411 |
| mSK826 | 31 | 145 | Protein kinase domain | PF00069 | 7.50E−71 | 25 | 273 | 1 | 294 | 294 | 520 |
| mSK836 | 32 | 146 | Protein kinase domain | PF00069 | 4.60E−59 | 39 | 216 | 1 | 183 | 294 | 261 |
| mSK838 | 33 | 147 | Protein kinase domain | PF00069 | 8.40E−90 | 14 | 262 | 1 | 294 | 294 | 476 |
| mSK840 | 34 | 148 | Protein kinase domain | PF00069 | 2.90E−75 | 39 | 282 | 1 | 294 | 294 | 514 |
| mSK843 | 35 | 149 | Protein kinase domain | PF00069 | 1.80E−56 | 14 | 262 | 1 | 294 | 294 | 322 |
| NuaK1 | 36 | 150 | Protein kinase domain | PF00069 | 1.70E−96 | 56 | 307 | 1 | 294 | 294 | 658 |
| QSK | 37 | 151 | Protein kinase domain | PF00069 | 7.40E−99 | 53 | 304 | 1 | 294 | 294 | 1356 |
| DCAMKL1 | 38 | 152 | Protein kinase domain | PF00069 | 8.50E−96 | 406 | 663 | 1 | 294 | 294 | 745 |
| DCAMKL1 | 38 | 152 | Doublecortin | PF03607 | 3.70E−55 | 74 | 138 | 1 | 67 | 67 | 745 |
| DCAMKL1 | 38 | 152 | Doublecortin | PF03607 | 3.70E−55 | 203 | 264 | 1 | 67 | 67 | 745 |
| MNK2 | 39 | 153 | Protein kinase domain | PF00069 | 7.90E−74 | 83 | 368 | 1 | 294 | 294 | 459 |
| smMLCK | 40 | 154 | Fibronectin type III domain | PF00041 | 9.90E−23 | 1362 | 1447 | 1 | 84 | 84 | 1950 |
| smMLCK | 40 | 154 | Immunoglobulin domain | PF00047 | 7.20E−78 | 56 | 117 | 1 | 50 | 50 | 1950 |
| smMLCK | 40 | 154 | Immunoglobulin domain | PF00047 | 7.20E−78 | 179 | 239 | 1 | 50 | 50 | 1950 |
| smMLCK | 40 | 154 | Immunoglobulin domain | PF00047 | 7.20E−78 | 425 | 486 | 1 | 50 | 50 | 1950 |
| smMLCK | 40 | 154 | Immunoglobulin domain | PF00047 | 7.20E−78 | 525 | 582 | 1 | 50 | 50 | 1950 |
| smMLCK | 40 | 154 | Immunoglobulin domain | PF00047 | 7.20E−78 | 634 | 694 | 1 | 50 | 50 | 1950 |
| smMLCK | 40 | 154 | Immunoglobulin domain | PF00047 | 7.20E−78 | 732 | 789 | 1 | 46 | 50 | 1950 |
| smMLCK | 40 | 154 | Immunoglobulin domain | PF00047 | 7.20E−78 | 1143 | 1203 | 1 | 50 | 50 | 1950 |
| smMLCK | 40 | 154 | Immunoglobulin domain | PF00047 | 7.20E−78 | 1854 | 1915 | 1 | 50 | 50 | 1950 |
| smMLCK | 40 | 154 | Protein kinase domain | PF00069 | 3.80E−97 | 1495 | 1750 | 1 | 294 | 294 | 1950 |
| TTN | 41 | 155 | Fibronectin type III domain | PF00041 | 0 | 16583 | 16669 | 1 | 84 | 84 | 36946 |
| TTN | 41 | 155 | Fibronectin type III domain | PF00041 | 0 | 16684 | 16770 | 1 | 84 | 84 | 36946 |
| TTN | 41 | 155 | Fibronectin type III domain | PF00041 | 0 | 16785 | 16871 | 1 | 84 | 84 | 36946 |
| TTN | 41 | 155 | Fibronectin type III domain | PF00041 | 0 | 16981 | 17066 | 1 | 84 | 84 | 36946 |
| TTN | 41 | 155 | Fibronectin type III domain | PF00041 | 0 | 17081 | 17166 | 1 | 84 | 84 | 36946 |
| TTN | 41 | 155 | Fibronectin type III domain | PF00041 | 0 | 17277 | 17362 | 1 | 84 | 84 | 36946 |
| TTN | 41 | 155 | Fibronectin type III domain | PF00041 | 0 | 17376 | 17462 | 1 | 84 | 84 | 36946 |
| TTN | 41 | 155 | Fibronectin type III domain | PF00041 | 0 | 17477 | 17562 | 1 | 84 | 84 | 36946 |
| TTN | 41 | 155 | Fibronectin type III domain | PF00041 | 0 | 17577 | 17662 | 1 | 84 | 84 | 36946 |
| TTN | 41 | 155 | Fibronectin type III domain | PF00041 | 0 | 17677 | 17763 | 1 | 84 | 84 | 36946 |
| TTN | 41 | 155 | Fibronectin type III domain | PF00041 | 0 | 17778 | 17865 | 1 | 84 | 84 | 36946 |
| TTN | 41 | 155 | Fibronectin type III domain | PF00041 | 0 | 17973 | 18058 | 1 | 84 | 84 | 36946 |
| TTN | 41 | 155 | Fibronectin type III domain | PF00041 | 0 | 18073 | 18159 | 1 | 84 | 84 | 36946 |
| TTN | 41 | 155 | Fibronectin type III domain | PF00041 | 0 | 18295 | 18382 | 1 | 84 | 84 | 36946 |
| TTN | 41 | 155 | Fibronectin type III domain | PF00041 | 0 | 18396 | 18481 | 1 | 84 | 84 | 36946 |
| TTN | 41 | 155 | Fibronectin type III domain | PF00041 | 0 | 18495 | 18581 | 1 | 84 | 84 | 36946 |
| TTN | 41 | 155 | Fibronectin type III domain | PF00041 | 0 | 18697 | 18781 | 1 | 84 | 84 | 36946 |
| TTN | 41 | 155 | Fibronectin type III domain | PF00041 | 0 | 18795 | 18881 | 1 | 84 | 84 | 36946 |
| TTN | 41 | 155 | Fibronectin type III domain | PF00041 | 0 | 18998 | 19082 | 1 | 84 | 84 | 36946 |
| TTN | 41 | 155 | Fibronectin type III domain | PF00041 | 0 | 19099 | 19190 | 1 | 84 | 84 | 36946 |
| TTN | 41 | 155 | Fibronectin type III domain | PF00041 | 0 | 19205 | 19293 | 1 | 84 | 84 | 36946 |
| TTN | 41 | 155 | Fibronectin type III domain | PF00041 | 0 | 19412 | 19497 | 1 | 84 | 84 | 36946 |
| TTN | 41 | 155 | Fibronectin type III domain | PF00041 | 0 | 19512 | 19603 | 1 | 84 | 84 | 36946 |
| TTN | 41 | 155 | Fibronectin type III domain | PF00041 | 0 | 19717 | 19803 | 1 | 84 | 84 | 36946 |
| TTN | 41 | 155 | Fibronectin type III domain | PF00041 | 0 | 19817 | 19907 | 1 | 84 | 84 | 36946 |
| TTN | 41 | 155 | Fibronectin type III domain | PF00041 | 0 | 19921 | 20007 | 1 | 84 | 84 | 36946 |
| TTN | 41 | 155 | Fibronectin type III domain | PF00041 | 0 | 20116 | 20202 | 1 | 84 | 84 | 36946 |
| TTN | 41 | 155 | Fibronectin type III domain | PF00041 | 0 | 20217 | 20303 | 1 | 84 | 84 | 36946 |
| TTN | 41 | 155 | Fibronectin type III domain | PF00041 | 0 | 20412 | 20495 | 1 | 84 | 84 | 36946 |
| TTN | 41 | 155 | Fibronectin type III domain | PF00041 | 0 | 20512 | 20598 | 1 | 84 | 84 | 36946 |
| TTN | 41 | 155 | Fibronectin type III domain | PF00041 | 0 | 20613 | 20701 | 1 | 84 | 84 | 36946 |
| TTN | 41 | 155 | Fibronectin type III domain | PF00041 | 0 | 20810 | 20895 | 1 | 84 | 84 | 36946 |
| TTN | 41 | 155 | Fibronectin type III domain | PF00041 | 0 | 20910 | 20994 | 1 | 84 | 84 | 36946 |
| TTN | 41 | 155 | Fibronectin type III domain | PF00041 | 0 | 21104 | 21190 | 1 | 84 | 84 | 36946 |
| TTN | 41 | 155 | Fibronectin type III domain | PF00041 | 0 | 21204 | 21289 | 1 | 84 | 84 | 36946 |
| TTN | 41 | 155 | Fibronectin type III domain | PF00041 | 0 | 21304 | 21391 | 1 | 84 | 84 | 36946 |
| TTN | 41 | 155 | Fibronectin type III domain | PF00041 | 0 | 21502 | 21586 | 1 | 84 | 84 | 36946 |
| TTN | 41 | 155 | Fibronectin type III domain | PF00041 | 0 | 21601 | 21684 | 1 | 84 | 84 | 36946 |
| TTN | 41 | 155 | Fibronectin type III domain | PF00041 | 0 | 21797 | 21882 | 1 | 84 | 84 | 36946 |
| TTN | 41 | 155 | Fibronectin type III domain | PF00041 | 0 | 21896 | 21982 | 1 | 84 | 84 | 36946 |
| TTN | 41 | 155 | Fibronectin type III domain | PF00041 | 0 | 21997 | 22089 | 1 | 84 | 84 | 36946 |
| TTN | 41 | 155 | Fibronectin type III domain | PF00041 | 0 | 22199 | 22284 | 1 | 84 | 84 | 36946 |
| TTN | 41 | 155 | Fibronectin type III domain | PF00041 | 0 | 22299 | 22385 | 1 | 84 | 84 | 36946 |
| TTN | 41 | 155 | Fibronectin type III domain | PF00041 | 0 | 22492 | 22578 | 1 | 84 | 84 | 36946 |
| TTN | 41 | 155 | Fibronectin type III domain | PF00041 | 0 | 22592 | 22678 | 1 | 84 | 84 | 36946 |
| TTN | 41 | 155 | Fibronectin type III domain | PF00041 | 0 | 22689 | 22778 | 1 | 84 | 84 | 36946 |
| TTN | 41 | 155 | Fibronectin type III domain | PF00041 | 0 | 22889 | 22974 | 1 | 84 | 84 | 36946 |
| TTN | 41 | 155 | Fibronectin type III domain | PF00041 | 0 | 22988 | 23074 | 1 | 84 | 84 | 36946 |
| TTN | 41 | 155 | Fibronectin type III domain | PF00041 | 0 | 23089 | 23176 | 1 | 84 | 84 | 36946 |
| TTN | 41 | 155 | Fibronectin type III domain | PF00041 | 0 | 23287 | 23372 | 1 | 84 | 84 | 36946 |
| TTN | 41 | 155 | Fibronectin type III domain | PF00041 | 0 | 23384 | 23467 | 1 | 84 | 84 | 36946 |
| TTN | 41 | 155 | Fibronectin type III domain | PF00041 | 0 | 23576 | 23664 | 1 | 84 | 84 | 36946 |
| TTN | 41 | 155 | Fibronectin type III domain | PF00041 | 0 | 23676 | 23762 | 1 | 84 | 84 | 36946 |
| TTN | 41 | 155 | Fibronectin type III domain | PF00041 | 0 | 23773 | 23861 | 1 | 84 | 84 | 36946 |
| TTN | 41 | 155 | Fibronectin type III domain | PF00041 | 0 | 23973 | 24058 | 1 | 84 | 84 | 36946 |
| TTN | 41 | 155 | Fibronectin type III domain | PF00041 | 0 | 24072 | 24158 | 1 | 84 | 84 | 36946 |
| TTN | 41 | 155 | Fibronectin type III domain | PF00041 | 0 | 24173 | 24260 | 1 | 84 | 84 | 36946 |
| TTN | 41 | 155 | Fibronectin type III domain | PF00041 | 0 | 24368 | 24453 | 1 | 84 | 84 | 36946 |
| TTN | 41 | 155 | Fibronectin type III domain | PF00041 | 0 | 24465 | 24549 | 1 | 84 | 84 | 36946 |
| TTN | 41 | 155 | Fibronectin type III domain | PF00041 | 0 | 24659 | 24745 | 1 | 84 | 84 | 36946 |
| TTN | 41 | 155 | Fibronectin type III domain | PF00041 | 0 | 24759 | 24845 | 1 | 84 | 84 | 36946 |
| TTN | 41 | 155 | Fibronectin type III domain | PF00041 | 0 | 24856 | 24944 | 1 | 84 | 84 | 36946 |
| TTN | 41 | 155 | Fibronectin type III domain | PF00041 | 0 | 25055 | 25141 | 1 | 84 | 84 | 36946 |
| TTN | 41 | 155 | Fibronectin type III domain | PF00041 | 0 | 25155 | 25241 | 1 | 84 | 84 | 36946 |
| TTN | 41 | 155 | Fibronectin type III domain | PF00041 | 0 | 25256 | 25343 | 1 | 84 | 84 | 36946 |
| TTN | 41 | 155 | Fibronectin type III domain | PF00041 | 0 | 25452 | 25537 | 1 | 84 | 84 | 36946 |
| TTN | 41 | 155 | Fibronectin type III domain | PF00041 | 0 | 25549 | 25591 | 1 | 42 | 84 | 36946 |
| TTN | 41 | 155 | Fibronectin type III domain | PF00041 | 0 | 25711 | 25794 | 1 | 84 | 84 | 36946 |
| TTN | 41 | 155 | Fibronectin type III domain | PF00041 | 0 | 25811 | 25897 | 1 | 84 | 84 | 36946 |
| TTN | 41 | 155 | Fibronectin type III domain | PF00041 | 0 | 25908 | 25996 | 1 | 84 | 84 | 36946 |
| TTN | 41 | 155 | Fibronectin type III domain | PF00041 | 0 | 26107 | 26193 | 1 | 84 | 84 | 36946 |
| TTN | 41 | 155 | Fibronectin type III domain | PF00041 | 0 | 26207 | 26293 | 1 | 84 | 84 | 36946 |
| TTN | 41 | 155 | Fibronectin type III domain | PF00041 | 0 | 26308 | 26395 | 1 | 84 | 84 | 36946 |
| TTN | 41 | 155 | Fibronectin type III domain | PF00041 | 0 | 26504 | 26589 | 1 | 84 | 84 | 36946 |
| TTN | 41 | 155 | Fibronectin type III domain | PF00041 | 0 | 26601 | 26685 | 1 | 84 | 84 | 36946 |
| TTN | 41 | 155 | Fibronectin type III domain | PF00041 | 0 | 26793 | 26876 | 1 | 84 | 84 | 36946 |
| TTN | 41 | 155 | Fibronectin type III domain | PF00041 | 0 | 26893 | 26979 | 1 | 84 | 84 | 36946 |
| TTN | 41 | 155 | Fibronectin type III domain | PF00041 | 0 | 26990 | 27078 | 1 | 84 | 84 | 36946 |
| TTN | 41 | 155 | Fibronectin type III domain | PF00041 | 0 | 27189 | 27275 | 1 | 84 | 84 | 36946 |
| TTN | 41 | 155 | Fibronectin type III domain | PF00041 | 0 | 27289 | 27375 | 1 | 84 | 84 | 36946 |
| TTN | 41 | 155 | Fibronectin type III domain | PF00041 | 0 | 27390 | 27477 | 1 | 84 | 84 | 36946 |
| TTN | 41 | 155 | Fibronectin type III domain | PF00041 | 0 | 27586 | 27671 | 1 | 84 | 84 | 36946 |
| TTN | 41 | 155 | Fibronectin type III domain | PF00041 | 0 | 27683 | 27767 | 1 | 84 | 84 | 36946 |
| TTN | 41 | 155 | Fibronectin type III domain | PF00041 | 0 | 27875 | 27958 | 1 | 84 | 84 | 36946 |
| TTN | 41 | 155 | Fibronectin type III domain | PF00041 | 0 | 27975 | 28061 | 1 | 84 | 84 | 36946 |
| TTN | 41 | 155 | Fibronectin type III domain | PF00041 | 0 | 28072 | 28160 | 1 | 84 | 84 | 36946 |
| TTN | 41 | 155 | Fibronectin type III domain | PF00041 | 0 | 28272 | 28358 | 1 | 84 | 84 | 36946 |
| TTN | 41 | 155 | Fibronectin type III domain | PF00041 | 0 | 28372 | 28458 | 1 | 84 | 84 | 36946 |
| TTN | 41 | 155 | Fibronectin type III domain | PF00041 | 0 | 28473 | 28560 | 1 | 84 | 84 | 36946 |
| TTN | 41 | 155 | Fibronectin type III domain | PF00041 | 0 | 28669 | 28754 | 1 | 84 | 84 | 36946 |
| TTN | 41 | 155 | Fibronectin type III domain | PF00041 | 0 | 28766 | 28850 | 1 | 84 | 84 | 36946 |
| TTN | 41 | 155 | Fibronectin type III domain | PF00041 | 0 | 28958 | 29043 | 1 | 84 | 84 | 36946 |
| TTN | 41 | 155 | Fibronectin type III domain | PF00041 | 0 | 29057 | 29143 | 1 | 84 | 84 | 36946 |
| TTN | 41 | 155 | Fibronectin type III domain | PF00041 | 0 | 29154 | 29242 | 1 | 84 | 84 | 36946 |
| TTN | 41 | 155 | Fibronectin type III domain | PF00041 | 0 | 29353 | 29439 | 1 | 84 | 84 | 36946 |
| TTN | 41 | 155 | Fibronectin type III domain | PF00041 | 0 | 29453 | 29539 | 1 | 84 | 84 | 36946 |
| TTN | 41 | 155 | Fibronectin type III domain | PF00041 | 0 | 29554 | 29639 | 1 | 84 | 84 | 36946 |
| TTN | 41 | 155 | Fibronectin type III domain | PF00041 | 0 | 29748 | 29833 | 1 | 84 | 84 | 36946 |
| TTN | 41 | 155 | Fibronectin type III domain | PF00041 | 0 | 29845 | 29929 | 1 | 84 | 84 | 36946 |
| TTN | 41 | 155 | Fibronectin type III domain | PF00041 | 0 | 30040 | 30123 | 1 | 84 | 84 | 36946 |
| TTN | 41 | 155 | Fibronectin type III domain | PF00041 | 0 | 30140 | 30226 | 1 | 84 | 84 | 36946 |
| TTN | 41 | 155 | Fibronectin type III domain | PF00041 | 0 | 30238 | 30325 | 1 | 84 | 84 | 36946 |
| TTN | 41 | 155 | Fibronectin type III domain | PF00041 | 0 | 30436 | 30522 | 1 | 84 | 84 | 36946 |
| TTN | 41 | 155 | Fibronectin type III domain | PF00041 | 0 | 30536 | 30622 | 1 | 84 | 84 | 36946 |
| TTN | 41 | 155 | Fibronectin type III domain | PF00041 | 0 | 30637 | 30724 | 1 | 84 | 84 | 36946 |
| TTN | 41 | 155 | Fibronectin type III domain | PF00041 | 0 | 30836 | 30921 | 1 | 84 | 84 | 36946 |
| TTN | 41 | 155 | Fibronectin type III domain | PF00041 | 0 | 30933 | 31017 | 1 | 84 | 84 | 36946 |
| TTN | 41 | 155 | Fibronectin type III domain | PF00041 | 0 | 31127 | 31213 | 1 | 84 | 84 | 36946 |
| TTN | 41 | 155 | Fibronectin type III domain | PF00041 | 0 | 31227 | 31313 | 1 | 84 | 84 | 36946 |
| TTN | 41 | 155 | Fibronectin type III domain | PF00041 | 0 | 31324 | 31411 | 1 | 84 | 84 | 36946 |
| TTN | 41 | 155 | Fibronectin type III domain | PF00041 | 0 | 31522 | 31608 | 1 | 84 | 84 | 36946 |
| TTN | 41 | 155 | Fibronectin type III domain | PF00041 | 0 | 31622 | 31709 | 1 | 84 | 84 | 36946 |
| TTN | 41 | 155 | Fibronectin type III domain | PF00041 | 0 | 31724 | 31810 | 1 | 84 | 84 | 36946 |
| TTN | 41 | 155 | Fibronectin type III domain | PF00041 | 0 | 31919 | 32004 | 1 | 84 | 84 | 36946 |
| TTN | 41 | 155 | Fibronectin type III domain | PF00041 | 0 | 32016 | 32100 | 1 | 84 | 84 | 36946 |
| TTN | 41 | 155 | Fibronectin type III domain | PF00041 | 0 | 32211 | 32299 | 1 | 84 | 84 | 36946 |
| TTN | 41 | 155 | Fibronectin type III domain | PF00041 | 0 | 32311 | 32397 | 1 | 84 | 84 | 36946 |
| TTN | 41 | 155 | Fibronectin type III domain | PF00041 | 0 | 32408 | 32499 | 1 | 84 | 84 | 36946 |
| TTN | 41 | 155 | Fibronectin type III domain | PF00041 | 0 | 32611 | 32698 | 1 | 84 | 84 | 36946 |
| TTN | 41 | 155 | Fibronectin type III domain | PF00041 | 0 | 32710 | 32797 | 1 | 84 | 84 | 36946 |
| TTN | 41 | 155 | Fibronectin type III domain | PF00041 | 0 | 32812 | 32899 | 1 | 84 | 84 | 36946 |
| TTN | 41 | 155 | Fibronectin type III domain | PF00041 | 0 | 33008 | 33093 | 1 | 84 | 84 | 36946 |
| TTN | 41 | 155 | Fibronectin type III domain | PF00041 | 0 | 33105 | 33191 | 1 | 84 | 84 | 36946 |
| TTN | 41 | 155 | Fibronectin type III domain | PF00041 | 0 | 33302 | 33388 | 1 | 84 | 84 | 36946 |
| TTN | 41 | 155 | Fibronectin type III domain | PF00041 | 0 | 33402 | 33488 | 1 | 84 | 84 | 36946 |
| TTN | 41 | 155 | Fibronectin type III domain | PF00041 | 0 | 33503 | 33589 | 1 | 84 | 84 | 36946 |
| TTN | 41 | 155 | Fibronectin type III domain | PF00041 | 0 | 33698 | 33781 | 1 | 84 | 84 | 36946 |
| TTN | 41 | 155 | Fibronectin type III domain | PF00041 | 0 | 33799 | 33886 | 1 | 84 | 84 | 36946 |
| TTN | 41 | 155 | Fibronectin type III domain | PF00041 | 0 | 33901 | 33987 | 1 | 84 | 84 | 36946 |
| TTN | 41 | 155 | Fibronectin type III domain | PF00041 | 0 | 34193 | 34280 | 1 | 84 | 84 | 36946 |
| TTN | 41 | 155 | Fibronectin type III domain | PF00041 | 0 | 34295 | 34381 | 1 | 84 | 84 | 36946 |
| TTN | 41 | 155 | Fibronectin type III domain | PF00041 | 0 | 34469 | 34486 | 70 | 84 | 84 | 36946 |
| TTN | 41 | 155 | Fibronectin type III domain | PF00041 | 0 | 34592 | 34676 | 1 | 84 | 84 | 36946 |
| TTN | 41 | 155 | Immunoglobulin domain | PF00047 | 0 | 58 | 117 | 1 | 47 | 50 | 36946 |
| TTN | 41 | 155 | Immunoglobulin domain | PF00047 | 0 | 156 | 213 | 1 | 47 | 50 | 36946 |
| TTN | 41 | 155 | Immunoglobulin domain | PF00047 | 0 | 998 | 1058 | 1 | 50 | 50 | 36946 |
| TTN | 41 | 155 | Immunoglobulin domain | PF00047 | 0 | 1136 | 1195 | 1 | 47 | 50 | 36946 |
| TTN | 41 | 155 | Immunoglobulin domain | PF00047 | 0 | 1345 | 1406 | 1 | 50 | 50 | 36946 |
| TTN | 41 | 155 | Immunoglobulin domain | PF00047 | 0 | 1515 | 1573 | 1 | 47 | 50 | 36946 |
| TTN | 41 | 155 | Immunoglobulin domain | PF00047 | 0 | 1614 | 1676 | 1 | 50 | 50 | 36946 |
| TTN | 41 | 155 | Immunoglobulin domain | PF00047 | 0 | 1774 | 1823 | 14 | 50 | 50 | 36946 |
| TTN | 41 | 155 | Immunoglobulin domain | PF00047 | 0 | 1899 | 1925 | 1 | 26 | 50 | 36946 |
| TTN | 41 | 155 | Immunoglobulin domain | PF00047 | 0 | 2136 | 2191 | 1 | 44 | 50 | 36946 |
| TTN | 41 | 155 | Immunoglobulin domain | PF00047 | 0 | 2233 | 2292 | 1 | 50 | 50 | 36946 |
| TTN | 41 | 155 | Immunoglobulin domain | PF00047 | 0 | 2325 | 2384 | 1 | 50 | 50 | 36946 |
| TTN | 41 | 155 | Immunoglobulin domain | PF00047 | 0 | 2432 | 2470 | 20 | 47 | 50 | 36946 |
| TTN | 41 | 155 | Immunoglobulin domain | PF00047 | 0 | 2503 | 2559 | 1 | 46 | 50 | 36946 |
| TTN | 41 | 155 | Immunoglobulin domain | PF00047 | 0 | 2591 | 2646 | 1 | 46 | 50 | 36946 |
| TTN | 41 | 155 | Immunoglobulin domain | PF00047 | 0 | 2678 | 2734 | 1 | 47 | 50 | 36946 |
| TTN | 41 | 155 | Immunoglobulin domain | PF00047 | 0 | 2853 | 2912 | 1 | 50 | 50 | 36946 |
| TTN | 41 | 155 | Immunoglobulin domain | PF00047 | 0 | 2940 | 2996 | 1 | 47 | 50 | 36946 |
| TTN | 41 | 155 | Immunoglobulin domain | PF00047 | 0 | 3025 | 3083 | 1 | 47 | 50 | 36946 |
| TTN | 41 | 155 | Immunoglobulin domain | PF00047 | 0 | 3116 | 3172 | 1 | 47 | 50 | 36946 |
| TTN | 41 | 155 | Immunoglobulin domain | PF00047 | 0 | 3222 | 3263 | 19 | 47 | 50 | 36946 |
| TTN | 41 | 155 | Immunoglobulin domain | PF00047 | 0 | 3303 | 3357 | 8 | 50 | 50 | 36946 |
| TTN | 41 | 155 | Immunoglobulin domain | PF00047 | 0 | 3403 | 3458 | 1 | 46 | 50 | 36946 |
| TTN | 41 | 155 | Immunoglobulin domain | PF00047 | 0 | 3523 | 3585 | 1 | 50 | 50 | 36946 |
| TTN | 41 | 155 | Immunoglobulin domain | PF00047 | 0 | 3686 | 3747 | 1 | 47 | 50 | 36946 |
| TTN | 41 | 155 | Immunoglobulin domain | PF00047 | 0 | 4126 | 4186 | 1 | 50 | 50 | 36946 |
| TTN | 41 | 155 | Immunoglobulin domain | PF00047 | 0 | 4338 | 4398 | 1 | 50 | 50 | 36946 |
| TTN | 41 | 155 | Immunoglobulin domain | PF00047 | 0 | 5318 | 5370 | 9 | 50 | 50 | 36946 |
| TTN | 41 | 155 | Immunoglobulin domain | PF00047 | 0 | 5507 | 5568 | 1 | 50 | 50 | 36946 |
| TTN | 41 | 155 | Immunoglobulin domain | PF00047 | 0 | 5641 | 5692 | 1 | 40 | 50 | 36946 |
| TTN | 41 | 155 | Immunoglobulin domain | PF00047 | 0 | 5755 | 5815 | 1 | 50 | 50 | 36946 |
| TTN | 41 | 155 | Immunoglobulin domain | PF00047 | 0 | 5881 | 5932 | 13 | 50 | 50 | 36946 |
| TTN | 41 | 155 | Immunoglobulin domain | PF00047 | 0 | 6497 | 6555 | 1 | 50 | 50 | 36946 |
| TTN | 41 | 155 | Immunoglobulin domain | PF00047 | 0 | 6590 | 6650 | 1 | 50 | 50 | 36946 |
| TTN | 41 | 155 | Immunoglobulin domain | PF00047 | 0 | 6685 | 6745 | 1 | 50 | 50 | 36946 |
| TTN | 41 | 155 | Immunoglobulin domain | PF00047 | 0 | 6778 | 6838 | 1 | 50 | 50 | 36946 |
| TTN | 41 | 155 | Immunoglobulin domain | PF00047 | 0 | 6872 | 6932 | 1 | 50 | 50 | 36946 |
| TTN | 41 | 155 | Immunoglobulin domain | PF00047 | 0 | 6965 | 7025 | 1 | 50 | 50 | 36946 |
| TTN | 41 | 155 | Immunoglobulin domain | PF00047 | 0 | 7058 | 7118 | 1 | 50 | 50 | 36946 |
| TTN | 41 | 155 | Immunoglobulin domain | PF00047 | 0 | 7151 | 7208 | 1 | 47 | 50 | 36946 |
| TTN | 41 | 155 | Immunoglobulin domain | PF00047 | 0 | 7247 | 7307 | 1 | 50 | 50 | 36946 |
| TTN | 41 | 155 | Immunoglobulin domain | PF00047 | 0 | 7351 | 7400 | 14 | 50 | 50 | 36946 |
| TTN | 41 | 155 | Immunoglobulin domain | PF00047 | 0 | 7434 | 7494 | 1 | 50 | 50 | 36946 |
| TTN | 41 | 155 | Immunoglobulin domain | PF00047 | 0 | 7527 | 7587 | 1 | 50 | 50 | 36946 |
| TTN | 41 | 155 | Immunoglobulin domain | PF00047 | 0 | 7620 | 7680 | 1 | 50 | 50 | 36946 |
| TTN | 41 | 155 | Immunoglobulin domain | PF00047 | 0 | 7713 | 7770 | 1 | 47 | 50 | 36946 |
| TTN | 41 | 155 | Immunoglobulin domain | PF00047 | 0 | 7809 | 7869 | 1 | 50 | 50 | 36946 |
| TTN | 41 | 155 | Immunoglobulin domain | PF00047 | 0 | 7902 | 7962 | 1 | 50 | 50 | 36946 |
| TTN | 41 | 155 | Immunoglobulin domain | PF00047 | 0 | 7996 | 8056 | 1 | 50 | 50 | 36946 |
| TTN | 41 | 155 | Immunoglobulin domain | PF00047 | 0 | 8089 | 8149 | 1 | 50 | 50 | 36946 |
| TTN | 41 | 155 | Immunoglobulin domain | PF00047 | 0 | 8182 | 8242 | 1 | 50 | 50 | 36946 |
| TTN | 41 | 155 | Immunoglobulin domain | PF00047 | 0 | 8275 | 8332 | 1 | 47 | 50 | 36946 |
| TTN | 41 | 155 | Immunoglobulin domain | PF00047 | 0 | 8371 | 8431 | 1 | 50 | 50 | 36946 |
| TTN | 41 | 155 | Immunoglobulin domain | PF00047 | 0 | 8464 | 8524 | 1 | 50 | 50 | 36946 |
| TTN | 41 | 155 | Immunoglobulin domain | PF00047 | 0 | 8558 | 8618 | 1 | 50 | 50 | 36946 |
| TTN | 41 | 155 | Immunoglobulin domain | PF00047 | 0 | 8651 | 8711 | 1 | 50 | 50 | 36946 |
| TTN | 41 | 155 | Immunoglobulin domain | PF00047 | 0 | 8744 | 8805 | 1 | 50 | 50 | 36946 |
| TTN | 41 | 155 | Immunoglobulin domain | PF00047 | 0 | 8838 | 8898 | 1 | 50 | 50 | 36946 |
| TTN | 41 | 155 | Immunoglobulin domain | PF00047 | 0 | 8934 | 8994 | 1 | 50 | 50 | 36946 |
| TTN | 41 | 155 | Immunoglobulin domain | PF00047 | 0 | 9027 | 9087 | 1 | 50 | 50 | 36946 |
| TTN | 41 | 155 | Immunoglobulin domain | PF00047 | 0 | 9120 | 9180 | 1 | 50 | 50 | 36946 |
| TTN | 41 | 155 | Immunoglobulin domain | PF00047 | 0 | 9213 | 9273 | 1 | 50 | 50 | 36946 |
| TTN | 41 | 155 | Immunoglobulin domain | PF00047 | 0 | 9309 | 9369 | 1 | 50 | 50 | 36946 |
| TTN | 41 | 155 | Immunoglobulin domain | PF00047 | 0 | 9405 | 9465 | 1 | 50 | 50 | 36946 |
| TTN | 41 | 155 | Immunoglobulin domain | PF00047 | 0 | 9499 | 9559 | 1 | 50 | 50 | 36946 |
| TTN | 41 | 155 | Immunoglobulin domain | PF00047 | 0 | 9592 | 9652 | 1 | 50 | 50 | 36946 |
| TTN | 41 | 155 | Immunoglobulin domain | PF00047 | 0 | 9685 | 9746 | 1 | 50 | 50 | 36946 |
| TTN | 41 | 155 | Immunoglobulin domain | PF00047 | 0 | 9779 | 9836 | 1 | 47 | 50 | 36946 |
| TTN | 41 | 155 | Immunoglobulin domain | PF00047 | 0 | 9875 | 9935 | 1 | 50 | 50 | 36946 |
| TTN | 41 | 155 | Immunoglobulin domain | PF00047 | 0 | 9968 | 10028 | 1 | 50 | 50 | 36946 |
| TTN | 41 | 155 | Immunoglobulin domain | PF00047 | 0 | 10061 | 10121 | 1 | 50 | 50 | 36946 |
| TTN | 41 | 155 | Immunoglobulin domain | PF00047 | 0 | 10154 | 10214 | 1 | 50 | 50 | 36946 |
| TTN | 41 | 155 | Immunoglobulin domain | PF00047 | 0 | 10250 | 10310 | 1 | 50 | 50 | 36946 |
| TTN | 41 | 155 | Immunoglobulin domain | PF00047 | 0 | 10344 | 10365 | 29 | 50 | 50 | 36946 |
| TTN | 41 | 155 | Immunoglobulin domain | PF00047 | 0 | 10403 | 10456 | 8 | 50 | 50 | 36946 |
| TTN | 41 | 155 | Immunoglobulin domain | PF00047 | 0 | 10489 | 10549 | 1 | 50 | 50 | 36946 |
| TTN | 41 | 155 | Immunoglobulin domain | PF00047 | 0 | 10582 | 10643 | 1 | 50 | 50 | 36946 |
| TTN | 41 | 155 | Immunoglobulin domain | PF00047 | 0 | 10676 | 10733 | 1 | 47 | 50 | 36946 |
| TTN | 41 | 155 | Immunoglobulin domain | PF00047 | 0 | 10772 | 10832 | 1 | 50 | 50 | 36946 |
| TTN | 41 | 155 | Immunoglobulin domain | PF00047 | 0 | 10865 | 10925 | 1 | 50 | 50 | 36946 |
| TTN | 41 | 155 | Immunoglobulin domain | PF00047 | 0 | 10958 | 11018 | 1 | 50 | 50 | 36946 |
| TTN | 41 | 155 | Immunoglobulin domain | PF00047 | 0 | 11051 | 11111 | 1 | 50 | 50 | 36946 |
| TTN | 41 | 155 | Immunoglobulin domain | PF00047 | 0 | 11147 | 11207 | 1 | 50 | 50 | 36946 |
| TTN | 41 | 155 | Immunoglobulin domain | PF00047 | 0 | 11243 | 11303 | 1 | 50 | 50 | 36946 |
| TTN | 41 | 155 | Immunoglobulin domain | PF00047 | 0 | 11340 | 11400 | 1 | 50 | 50 | 36946 |
| TTN | 41 | 155 | Immunoglobulin domain | PF00047 | 0 | 11436 | 11496 | 1 | 50 | 50 | 36946 |
| TTN | 41 | 155 | Immunoglobulin domain | PF00047 | 0 | 11544 | 11605 | 1 | 50 | 50 | 36946 |
| TTN | 41 | 155 | Immunoglobulin domain | PF00047 | 0 | 11760 | 11804 | 16 | 47 | 50 | 36946 |
| TTN | 41 | 155 | Immunoglobulin domain | PF00047 | 0 | 11835 | 11894 | 1 | 50 | 50 | 36946 |
| TTN | 41 | 155 | Immunoglobulin domain | PF00047 | 0 | 11924 | 11980 | 1 | 47 | 50 | 36946 |
| TTN | 41 | 155 | Immunoglobulin domain | PF00047 | 0 | 14626 | 14685 | 1 | 50 | 50 | 36946 |
| TTN | 41 | 155 | Immunoglobulin domain | PF00047 | 0 | 14720 | 14776 | 1 | 47 | 50 | 36946 |
| TTN | 41 | 155 | Immunoglobulin domain | PF00047 | 0 | 14813 | 14866 | 1 | 41 | 50 | 36946 |
| TTN | 41 | 155 | Immunoglobulin domain | PF00047 | 0 | 14993 | 15051 | 1 | 50 | 50 | 36946 |
| TTN | 41 | 155 | Immunoglobulin domain | PF00047 | 0 | 15081 | 15132 | 1 | 41 | 50 | 36946 |
| TTN | 41 | 155 | Immunoglobulin domain | PF00047 | 0 | 15170 | 15226 | 1 | 48 | 50 | 36946 |
| TTN | 41 | 155 | Immunoglobulin domain | PF00047 | 0 | 15347 | 15403 | 1 | 47 | 50 | 36946 |
| TTN | 41 | 155 | Immunoglobulin domain | PF00047 | 0 | 15436 | 15488 | 1 | 42 | 50 | 36946 |
| TTN | 41 | 155 | Immunoglobulin domain | PF00047 | 0 | 15525 | 15582 | 1 | 48 | 50 | 36946 |
| TTN | 41 | 155 | Immunoglobulin domain | PF00047 | 0 | 15703 | 15757 | 1 | 45 | 50 | 36946 |
| TTN | 41 | 155 | Immunoglobulin domain | PF00047 | 0 | 15792 | 15851 | 1 | 50 | 50 | 36946 |
| TTN | 41 | 155 | Immunoglobulin domain | PF00047 | 0 | 15881 | 15940 | 1 | 50 | 50 | 36946 |
| TTN | 41 | 155 | Immunoglobulin domain | PF00047 | 0 | 15970 | 16029 | 1 | 50 | 50 | 36946 |
| TTN | 41 | 155 | Immunoglobulin domain | PF00047 | 0 | 16059 | 16118 | 1 | 50 | 50 | 36946 |
| TTN | 41 | 155 | Immunoglobulin domain | PF00047 | 0 | 16147 | 16168 | 1 | 23 | 50 | 36946 |
| TTN | 41 | 155 | Immunoglobulin domain | PF00047 | 0 | 16241 | 16296 | 1 | 46 | 50 | 36946 |
| TTN | 41 | 155 | Immunoglobulin domain | PF00047 | 0 | 16331 | 16389 | 2 | 50 | 50 | 36946 |
| TTN | 41 | 155 | Immunoglobulin domain | PF00047 | 0 | 16419 | 16475 | 1 | 47 | 50 | 36946 |
| TTN | 41 | 155 | Immunoglobulin domain | PF00047 | 0 | 16507 | 16560 | 1 | 46 | 50 | 36946 |
| TTN | 41 | 155 | Immunoglobulin domain | PF00047 | 0 | 16911 | 16959 | 12 | 47 | 50 | 36946 |
| TTN | 41 | 155 | Immunoglobulin domain | PF00047 | 0 | 17198 | 17254 | 1 | 46 | 50 | 36946 |
| TTN | 41 | 155 | Immunoglobulin domain | PF00047 | 0 | 17894 | 17951 | 1 | 47 | 50 | 36946 |
| TTN | 41 | 155 | Immunoglobulin domain | PF00047 | 0 | 18187 | 18273 | 1 | 47 | 50 | 36946 |
| TTN | 41 | 155 | Immunoglobulin domain | PF00047 | 0 | 18619 | 18674 | 1 | 46 | 50 | 36946 |
| TTN | 41 | 155 | Immunoglobulin domain | PF00047 | 0 | 18913 | 18976 | 1 | 47 | 50 | 36946 |
| TTN | 41 | 155 | Immunoglobulin domain | PF00047 | 0 | 19325 | 19390 | 1 | 47 | 50 | 36946 |
| TTN | 41 | 155 | Immunoglobulin domain | PF00047 | 0 | 19631 | 19695 | 1 | 47 | 50 | 36946 |
| TTN | 41 | 155 | Immunoglobulin domain | PF00047 | 0 | 20039 | 20094 | 1 | 47 | 50 | 36946 |
| TTN | 41 | 155 | Immunoglobulin domain | PF00047 | 0 | 20333 | 20392 | 1 | 50 | 50 | 36946 |
| TTN | 41 | 155 | Immunoglobulin domain | PF00047 | 0 | 20733 | 20788 | 1 | 47 | 50 | 36946 |
| TTN | 41 | 155 | Immunoglobulin domain | PF00047 | 0 | 21025 | 21081 | 1 | 46 | 50 | 36946 |
| TTN | 41 | 155 | Immunoglobulin domain | PF00047 | 0 | 21423 | 21479 | 1 | 46 | 50 | 36946 |
| TTN | 41 | 155 | Immunoglobulin domain | PF00047 | 0 | 21716 | 21774 | 1 | 46 | 50 | 36946 |
| TTN | 41 | 155 | Immunoglobulin domain | PF00047 | 0 | 22121 | 22177 | 1 | 47 | 50 | 36946 |
| TTN | 41 | 155 | Immunoglobulin domain | PF00047 | 0 | 22413 | 22470 | 1 | 47 | 50 | 36946 |
| TTN | 41 | 155 | Immunoglobulin domain | PF00047 | 0 | 22820 | 22867 | 13 | 47 | 50 | 36946 |
| TTN | 41 | 155 | Immunoglobulin domain | PF00047 | 0 | 23508 | 23554 | 14 | 47 | 50 | 36946 |
| TTN | 41 | 155 | Immunoglobulin domain | PF00047 | 0 | 23893 | 23951 | 1 | 47 | 50 | 36946 |
| TTN | 41 | 155 | Immunoglobulin domain | PF00047 | 0 | 24593 | 24636 | 16 | 46 | 50 | 36946 |
| TTN | 41 | 155 | Immunoglobulin domain | PF00047 | 0 | 24976 | 25032 | 1 | 46 | 50 | 36946 |
| TTN | 41 | 155 | Immunoglobulin domain | PF00047 | 0 | 25388 | 25429 | 16 | 46 | 50 | 36946 |
| TTN | 41 | 155 | Immunoglobulin domain | PF00047 | 0 | 25632 | 25689 | 1 | 47 | 50 | 36946 |
| TTN | 41 | 155 | Immunoglobulin domain | PF00047 | 0 | 26028 | 26084 | 1 | 46 | 50 | 36946 |
| TTN | 41 | 155 | Immunoglobulin domain | PF00047 | 0 | 26427 | 26482 | 1 | 47 | 50 | 36946 |
| TTN | 41 | 155 | Immunoglobulin domain | PF00047 | 0 | 26714 | 26770 | 1 | 46 | 50 | 36946 |
| TTN | 41 | 155 | Immunoglobulin domain | PF00047 | 0 | 27110 | 27137 | 1 | 27 | 50 | 36946 |
| TTN | 41 | 155 | Immunoglobulin domain | PF00047 | 0 | 27509 | 27563 | 1 | 46 | 50 | 36946 |
| TTN | 41 | 155 | Immunoglobulin domain | PF00047 | 0 | 27796 | 27853 | 1 | 47 | 50 | 36946 |
| TTN | 41 | 155 | Immunoglobulin domain | PF00047 | 0 | 28192 | 28250 | 1 | 47 | 50 | 36946 |
| TTN | 41 | 155 | Immunoglobulin domain | PF00047 | 0 | 28605 | 28646 | 16 | 46 | 50 | 36946 |
| TTN | 41 | 155 | Immunoglobulin domain | PF00047 | 0 | 28892 | 28935 | 16 | 46 | 50 | 36946 |
| TTN | 41 | 155 | Immunoglobulin domain | PF00047 | 0 | 29274 | 29349 | 1 | 50 | 50 | 36946 |
| TTN | 41 | 155 | Immunoglobulin domain | PF00047 | 0 | 29671 | 29726 | 1 | 47 | 50 | 36946 |
| TTN | 41 | 155 | Immunoglobulin domain | PF00047 | 0 | 30357 | 30413 | 1 | 46 | 50 | 36946 |
| TTN | 41 | 155 | Immunoglobulin domain | PF00047 | 0 | 30756 | 30812 | 1 | 46 | 50 | 36946 |
| TTN | 41 | 155 | Immunoglobulin domain | PF00047 | 0 | 31048 | 31105 | 1 | 47 | 50 | 36946 |
| TTN | 41 | 155 | Immunoglobulin domain | PF00047 | 0 | 31443 | 31500 | 1 | 47 | 50 | 36946 |
| TTN | 41 | 155 | Immunoglobulin domain | PF00047 | 0 | 31855 | 31897 | 16 | 47 | 50 | 36946 |
| TTN | 41 | 155 | Immunoglobulin domain | PF00047 | 0 | 32132 | 32189 | 1 | 47 | 50 | 36946 |
| TTN | 41 | 155 | Immunoglobulin domain | PF00047 | 0 | 32532 | 32589 | 1 | 47 | 50 | 36946 |
| TTN | 41 | 155 | Immunoglobulin domain | PF00047 | 0 | 32931 | 32986 | 1 | 47 | 50 | 36946 |
| TTN | 41 | 155 | Immunoglobulin domain | PF00047 | 0 | 33621 | 33675 | 1 | 46 | 50 | 36946 |
| TTN | 41 | 155 | Immunoglobulin domain | PF00047 | 0 | 34017 | 34077 | 1 | 50 | 50 | 36946 |
| TTN | 41 | 155 | Immunoglobulin domain | PF00047 | 0 | 34113 | 34170 | 1 | 46 | 50 | 36946 |
| TTN | 41 | 155 | Immunoglobulin domain | PF00047 | 0 | 34412 | 34470 | 1 | 46 | 50 | 36946 |
| TTN | 41 | 155 | Immunoglobulin domain | PF00047 | 0 | 34512 | 34569 | 1 | 46 | 50 | 36946 |
| TTN | 41 | 155 | Immunoglobulin domain | PF00047 | 0 | 35052 | 35113 | 1 | 50 | 50 | 36946 |
| TTN | 41 | 155 | Immunoglobulin domain | PF00047 | 0 | 35173 | 35233 | 1 | 47 | 50 | 36946 |
| TTN | 41 | 155 | Immunoglobulin domain | PF00047 | 0 | 35278 | 35336 | 1 | 47 | 50 | 36946 |
| TTN | 41 | 155 | Immunoglobulin domain | PF00047 | 0 | 35871 | 35918 | 16 | 50 | 50 | 36946 |
| TTN | 41 | 155 | Immunoglobulin domain | PF00047 | 0 | 36045 | 36105 | 1 | 50 | 50 | 36946 |
| TTN | 41 | 155 | Immunoglobulin domain | PF00047 | 0 | 36203 | 36263 | 4 | 50 | 50 | 36946 |
| TTN | 41 | 155 | Immunoglobulin domain | PF00047 | 0 | 36388 | 36448 | 1 | 50 | 50 | 36946 |
| TTN | 41 | 155 | Immunoglobulin domain | PF00047 | 0 | 36575 | 36633 | 1 | 50 | 50 | 36946 |
| TTN | 41 | 155 | Immunoglobulin domain | PF00047 | 0 | 36670 | 36727 | 1 | 47 | 50 | 36946 |
| TTN | 41 | 155 | Immunoglobulin domain | PF00047 | 0 | 36865 | 36925 | 1 | 47 | 50 | 36946 |
| TTN | 41 | 155 | Protein kinase domain | PF00069 | 1.90E−52 | 34721 | 34975 | 1 | 294 | 294 | 36946 |
| TTN | 41 | 155 | PPAK motif | PF02818 | 0 | 784 | 805 | 4 | 28 | 28 | 36946 |
| TTN | 41 | 155 | PPAK motif | PF02818 | 0 | 11508 | 11526 | 10 | 28 | 28 | 36946 |
| TTN | 41 | 155 | PPAK motif | PF02818 | 0 | 12053 | 12080 | 1 | 28 | 28 | 36946 |
| TTN | 41 | 155 | PPAK motif | PF02818 | 0 | 12081 | 12092 | 1 | 12 | 28 | 36946 |
| TTN | 41 | 155 | PPAK motif | PF02818 | 0 | 12136 | 12154 | 8 | 28 | 28 | 36946 |
| TTN | 41 | 155 | PPAK motif | PF02818 | 0 | 12233 | 12252 | 7 | 27 | 28 | 36946 |
| TTN | 41 | 155 | PPAK motif | PF02818 | 0 | 12253 | 12276 | 1 | 28 | 28 | 36946 |
| TTN | 41 | 155 | PPAK motif | PF02818 | 0 | 12282 | 12306 | 4 | 28 | 28 | 36946 |
| TTN | 41 | 155 | PPAK motif | PF02818 | 0 | 12338 | 12358 | 5 | 25 | 28 | 36946 |
| TTN | 41 | 155 | PPAK motif | PF02818 | 0 | 12361 | 12388 | 1 | 28 | 28 | 36946 |
| TTN | 41 | 155 | PPAK motif | PF02818 | 0 | 12389 | 12416 | 1 | 28 | 28 | 36946 |
| TTN | 41 | 155 | PPAK motif | PF02818 | 0 | 12418 | 12445 | 1 | 28 | 28 | 36946 |
| TTN | 41 | 155 | PPAK motif | PF02818 | 0 | 12446 | 12472 | 1 | 28 | 28 | 36946 |
| TTN | 41 | 155 | PPAK motif | PF02818 | 0 | 12473 | 12496 | 1 | 28 | 28 | 36946 |
| TTN | 41 | 155 | PPAK motif | PF02818 | 0 | 12497 | 12523 | 1 | 28 | 28 | 36946 |
| TTN | 41 | 155 | PPAK motif | PF02818 | 0 | 12542 | 12557 | 13 | 28 | 28 | 36946 |
| TTN | 41 | 155 | PPAK motif | PF02818 | 0 | 12568 | 12591 | 4 | 28 | 28 | 36946 |
| TTN | 41 | 155 | PPAK motif | PF02818 | 0 | 12593 | 12617 | 2 | 26 | 28 | 36946 |
| TTN | 41 | 155 | PPAK motif | PF02818 | 0 | 12618 | 12645 | 1 | 28 | 28 | 36946 |
| TTN | 41 | 155 | PPAK motif | PF02818 | 0 | 12646 | 12673 | 1 | 28 | 28 | 36946 |
| TTN | 41 | 155 | PPAK motif | PF02818 | 0 | 12674 | 12701 | 1 | 28 | 28 | 36946 |
| TTN | 41 | 155 | PPAK motif | PF02818 | 0 | 12702 | 12724 | 1 | 28 | 28 | 36946 |
| TTN | 41 | 155 | PPAK motif | PF02818 | 0 | 12776 | 12796 | 8 | 28 | 28 | 36946 |
| TTN | 41 | 155 | PPAK motif | PF02818 | 0 | 12797 | 12821 | 1 | 28 | 28 | 36946 |
| TTN | 41 | 155 | PPAK motif | PF02818 | 0 | 12823 | 12848 | 2 | 28 | 28 | 36946 |
| TTN | 41 | 155 | PPAK motif | PF02818 | 0 | 12853 | 12878 | 1 | 28 | 28 | 36946 |
| TTN | 41 | 155 | PPAK motif | PF02818 | 0 | 12879 | 12887 | 20 | 28 | 28 | 36946 |
| TTN | 41 | 155 | PPAK motif | PF02818 | 0 | 12947 | 12974 | 1 | 28 | 28 | 36946 |
| TTN | 41 | 155 | PPAK motif | PF02818 | 0 | 12975 | 13000 | 1 | 28 | 28 | 36946 |
| TTN | 41 | 155 | PPAK motif | PF02818 | 0 | 13001 | 13028 | 1 | 28 | 28 | 36946 |
| TTN | 41 | 155 | PPAK motif | PF02818 | 0 | 13029 | 13056 | 1 | 28 | 28 | 36946 |
| TTN | 41 | 155 | PPAK motif | PF02818 | 0 | 13057 | 13080 | 1 | 28 | 28 | 36946 |
| TTN | 41 | 155 | PPAK motif | PF02818 | 0 | 13082 | 13100 | 7 | 28 | 28 | 36946 |
| TTN | 41 | 155 | PPAK motif | PF02818 | 0 | 13106 | 13131 | 1 | 28 | 28 | 36946 |
| TTN | 41 | 155 | PPAK motif | PF02818 | 0 | 13153 | 13178 | 1 | 28 | 28 | 36946 |
| TTN | 41 | 155 | PPAK motif | PF02818 | 0 | 13180 | 13184 | 1 | 5 | 28 | 36946 |
| TTN | 41 | 155 | PPAK motif | PF02818 | 0 | 13216 | 13240 | 4 | 28 | 28 | 36946 |
| TTN | 41 | 155 | PPAK motif | PF02818 | 0 | 13241 | 13265 | 1 | 28 | 28 | 36946 |
| TTN | 41 | 155 | PPAK motif | PF02818 | 0 | 13266 | 13291 | 1 | 28 | 28 | 36946 |
| TTN | 41 | 155 | PPAK motif | PF02818 | 0 | 13292 | 13313 | 1 | 26 | 28 | 36946 |
| TTN | 41 | 155 | PPAK motif | PF02818 | 0 | 13315 | 13331 | 1 | 18 | 28 | 36946 |
| TTN | 41 | 155 | PPAK motif | PF02818 | 0 | 13332 | 13345 | 13 | 28 | 28 | 36946 |
| TTN | 41 | 155 | PPAK motif | PF02818 | 0 | 13346 | 13368 | 1 | 28 | 28 | 36946 |
| TTN | 41 | 155 | PPAK motif | PF02818 | 0 | 13446 | 13473 | 1 | 28 | 28 | 36946 |
| TTN | 41 | 155 | PPAK motif | PF02818 | 0 | 13538 | 13560 | 6 | 28 | 28 | 36946 |
| TTN | 41 | 155 | PPAK motif | PF02818 | 0 | 13561 | 13588 | 1 | 28 | 28 | 36946 |
| TTN | 41 | 155 | PPAK motif | PF02818 | 0 | 13614 | 13639 | 1 | 28 | 28 | 36946 |
| TTN | 41 | 155 | PPAK motif | PF02818 | 0 | 13641 | 13667 | 2 | 28 | 28 | 36946 |
| TTN | 41 | 155 | PPAK motif | PF02818 | 0 | 13668 | 13695 | 1 | 28 | 28 | 36946 |
| TTN | 41 | 155 | PPAK motif | PF02818 | 0 | 13696 | 13723 | 1 | 28 | 28 | 36946 |
| TTN | 41 | 155 | PPAK motif | PF02818 | 0 | 13724 | 13751 | 1 | 28 | 28 | 36946 |
| TTN | 41 | 155 | PPAK motif | PF02818 | 0 | 13752 | 13780 | 1 | 28 | 28 | 36946 |
| TTN | 41 | 155 | PPAK motif | PF02818 | 0 | 13781 | 13807 | 1 | 28 | 28 | 36946 |
| TTN | 41 | 155 | PPAK motif | PF02818 | 0 | 13812 | 13834 | 5 | 28 | 28 | 36946 |
| TTN | 41 | 155 | PPAK motif | PF02818 | 0 | 13839 | 13862 | 5 | 28 | 28 | 36946 |
| TTN | 41 | 155 | PPAK motif | PF02818 | 0 | 13863 | 13890 | 1 | 28 | 28 | 36946 |
| TTN | 41 | 155 | PPAK motif | PF02818 | 0 | 13891 | 13918 | 1 | 28 | 28 | 36946 |
| TTN | 41 | 155 | PPAK motif | PF02818 | 0 | 13919 | 13946 | 1 | 28 | 28 | 36946 |
| TTN | 41 | 155 | PPAK motif | PF02818 | 0 | 13947 | 13974 | 1 | 28 | 28 | 36946 |
| TTN | 41 | 155 | PPAK motif | PF02818 | 0 | 13975 | 14002 | 1 | 28 | 28 | 36946 |
| TTN | 41 | 155 | PPAK motif | PF02818 | 0 | 14003 | 14030 | 1 | 28 | 28 | 36946 |
| TTN | 41 | 155 | PPAK motif | PF02818 | 0 | 14031 | 14059 | 1 | 28 | 28 | 36946 |
| TTN | 41 | 155 | PPAK motif | PF02818 | 0 | 14060 | 14087 | 1 | 28 | 28 | 36946 |
| TTN | 41 | 155 | PPAK motif | PF02818 | 0 | 14088 | 14112 | 1 | 28 | 28 | 36946 |
| TTN | 41 | 155 | PPAK motif | PF02818 | 0 | 14113 | 14138 | 1 | 28 | 28 | 36946 |
| TTN | 41 | 155 | PPAK motif | PF02818 | 0 | 14139 | 14166 | 1 | 28 | 28 | 36946 |
| TTN | 41 | 155 | PPAK motif | PF02818 | 0 | 14171 | 14196 | 1 | 28 | 28 | 36946 |
| TTN | 41 | 155 | PPAK motif | PF02818 | 0 | 14197 | 14201 | 1 | 5 | 28 | 36946 |
| TTN | 41 | 155 | PPAK motif | PF02818 | 0 | 14224 | 14246 | 4 | 28 | 28 | 36946 |
| TTN | 41 | 155 | PPAK motif | PF02818 | 0 | 14247 | 14274 | 1 | 28 | 28 | 36946 |
| TTN | 41 | 155 | PPAK motif | PF02818 | 0 | 14276 | 14302 | 2 | 28 | 28 | 36946 |
| TTN | 41 | 155 | PPAK motif | PF02818 | 0 | 14303 | 14328 | 1 | 26 | 28 | 36946 |
| TTN | 41 | 155 | PPAK motif | PF02818 | 0 | 14330 | 14354 | 1 | 25 | 28 | 36946 |
| TTN | 41 | 155 | PPAK motif | PF02818 | 0 | 14356 | 14381 | 2 | 28 | 28 | 36946 |
| TTN | 41 | 155 | PPAK motif | PF02818 | 0 | 14399 | 14426 | 1 | 28 | 28 | 36946 |
| TTN | 41 | 155 | PPAK motif | PF02818 | 0 | 14432 | 14457 | 1 | 28 | 28 | 36946 |
| TTN | 41 | 155 | PPAK motif | PF02818 | 0 | 14461 | 14482 | 3 | 28 | 28 | 36946 |
| TTN | 41 | 155 | PPAK motif | PF02818 | 0 | 14484 | 14509 | 1 | 28 | 28 | 36946 |
| TTN | 41 | 155 | PPAK motif | PF02818 | 0 | 14513 | 14535 | 6 | 28 | 28 | 36946 |
| TTN | 41 | 155 | PPAK motif | PF02818 | 0 | 14540 | 14567 | 1 | 28 | 28 | 36946 |
| TTN | 41 | 155 | PPAK motif | PF02818 | 0 | 17076 | 17087 | 1 | 12 | 28 | 36946 |
| TTN | 41 | 155 | PPAK motif | PF02818 | 0 | 18214 | 18236 | 5 | 28 | 28 | 36946 |
| TTN | 41 | 155 | PPAK motif | PF02818 | 0 | 19653 | 19665 | 1 | 14 | 28 | 36946 |
| skMLCK | 42 | 156 | Protein kinase domain | PF00069 | 7.90E−78 | 302 | 557 | 1 | 294 | 294 | 613 |
| SgK085 | 43 | 157 | Protein kinase domain | PF00069 | 1.20E−81 | 105 | 360 | 1 | 294 | 294 | 390 |
| PIM2 | 44 | 158 | Protein kinase domain | PF00069 | 4.70E−70 | 132 | 386 | 1 | 294 | 294 | 411 |
| Trio | 45 | 159 | SH3 domain | PF00018 | 7.00E−08 | 1659 | 1699 | 1 | 39 | 58 | 3103 |
| Trio | 45 | 159 | SH3 domain | PF00018 | 7.00E−08 | 2566 | 2586 | 9 | 29 | 58 | 3103 |
| Trio | 45 | 159 | Immunoglobulin domain | PF00047 | 1.70E−08 | 2703 | 2765 | 1 | 50 | 50 | 3103 |
| Trio | 45 | 159 | Protein kinase domain | PF00069 | 1.50E−66 | 2800 | 3054 | 1 | 294 | 294 | 3103 |
| Trio | 45 | 159 | PH domain | PF00169 | 1.80E−25 | 1480 | 1591 | 1 | 85 | 85 | 3103 |
| Trio | 45 | 159 | PH domain | PF00169 | 1.80E−25 | 2158 | 2271 | 1 | 85 | 85 | 3103 |
| Trio | 45 | 159 | Spectrin repeat | PF00435 | 2.90E−21 | 218 | 265 | 1 | 48 | 108 | 3103 |
| Trio | 45 | 159 | Spectrin repeat | PF00435 | 2.90E−21 | 309 | 338 | 79 | 108 | 108 | 3103 |
| Trio | 45 | 159 | Spectrin repeat | PF00435 | 2.90E−21 | 340 | 446 | 1 | 108 | 108 | 3103 |
| Trio | 45 | 159 | Spectrin repeat | PF00435 | 2.90E−21 | 597 | 672 | 30 | 108 | 108 | 3103 |
| Trio | 45 | 159 | Spectrin repeat | PF00435 | 2.90E−21 | 676 | 784 | 6 | 108 | 108 | 3103 |
| Trio | 45 | 159 | Spectrin repeat | PF00435 | 2.90E−21 | 907 | 1012 | 1 | 108 | 108 | 3103 |
| Trio | 45 | 159 | Spectrin repeat | PF00435 | 2.90E−21 | 1138 | 1244 | 1 | 108 | 108 | 3103 |
| Trio | 45 | 159 | RhoGEF domain | PF00621 | 3.50E−73 | 1296 | 1466 | 1 | 207 | 207 | 3103 |
| Trio | 45 | 159 | RhoGEF domain | PF00621 | 3.50E−73 | 1973 | 2144 | 1 | 207 | 207 | 3103 |
| Trad | 46 | 160 | SH3 domain | PF00018 | 0.000002 | 1625 | 1680 | 1 | 53 | 58 | 2966 |
| Trad | 46 | 160 | SH3 domain | PF00018 | 0.000002 | 2346 | 2357 | 47 | 58 | 58 | 2966 |
| Trad | 46 | 160 | Fibronectin type III domain | PF00041 | 1.70E−08 | 2543 | 2583 | 1 | 40 | 84 | 2966 |
| Trad | 46 | 160 | Fibronectin type III domain | PF00041 | 1.70E−08 | 2599 | 2628 | 56 | 84 | 84 | 2966 |
| Trad | 46 | 160 | Immunoglobulin domain | PF00047 | 1.40E−09 | 2459 | 2524 | 1 | 50 | 50 | 2966 |
| Trad | 46 | 160 | Protein kinase domain | PF00069 | 1.00E−65 | 2658 | 2912 | 1 | 294 | 294 | 2966 |
| Trad | 46 | 160 | PH domain | PF00169 | 1.80E−20 | 1445 | 1556 | 1 | 85 | 85 | 2966 |
| Trad | 46 | 160 | PH domain | PF00169 | 1.80E−20 | 2091 | 2200 | 1 | 85 | 85 | 2966 |
| Trad | 46 | 160 | Spectrin repeat | PF00435 | 3.40E−12 | 166 | 213 | 1 | 48 | 108 | 2966 |
| Trad | 46 | 160 | Spectrin repeat | PF00435 | 3.40E−12 | 257 | 286 | 79 | 108 | 108 | 2966 |
| Trad | 46 | 160 | Spectrin repeat | PF00435 | 3.40E−12 | 288 | 394 | 1 | 108 | 108 | 2966 |
| Trad | 46 | 160 | Spectrin repeat | PF00435 | 3.40E−12 | 514 | 620 | 1 | 108 | 108 | 2966 |
| Trad | 46 | 160 | Spectrin repeat | PF00435 | 3.40E−12 | 624 | 666 | 6 | 48 | 108 | 2966 |
| Trad | 46 | 160 | Spectrin repeat | PF00435 | 3.40E−12 | 781 | 855 | 32 | 108 | 108 | 2966 |
| Trad | 46 | 160 | Spectrin repeat | PF00435 | 3.40E−12 | 868 | 915 | 1 | 47 | 108 | 2966 |
| Trad | 46 | 160 | Spectrin repeat | PF00435 | 3.40E−12 | 936 | 982 | 60 | 108 | 108 | 2966 |
| Trad | 46 | 160 | Spectrin repeat | PF00435 | 3.40E−12 | 1107 | 1199 | 1 | 94 | 108 | 2966 |
| Trad | 46 | 160 | RhoGEF domain | PF00621 | 9.20E−76 | 1262 | 1431 | 1 | 207 | 207 | 2966 |
| Trad | 46 | 160 | RhoGEF domain | PF00621 | 9.20E−76 | 1907 | 2077 | 1 | 207 | 207 | 2966 |
| SPEG | 47 | 161 | Fibronectin type III domain | PF00041 | 1.20E−07 | 1287 | 1376 | 1 | 84 | 84 | 3262 |
| SPEG | 47 | 161 | Fibronectin type III domain | PF00041 | 1.20E−07 | 2681 | 2763 | 1 | 84 | 84 | 3262 |
| SPEG | 47 | 161 | Immunoglobulin domain | PF00047 | 3.90E−51 | 59 | 112 | 1 | 50 | 50 | 3262 |
| SPEG | 47 | 161 | Immunoglobulin domain | PF00047 | 3.90E−51 | 741 | 801 | 1 | 50 | 50 | 3262 |
| SPEG | 47 | 161 | Immunoglobulin domain | PF00047 | 3.90E−51 | 888 | 949 | 1 | 50 | 50 | 3262 |
| SPEG | 47 | 161 | Immunoglobulin domain | PF00047 | 3.90E−51 | 987 | 1048 | 1 | 50 | 50 | 3262 |
| SPEG | 47 | 161 | Immunoglobulin domain | PF00047 | 3.90E−51 | 1083 | 1139 | 1 | 46 | 50 | 3262 |
| SPEG | 47 | 161 | Immunoglobulin domain | PF00047 | 3.90E−51 | 1207 | 1263 | 1 | 46 | 50 | 3262 |
| SPEG | 47 | 161 | Immunoglobulin domain | PF00047 | 3.90E−51 | 1413 | 1471 | 8 | 50 | 50 | 3262 |
| SPEG | 47 | 161 | Immunoglobulin domain | PF00047 | 3.90E−51 | 1504 | 1564 | 1 | 50 | 50 | 3262 |
| SPEG | 47 | 161 | Immunoglobulin domain | PF00047 | 3.90E−51 | 2601 | 2662 | 1 | 50 | 50 | 3262 |
| SPEG | 47 | 161 | Protein kinase domain | PF00069 | 6.80E−117 | 1606 | 1859 | 1 | 294 | 294 | 3262 |
| SPEG | 47 | 161 | Protein kinase domain | PF00069 | 6.80E−117 | 2961 | 3213 | 1 | 294 | 294 | 3262 |
| Obscurin | 48 | 162 | Fibronectin type III domain | PF00041 | 1.50E−27 | 509 | 597 | 1 | 84 | 84 | 8523 |
| Obscurin | 48 | 162 | Fibronectin type III domain | PF00041 | 1.50E−27 | 5102 | 5187 | 1 | 84 | 84 | 8523 |
| Obscurin | 48 | 162 | Immunoglobulin domain | PF00047 | 0 | 19 | 79 | 1 | 50 | 50 | 8523 |
| Obscurin | 48 | 162 | Immunoglobulin domain | PF00047 | 0 | 119 | 177 | 1 | 46 | 50 | 8523 |
| Obscurin | 48 | 162 | Immunoglobulin domain | PF00047 | 0 | 246 | 307 | 1 | 50 | 50 | 8523 |
| Obscurin | 48 | 162 | Immunoglobulin domain | PF00047 | 0 | 341 | 400 | 1 | 50 | 50 | 8523 |
| Obscurin | 48 | 162 | Immunoglobulin domain | PF00047 | 0 | 717 | 776 | 1 | 50 | 50 | 8523 |
| Obscurin | 48 | 162 | Immunoglobulin domain | PF00047 | 0 | 809 | 868 | 1 | 50 | 50 | 8523 |
| Obscurin | 48 | 162 | Immunoglobulin domain | PF00047 | 0 | 901 | 960 | 1 | 50 | 50 | 8523 |
| Obscurin | 48 | 162 | Immunoglobulin domain | PF00047 | 0 | 993 | 1052 | 1 | 50 | 50 | 8523 |
| Obscurin | 48 | 162 | Immunoglobulin domain | PF00047 | 0 | 1085 | 1144 | 1 | 50 | 50 | 8523 |
| Obscurin | 48 | 162 | Immunoglobulin domain | PF00047 | 0 | 1177 | 1236 | 1 | 50 | 50 | 8523 |
| Obscurin | 48 | 162 | Immunoglobulin domain | PF00047 | 0 | 1269 | 1328 | 1 | 50 | 50 | 8523 |
| Obscurin | 48 | 162 | Immunoglobulin domain | PF00047 | 0 | 1361 | 1420 | 1 | 50 | 50 | 8523 |
| Obscurin | 48 | 162 | Immunoglobulin domain | PF00047 | 0 | 1453 | 1512 | 1 | 50 | 50 | 8523 |
| Obscurin | 48 | 162 | Immunoglobulin domain | PF00047 | 0 | 1545 | 1604 | 1 | 50 | 50 | 8523 |
| Obscurin | 48 | 162 | Immunoglobulin domain | PF00047 | 0 | 1637 | 1696 | 1 | 50 | 50 | 8523 |
| Obscurin | 48 | 162 | Immunoglobulin domain | PF00047 | 0 | 1731 | 1791 | 1 | 50 | 50 | 8523 |
| Obscurin | 48 | 162 | Immunoglobulin domain | PF00047 | 0 | 1821 | 1880 | 1 | 50 | 50 | 8523 |
| Obscurin | 48 | 162 | Immunoglobulin domain | PF00047 | 0 | 1911 | 1964 | 1 | 44 | 50 | 8523 |
| Obscurin | 48 | 162 | Immunoglobulin domain | PF00047 | 0 | 2176 | 2234 | 1 | 50 | 50 | 8523 |
| Obscurin | 48 | 162 | Immunoglobulin domain | PF00047 | 0 | 2264 | 2318 | 1 | 45 | 50 | 8523 |
| Obscurin | 48 | 162 | Immunoglobulin domain | PF00047 | 0 | 2353 | 2412 | 1 | 50 | 50 | 8523 |
| Obscurin | 48 | 162 | Immunoglobulin domain | PF00047 | 0 | 2532 | 2591 | 1 | 50 | 50 | 8523 |
| Obscurin | 48 | 162 | Immunoglobulin domain | PF00047 | 0 | 2621 | 2675 | 1 | 45 | 50 | 8523 |
| Obscurin | 48 | 162 | Immunoglobulin domain | PF00047 | 0 | 2710 | 2769 | 1 | 50 | 50 | 8523 |
| Obscurin | 48 | 162 | Immunoglobulin domain | PF00047 | 0 | 2799 | 2855 | 1 | 45 | 50 | 8523 |
| Obscurin | 48 | 162 | Immunoglobulin domain | PF00047 | 0 | 2890 | 2949 | 1 | 50 | 50 | 8523 |
| Obscurin | 48 | 162 | Immunoglobulin domain | PF00047 | 0 | 2979 | 3038 | 1 | 50 | 50 | 8523 |
| Obscurin | 48 | 162 | Immunoglobulin domain | PF00047 | 0 | 3068 | 3126 | 1 | 47 | 50 | 8523 |
| Obscurin | 48 | 162 | Immunoglobulin domain | PF00047 | 0 | 3159 | 3218 | 1 | 50 | 50 | 8523 |
| Obscurin | 48 | 162 | Immunoglobulin domain | PF00047 | 0 | 3248 | 3307 | 1 | 50 | 50 | 8523 |
| Obscurin | 48 | 162 | Immunoglobulin domain | PF00047 | 0 | 3337 | 3395 | 1 | 50 | 50 | 8523 |
| Obscurin | 48 | 162 | Immunoglobulin domain | PF00047 | 0 | 3425 | 3483 | 1 | 50 | 50 | 8523 |
| Obscurin | 48 | 162 | Immunoglobulin domain | PF00047 | 0 | 3513 | 3571 | 1 | 50 | 50 | 8523 |
| Obscurin | 48 | 162 | Immunoglobulin domain | PF00047 | 0 | 3601 | 3655 | 1 | 46 | 50 | 8523 |
| Obscurin | 48 | 162 | Immunoglobulin domain | PF00047 | 0 | 3690 | 3748 | 1 | 50 | 50 | 8523 |
| Obscurin | 48 | 162 | Immunoglobulin domain | PF00047 | 0 | 3778 | 3836 | 1 | 50 | 50 | 8523 |
| Obscurin | 48 | 162 | Immunoglobulin domain | PF00047 | 0 | 3866 | 3924 | 1 | 50 | 50 | 8523 |
| Obscurin | 48 | 162 | Immunoglobulin domain | PF00047 | 0 | 3954 | 4012 | 1 | 50 | 50 | 8523 |
| Obscurin | 48 | 162 | Immunoglobulin domain | PF00047 | 0 | 4042 | 4100 | 1 | 50 | 50 | 8523 |
| Obscurin | 48 | 162 | Immunoglobulin domain | PF00047 | 0 | 4130 | 4188 | 1 | 50 | 50 | 8523 |
| Obscurin | 48 | 162 | Immunoglobulin domain | PF00047 | 0 | 4218 | 4276 | 1 | 50 | 50 | 8523 |
| Obscurin | 48 | 162 | Immunoglobulin domain | PF00047 | 0 | 4306 | 4364 | 1 | 50 | 50 | 8523 |
| Obscurin | 48 | 162 | Immunoglobulin domain | PF00047 | 0 | 4394 | 4452 | 1 | 50 | 50 | 8523 |
| Obscurin | 48 | 162 | Immunoglobulin domain | PF00047 | 0 | 4482 | 4541 | 1 | 50 | 50 | 8523 |
| Obscurin | 48 | 162 | Immunoglobulin domain | PF00047 | 0 | 4571 | 4630 | 1 | 50 | 50 | 8523 |
| Obscurin | 48 | 162 | Immunoglobulin domain | PF00047 | 0 | 4660 | 4721 | 1 | 50 | 50 | 8523 |
| Obscurin | 48 | 162 | Immunoglobulin domain | PF00047 | 0 | 4751 | 4808 | 1 | 45 | 50 | 8523 |
| Obscurin | 48 | 162 | Immunoglobulin domain | PF00047 | 0 | 4843 | 4901 | 1 | 47 | 50 | 8523 |
| Obscurin | 48 | 162 | Immunoglobulin domain | PF00047 | 0 | 4933 | 4991 | 1 | 50 | 50 | 8523 |
| Obscurin | 48 | 162 | Immunoglobulin domain | PF00047 | 0 | 5025 | 5084 | 1 | 47 | 50 | 8523 |
| Obscurin | 48 | 162 | Immunoglobulin domain | PF00047 | 0 | 5233 | 5276 | 18 | 50 | 50 | 8523 |
| Obscurin | 48 | 162 | Immunoglobulin domain | PF00047 | 0 | 5488 | 5549 | 1 | 50 | 50 | 8523 |
| Obscurin | 48 | 162 | Immunoglobulin domain | PF00047 | 0 | 5718 | 5779 | 1 | 50 | 50 | 8523 |
| Obscurin | 48 | 162 | Immunoglobulin domain | PF00047 | 0 | 5860 | 5911 | 10 | 50 | 50 | 8523 |
| Obscurin | 48 | 162 | Immunoglobulin domain | PF00047 | 0 | 5961 | 6027 | 1 | 50 | 50 | 8523 |
| Obscurin | 48 | 162 | Immunoglobulin domain | PF00047 | 0 | 6604 | 6665 | 1 | 50 | 50 | 8523 |
| Obscurin | 48 | 162 | Immunoglobulin domain | PF00047 | 0 | 6698 | 6760 | 1 | 50 | 50 | 8523 |
| Obscurin | 48 | 162 | Immunoglobulin domain | PF00047 | 0 | 6951 | 7011 | 1 | 50 | 50 | 8523 |
| Obscurin | 48 | 162 | Immunoglobulin domain | PF00047 | 0 | 8031 | 8092 | 1 | 50 | 50 | 8523 |
| Obscurin | 48 | 162 | Protein kinase domain | PF00069 | 5.10E−111 | 7047 | 7300 | 1 | 294 | 294 | 8523 |
| Obscurin | 48 | 162 | Protein kinase domain | PF00069 | 5.10E−111 | 8227 | 8479 | 1 | 294 | 294 | 8523 |
| Obscurin | 48 | 162 | PH domain | PF00169 | 6.10E−12 | 6472 | 6580 | 1 | 85 | 85 | 8523 |
| Obscurin | 48 | 162 | IQ calmodulin-binding | PF00612 | 0.000004 | 5449 | 5469 | 1 | 21 | 21 | 8523 |
| | | | motif | | | | | | | | |
| Obscurin | 48 | 162 | RhoGEF domain | PF00621 | 4.00E−11 | 6273 | 6452 | 1 | 207 | 207 | 8523 |
| TSSK5 | 49 | 163 | Protein kinase domain | PF00069 | 1.10E−69 | 27 | 302 | 1 | 294 | 294 | 372 |
| CK1g3 | 50 | 164 | Protein kinase domain | PF00069 | 9.90E−34 | 43 | 284 | 1 | 264 | 294 | 448 |
| TTBK2 | 51 | 165 | Protein kinase domain | PF00069 | 5.00E−26 | 21 | 147 | 1 | 131 | 294 | 1243 |
| TTBK2 | 51 | 165 | Protein kinase domain | PF00069 | 5.00E−26 | 163 | 255 | 144 | 264 | 294 | 1243 |
| TTBK1 | 52 | 166 | Protein kinase domain | PF00069 | 8.80E−32 | 34 | 268 | 1 | 264 | 294 | 1308 |
| CHED | 53 | 167 | Protein kinase domain | PF00069 | 4.50E−93 | 705 | 998 | 1 | 294 | 294 | 1511 |
| PFTAIRE2 | 54 | 168 | Protein kinase domain | PF00069 | 4.00E−77 | 101 | 385 | 1 | 294 | 294 | 433 |
| CDKL5 | 55 | 169 | Protein kinase domain | PF00069 | 8.30E−84 | 13 | 297 | 1 | 294 | 294 | 904 |
| CDKL4 | 56 | 170 | Protein kinase domain | PF00069 | 8.40E−101 | 4 | 286 | 1 | 294 | 294 | 342 |
| DYRK4 | 57 | 171 | Protein kinase domain | PF00069 | 6.80E−56 | 173 | 475 | 1 | 294 | 294 | 592 |
| HIPK4 | 58 | 172 | Protein kinase domain | PF00069 | 9.50E−54 | 11 | 272 | 1 | 294 | 294 | 541 |
| ERK4 | 59 | 173 | Protein kinase domain | PF00069 | 5.50E−86 | 20 | 312 | 1 | 294 | 294 | 583 |
| AAK1 | 60 | 174 | Protein kinase domain | PF00069 | 1.40E−40 | 46 | 310 | 1 | 294 | 294 | 958 |
| NEK1 | 61 | 175 | Protein kinase domain | PF00069 | 4.60E−87 | 4 | 258 | 1 | 294 | 294 | 1275 |
| NEK5 | 62 | 176 | Protein kinase domain | PF00069 | 1.90E−88 | 4 | 255 | 1 | 294 | 294 | 778 |
| NEK10 | 63 | 177 | Protein kinase domain | PF00069 | 9.30E−68 | 519 | 783 | 1 | 294 | 294 | 1111 |
| SgK069 | 64 | 178 | Protein kinase domain | PF00069 | 3.80E−39 | 62 | 205 | 1 | 146 | 294 | 362 |
| SgK069 | 64 | 178 | Protein kinase domain | PF00069 | 3.80E−39 | 221 | 315 | 164 | 279 | 294 | 362 |
| SgK110 | 65 | 179 | Protein kinase domain | PF00069 | 1.30E−30 | 38 | 183 | 1 | 149 | 294 | 207 |
| SgK223 | 66 | 180 | Protein kinase domain | PF00069 | 1.10E−07 | 1098 | 1134 | 99 | 135 | 294 | 1373 |
| SgK223 | 66 | 180 | Protein kinase domain | PF00069 | 1.10E−07 | 1225 | 1297 | 189 | 294 | 294 | 1373 |
| SgK269 | 67 | 181 | Protein kinase domain | PF00069 | 0.000006 | 1479 | 1516 | 99 | 136 | 294 | 1735 |
| SgK269 | 67 | 181 | Protein kinase domain | PF00069 | 0.000006 | 1566 | 1657 | 168 | 294 | 294 | 1735 |
| CLIK1 | 68 | 182 | Protein kinase domain | PF00069 | 3.70E−55 | 207 | 528 | 1 | 288 | 294 | 539 |
| SgK307 | 69 | 183 | Ankyrin Repeat | PF00023 | 5.60E−11 | 55 | 87 | 1 | 33 | 33 | 1450 |
| SgK307 | 69 | 183 | Ankyrin Repeat | PF00023 | 5.60E−11 | 88 | 120 | 1 | 33 | 33 | 1450 |
| SgK307 | 69 | 183 | Protein kinase domain | PF00069 | 5.50E−16 | 295 | 369 | 54 | 124 | 294 | 1450 |
| SgK307 | 69 | 183 | Protein kinase domain | PF00069 | 5.50E−16 | 415 | 484 | 165 | 270 | 294 | 1450 |
| SgK424 | 70 | 184 | Ankyrin Repeat | PF00023 | 0.000019 | 25 | 57 | 1 | 33 | 33 | 469 |
| SgK424 | 70 | 184 | Ankyrin Repeat | PF00023 | 0.000019 | 99 | 111 | 21 | 33 | 33 | 469 |
| NRBP2 | 71 | 185 | Protein kinase domain | PF00069 | 4.70E−21 | 92 | 190 | 52 | 143 | 294 | 499 |
| NRBP2 | 71 | 185 | Protein kinase domain | PF00069 | 4.70E−21 | 230 | 304 | 178 | 294 | 294 | 499 |
| SgK493 | 72 | 186 | No domain identified | | | | | | | | |
| SgK496 | 73 | 187 | Protein kinase domain | PF00069 | 1.50E−41 | 650 | 895 | 1 | 280 | 294 | 927 |
| SgK071 | 74 | 188 | Protein kinase domain | PF00069 | 1.40E−22 | 55 | 184 | 18 | 142 | 294 | 626 |
| SgK071 | 74 | 188 | Protein kinase domain | PF00069 | 1.40E−22 | 227 | 255 | 166 | 197 | 294 | 626 |
| SgK071 | 74 | 188 | Protein kinase domain | PF00069 | 1.40E−22 | 303 | 323 | 271 | 294 | 294 | 626 |
| SgK384 | 75 | 189 | Protein kinase domain | PF00069 | 9.70E−82 | 27 | 283 | 1 | 294 | 294 | 599 |
| SgK384 | 75 | 189 | POLO box duplicated region | PF00659 | 3.60E−13 | 424 | 487 | 1 | 77 | 77 | 599 |
| SgK384 | 75 | 189 | POLO box duplicated region | PF00659 | 3.60E−13 | 522 | 537 | 3 | 18 | 77 | 599 |
| Fused | 76 | 190 | Protein kinase domain | PF00069 | 1.10E−99 | 4 | 254 | 1 | 294 | 294 | 1316 |
| ULK3 | 77 | 191 | Protein kinase domain | PF00069 | 1.60E−89 | 14 | 270 | 1 | 294 | 294 | 472 |
| ULK3 | 77 | 191 | MIT domain | PF04212 | 4.80E−18 | 277 | 346 | 1 | 74 | 74 | 472 |
| ULK3 | 77 | 191 | MIT domain | PF04212 | 4.80E−18 | 372 | 442 | 1 | 74 | 74 | 472 |
| ULK4 | 78 | 192 | Protein kinase domain | PF00069 | 3.70E−53 | 4 | 280 | 1 | 294 | 294 | 1275 |
| PIK3R4 | 79 | 193 | Protein kinase domain | PF00069 | 0.000002 | 104 | 158 | 79 | 135 | 294 | 1358 |
| PIK3R4 | 79 | 193 | Protein kinase domain | PF00069 | 0.000002 | 240 | 306 | 184 | 287 | 294 | 1358 |
| PIK3R4 | 79 | 193 | HEAT repeat | PF02985 | 0.000002 | 410 | 448 | 1 | 39 | 39 | 1358 |
| PIK3R4 | 79 | 193 | HEAT repeat | PF02985 | 0.000002 | 455 | 493 | 1 | 39 | 39 | 1358 |
| PIK3R4 | 79 | 193 | WD domain, G-beta repeat | PF00400 | 3.60E−21 | 983 | 1021 | 1 | 39 | 39 | 1358 |
| PIK3R4 | 79 | 193 | WD domain, G-beta repeat | PF00400 | 3.60E−21 | 1229 | 1269 | 1 | 39 | 39 | 1358 |
| PIK3R4 | 79 | 193 | WD domain, G-beta repeat | PF00400 | 3.60E−21 | 1319 | 1358 | 1 | 39 | 39 | 1358 |
| Wee1B | 80 | 194 | Protein kinase domain | PF00069 | 7.40E−42 | 208 | 475 | 1 | 288 | 294 | 555 |
| Wnk2 | 81 | 195 | Protein kinase domain | PF00069 | 3.70E−60 | 170 | 428 | 1 | 294 | 294 | 2132 |
| Wnk1 | 82 | 196 | Protein kinase domain | PF00069 | 3.40E−63 | 221 | 479 | 1 | 294 | 294 | 2377 |
| Wnk3 | 83 | 197 | Protein kinase domain | PF00069 | 1.30E−65 | 146 | 404 | 1 | 294 | 294 | 1751 |
| HSER | 84 | 198 | Protein kinase domain | PF00069 | 4.60E−19 | 537 | 628 | 59 | 146 | 294 | 1066 |
| HSER | 84 | 198 | Protein kinase domain | PF00069 | 4.60E−19 | 640 | 734 | 166 | 288 | 294 | 1066 |
| HSER | 84 | 198 | ANF_receptor | PF01094 | 6.30E−62 | 36 | 277 | 1 | 258 | 456 | 1066 |
| HSER | 84 | 198 | ANF_receptor | PF01094 | 6.30E−62 | 313 | 400 | 349 | 456 | 456 | 1066 |
| HSER | 84 | 198 | Guanylate cyclase catalytic | PF00211 | 2.10E−95 | 808 | 995 | 1 | 226 | 226 | 1066 |
| | | | domain | | | | | | | | |
| CYGX | 85 | 199 | Protein kinase domain | PF00069 | 6.70E−31 | 413 | 621 | 52 | 294 | 294 | 908 |
| CYGX | 85 | 199 | ANF_receptor | PF01094 | 7.10E−50 | 2 | 252 | 148 | 456 | 456 | 908 |
| CYGX | 85 | 199 | Guanylate cyclase catalytic | PF00211 | 1.30E−87 | 687 | 874 | 1 | 226 | 226 | 908 |
| | | | domain | | | | | | | | |
| KSGC | 86 | 200 | Protein kinase domain | PF00069 | 3.30E−28 | 600 | 827 | 44 | 292 | 294 | 1101 |
| KSGC | 86 | 200 | ANF_receptor | PF01094 | 3.80E−63 | 46 | 437 | 1 | 456 | 456 | 1101 |
| KSGC | 86 | 200 | Guanylate cyclase catalytic | PF00211 | 1.20E−86 | 893 | 1079 | 1 | 226 | 226 | 1101 |
| | | | domain | | | | | | | | |
| MAP3K6 | 87 | 201 | Protein kinase domain | PF00069 | 1.80E−73 | 654 | 907 | 6 | 294 | 294 | 1291 |
| MAP3K8 | 88 | 202 | Ankyrin Repeat | PF00023 | 0.000181 | 43 | 65 | 1 | 23 | 33 | 1388 |
| MAP3K8 | 88 | 202 | Ankyrin Repeat | PF00023 | 0.000181 | 72 | 117 | 1 | 33 | 33 | 1388 |
| MAP3K8 | 88 | 202 | Protein kinase domain | PF00069 | 2.40E−86 | 1121 | 1384 | 1 | 294 | 294 | 1388 |
| MAP3K7 | 89 | 203 | Protein kinase domain | PF00069 | 6.70E−79 | 610 | 861 | 6 | 294 | 294 | 1334 |
| OSR1 | 90 | 204 | Protein kinase domain | PF00069 | 2.70E−80 | 17 | 291 | 1 | 294 | 294 | 527 |
| ZC1 | 91 | 205 | Protein kinase domain | PF00069 | 1.20E−85 | 25 | 288 | 1 | 294 | 294 | 1328 |
| ZC1 | 91 | 205 | CNH domain | PF00780 | 5.80E−102 | 1015 | 1306 | 1 | 362 | 362 | 1328 |
| ZC2 | 92 | 206 | Protein kinase domain | PF00069 | 1.30E−83 | 25 | 288 | 1 | 294 | 294 | 1351 |
| ZC2 | 92 | 206 | CNH domain | PF00780 | 3.00E−96 | 1038 | 1329 | 1 | 362 | 362 | 1351 |
| ZC4 | 93 | 207 | Protein kinase domain | PF00069 | 8.10E−74 | 25 | 313 | 1 | 294 | 294 | 1539 |
| ZC4 | 93 | 207 | CNH domain | PF00780 | 1.40E−82 | 1166 | 1311 | 1 | 191 | 362 | 1539 |
| ZC4 | 93 | 207 | CNH domain | PF00780 | 1.40E−82 | 1358 | 1513 | 190 | 362 | 362 | 1539 |
| MYO3B | 94 | 208 | Protein kinase domain | PF00069 | 8.60E−78 | 15 | 281 | 1 | 294 | 294 | 1613 |
| MYO3B | 94 | 208 | IQ calmodulin-binding motif | PF00612 | 3.50E−08 | 1043 | 1063 | 1 | 21 | 21 | 1613 |
| MYO3B | 94 | 208 | IQ calmodulin-binding motif | PF00612 | 3.50E−08 | 1070 | 1090 | 1 | 21 | 21 | 1613 |
| MYO3B | 94 | 208 | Myosin head (motor domain) | PF00063 | 1.70E−232 | 333 | 1028 | 1 | 734 | 734 | 1613 |
| PAK6 | 95 | 209 | Protein kinase domain | PF00069 | 6.10E−78 | 408 | 659 | 1 | 294 | 294 | 682 |
| PAK6 | 95 | 209 | P21-Rho-binding domain | PF00786 | 9.10E−11 | 12 | 46 | 1 | 37 | 64 | 682 |
| STLK5 | 96 | 210 | Protein kinase domain | PF00069 | 1.60E−30 | 69 | 212 | 1 | 143 | 294 | 431 |
| STLK5 | 96 | 210 | Protein kinase domain | PF00069 | 1.60E−30 | 241 | 298 | 164 | 256 | 294 | 431 |
| STLK5 | 96 | 210 | Protein kinase domain | PF00069 | 1.60E−30 | 355 | 379 | 267 | 294 | 294 | 431 |
| TAO2 | 97 | 211 | Protein kinase domain | PF00069 | 1.50E−72 | 28 | 281 | 1 | 294 | 294 | 1240 |
| TAO3 | 98 | 212 | Protein kinase domain | PF00069 | 1.00E−73 | 24 | 277 | 1 | 294 | 294 | 898 |
| TIF1g | 99 | 213 | Bromodomain | PF00439 | 9.50E−20 | 994 | 1066 | 19 | 92 | 92 | 1142 |
| TIF1g | 99 | 213 | PHD finger | PF00628 | 1.50E−14 | 196 | 202 | 1 | 7 | 51 | 1142 |
| TIF1g | 99 | 213 | PHD finger | PF00628 | 1.50E−14 | 250 | 260 | 41 | 51 | 51 | 1142 |
| TIF1g | 99 | 213 | PHD finger | PF00628 | 1.50E−14 | 904 | 947 | 1 | 49 | 51 | 1142 |
| TIF1g | 99 | 213 | B-box zinc finger | PF00643 | 1.20E−24 | 228 | 275 | 1 | 48 | 48 | 1142 |
| TIF1g | 99 | 213 | B-box zinc finger | PF00643 | 1.20E−24 | 287 | 328 | 1 | 48 | 48 | 1142 |
| ErbB3 | 100 | 214 | Protein kinase domain | PF00069 | 2.50E−62 | 707 | 959 | 1 | 288 | 294 | 1339 |
| ErbB3 | 100 | 214 | Furin-like cysteine-rich | PF00757 | 2.10E−93 | 180 | 332 | 1 | 183 | 183 | 1339 |
| | | | region | | | | | | | | |
| ErbB3 | 100 | 214 | Furin-like cysteine-rich | PF00757 | 2.10E−93 | 490 | 515 | 1 | 44 | 183 | 1339 |
| | | | region | | | | | | | | |
| ErbB3 | 100 | 214 | Furin-like cysteine-rich | PF00757 | 2.10E−93 | 517 | 533 | 92 | 108 | 183 | 1339 |
| | | | region | | | | | | | | |
| ErbB3 | 100 | 214 | Furin-like cysteine-rich | PF00757 | 2.10E−93 | 607 | 629 | 1 | 46 | 183 | 1339 |
| | | | region | | | | | | | | |
| ErbB3 | 100 | 214 | Receptor L domain | PF01030 | 6.90E−101 | 55 | 178 | 1 | 154 | 154 | 1339 |
| ErbB3 | 100 | 214 | Receptor L domain | PF01030 | 6.90E−101 | 353 | 485 | 1 | 154 | 154 | 1339 |
| EphA5 | 101 | 215 | Fibronectin type III domain | PF00041 | 1.30E−32 | 360 | 456 | 1 | 84 | 84 | 1041 |
| EphA5 | 101 | 215 | Fibronectin type III domain | PF00041 | 1.30E−32 | 471 | 554 | 1 | 84 | 84 | 1041 |
| EphA5 | 101 | 215 | Protein kinase domain | PF00069 | 8.60E−81 | 678 | 935 | 1 | 292 | 294 | 1041 |
| EphA5 | 101 | 215 | Ephrin receptor ligand | PF01404 | 1.90E−139 | 62 | 235 | 1 | 177 | 177 | 1041 |
| | | | binding domain | | | | | | | | |
| EphA5 | 101 | 215 | SAM domain (Sterile alpha | PF00536 | 9.20E−25 | 966 | 1031 | 1 | 68 | 68 | 1041 |
| | | | motif) | | | | | | | | |
| EphA6 | 102 | 216 | Fibronectin type III domain | PF00041 | 7.90E−24 | 426 | 519 | 1 | 84 | 84 | 1130 |
| EphA6 | 102 | 216 | Fibronectin type III domain | PF00041 | 7.90E−24 | 534 | 621 | 1 | 84 | 84 | 1130 |
| EphA6 | 102 | 216 | Protein kinase domain | PF00069 | 6.10E−72 | 725 | 1024 | 1 | 294 | 294 | 1130 |
| EphA6 | 102 | 216 | Ephrin receptor ligand | PF01404 | 1.60E−140 | 128 | 301 | 1 | 177 | 177 | 1130 |
| | | | binding domain | | | | | | | | |
| EphA6 | 102 | 216 | SAM domain (Sterile alpha | PF00536 | 9.80E−26 | 1053 | 1117 | 1 | 68 | 68 | 1130 |
| | | | motif) | | | | | | | | |
| LMR2 | 103 | 217 | Protein kinase domain | PF00069 | 2.00E−21 | 136 | 404 | 1 | 294 | 294 | 1469 |
| LMR3 | 104 | 218 | Protein kinase domain | PF00069 | 2.00E−48 | 133 | 408 | 1 | 294 | 294 | 1431 |
| FGR | 105 | 219 | SH3 domain | PF00018 | 2.20E−25 | 68 | 124 | 1 | 58 | 58 | 517 |
| FGR | 105 | 219 | Protein kinase domain | PF00069 | 1.20E−75 | 251 | 500 | 1 | 294 | 294 | 517 |
| FGR | 105 | 219 | SH2 domain | PF00017 | 6.30E−53 | 132 | 214 | 1 | 79 | 79 | 517 |
| LRRK2 | 106 | 220 | Protein kinase domain | PF00069 | 1.10E−33 | 1836 | 2079 | 7 | 288 | 294 | 2478 |
| LRRK2 | 106 | 220 | Leucine Rich Repeat | PF00560 | 4.60E−28 | 796 | 822 | 5 | 25 | 25 | 2478 |
| LRRK2 | 106 | 220 | Leucine Rich Repeat | PF00560 | 4.60E−28 | 983 | 1005 | 1 | 25 | 25 | 2478 |
| LRRK2 | 106 | 220 | Leucine Rich Repeat | PF00560 | 4.60E−28 | 1012 | 1035 | 1 | 25 | 25 | 2478 |
| LRRK2 | 106 | 220 | Leucine Rich Repeat | PF00560 | 4.60E−28 | 1036 | 1058 | 1 | 25 | 25 | 2478 |
| LRRK2 | 106 | 220 | Leucine Rich Repeat | PF00560 | 4.60E−28 | 1084 | 1107 | 1 | 25 | 25 | 2478 |
| LRRK2 | 106 | 220 | Leucine Rich Repeat | PF00560 | 4.60E−28 | 1108 | 1129 | 1 | 25 | 25 | 2478 |
| LRRK2 | 106 | 220 | Leucine Rich Repeat | PF00560 | 4.60E−28 | 1130 | 1153 | 1 | 25 | 25 | 2478 |
| LRRK2 | 106 | 220 | Leucine Rich Repeat | PF00560 | 4.60E−28 | 1174 | 1196 | 1 | 25 | 25 | 2478 |
| LRRK2 | 106 | 220 | Leucine Rich Repeat | PF00560 | 4.60E−28 | 1197 | 1218 | 1 | 25 | 25 | 2478 |
| LRRK2 | 106 | 220 | Leucine Rich Repeat | PF00560 | 4.60E−28 | 1221 | 1242 | 1 | 25 | 25 | 2478 |
| LRRK2 | 106 | 220 | Leucine Rich Repeat | PF00560 | 4.60E−28 | 1246 | 1268 | 1 | 25 | 25 | 2478 |
| LRRK2 | 106 | 220 | Leucine Rich Repeat | PF00560 | 4.60E−28 | 1269 | 1292 | 1 | 25 | 25 | 2478 |
| LRRK1 | 107 | 221 | Ankyrin Repeat | PF00023 | 0.000041 | 81 | 97 | 6 | 22 | 33 | 2004 |
| LRRK1 | 107 | 221 | Ankyrin Repeat | PF00023 | 0.000041 | 116 | 131 | 8 | 23 | 33 | 2004 |
| LRRK1 | 107 | 221 | Ankyrin Repeat | PF00023 | 0.000041 | 147 | 175 | 6 | 33 | 33 | 2004 |
| LRRK1 | 107 | 221 | Ankyrin Repeat | PF00023 | 0.000041 | 187 | 208 | 5 | 26 | 33 | 2004 |
| LRRK1 | 107 | 221 | Protein kinase domain | PF00069 | 1.90E−43 | 1232 | 1507 | 1 | 289 | 294 | 2004 |
| LRRK1 | 107 | 221 | Leucine Rich Repeat | PF00560 | 4.20E−20 | 269 | 292 | 1 | 25 | 25 | 2004 |
| LRRK1 | 107 | 221 | Leucine Rich Repeat | PF00560 | 4.20E−20 | 293 | 308 | 1 | 17 | 25 | 2004 |
| LRRK1 | 107 | 221 | Leucine Rich Repeat | PF00560 | 4.20E−20 | 320 | 342 | 1 | 25 | 25 | 2004 |
| LRRK1 | 107 | 221 | Leucine Rich Repeat | PF00560 | 4.20E−20 | 371 | 394 | 1 | 25 | 25 | 2004 |
| LRRK1 | 107 | 221 | Leucine Rich Repeat | PF00560 | 4.20E−20 | 395 | 418 | 1 | 25 | 25 | 2004 |
| LRRK1 | 107 | 221 | Leucine Rich Repeat | PF00560 | 4.20E−20 | 441 | 461 | 1 | 22 | 25 | 2004 |
| LRRK1 | 107 | 221 | Leucine Rich Repeat | PF00560 | 4.20E−20 | 464 | 480 | 1 | 18 | 25 | 2004 |
| LRRK1 | 107 | 221 | Leucine Rich Repeat | PF00560 | 4.20E−20 | 488 | 507 | 1 | 25 | 25 | 2004 |
| LRRK1 | 107 | 221 | Leucine Rich Repeat | PF00560 | 4.20E−20 | 539 | 561 | 1 | 25 | 25 | 2004 |
| LRRK1 | 107 | 221 | Leucine Rich Repeat | PF00560 | 4.20E−20 | 562 | 585 | 1 | 25 | 25 | 2004 |
| LRRK1 | 107 | 221 | Leucine Rich Repeat | PF00560 | 4.20E−20 | 586 | 604 | 1 | 20 | 25 | 2004 |
| LRRK1 | 107 | 221 | Leucine Rich Repeat | PF00560 | 4.20E−20 | 1174 | 1196 | 1 | 25 | 25 | 2004 |
| LZK | 108 | 222 | Protein kinase domain | PF00069 | 5.30E−79 | 167 | 408 | 1 | 294 | 294 | 959 |
| MLK2 | 109 | 223 | SH3 domain | PF00018 | 1.80E−15 | 19 | 79 | 1 | 58 | 58 | 940 |
| MLK2 | 109 | 223 | Protein kinase domain | PF00069 | 4.90E−85 | 98 | 359 | 1 | 294 | 294 | 940 |
| BRAF | 110 | 224 | Protein kinase domain | PF00069 | 4.90E−83 | 475 | 734 | 1 | 294 | 294 | 784 |
| BRAF | 110 | 224 | Phorbol esters/diacylglycerol | PF00130 | 1.00E−16 | 252 | 297 | 1 | 51 | 51 | 784 |
| | | | binding domain (C1 domain) | | | | | | | | |
| BRAF | 110 | 224 | Raf-like Ras-binding domain | PF02196 | 1.90E−37 | 172 | 244 | 1 | 77 | 77 | 784 |
| KSR2 | 111 | 225 | Protein kinase domain | PF00069 | 9.60E−41 | 682 | 941 | 1 | 289 | 294 | 965 |
| KSR2 | 111 | 225 | Phorbol esters/diacylglycerol | PF00130 | 0.000128 | 420 | 463 | 1 | 51 | 51 | 965 |
| | | | binding domain (C1 domain) | | | | | | | | |
| DGKd | 112 | 226 | Phorbol esters/diacylglycerol | PF00130 | 5.50E−17 | 106 | 155 | 1 | 51 | 51 | 1158 |
| | | | binding domain (C1 domain) | | | | | | | | |
| DGKd | 112 | 226 | Phorbol esters/diacylglycerol | PF00130 | 5.50E−17 | 178 | 228 | 1 | 51 | 51 | 1158 |
| | | | binding domain (C1 domain) | | | | | | | | |
| DGKd | 112 | 226 | PH domain | PF00169 | 6.60E−15 | 2 | 88 | 7 | 85 | 85 | 1158 |
| DGKd | 112 | 226 | Diacylglycerol kinase | PF00609 | 1.10E−60 | 707 | 864 | 1 | 190 | 190 | 1158 |
| | | | accessory domain | | | | | | | | |
| DGKd | 112 | 226 | Diacylglycerol kinase | PF00781 | 1.80E−62 | 263 | 388 | 1 | 154 | 154 | 1158 |
| | | | catalytic domain | | | | | | | | |
| DGKd | 112 | 226 | SAM domain (Sterile alpha | PF00536 | 2.30E−21 | 1087 | 1150 | 1 | 68 | 68 | 1158 |
| | | | motif) | | | | | | | | |
| DGKi | 113 | 227 | Ankyrin Repeat | PF00023 | 2.80E−10 | 934 | 958 | 1 | 25 | 33 | 1041 |
| DGKi | 113 | 227 | Ankyrin Repeat | PF00023 | 2.80E−10 | 970 | 1002 | 1 | 33 | 33 | 1041 |
| DGKi | 113 | 227 | Diacylglycerol kinase | PF00609 | 2.90E−57 | 521 | 678 | 1 | 190 | 190 | 1041 |
| | | | accessory domain | | | | | | | | |
| DGKi | 113 | 227 | Diacylglycerol kinase | PF00781 | 4.40E−59 | 371 | 495 | 1 | 154 | 154 | 1041 |
| | | | catalytic domain | | | | | | | | |
| DGKq | 114 | 228 | Diacylglycerol kinase | PF00609 | 1.90E−87 | 733 | 885 | 1 | 190 | 190 | 934 |
| | | | accessory domain | | | | | | | | |
| DGKq | 114 | 228 | Diacylglycerol kinase | PF00781 | 4.10E−63 | 580 | 707 | 1 | 154 | 154 | 934 |
| | | | catalytic domain | | | | | | | | |
| DGKq | 114 | 228 | Phorbol esters/diacylglycerol | PF00130 | 1.00E−27 | 55 | 102 | 1 | 51 | 51 | 934 |
| | | | binding domain (C1 domain) | | | | | | | | |
| DGKq | 114 | 228 | Phorbol esters/diacylglycerol | PF00130 | 1.00E−27 | 116 | 162 | 1 | 51 | 51 | 934 |
| | | | binding domain (C1 domain) | | | | | | | | |
| DGKq | 114 | 228 | Phorbol esters/diacylglycerol | PF00130 | 1.00E−27 | 178 | 228 | 1 | 51 | 51 | 934 |
| | | | binding domain (C1 domain) | | | | | | | | |
| DGKq | 114 | 228 | Ras association | PF00788 | 6.60E−24 | 387 | 486 | 1 | 113 | 113 | 934 |
| | | | (RalGDS/AF-6) domain | | | | | | | | |
| DGKq | 114 | 228 | Ras association | PF00788 | 6.60E−24 | 565 | 586 | 60 | 89 | 113 | 934 |
| | | | (RalGDS/AF-6) domain | | | | | | | | |

Table 4 provides the chromosomal location of the sequences, described in the following columns: Gene_Name, ID#na, ID#aa, Chromosome, Band Name, Genomic Coordinate Start, Genomic Coordinate end”. The first three columns are identical to the equivalent columns in Table 1. “Chromosome” lists the chromosome to which the mouse gene was mapped, and “Band Name” lists the band within the chromosome to which the gene was mapped. To provide more detailed mapping, the beginning and ending nucleotides of the gene mapped to the mouse genomic assembly (February 2003, genome.ucsc.edu) are provided as “Genomic Coordinate Start” and “Genomic Coordinate End”. This mapping information can be used to link mouse genes to genetically mapped traits, including disease susceptibility and modified loci. Resources such as the website of the Jackson laboratory, www.informatics.jax.org/ can be used to search a given chromosomal locus against a large database of mapped traits.

TABLE 4

| Gene_{—} | | | | | | Genomic Coordinate | |
|---|---|---|---|---|---|---|---|
| Name | ID#na | ID#aa | Chromosome | Band | Name | Start | Genomic Coordinate End |
| mSK840 | 34 | 148 | Unknown | | | 40058520 | 40060064 |
| mSK813 | 25 | 139 | | 5 | A1 | 8803350 | 8804386 |
| mSK801 | 18 | 132 | | 10 | E3.1 | 86129275 | 86130749 |
| mSK838 | 33 | 147 | Unknown | | | 1.11E+08 | 1.11E+08 |
| mSK836 | 32 | 146 | Unknown | | | 98125490 | 98126283 |
| mSK807 | 21 | 135 | | 17 | B5 | 52062939 | 52064372 |
| mSK826 | 31 | 145 | | 17 | B5 | 52281242 | 52282804 |
| mSK822 | 29 | 143 | | 5 | A1 | 5682924 | 5684777 |
| mSK823 | 30 | 144 | | 16 | A2 | 12247230 | 12248457 |
| AAK1 | 60 | 174 | | 6 | E2 | 87352915 | 87490724 |
| ADCK4 | 9 | 123 | | 7 | A2 | 18803500 | 18828331 |
| ADCK5 | 10 | 124 | | 15 | D3 | 76809739 | 76829324 |
| AlphaK2 | 11 | 125 | | 18 | E1.3 | 65678368 | 65762585 |
| AlphaK3 | 12 | 126 | | 3 | F3 | 1.28E+08 | 1.28E+08 |
| AMPKa1 | 15 | 129 | | 15 | A1 | 4972560 | 5007441 |
| ATR | 14 | 128 | | 9 | C5 | 95840959 | 95934822 |
| BCR | 13 | 127 | | 10 | E1 | 74818264 | 74942231 |
| BRAF | 110 | 224 | | 6 | B2 | 39598557 | 39710045 |
| CDKL4 | 56 | 170 | | 17 | C3.1 | 79167222 | 79202454 |
| CDKL5 | 55 | 169 | X | | | 1.41E+08 | 1.41E+08 |
| CHED | 53 | 167 | | 13 | A3 | 17128253 | 17216747 |
| CK1g3 | 50 | 164 | | 18 | D | 54260451 | 54318906 |
| CLIK1 | 68 | 182 | | 2 | E4 | 1.31E+08 | 1.31E+08 |
| CYGX | 85 | 199 | | 7 | D1 | 86885718 | 86919278 |
| DCAMKL1 | 38 | 152 | | 3 | C1 | 55417995 | 55708086 |
| DMPK2 | 1 | 115 | | 19 | A1 | 4349504 | 4367998 |
| DYRK4 | 57 | 171 | | 6 | F | 1.28E+08 | 1.28E+08 |
| EphA5 | 101 | 215 | | 5 | C3 | 83261624 | 83629264 |
| EphA6 | 102 | 216 | | 16 | D3 | 60017301 | 60969918 |
| ErbB3 | 100 | 214 | | 10 | | 1.29E+08 | 1.29E+08 |
| ERK4 | 59 | 173 | | 18 | E3 | 74379471 | 74419963 |
| FGR | 105 | 219 | | 4 | H1 | 1.31E+08 | 1.31E+08 |
| Fused | 76 | 190 | | 1 | B | 75323325 | 75357938 |
| HIPK4 | 58 | 172 | | 7 | A2 | 19097192 | 19104845 |
| HSER | 84 | 198 | | 6 | G1 | 1.37E+08 | 1.37E+08 |
| KSR2 | 111 | 225 | | 5 | D1 | 1.15E+08 | 1.15E+08 |
| LATS1 | 5 | 119 | | 10 | A1 | 7360382 | 7383385 |
| LMR2 | 103 | 217 | | 5 | E1 | 1.42E+08 | 1.42E+08 |
| LMR3 | 104 | 218 | | 7 | B1 | 34425932 | 34446495 |
| LRRK1 | 107 | 221 | | 7 | B3 | 54384355 | 54513746 |
| LRRK2 | 106 | 220 | | 15 | E2.2 | 92009360 | 92152361 |
| LZK | 108 | 222 | | 16 | B1 | 21542876 | 21677814 |
| MAP3K6 | 87 | 201 | | 4 | H1 | 1.31E+08 | 1.31E+08 |
| MAP3K7 | 89 | 203 | X | | | 1.4E+08 | 1.4E+08 |
| MAP3K8 | 88 | 202 | | 1 | F2 | 1.28E+08 | 1.28E+08 |
| MAST1 | 4 | 118 | | 8 | E3 | 84197422 | 84223752 |
| MAST3 | 3 | 117 | | 8 | D2 | 69807090 | 69824805 |
| MLK2 | 109 | 223 | | 7 | A2 | 19228735 | 19246743 |
| MNK2 | 39 | 153 | | 10 | E2 | 80495182 | 80505314 |
| MRCKb | 2 | 116 | | 12 | E1 | 1.05E+08 | 1.05E+08 |
| MYO3B | 94 | 208 | | 2 | C3 | 71042476 | 71306595 |
| NEK1 | 61 | 175 | | 8 | C | 59864996 | 60003408 |
| NEK10 | 63 | 177 | | 14 | A1 | 10133105 | 10315949 |
| NRBP2 | 71 | 185 | | 15 | D3 | 76318086 | 76324114 |
| NuaK1 | 36 | 150 | | 10 | E3.1 | 83967390 | 84036260 |
| Obscurin | 48 | 162 | | 11 | B3 | 59625580 | 59767608 |
| OSR1 | 90 | 204 | | 9 | E1 | 1.19E+08 | 1.19E+08 |
| PFTAIRE2 | 54 | 168 | | 1 | A7.1 | 59896060 | 59991807 |
| PIK3R4 | 79 | 193 | | 9 | D2 | 1.06E+08 | 1.06E+08 |
| PIM2 | 44 | 158 | X | | | 4046288 | 4051524 |
| PKN1 | 6 | 120 | | 8 | E2 | 82947703 | 82971074 |
| NEK5 | 62 | 176 | | 8 | A3 | 20813711 | 20865690 |
| PAK6 | 95 | 209 | | 2 | E2 | 1.2E+08 | 1.2E+08 |
| Wnk3 | 83 | 197 | X | | | 1.31E+08 | 1.31E+08 |
| QSK | 37 | 151 | | 9 | B2 | 46061725 | 46269266 |
| RSKL1 | 8 | 122 | | 1 | | 1.91E+08 | 1.91E+08 |
| SgK069 | 64 | 178 | | 7 | A1 | 4350169 | 4357441 |
| SgK071 | 74 | 188 | | 2 | A5 | 27198128 | 27217842 |
| SgK085 | 43 | 157 | | 13 | B2 | 32158136 | 32227151 |
| SgK110 | 65 | 179 | | 7 | A1 | 4360875 | 4364020 |
| SgK223 | 66 | 180 | | 8 | B2 | 35011042 | 35063725 |
| SgK269 | 67 | 181 | | 9 | B3.2 | 56361043 | 56574342 |
| SgK307 | 69 | 183 | | 11 | C2 | 88181839 | 88332597 |
| SgK424 | 70 | 184 | | 7 | A3 | 22124703 | 22129866 |
| SgK493 | 72 | 186 | | 17 | C3.2 | 81878502 | 81887438 |
| SGK494 | 7 | 121 | | 11 | C1 | 79076053 | 79080078 |
| SgK496 | 73 | 187 | | 1 | F3 | 1.33E+08 | 1.33E+08 |
| skMLCK | 42 | 156 | | 2 | G2 | 1.55E+08 | 1.55E+08 |
| smMLCK | 40 | 154 | | 16 | B3.3 | 34577543 | 34793209 |
| SPEG | 47 | 161 | | 1 | C1 | 76072916 | 76129585 |
| STLK5 | 96 | 210 | | 11 | D1 | 1.07E+08 | 1.07E+08 |
| TAO2 | 97 | 211 | | 7 | E3 | 1.16E+08 | 1.16E+08 |
| TAO3 | 98 | 212 | | 5 | C7 | 1.14E+08 | 1.15E+08 |
| TIF1g | 99 | 213 | | 3 | E3 | 1.04E+08 | 1.04E+08 |
| Trad | 46 | 160 | | 16 | B3.3 | 33761991 | 34185222 |
| Trio | 45 | 159 | | 15 | B | 27685910 | 27979596 |
| TTBK1 | 52 | 166 | | 17 | B5 | 44695383 | 44737979 |
| TTBK2 | 51 | 165 | | 2 | E2 | 1.22E+08 | 1.22E+08 |
| TTN | 41 | 155 | | | | | |
| ULK3 | 77 | 191 | | 9 | B3.2 | 57755506 | 57761245 |
| ULK4 | 78 | 192 | | 9 | E1 | 1.21E+08 | 1.22E+08 |
| Wee1B | 80 | 194 | | 6 | B2 | 40436112 | 40457399 |
| Wnk1 | 82 | 196 | | 6 | F | 1.21E+08 | 1.21E+08 |
| Wnk2 | 81 | 195 | | 13 | C1 | 48577228 | 48685142 |
| ZC1 | 91 | 205 | | 1 | A4 | 40327787 | 40452664 |
| ZC2 | 92 | 206 | | 3 | A3 | 28060651 | 28469872 |
| ZC4 | 93 | 207 | X | | | 1.21E+08 | 1.21E+08 |
| KSGC | 86 | 200 | | 19 | D1 | 54955950 | 54999845 |
| SgK384 | 75 | 189 | | 10 | E2 | 80178484 | 80189649 |
| TSSK5 | 49 | 163 | | 15 | D3 | 76603770 | 76607059 |
| DGKd | 112 | 226 | | 1 | C3 | 88074525 | 88139521 |
| DGKi | 113 | 227 | | 6 | B2 | 36828079 | 37281470 |
| DGKq | 114 | 228 | | 5 | C7 | 1.06E+08 | 1.06E+08 |
| mSK843 | 35 | 149 | | 11 | A3 | 21588602 | 21589567 |
| mSK804 | 19 | 133 | | 10 | E3.1 | 86129275 | 86130749 |
| mSK798 | 17 | 131 | Unknown | | | 51249573 | 51250574 |
| mSK817 | 28 | 142 | | 7 | A2 | 15371293 | 15371829 |
| mSK805 | 20 | 134 | | 6 | E3 | 91892181 | 91893257 |
| mSK794 | 16 | 130 | | 11 | A3 | 21588122 | 21589567 |
| mSK814 | 26 | 140 | | 1 | A6 | 53951048 | 53951677 |
| mSK811 | 24 | 138 | | 13 | A3 | 21320755 | 21321673 |
| mSK815 | 27 | 141 | | 7 | A2 | 15221141 | 15222294 |
| mSK809 | 23 | 137 | | 17 | B5 | 52005042 | 52006468 |
| mSK808 | 22 | 136 | | 2 | E3 | 1.29E+08 | 1.29E+08 |

### EXAMPLES

The examples below are not limiting and are merely representative of various aspects and features of the present invention. The examples below demonstrate the isolation and characterization of the nucleic acid molecules according to the invention, as well as the polypeptides they encode.

### Example 1

### Identification and Characterization of Genomic Fragments Encoding Protein Kinases

Novel kinases were identified from the public Human Genome Sequencing project (www.ncbi.nlm.nih.gov/) using a hidden Markov model (HMM) built with 70 mammalian and yeast kinase catalytic domain sequences. These sequences were chosen from a comprehensive collection of kinases such that no two sequences had more than 50% sequence identity. The genomic database entries were translated in six open reading frames and searched against the model using a Timelogic Decypher box with a Field programmable array (FPGA) accelerated version of HMMR2.1. The DNA sequences encoding the predicted protein sequences aligning to the HMMR profile were extracted from the original genomic database. The nucleic acid sequences were then clustered using the Pangea Clustering tool to eliminate repetitive entries. The putative protein kinase sequences were then sequentially run through a series of queries and filters to identify novel protein kinase sequences. Specifically, the HMMR identified sequences were searched using BLASTN and BLASTX against a nucleotide and amino acid repository containing all known mouse and human protein kinases and all subsequent new protein kinase sequences as they are identified. The output was parsed into a spreadsheet to facilitate elimination of known genes by manual inspection. Two models were developed, a “complete” model and a “partial” or Smith Waterman model. The partial model was used to identify sub-catalytic kinase domains, whereas the complete model was used to identify complete catalytic domains. The selected hits were then queried using BLASTN against the public nrna and EST databases to confirm they are indeed unique. In some cases the novel genes were judged to be homologues of previously identified rodent or vertebrate protein kinases.

Extension of partial DNA sequences to encompass the full-length open-reading frame was carried out by several methods. Iterative blastn searching of the cDNA databases listed in Table 5 was used to find cDNAs that extended the genomic sequences. “ZooSeq” databases are from Incyte Genomics, Inc (www.incyte.com/). NCBI databases are from the National Center for Biotechnology Information (www.ncbi.nlm.nih.gov/). All blastn searches were conducted using a penalty for a nucleotide mismatch of −3 and reward for a nucleotide match of 1. The gapped blast algorithm is described in: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

Extension of partial DNA sequences to encompass the full-length open-reading frame was also carried out by iterative searches of genomic databases. The first method made use of the Smith-Waterman algorithm to carry out protein-protein searches of a close protein homologue to the partial. The target databases consisted of Genscan and open-reading frame (ORF) predictions of all mouse genomic sequence derived from the mouse genome project. The complete set of genomic databases searched is shown in Table 6, below. Genomic sequences encoding potential extensions were further assessed by blastx analysis against the NCBI nonredundant database to confirm the novelty of the hit. The extending genomic sequences were incorporated into the cDNA sequence after removal of potential introns using the Seqman program from DNAStar. The default parameters used for Smith-Waterman searches were as shown next. Matrix: blosum 62; gap-opening penalty: 12; gap extension penalty: 2. Genscan predictions were made using the Genscan program as detailed in Chris Burge and Sam Karlin “Prediction of Complete Gene Structures in Human Genomic DNA”, JMB (1997) 268(1):78-94). ORF predictions from genomic DNA were made using a standard 6-frame translation.

Another method for defining DNA extensions from genomic sequence used iterative searches of genomic databases through the Genscan program to predict exon splicing. These predicted genes were then assessed to see if they represented “real” extensions of the partial genes based on homology to related kinases.

Another method involved using the Genewise program (www.sanger.ac.uk/Software/Wise2) to predict potential ORFs based on homology to the closest orthologue/homologue. Genewise requires two inputs, the homologous protein, and genomic DNA containing the gene of interest. The genomic DNA was identified by blastn searches of Celera and Human Genome Project databases. The orthologs were identified by blastp searches of the NCBI non-redundant protein database (NRAA). Genewise compares the protein sequence to a genomic DNA sequence, allowing for introns and frameshifting errors.

TABLE 5
Databases used for cDNA-based sequence extensions

| Database | Database Date |
|---|---|
| ZooSeq Mouse | September 2002 |
| Pharmacia mouse EST | March 2003 |
| collection | |
| NCBI human Ests | March 2003 |
| NCBI murine Ests | March 2003 |
| NCBI nonredundant | March 2003 |

TABLE 6
Databases used for genomic-based sequence extensions

| | Number of | Database |
|---|---|---|
| Database | entries | Date |
| Mouse Genome Project draft | 23 | March 2003 |
| assembly | | |

Results:

For genes that were extended using Genewise, the accession numbers of the protein ortholog and the genomic DNA are given. (Genewise uses the ortholog to assemble the coding sequence of the target gene from the genomic sequence). The amino acid sequences for the orthologs were obtained from the NCBI non-redundant database of proteins (www.ncbi.nlm.nih.gov/Entrez/protein.html) and from internal sources, including KinBase (kinase.com). The genomic DNA came from the public mouse genome project, as indicated below. cDNA sources are also listed below. All of the genomic sequences were used as input for Genscan predictions to predict splice sites [Burge and Karlin, JMB (1997) 268(1):78-94)]. Abbreviations: HGP: Human Genome Project; NCBI, National Center for Biotechnology Information.

### Example 2

### Isolation of cDNAs Encoding Mammalian Protein Kinases

Materials and Methods

Identification of Novel Clones

Total RNAs are isolated using the Guanidine Salts/Phenyl extraction protocol of Chomczynski and Sacchi (P. Chomczynski and N. Sacchi, Anal. Biochem. 162, 156 (1987)) from primary mammalian tumors, normal and tumor cell lines, normal mammalian tissues, and sorted mammalian hematopoietic cells. These RNAs are used to generate single-stranded cDNA using the Superscript Preamplification System (GIBCO BRL, Gaithersburg, Md.; Gerard, GF et al. (1989), FOCUS 11, 66) under conditions recommended by the manufacturer. A typical reaction uses 10 μg total RNA with 1.5 μg oligo(dT)₁₂₋₁₈ in a reaction volume of 60 μL. The product is treated with RNaseH and diluted to 100 μL with H₂O. For subsequent PCR amplification, 1-4 μL of this sscDNA is used in each reaction.

Degenerate oligonucleotides are synthesized on an Applied Biosystems 3948 DNA synthesizer using established phosphoramidite chemistry, precipitated with ethanol and used unpurified for PCR. These primers are derived from the sense and antisense strands of conserved motifs within the catalytic domain of several protein kinases. Degenerate nucleotide residue designations are: N=A, C, G, or T; R=A or G; Y═C or T; H=A, C or T not G; D=A, G or T not C; S═C or G; and W=A or T.

PCR reactions are performed using degenerate primers applied to multiple single-stranded cDNAs. The primers are added at a final concentration of 5 μM each to a mixture containing 10 mM Tris HCl, pH 8.3, 50 mM KCl, 1.5 mM MgCl₂, 200 μM each deoxynucleoside triphosphate, 0.001% gelatin, 1.5 U AmpliTaq DNA Polymerase (Perkin-Elmer/Cetus), and 1-4 μL cDNA. Following 3 min denaturation at 95° C., the cycling conditions are 94° C. for 30 s, 50° C. for 1 min, and 72° C. for 1 min 45 s for 35 cycles. PCR fragments migrating between 300-350 bp are isolated from 2% agarose gels using the GeneClean Kit (Bio101), and T-A cloned into the pCRII vector (Invitrogen Corp. U.S.A.) according to the manufacturer's protocol.

Colonies are selected for mini plasmid DNA-preparations using Qiagen columns and the plasmid DNA is sequenced using a cycle sequencing dye-terminator kit with AmpliTaq DNA Polymerase, FS (ABI, Foster City, Calif.). Sequencing reaction products are run on an ABI Prism 377 DNA Sequencer, and analyzed using the BLAST alignment algorithm (Altschul, S. F. et al., J. Mol. Biol. 215: 403-10).

Additional PCR strategies are employed to connect various PCR fragments or ESTs using exact or near exact oligonucleotide primers. PCR conditions are as described above except the annealing temperatures are calculated for each oligo pair using the formula:
Tm=4(G+C)+2(A+T).

Isolation of cDNA Clones:

Mammalian cDNA libraries are probed with PCR or EST fragments corresponding to kinase-related genes. Probes are ³²P-labeled by random priming and used at 2×10⁶ cpm/mL following standard techniques for library screening. Pre-hybridization (3 h) and hybridization (overnight) are conducted at 42° C. in 5×SSC, 5× Denhart's solution, 2.5% dextran sulfate, 50 mM Na₂PO₄/NaHPO₄, pH 7.0, 50% formamide with 100 mg/mL denatured salmon sperm DNA. Stringent washes are performed at 65° C. in 0.1×SSC and 0.1% SDS. DNA sequencing was carried out on both strands using a cycle sequencing dye-terminator kit with AmpliTaq DNA Polymerase, FS (ABI, Foster City, Calif.). Sequencing reaction products are run on an ABI Prism 377 DNA Sequencer.

### Example 3

### Expression Analysis of Mammalian Protein Kinases

Materials and Methods

Northern Blot Analysis

Northern blots are prepared by running 10 μg total RNA isolated from 60 mammalian tumor cell lines (such as HOP-92, EKVX, NCI-H23, NCI-H226, NCI-H322M, NCI-H460, NCI-H522, A549, HOP-62, OVCAR-3, OVCAR-4, OVCAR-5, OVCAR-8, IGROV1, SK-OV-3, SNB-19, SNB-75, U251, SF-268, SF-295, SF-539, CCRF-CEM, K-562, MOLT-4, HL-60, RPMI 8226, SR, DU-145, PC-3, HT-29, HCC-2998, HCT-116, SW620, Colo 205, HTC15, KM-12, UO-31, SN12C, A498, CaKil, RXF-393, ACHN, 786-0, TK-10, LOX IMVI, Malme-3M, SK-MEL-2, SK-MEL-5, SK-MEL-28, UACC-62, UACC-257, M14, MCF-7, MCF-7/ADR RES, Hs578T, MDA-MB-231, MDA-MB-435, MDA-N, BT-549, T47D), from mammalian adult tissues (such as thymus, lung, duodenum, colon, testis, brain, cerebellum, cortex, salivary gland, liver, pancreas, kidney, spleen, stomach, uterus, prostate, skeletal muscle, placenta, mammary gland, bladder, lymph node, adipose tissue), and 2 mammalian fetal normal tissues (fetal liver, fetal brain), on a denaturing formaldehyde 1.2% agarose gel and transferring to nylon membranes.

Filters are hybridized with random primed [α³²P]dCTP-labeled probes synthesized from the inserts of several of the kinase genes. Hybridization is performed at 42° C. overnight in 6×SSC, 0.1% SDS, 1× Denhardt's solution, 100 μg/mL denatured herring sperm DNA with 1-2×10⁶ cpm/mL of ³²P-labeled DNA probes. The filters are washed in 0.1×SSC/0.1% SDS, 65° C., and exposed on a Molecular Dynamics phosphorimager.

Quantitative PCR Analysis

RNA is isolated from a variety of normal mammalian tissues and cell lines. Single stranded cDNA is synthesized from 10 μg of each RNA as described above using the Superscript Preamplification System (GibcoBRL). These single strand templates are then used in a 25 cycle PCR reaction with primers specific to each clone. Reaction products are electrophoresed on 2% agarose gels, stained with ethidium bromide and photographed on a UV light box. The relative intensity of the STK-specific bands were estimated for each sample.

DNA Array Based Expression Analysis

Plasmid DNA array blots are prepared by loading 0.5 μg denatured plasmid for each kinase on a nylon membrane. The [γ³²P]dCTP labeled single stranded DNA probes are synthesized from the total RNA isolated from several mammalian immune tissue sources or tumor cells (such as thymus, dendrocytes, mast cells, monocytes, B cells (primary, Jurkat, RPMI8226, SR), T cells (CD8/CD4+, TH1, TH2, CEM, MOLT4), K562 (megakaryocytes). Hybridization is performed at 42° C. for 16 hours in 6×SSC, 0.1% SDS, 1× Denhardt's solution, 100 μg/mL denatured herring sperm DNA with 10⁶ cpm/mL of [γ³²P]dCTP labeled single stranded probe. The filters are washed in 0.1×SSC/0.1% SDS, 65° C., and exposed for quantitative analysis on a Molecular Dynamics phosphorimager.

### Example 4

### Protein Kinase Gene Expression

Materials and Methods

Expression Vector Construction

Expression constructs are generated for some of the mammalian cDNAs including: a) full-length clones in a pCDNA expression vector; b) a GST-fusion construct containing the catalytic domain of the novel kinase fused to the C-terminal end of a GST expression cassette; and c) a full-length clone containing a Lys to Ala (K to A) mutation at the predicted ATP binding site within the kinase domain, inserted in the pCDNA vector.

The “K to A” mutants of the kinase might function as dominant negative constructs, and will be used to elucidate the function of these novel STKs.

### Example 5

### Generation of Specific Immunoreagents to Protein Kinases

Materials and Methods

Specific immunoreagents are raised in rabbits against KLH- or MAP-conjugated synthetic peptides corresponding to isolated kinase polypeptides. C-terminal peptides were conjugated to KLH with glutaraldehyde, leaving a free C-terminus. Internal peptides were MAP-conjugated with a blocked N-terminus. Additional immunoreagents can also be generated by immunizing rabbits with the bacterially expressed GST-fusion proteins containing the cytoplasmic domains of each novel PTK or STK.

The various immune sera are first tested for reactivity and selectivity to recombinant protein, prior to testing for endogenous sources.

Western Blots

Proteins in SDS PAGE are transferred to immobilon membrane. The washing buffer is PBST (standard phosphate-buffered saline pH 7.4+0.1% Triton X-100). Blocking and antibody incubation buffer is PBST+5% milk. Antibody dilutions varied from 1:1000 to 1:2000.

### Example 6

### Recombinant Expression and Biological Assays for Protein Kinases

Materials and Methods

Transient Expression of Kinases in Mammalian Cells

The pcDNA expression plasmids (10 μg DNA/100 mm plate) containing the kinase constructs are introduced into 293 cells with lipofectamine (Gibco BRL). After 72 hours, the cells are harvested in 0.5 mL solubilization buffer (20 mM HEPES, pH 7.35, 150 mM NaCl, 10% glycerol, 1% Triton X-100, 1.5 mM MgCl₂, 1 mM EGTA, 2 mM phenylmethylsulfonyl fluoride, 1 μg/mL aprotinin). Sample aliquots are resolved by SDS polyacrylamide gel electrophoresis (PAGE) on 6% acrylamide/0.5% bis-acrylamide gels and electrophoretically transferred to nitrocellulose. Non-specific binding is blocked by preincubating blots in Blotto (phosphate buffered saline containing 5% w/v non-fat dried milk and 0.2% v/v nonidet P-40 (Sigma)), and recombinant protein was detected using the various anti-peptide or anti-GST-fusion specific antisera.

In Vitro Kinase Assays

Three days after transfection with the kinase expression constructs, a 10 cm plate of 293 cells is washed with PBS and solubilized on ice with 2 mL PBSTDS containing phosphatase inhibitors (10 mM NaHPO₄, pH 7.25, 150 mM NaCl, 1% Triton X-100, 0.5% deoxycholate, 0.1% SDS, 0.2% sodium azide, 1 mM NaF, 1 mM EGTA, 4 mM sodium orthovanadate, 1% aprotinin, 5 μg/mL leupeptin). Cell debris was removed by centrifugation (12000×g, 15 min, 4° C.) and the lysate was precleared by two successive incubations with 50 μL of a 1:1 slurry of protein A sepharose for 1 hour each. One-half mL of the cleared supernatant was reacted with 101 L of protein A purified kinase-specific antisera (generated from the GST fusion protein or antipeptide antisera) plus 50 μL of a 1:1 slurry of protein A-sepharose for 2 hr at 4° C. The beads were then washed 2 times in PBSTDS, and 2 times in HNTG (20 mM HEPES, pH 7.5/150 mM NaCl, 0,1% Triton X-100, 10% glycerol).

The immunopurified kinases on sepharose beads are resuspended in 20 μL HNTG plus 30 mM MgCl₂, 10 mM MnCl₂, and 20 μCi [α³²P]ATP (3000 Ci/mmol). The kinase reactions are run for 30 min at room temperature, and stopped by addition of HNTG supplemented with 50 mM EDTA. The samples are washed 6 times in HNTG, boiled 5 min in SDS sample buffer and analyzed by 6% SDS-PAGE followed by autoradiography. Phosphoamino acid analysis is performed by standard 2D methods on ³²P-labeled bands excised from the SDS-PAGE gel.

Similar assays are performed on bacterially expressed GST-fusion constructs of the kinases.

### Example 7

### Chromosomal Localization of Protein Kinases

Materials and Methods

Chromosomal location can identify candidate targets for a tumor amplicon or a tumor-suppressor locus. Summaries of prevalent tumor amplicons are available in the literature, and can identify tumor types to experimentally be confirmed to contain amplified copies of a kinase gene which localizes to an adjacent region. Several sources were used to find information about the chromosomal localization of each of the genes described in this patent.

Several sources were used to find information about the chromosomal localization of each of the genes described in this patent. First, the Celera Browser was used to map the genes. A second source was through BLAT searching of the Human Genome using the University of California, Santa Cruz web tools (genome.ucsc.edu/). Alternatively, the accession number of a genomic contig (identified by BLAST against NRNA) was used to query the Entrez Genome Browser (www.ncbi.nlm.nih.gov/PMGifs/Genomes/MapViewerHelp.html), and the cytogenetic localization was read from the NCBI data. References for association of the mapped sites with chromosomal amplifications found in human cancer can be found in: Knuutila, et al., Am J Pathol, 1998, 152:1107-1123. Information on mapped positions was also obtained by searching published literature (at NCBI, www.ncbi.nlm.nih.gov/entrez/query.fcgi) for documented association of the mapped position with human disease.

Results

The chromosomal regions for mapped genes are listed Table 4, and are discussed in the section Nucleic Acids above. The chromosomal positions were cross-checked with the Online Mendelian Inheritance in Man database (OMIM, www.ncbi.nlm.nih.gov/htbin-post/Omim)., which tracks genetic information for many human diseases, including cancer. References for association of the mapped sites with chromosomal abnormalities found in human cancer can be found in: Knuutila, et al., Am J Pathol, 1998, 152:1107-1123. A third source of information on mapped positions was searching published literature (at NCBI, www.ncbi.nlm.nih.gov/entrez/query.fcgi) for documented association of the mapped position with human disease.

Several sources were used to find information about the chromosomal localization of each of the genes described in this patent. First, cytogenetic map locations of these contigs were found in the title or text of their Genbank record, or by inspection through the NCBI human genome map viewer (www.ncbi.nlm.nih.gov/cgi-bin/Entrez/hum_srch?).

Alternatively, the accession number of a genomic contig (identified by BLAST against NRNA) was used to query the Entrez Genome Browser (www.ncbi.nlm.nih.gov/PMGifs/Genomes/MapViewerHelp.html), and the cytogenetic localization was read from the NCBI data. A thorough search of available literature for the cytogenetic region is also made using Medline (www.ncbi.nlm.nih.gov/PubMed/medline.html). References for association of the mapped sites with chromosomal amplifications found in human cancer can be found in: Knuutila, et al., Am J Pathol, 1998, 152:1107-1123.

Alternatively, the accession number for the nucleic acid sequence is used to query the Unigene database. The site containing the Unigene search engine is: www.ncbi.nlm.nih.gov/UniGene/Hs.Home.html. Information on map position within the Unigene database is imported from several sources, including the Online Mendelian Inheritance in Man (OMIM, www.ncbi.nlm.nih.gov/Omim/searchomim.html), The Genome Database(gdb.infobiogen.fr/gdb/simpleSearch.html), and the Whitehead Institute human physical map (carbon.wi.mit.edu:8000/cgi-bin/contig/sts_info?database=release).

Once a cytogenetic region has been identified by one of these approaches, disease association can be established by searching OMIM with the cytogenetic location. OMIM maintains a searchable catalog of cytogenetic map locations organized by disease. A thorough search of available literature for the cytogenetic region is also made using Medline (www.ncbi.nlm.nih.gov/PubMed/medline.html). As noted above, references for association of the mapped sites with chromosomal abnormalities found in human cancer can be found in: Knuutila, et al., Am J Pathol, 1998, 152:1107-1123.

### Example 8

### Detection Of Protein Protein Interaction Through Phage Display

Materials And Methods

Phage display provides a method for isolating molecular interactions based on affinity for a desired bait. cDNA fragments cloned as fusions to phage coat proteins are displayed on the surface of the phage. Phage(s) interacting with a bait are enriched by affinity purification and the insert DNA from individual clones is analyzed.

T7 Phage Display Libraries

All libraries were constructed in the T7Select1-1b vector (Novagen) according to the manufacturer's directions.

Bait Presentation

Protein domains to be used as baits are generated as C-terminal fusions to GST and expressed in E. coli. Peptides are chemically synthesized and biotinylated at the N-terminus using a long chain spacer biotin reagent.

Selection

Aliquots of refreshed libraries (10¹⁰-10¹² pfu) supplemented with PanMix and a cocktail of E. coli inhibitors (Sigma P-8465) are incubated for 1-2 hrs at room temperature with the immobilized baits. Unbound phage is extensively washed (at least 4 times) with wash buffer.

After 3-4 rounds of selection, bound phage is eluted in 100 μL of 1% SDS and plated on agarose plates to obtain single plaques.

Identification of Insert DNAs

Individual plaques are picked into 25 μL of 10 mM EDTA and the phage is disrupted by heating at 70° C. for 10 min. 2 μL of the disrupted phage are added to 50 μL PCR reaction mix. The insert DNA is amplified by 35 rounds of thermal cycling (94° C., 50 sec; 50° C., 1 min; 72° C., 1 min).

Composition of Buffer
10×PanMix
5% Triton X-100
10% non-fat dry milk (Carnation)
10 mM EGTA
250 mM NaF
250 μg/mL Heparin (sigma)
250 μg/mL sheared, boiled salmon sperm DNA (sigma)
0.05% Na azide
Prepared in PBS

Wash Buffer

PBS supplemented with:

0.5% NP-40
25 μg/mL heparin
PCR reaction mix
1.0 mL 10× PCR buffer (Perkin-Elmer, with 15 mM Mg)
0.2 mL each dNTPs (10 mM stock)
0.1 mL T7UP primer (15 pmol/μL) GGAGCTGTCGTATTCCAGTC
0.1 mL T7DN primer (15 pmol/μL) AACCCCTCAAGACCCGTTTAG
0.2 mL 25 mM MgCl₂ or MgSO₄ to compensate for EDTA
Q.S. to 10 mL with distilled water
Add 1 unit of Taq polymerase per 50 μL reaction

Library: T7 Select1-H441

### Example 9

### HUV-EC-C Assay

The following protocol may also be used to measure a compound's activity against PDGF-R, FGF-R, VEGF, aFGF or Flk-1/KDR, all of which are naturally expressed by HUV-EC cells.

Day 0

Wash and trypsinize HUV-EC-C cells (human umbilical vein endothelial cells, (American Type Culture Collection; catalogue no. 1730 CRL). Wash with Dulbecco's phosphate-buffered saline (D-PBS; obtained from Gibco BRL; catalogue no. 14190-029) 2 times at about 1 ml/10 cm² of tissue culture flask. Trypsinize with 0.05% trypsin-EDTA in non-enzymatic cell dissociation solution (Sigma Chemical Company; catalogue no. C-1544). The 0.05% trypsin was made by diluting 0.25% trypsin/1 mM EDTA (Gibco; catalogue no. 25200-049) in the cell dissociation solution. Trypsinize with about 1 ml/25-30 cm² of tissue culture flask for about 5 minutes at 37° C. After cells have detached from the flask, add an equal volume of assay medium and transfer to a 50 ml sterile centrifuge tube (Fisher Scientific; catalogue no. 05-539-6).

Wash the cells with about 35 ml assay medium in the 50 ml sterile centrifuge tube by adding the assay medium, centrifuge for 10 minutes at approximately 200 g, aspirate the supernatant, and resuspend with 35 ml D-PBS. Repeat the wash two more times with D-PBS, resuspend the cells in about 1 ml assay medium/15 cm² of tissue culture flask. Assay medium consists of F12K medium (Gibco BRL; catalogue no. 21127-014)+0.5% heat-inactivated fetal bovine serum. Count the cells with a Coulter Counter™ Coulter Electronics, Inc.) and add assay medium to the cells to obtain a concentration of 0.8-1.0×10⁵ cells/ml.

Add cells to 96-well flat-bottom plates at 100 μl/well or 0.8-1.0×10⁴ cells/well; incubate ˜24 h at 37° C., 5% CO₂.

Day 1

Make up two-fold drug titrations in separate 96-well plates, generally 50 μM on down to 0 μM. Use the same assay medium as mentioned in day 0, step 2, above. Titrations are made by adding 90 μl/well of drug at 200 μM (4× the final well concentration) to the top well of a particular plate column. Since the stock drug concentration is usually 20 mM in DMSO, the 200 μM drug concentration contains 2% DMSO.

Therefore, diluent made up to 2% DMSO in assay medium (F12K+0.5% fetal bovine serum) is used as diluent for the drug titrations in order to dilute the drug but keep the DMSO concentration constant. Add this diluent to the remaining wells in the column at 60 μl/well. Take 60 μl from the 120 μl of 200 μM drug dilution in the top well of the column and mix with the 60 μl in the second well of the column. Take 60 μl from this well and mix with the 60 μl in the third well of the column, and so on until two-fold titrations are completed. When the next-to-the-last well is mixed, take 60 μl of the 120 μl in this well and discard it. Leave the last well with 60 μl of DMSO/media diluent as a non-drug-containing control. Make 9 columns of titrated drug, enough for triplicate wells each for 1) VEGF (obtained from Pepro Tech Inc., catalogue no. 100-200, 2) endothelial cell growth factor (ECGF) (also known as acidic fibroblast growth factor, or aFGF) (obtained from Boehringer Mannheim Biochemica, catalogue no. 1439 600); or, 3) human PDGF B/B (1276-956, Boehringer Mannheim, Germany) and assay media control. ECGF comes as a preparation with sodium heparin.

Transfer 50 μl/well of the drug dilutions to the 96-well assay plates containing the 0.8-1.0×10⁴ cells/100 μl/well of the HUV-EC-C cells from day 0 and incubate ˜2 h at 37° C., 5%. CO₂.

In triplicate, add 50 μl/well of 80 μg/ml VEGF, 20 ng/ml ECGF, or media control to each drug condition. As with the drugs, the growth factor concentrations are 4× the desired final concentration. Use the assay media from day 0, step 2, to make the concentrations of growth factors. Incubate approximately 24 hours at 37° C., 5% CO₂. Each well will have 50 μl drug dilution, 50 μl growth factor or media, and 100 μl cells, =200 μl/well total. Thus the 4× concentrations of drugs and growth factors become 1× once everything has been added to the wells.

Day 2

Add ³H-thymidine (Amersham; catalogue no. TRK-686) at 1 μCi/well (10 μl/well of 100 μCi/ml solution made up in RPMI media+10% heat-inactivated fetal bovine serum) and incubate ˜24 h at 37° C., 5% CO₂. Note: ³H-thymidine is made up in RPMI media because all of the other applications for which we use the ³H-thymidine involve experiments done in RPMI. The media difference at this step is probably not significant. RPMI was obtained from Gibco BRL, catalogue no. 11875-051.

Day 3

Freeze plates overnight at −20° C.

Day 4

Thaw plates and harvest with a 96-well plate harvester (Tomtec Harvester 96^{(R)}) onto filter mats (Wallac; catalogue no. 1205-401); read counts on a Wallac Betaplate™ liquid scintillation counter.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations that are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

In view of the degeneracy of the genetic code, other combinations of nucleic acids also encode the claimed peptides and proteins of the invention. For example, all four nucleic acid sequences GCT, GCC, GCA, and GCG encode the amino acid alanine. Therefore, if for an amino acid there exists an average of three codons, a polypeptide of 100 amino acids in length will, on average, be encoded by 3100, or 5×1047, nucleic acid sequences. Thus, a nucleic acid sequence can be modified to form a second nucleic acid sequence, encoding the same polypeptide as encoded by the first nucleic acid sequences, using routine procedures and without undue experimentation. Thus, all possible nucleic acids that encode the claimed peptides and proteins are also fully described herein, as if all were written out in full taking into account the codon usage, especially that preferred in humans. Furthermore, changes in the amino acid sequences of polypeptides, or in the corresponding nucleic acid sequence encoding such polypeptide, may be designed or selected to take place in an area of the sequence where the significant activity of the polypeptide remains unchanged. For example, an amino acid change may take place within a β-turn, away from the active site of the polypeptide. Also changes such as deletions (e.g. removal of a segment of the polypeptide, or in the corresponding nucleic acid sequence encoding such polypeptide, which does not affect the active site) and additions (e.g. addition of more amino acids to the polypeptide sequence without affecting the function of the active site, such as the formation of GST-fusion proteins, or additions in the corresponding nucleic acid sequence encoding such polypeptide without affecting the function of the active site) are also within the scope of the present invention. Such changes to the polypeptides can be performed by those with ordinary skill in the art using routine procedures and without undue experimentation. Thus, all possible nucleic and/or amino acid sequences that can readily be determined not to affect a significant activity of the peptide or protein of the invention are also fully described herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. An isolated, enriched or purified nucleic acid molecule encoding a kinase polypeptide, wherein said nucleic acid molecule comprises a nucleotide sequence that:
(a) encodes a polypeptide having an amino acid selected from the group consisting of those set forth in SEQ ID NO:115 though 228 through 235;
(b) is the complement of the nucleotide sequence of (a);
(c) hybridizes under stringent conditions to the nucleotide molecule of (a) and encodes a kinase polypeptide;
(d) encodes a polypeptide having an amino acid sequence of at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through 235; or
(e) is the complement of the nucleotide sequence of (d).

2. The nucleic acid molecule of claim 1, further comprising a vector or promoter effective to initiate transcription in a host cell.

3. The nucleic acid molecule of claim 1, wherein said nucleic acid molecule is isolated, enriched, or purified from a mammal.

4. The nucleic acid molecule of claim 1, wherein said nucleic acid molecule is a cDNA molecule.

5. The nucleic acid molecule of claim 1, wherein said nucleic acid molecule is a genomic DNA molecule.

6. The nucleic acid molecule of claim 3, wherein said mammal is a mouse.

7. A nucleic acid molecule of claim 1 comprising a nucleic acid having a nucleotide sequence which hybridizes under stringent conditions to a nucleotide sequence encoding a kinase polypeptide having an amino acid sequence selected from the group consisting of those set forth in SEQ ID NO:115 through 235.

8. An isolated, enriched or purified nucleic acid molecule encoding a fusion polypeptide comprising at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through 235, and a heterologous polypeptide.

9. A nucleic acid molecule of claim 1 comprising a nucleic acid having a nucleotide sequence which hybridizes under stringent conditions to a nucleotide sequence selected from the group consisting of those set forth in SEQ ID NO:1 through 114.

10. An isolated, enriched, or purified kinase polypeptide, wherein said polypeptide comprises:
(a) an amino acid sequence at least about 90% identical to a sequence selected from the group consisting of those set forth in SEQ ID NO:115 through 235; or
(b) an amino acid sequence selected from the group consisting of at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through 235.

11. The kinase polypeptide of claim 10, wherein said polypeptide is isolated, purified, or enriched from a mammal.

12. The kinase of claim 11, wherein said mammal is a mouse.

13. A fusion polypeptide comprising the polypeptide of claim 10 and a heterologous polypeptide.

14. An antibody or antibody fragment having specific binding affinity to a kinase polypeptide or to a domain of said polypeptide, wherein said polypeptide comprises an amino acid sequence selected from those set forth in SEQ ID NO:115 through 235.

15. A hybridoma which produces the antibody of claim 14.

16. A kit comprising an antibody which binds to a polypeptide of claim 10 and a negative control antibody.

17. A method for identifying a substance that modulates the activity of a kinase polypeptide comprising the steps of:
(a) contacting the kinase polypeptide substantially identical to an amino acid sequence selected from the group consisting of those set forth in SEQ ID NO:115 through 235 with a test substance;
(b) measuring the activity of said polypeptide; and
(c) determining whether said substance modulates the activity of said polypeptide.

18. The method of claim 17, further comprising attaching the kinase polypeptide to a solid support.

19. A method for identifying a substance that modulates the activity of a kinase polypeptide in a cell comprising the steps of:
(a) expressing a kinase polypeptide having a sequence that is at least about 90% identical to an amino acid sequence selected from the group consisting of those set forth in SEQ ID NO:115 through 235 in said cell;
(b) adding a test substance to said cell; and
(c) monitoring kinase activity in the cell.

20. A method for treating a disease or disorder by administering to a patient in need of such treatment a substance that modulates the activity of a kinase substantially identical to an amino acid sequence selected from the group consisting of those set forth in SEQ ID NO:115 through 235.

21. A method for detection of a kinase nucleic acid in a sample as a diagnostic tool for a disease or disorder, wherein said method comprises:
(a) contacting said sample with a nucleic acid probe which hybridizes under hybridization assay conditions to a nucleic acid target region of a nucleic acid sequence selected from the group consisting of SEQ ID NO:1 through 114, said probe comprising said nucleic acid sequence or fragments thereof, or the complement of said sequence or fragments; and
(b) detecting the presence or amount of the target region:probe hybrid as an indication of said disease or disorder.

22. The method of claim 21, wherein said disease or disorder is selected from the group consisting of cancers, immune-related diseases and disorders, cardiovascular disease, brain or neuronal-associated diseases, metabolic disorders and inflammatory disorders.

23. The method of claim 22, wherein said disease or disorder is selected from the group consisting of cancers of tissues; cancers of blood or hematopoietic origin; cancers of the breast, colon, lung, prostate, cervix, brain, ovary, bladder or kidney.

24. The method of claim 22, wherein said disease or disorder is selected from the group consisting of central or peripheral nervious system disease, migraines, pain; sexual dysfunction; mood disorders; attention disorders; cognition disorders; hypotension; hypertension; psychotic disorders; neurological disorders and dyskinesias.

25. The method of claim 22, wherein said disease or disorder is selected from the group consisting of inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

26. A method for detection of a kinase nucleic acid in a sample as a diagnostic tool for a disease or disorder, wherein said method comprises:
(a) contacting said sample with nucleic acid primers capable of hybridizing to a nucleic acid sequence selected from the group consisting of SEQ ID NO:1 through 114;
(b) selectively amplifying at least a portion of a nucleic acid sequence selected from the group consisting of SEQ ID NO:1 through 114; and
(b) detecting the amplified DNA as an indication of said disease or disorder.

27. The method of claim 26, wherein said disease or disorder is selected from the group consisting of cancers, immune-related diseases and disorders, cardiovascular disease, brain or neuronal-associated diseases, metabolic disorders and inflammatory disorders.

28. The method of claim 27, wherein said disease or disorder is selected from the group consisting of cancers of tissues; cancers of blood or hematopoietic origin; cancers of the breast, colon, lung, prostate, cervix, brain, ovary, bladder or kidney.

29. The method of claim 27, wherein said disease or disorder is selected from the group consisting of central or peripheral nervious system disease, migraines, pain; sexual dysfunction; mood disorders; attention disorders; cognition disorders; hypotension;
hypertension; psychotic disorders; neurological disorders and dyskinesias.

30. The method of claim 27, wherein said disease or disorder is selected from the group consisting of inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

31. An isolated, enriched or purified nucleic acid molecule consisting essentially of about 10-30 contiguous nucleotide bases of a nucleic acid sequence that encodes a polypeptide selected from the group consisting of SEQ ID NO:115 through 235.

32. The isolated, enriched or purified nucleic acid molecule of claim 31 consisting essentially of about 10-30 contiguous nucleotide bases of a nucleic acid sequence selected from the group consisting of SEQ ID NO:1 through 114.

33. A recombinant cell comprising the nucleic acid molecule of claim 1.

34. A method for producing a kinase polypeptide comprising:
(a) culturing the recombinant cell of claim 33 under conditions that would allow expression of said nucleic acid molecule; and
(b) isolating the expressed kinase polypeptide, wherein said kinase polypeptide comprises:
(i) an amino acid sequence at least about 90% identical to a sequence selected from the group consisting of those set forth in SEQ ID NO:115 through 235; or
(ii) an amino acid sequence selected from the group consisting of at least one domain selected from the group consisting of an N-terminal domain, a C-terminal catalytic domain, a catalytic domain, a C-terminal domain, a coiled-coil structure region, a proline-rich region, a spacer region and a C-terminal tail of SEQ ID NO:115 through 235.

35. A vector comprising the nucleic acid molecule of claim 1.

36. A method for identification of a nucleic acid encoding a kinase polypeptide in a sample, wherein said method comprises:
(a) contacting said sample with the nucleic acid molecule of claim 32; and
(b) isolating a nucleic acid that hybridizes to the nucleic acid molecule of claim 32, thereby identifying said nucleic acid encoding a kinase polypeptide.

37. A method for identification of a human orthologue of a murine kinase polypeptide, wherein said method comprises:
(a) contacting a human sample with the nucleic acid molecule of claim 32; and
(b) isolating a nucleic acid that hybridizes to the nucleic acid molecule of claim 32, thereby identifying a nucleic acid encoding a human orthologue of a murine kinase polypeptide.

38. A transgenic mouse comprising a nucleic acid sequence that encodes a polypeptide substantially identical to an amino acid sequence selected from the group consisting of those set forth in SEQ ID NO:115 through 235; wherein said mouse exhibits a phenotype, relative to a wild-type phenotype, comprising modulation of kinase activity of said polypeptide.

39. A cell or cell line obtained from a transgenic mouse, wherein said transgenic mouse comprises a nucleic acid sequence that encodes a polypeptide substantially identical to an amino acid sequence selected from the group consisting of those set forth in SEQ ID NO:115 through 235; wherein said mouse exhibits a phenotype, relative to a wild-type phenotype, comprising modulation of kinase activity of said polypeptide.

40. A method for identifying a substance that modulates the activity of a kinase polypeptide, wherein said method comprises:
(a) determining in a sample obtained from the transgenic mouse of claim 38 the presence and/or quantity of kinase activity attributable to the polypeptide encoded by the nucleic acid used to create said transgenic mouse;
(b) administering a test substance to said transgenic mouse; and
(c) determining whether said test substance modulates the kinase activity as determined in step (a).

41. A method for identifying a substance that modulates the activity of a kinase polypeptide, wherein said method comprises:
(a) determining in a cell line obtained from the transgenic mouse of claim 38 the presence and/or quantity of kinase activity attributable to the polypeptide encoded by the nucleic acid used to create said transgenic mouse;
(b) contacting said cell line with a test substance; and
(c) determining whether said test substance modulates the kinase activity as determined in step (a).

42. A method for treating a disease or disorder by administering to a patient in need of such treatment a substance that modulates the activity of a kinase identified by the method of claim 40.

43. A method for treating a disease or disorder by administering to a patient in need of such treatment a substance that modulates the activity of a kinase identified by the method of claim 41.

44. A knock-out mouse whose genome is disrupted by recombination at a nucleic acid sequence that encodes a polypeptide substantially identical to an amino acid sequence selected from the group consisting of those set forth in SEQ ID NO:115 through 235; so as to produce a phenotype, relative to a wild-type phenotype, comprising absence of kinase activity of said polypeptide.

45. A cell or cell line obtained from a knock-out mouse, wherein the genome of said knock-out mouse is disrupted by recombination at a nucleic acid sequence that encodes a polypeptide substantially identical to an amino acid sequence selected from the group consisting of those set forth in SEQ ID NO:115 through 235; so as to produce a phenotype, relative to a wild-type phenotype, comprising absence of kinase activity of said polypeptide.
